(19) United States
(12) Patent Application Publication
(10) Pub. No.: US 2026/0176336 A1
(43) Pub. Date: Jun. 25, 2026

(54) Binding Constructs

(71) Applicant: Mestag Therapeutics Ltd, Saffron Walden (GB)

(72) Inventors: Ray Jupp, Saffron Walden, Cambridge (GB); James Legg, Saffron Walden, Cambridge (GB); Fevzi Demircioglu, Saffron Walden, Cambridge (GB); Marta Lewandowska, Saffron Walden, Cambridge (GB); Lucy Penfold, Saffron Walden, Cambridge (GB); Bruno Dombrecht, Gent (BE)

(21) Appl. No.: 19/551,391

(22) Filed: Feb. 26, 2026

Related U.S. Application Data
(63) Continuation of application No. PCT/EP2025/054117, filed on Feb. 14, 2025.

(30) Foreign Application Priority Data
Feb. 16, 2024 (EP) 24158235.2
Feb. 27, 2024 (EP) 24160115.2
Oct. 30, 2024 (EP) 24209830.9

(51) Int. Cl. C07K 16/00; C07K 16/28

(52) U.S. Cl. CPC C07K 16/00; C07K 16/2827; C07K 2317/21; C07K 2317/56; C07K 2317/565

(57) ABSTRACT

The invention provides inter alia a construct comprising a FAP binding agent and an LTBR binding agent.

### CROSS REFERENCE TO RELATED APPLICATIONS

This application is a continuation of International Patent Application PCT/EP2025/054117, filed Feb. 14, 2025, designating the United States of America and published in English as International Patent Publication WO2025/172587 on Aug. 21, 2025, which claims the benefit under Article 8 of the Patent Cooperation Treaty to European Patent Application Serial No. 24158235.2, filed Feb. 16, 2024, European Patent Application Serial No. 24160115.2 filed Feb. 27, 2024 and European Patent Application Serial No. 24209830.9 filed Oct. 30, 2024, the entireties of which are hereby incorporated by reference.

### INCORPORATION BY REFERENCE

The ST.26 XML Sequence listing named “MST-P3642PCT—Sequence listing.xml”, created on Feb. 10, 2025, and having a size of 208,896 bytes, is hereby incorporated herein by this reference in its entirety.

### FIELD OF THE INVENTION

The invention relates to constructs comprising a FAP binding agent and an LTBR binding agent, particularly for the treatment of diseases such as cancer, and related aspects. The invention also relates to FAP binding agents and LTBR binding agents.

### BACKGROUND OF THE INVENTION

Cancer can affect multiple cell types and tissues but the underlying cause is a breakdown in the control of cell division. This process is highly complex, requiring careful coordination of multiple pathways, many of which remain to be fully characterised.

Cancer immunotherapy involves the use of a subject's own immune system to treat or prevent cancer. These therapies are designed to provoke the body's immune system to eliminate cancer cells and exploit the fact that cancers accumulate genetic mutations resulting in the expression of tumor antigens. These tumor antigens consist of tumor-associated antigens such as proteins and tumor-specific antigens or neoantigens, which are peptides that are presented by major histocompatibility molecules (Xie et al 2023). However, malignant tumors can evade detection by the immune system via tumor cell intrinsic mechanisms and mechanisms linked to the tumor microenvironment leading to therapy resistance (Sharma et al 2017).

Durable responses to immunotherapy have only been observed in a minority of cancer patients and thus there is a need for the development of new therapies. Key challenges in mounting durable anti-tumor responses include limited infiltration of immune effector cells in the tumor, immune cell dysfunction and the lack of priming of anti-tumor immune cells. Recently, it has been shown that the presence of high endothelial venules (HEV) and tertiary lymphoid structures (TLS) in cancer patients is associated with better survival and with better response to therapy (Cabrita et al 2020; Vanhersecke et al 2021; Asrir et al 2022; Zhang et al.; Front Immunol, 2022). HEVs are specialized structures in the vasculature that serve as portals for immune cell entry into the tumor and TLS are ectopic immune cell aggregates that provide a favourable local immune environment and facilitate local priming of immune cells (Sautès-Fridman et al 2019; Schumacher et al 2022). Therapeutic strategies aimed at enhancing both HEV and TLS in patients would therefore be advantageous to improve antitumour immune responses.

Lymphotoxin beta receptor (LTBR), also known as tumour necrosis factor receptor superfamily member 3 (TNFRSF3), is a cell surface receptor for lymphotoxin involved in cytokine release and apoptosis. It is a member of the tumour necrosis factor receptor superfamily. LTBR plays a central role in the development and homeostasis of secondary lymphoid organs (SLO), TLS and HEV by regulating the expression of several homeostatic lymphoid cytokines (e.g. CCL5, CCL19, CCL21 and CXCL13) and adhesion molecules (ICAM-1, VCAM-1, MAdCAM1) via the classical and alternative NFKB pathways (Dejardin et al. 2002, Schneider et al. 2004, Fernandes et al. 2016). LTBR is activated by two different trimeric ligands, LIGHT (TNFSF14) and lymphotoxin α1β2 (LTα1β2). Whereas LTα1β2 is specific for LTBR, LIGHT also binds to and activates HVEM (TNFRSF14), a receptor expressed on and implicated in the regulation of immune cells (Pasero et al. 2012).

It has been found that activation of LTBR by its ligands leads to ectopic TLS and HEV (Schrama et al. 2001; Tang et al. 2017; Allen et al 2017; Asrir et al 2022). Presence of TLS and HEV in the tumour microenvironment typically correlates with immune infiltration and is also associated with better prognosis, suggesting that TLS and HEV are involved in anti-tumour immune responses (Dieu-Nosjean et al. 2008; Weinstein and Storkus 2015; Vanhersecke et al 2021; Asrir et al 2022). Therefore, activation of LTBR has the potential to promote formation of HEV and TLS in the tumour microenvironment and induce anti-tumour responses and improve current cancer therapies.

Recent evidence suggests that activation of LTBR stimulates the maturation of resident fibroblasts into immunologically competent Fibroblast Reticular Cells (FRC) (Sautès-Fridman et al. 2019). FRC are a specialized subset of highly fibroblast activation protein alpha (FAP)-positive stromal cells found in lymphoid organs (e.g. TLS), which play an essential role in immune response regulation and immune cell trafficking, retention, and activation by intimate crosstalk with various immune cells, including T cells, B cells, and dendritic cells.

Due to the broad expression of LTBR in organisms, an agonistic LTBR binding construct capable of inducing TLS and creating an activating immune environment bears a substantial risk of causing systemic immune-related adverse events. Johansson-Percival et al. reported weight loss in mice after systemic administration of an LTBR activating compound, VTP-LIGHT (Johansson-Percival et al. 2017) and overexpression of lymphotoxin α and lymphotoxin β induced hepatotoxicity in mice (Haybaeck et al 2009). In US20120014947 it is noted that lymphotoxin signalling may cause liver toxicity, in particular it is noted that knock out of LTBR or inhibition of LTBR signalling in hepatitis models alleviates increases in liver enzymes (AST and ALT) which are associated with hepatotoxicity. A Phase 1 open-label, dose-escalation study, using an LTBR humanized agonistic antibody (hCBE11) in patients with advanced solid tumors aimed at determining safety and tolerability was terminated before enrolment was complete (ClinicalTrials.gov, NCT00105170).

Therefore, as postulated in the prior art, a therapeutic modality which activates LTBR specifically in the tumour but not in other tissues is needed to reduce the risk of toxicity and to generate a well-tolerated drug that can be employed for combination therapies (Tang et al. 2017).

US2021/0188990 discloses multispecific binding molecules, such as bispecific antibodies, with a first specificity for LTBR and a second specificity for extra-domain B of fibronectin (EDB) (US2021/0188990, paragraph 17) in particular for the purpose of killing cancer cells ((US2021/0188990, paragraph 2). This disclosure indicates that tumor antigen-dependent activation of LTBR using a bispecific antibody with one arm binding to LTBR and another arm (the ‘targeting’ arm) binding to a tumour associated antigen (TAA) is more effective when using a targeting arm that binds to an extracellular matrix antigen than to a cell surface antigen co-expressed on the same cell as LTBR. In contrast, the present inventors have shown that a bispecific antibody with a first specificity for LTBR and a second specificity for FAP is capable of activating LTBR in a tumor-antigen dependent manner.

WO2023117834 discloses agonistic LTBR antibodies and bispecific FAP/LTBR antibodies comprising them. Comparative data in respect of FAP/LTBR bispecific antibodies disclosed in WO2023117834 are provided in Example 4 below.

In providing a bispecific agent targeting LTBR, it may be advantageous for the component(s) binding LTBR to have a relatively low affinity, while nonetheless achieving high activity once incorporated into bispecific format. In other words, a high bispecific activity to monospecific affinity ratio may be desirable. A lower binding affinity to the target might be beneficial for an agonist since it has been reported in the literature that low- rather than high-affinity antibodies deliver a greater activity through increased clustering (Yu et al., Nature 2023). Furthermore, a lower binding affinity to the target might be beneficial for an agonist in order to reduce any off-tumour binding.

There remains a need for new approaches and therapies to treat cancer. Such approaches may demonstrate high response rates and durable responses, reduction in the dose required for effect, an improved safety profile/reduced side effects, or the like.

### SUMMARY OF THE INVENTION

The present invention relates to a plurality of aspects involving targeting FAP and/or LTBR. The inventors have established that certain FAP/LTBR bispecific constructs can achieve conditional activation of LTBR depending on the presence of FAP, providing advantages including potentially reducing toxicity. Certain FAP/LTBR bispecific constructs can activate LTBR on cells which are double-positive for FAP and LTBR (e.g. CAFs and FRCs). The inventors have also established that certain FAP/LTBR bispecific constructs can target two or more cells which are each single-positive for FAP and LTBR and activate LTBR. Furthermore, certain FAP/LTBR bispecific constructs can achieve adhesion molecule upregulation and/or chemokine induction (as demonstrated in Examples 4.1 to 4.10). These and other advantageous properties for certain constructs have been shown to translate to in vivo effects such as HEV and TLS formation, increased immune cells and tumour growth inhibition/regression (as demonstrated in Examples 4.11 to 4.13).

The inventors have also identified anti-FAP binding polypeptides and anti-LTBR binding polypeptides which (a) benefit from advantageous properties when deployed in monovalent, monospecific form (as demonstrated in Examples 1 to 3) and (b) represent components having utility in producing the advantageous bispecific constructs described above.

Generally, the invention provides in one aspect a construct comprising a FAP binding agent and an LTBR binding agent. In a further aspect the invention provides a construct according to the invention for use in the treatment of cancer. In a further aspect the invention provides a polynucleotide encoding the construct of the invention. The invention also provides (i) certain anti-LTBR binding polypeptides and (ii) certain anti-FAP binding polypeptides.

Certain embodiments of the invention may be expected to benefit from one or more of the following advantages over the prior art:
(a) increased affinity
(b) increased avidity
(c) agonism of LTBR (e.g. activation of NFKB pathways, such as the classical or alternative NFKB pathways)
(d) clustering of LTBR
(e) reduced toxicity (e.g. liver toxicity)
(f) reduced doses
(g) improved biodistribution
(h) improved monomer affinity to bispecific potency ratio
(i) improved therapeutic efficacy
(j) binding to novel, advantageous epitopes
(k) localisation to, and conditional activation (i.e. in a FAP-dependent manner) at the target site
(l) upregulation of intercellular adhesion molecules (e.g. ICAM-1)
(m) binding LTBR and FAP on the same cell simultaneously and activating LTBR on this cell
(n) increased secretion of CCL5 (e.g. from RPMI-7951 cells)
(o) increased yield and/or purity
(p) improved ease of purification
(q) reduced immunogenicity
(r) increased biodistribution
(s) improved half-life and/or reduced rate of clearance
(t) reduction in Fc receptor binding.

Further aspects of the invention will become clear from the below.

Summary of sequences

| | |
|---|---|
| SEQ ID NO: 1 | Polypeptide sequence of BHA10 heavy chain variable region |
| SEQ ID NO: 2 | Polypeptide sequence of BHA10 light chain variable region |
| SEQ ID NO: 3 | Polypeptide sequence of a G₄S peptide linker |
| SEQ ID NO: 4 | Polypeptide sequence of a (G₄S)₄ peptide linker |
| SEQ ID NO: 5 | Polypeptide sequence of 5G11 heavy chain variable region |
| SEQ ID NO: 6 | Polypeptide sequence of 5G11 light chain variable region |
| SEQ ID NO: 7 | Polypeptide sequence of 28H1 heavy chain variable region |
| SEQ ID NO: 8 | Polypeptide sequence of 28H1 light chain variable region |
| SEQ ID NO: 9 | Polypeptide sequence of full length human LTBR |
| SEQ ID NO: 10 | Polypeptide sequence of human LTBR extracellular domain |
| SEQ ID NO: 11 | Polypeptide sequence of full length Macaca fascicularis LTBR |
| SEQ ID NO: 12 | Polypeptide sequence of Macaca fascicularis LTBR extracellular domain |
| SEQ ID NO: 13 | Polypeptide sequence of full length mouse LTBR |
| SEQ ID NO: 14 | Polypeptide sequence of mouse LTBR extracellular domain |
| SEQ ID NO: 15 | Polypeptide sequence of full length human FAP |
| SEQ ID NO: 16 | Polypeptide sequence of human FAP extracellular domain |
| SEQ ID NO: 17 | Polypeptide sequence of full length Macaca fascicularis FAP |
| SEQ ID NO: 18 | Polypeptide sequence of Macaca fascicularis FAP extracellular domain |
| SEQ ID NO: 19 | Polypeptide sequence of full length mouse FAP |
| SEQ ID NO: 20 | Polypeptide sequence of mouse FAP extracellular domain |
| SEQ ID NO: 21 | Polypeptide sequence of human LIGHT (including ECD) |
| SEQ ID NO: 22 | Polypeptide sequence of human LIGHT ECD |
| SEQ ID NO: 23 | Polypeptide sequence of Clone 1.5 heavy chain variable region |
| SEQ ID NO: 24 | Polypeptide sequence of Clone 1.6 heavy chain variable region |
| SEQ ID NO: 25 | Polypeptide sequence of Clone 1.7 heavy chain variable region |
| SEQ ID NO: 26 | Polypeptide sequence of Clone 1.8 heavy chain variable region |
| SEQ ID NO: 27 | Polypeptide sequence of Clone 2.1 heavy chain variable region |
| SEQ ID NO: 28 | Polypeptide sequence of Clone 2.1 light chain variable region |
| SEQ ID NO: 29 | Polypeptide sequence of Clone 2.2 heavy chain variable region |
| SEQ ID NO: 30 | Polypeptide sequence of Clone 2.2 light chain variable region |
| SEQ ID NO: 31 | Polypeptide sequence of Clone 2.3 heavy chain variable region |
| SEQ ID NO: 32 | Polypeptide sequence of Clone 2.3 light chain variable region |
| SEQ ID NO: 33 | Polypeptide sequence of Clone 2.4 heavy chain variable region |
| SEQ ID NO: 34 | Polypeptide sequence of Clone 2.4 light chain variable region |
| SEQ ID NO: 35 | Polypeptide sequence of Clone 2.5 heavy chain variable region |
| SEQ ID NO: 36 | Polypeptide sequence of Clone 2.5 light chain variable region |
| SEQ ID NO: 37 | Polypeptide sequence of Clone 2.6 heavy chain variable region |
| SEQ ID NO: 38 | Polypeptide sequence of Clone 2.6 light chain variable region |
| SEQ ID NO: 39 | Polypeptide sequence of Clone 2.7 heavy chain variable region |
| SEQ ID NO: 40 | Polypeptide sequence of Clone 2.7 light chain variable region |
| SEQ ID NO: 41 | Polypeptide sequence of Clone 2.8 heavy chain variable region |
| SEQ ID NO: 42 | Polypeptide sequence of Clone 2.8 light chain variable region |
| SEQ ID NO: 43 | Polypeptide sequence of Clone 3.5 heavy chain variable region |
| SEQ ID NO: 44 | Polypeptide sequence of Clone 3.5 light chain variable region |
| SEQ ID NO: 45 | Polypeptide sequence of Clone 3.6 heavy chain variable region |
| SEQ ID NO: 46 | Polypeptide sequence of Clone 3.6 light chain variable region |
| SEQ ID NO: 47 | Polypeptide sequence of Clone 3.7 heavy chain variable region |
| SEQ ID NO: 48 | Polypeptide sequence of Clone 3.7 light chain variable region |
| SEQ ID NO: 49 | Polypeptide sequence of Clone 3.4 heavy chain variable region |
| SEQ ID NO: 50 | Polypeptide sequence of Clone 3.4 light chain variable region |
| SEQ ID NO: 51 | Polypeptide sequence of Clone 3.1 heavy chain variable region |
| SEQ ID NO: 52 | Polypeptide sequence of Clone 3.1 light chain variable region |
| SEQ ID NO: 53 | Polypeptide sequence of Clone 3.2 heavy chain variable region |
| SEQ ID NO: 54 | Polypeptide sequence of Clone 3.2 light chain variable region |
| SEQ ID NO: 55 | Polypeptide sequence of Clone 3.3 heavy chain variable region |
| SEQ ID NO: 56 | Polypeptide sequence of Clone 3.3 light chain variable region |
| SEQ ID NO: 57 | Polypeptide sequence of Clone 1.5 HCDR1 |
| SEQ ID NO: 58 | Polypeptide sequence of Clone 1.5 HCDR2 |
| SEQ ID NO: 59 | Polypeptide sequence of Clone 1.5 HCDR3 |
| SEQ ID NO: 60 | Polypeptide sequence of Clone 1.6 HCDR1 |
| SEQ ID NO: 61 | Polypeptide sequence of Clone 1.6 HCDR2 |
| SEQ ID NO: 62 | Polypeptide sequence of Clone 1.6 HCDR3 |
| SEQ ID NO: 63 | Polypeptide sequence of Clone 1.7 HCDR1 |
| SEQ ID NO: 64 | Polypeptide sequence of Clone 1.7 HCDR2 |
| SEQ ID NO: 65 | Polypeptide sequence of Clone 1.7 HCDR3 |
| SEQ ID NO: 66 | Polypeptide sequence of Clone 1.8 HCDR1 |
| SEQ ID NO: 67 | Polypeptide sequence of Clone 1.8 HCDR2 |
| SEQ ID NO: 68 | Polypeptide sequence of Clone 1.8 HCDR3 |
| SEQ ID NO: 69 | Polypeptide sequence of Clone 2.1 HCDR1 |
| SEQ ID NO: 70 | Polypeptide sequence of Clone 2.1 HCDR2 |
| SEQ ID NO: 71 | Polypeptide sequence of Clone 2.1 HCDR3 |
| SEQ ID NO: 72 | Polypeptide sequence of Clone 2.1 LCDR1 |
| SEQ ID NO: 73 | Polypeptide sequence of Clone 2.1 LCDR2 |
| SEQ ID NO: 74 | Polypeptide sequence of Clone 2.1 LCDR3 |
| SEQ ID NO: 75 | Polypeptide sequence of Clone 2.2 HCDR1 |
| SEQ ID NO: 76 | Polypeptide sequence of Clone 2.2 HCDR2 |
| SEQ ID NO: 77 | Polypeptide sequence of Clone 2.2 HCDR3 |
| SEQ ID NO: 78 | Polypeptide sequence of Clone 2.2 LCDR1 |
| SEQ ID NO: 79 | Polypeptide sequence of Clone 2.2 LCDR2 |
| SEQ ID NO: 80 | Polypeptide sequence of Clone 2.2 LCDR3 |
| SEQ ID NO: 81 | Polypeptide sequence of Clone 2.3 HCDR1 |
| SEQ ID NO: 82 | Polypeptide sequence of Clone 2.3 HCDR2 |
| SEQ ID NO: 83 | Polypeptide sequence of Clone 2.3 HCDR3 |
| SEQ ID NO: 84 | Polypeptide sequence of Clone 2.3 LCDR1 |
| SEQ ID NO: 85 | Polypeptide sequence of Clone 2.3 LCDR2 |
| SEQ ID NO: 86 | Polypeptide sequence of Clone 2.3 LCDR3 |
| SEQ ID NO: 87 | Polypeptide sequence of Clone 2.4 HCDR1 |
| SEQ ID NO: 88 | Polypeptide sequence of Clone 2.4 HCDR2 |
| SEQ ID NO: 89 | Polypeptide sequence of Clone 2.4 HCDR3 |
| SEQ ID NO: 90 | Polypeptide sequence of Clone 2.4 LCDR1 |
| SEQ ID NO: 91 | Polypeptide sequence of Clone 2.4 LCDR2 |
| SEQ ID NO: 92 | Polypeptide sequence of Clone 2.4 LCDR3 |
| SEQ ID NO: 93 | Polypeptide sequence of Clone 2.5 HCDR1 |
| SEQ ID NO: 94 | Polypeptide sequence of Clone 2.5 HCDR2 |
| SEQ ID NO: 95 | Polypeptide sequence of Clone 2.5 HCDR3 |
| SEQ ID NO: 96 | Polypeptide sequence of Clone 2.5 LCDR1 |
| SEQ ID NO: 97 | Polypeptide sequence of Clone 2.5 LCDR2 |
| SEQ ID NO: 98 | Polypeptide sequence of Clone 2.5 LCDR3 |
| SEQ ID NO: 99 | Polypeptide sequence of Clone 2.6 HCDR1 |
| SEQ ID NO: 100 | Polypeptide sequence of Clone 2.6 HCDR2 |
| SEQ ID NO: 101 | Polypeptide sequence of Clone 2.6 HCDR3 |
| SEQ ID NO: 102 | Polypeptide sequence of Clone 2.6 LCDR1 |
| SEQ ID NO: 103 | Polypeptide sequence of Clone 2.6 LCDR2 |
| SEQ ID NO: 104 | Polypeptide sequence of Clone 2.6 LCDR3 |
| SEQ ID NO: 105 | Polypeptide sequence of Clone 2.7 HCDR1 |
| SEQ ID NO: 106 | Polypeptide sequence of Clone 2.7 HCDR2 |
| SEQ ID NO: 107 | Polypeptide sequence of Clone 2.7 HCDR3 |
| SEQ ID NO: 108 | Polypeptide sequence of Clone 2.7 LCDR1 |
| SEQ ID NO: 109 | Polypeptide sequence of Clone 2.7 LCDR2 |
| SEQ ID NO: 110 | Polypeptide sequence of Clone 2.7 LCDR3 |
| SEQ ID NO: 111 | Polypeptide sequence of Clone 2.8 HCDR1 |
| SEQ ID NO: 112 | Polypeptide sequence of Clone 2.8 HCDR2 |
| SEQ ID NO: 113 | Polypeptide sequence of Clone 2.8 HCDR3 |
| SEQ ID NO: 114 | Polypeptide sequence of Clone 2.8 LCDR1 |
| SEQ ID NO: 115 | Polypeptide sequence of Clone 2.8 LCDR2 |
| SEQ ID NO: 116 | Polypeptide sequence of Clone 2.8 LCDR3 |
| SEQ ID NO: 117 | Polypeptide sequence of Clone 3.5 HCDR1 |
| SEQ ID NO: 118 | Polypeptide sequence of Clone 3.5 HCDR2 |
| SEQ ID NO: 119 | Polypeptide sequence of Clone 3.5 HCDR3 |
| SEQ ID NO: 120 | Polypeptide sequence of Clone 3.5 LCDR1 |
| SEQ ID NO: 121 | Polypeptide sequence of Clone 3.5 LCDR2 |
| SEQ ID NO: 122 | Polypeptide sequence of Clone 3.5 LCDR3 |
| SEQ ID NO: 123 | Polypeptide sequence of Clone 3.6 HCDR1 |
| SEQ ID NO: 124 | Polypeptide sequence of Clone 3.6 HCDR2 |
| SEQ ID NO: 125 | Polypeptide sequence of Clone 3.6 HCDR3 |
| SEQ ID NO: 126 | Polypeptide sequence of Clone 3.6 LCDR1 |
| SEQ ID NO: 127 | Polypeptide sequence of Clone 3.6 LCDR2 |
| SEQ ID NO: 128 | Polypeptide sequence of Clone 3.6 LCDR3 |
| SEQ ID NO: 129 | Polypeptide sequence of Clone 3.7 HCDR1 |
| SEQ ID NO: 130 | Polypeptide sequence of Clone 3.7 HCDR2 |
| SEQ ID NO: 131 | Polypeptide sequence of Clone 3.7 HCDR3 |
| SEQ ID NO: 132 | Polypeptide sequence of Clone 3.7 LCDR1 |
| SEQ ID NO: 133 | Polypeptide sequence of Clone 3.7 LCDR2 |
| SEQ ID NO: 134 | Polypeptide sequence of Clone 3.7 LCDR3 |
| SEQ ID NO: 135 | Polypeptide sequence of Clone 3.4 HCDR1 |
| SEQ ID NO: 136 | Polypeptide sequence of Clone 3.4 HCDR2 |
| SEQ ID NO: 137 | Polypeptide sequence of Clone 3.4 HCDR3 |
| SEQ ID NO: 138 | Polypeptide sequence of Clone 3.4 LCDR1 |
| SEQ ID NO: 139 | Polypeptide sequence of Clone 3.4 LCDR2 |
| SEQ ID NO: 140 | Polypeptide sequence of Clone 3.4 LCDR3 |
| SEQ ID NO: 141 | Polypeptide sequence of Clone 3.1 HCDR1 |
| SEQ ID NO: 142 | Polypeptide sequence of Clone 3.1 HCDR2 |
| SEQ ID NO: 143 | Polypeptide sequence of Clone 3.1 HCDR3 |
| SEQ ID NO: 144 | Polypeptide sequence of Clone 3.1 LCDR1 |
| SEQ ID NO: 145 | Polypeptide sequence of Clone 3.1 LCDR2 |
| SEQ ID NO: 146 | Polypeptide sequence of Clone 3.1 LCDR3 |
| SEQ ID NO: 147 | Polypeptide sequence of Clone 3.2 HCDR1 |
| SEQ ID NO: 148 | Polypeptide sequence of Clone 3.2 HCDR2 |
| SEQ ID NO: 149 | Polypeptide sequence of Clone 3.2 HCDR3 |
| SEQ ID NO: 150 | Polypeptide sequence of Clone 3.2 LCDR1 |
| SEQ ID NO: 151 | Polypeptide sequence of Clone 3.2 LCDR2 |
| SEQ ID NO: 152 | Polypeptide sequence of Clone 3.2 LCDR3 |
| SEQ ID NO: 153 | Polypeptide sequence of Clone 3.3 HCDR1 |
| SEQ ID NO: 154 | Polypeptide sequence of Clone 3.3 HCDR2 |
| SEQ ID NO: 155 | Polypeptide sequence of Clone 3.3 HCDR3 |
| SEQ ID NO: 156 | Polypeptide sequence of Clone 3.3 LCDR1 |
| SEQ ID NO: 157 | Polypeptide sequence of Clone 3.3 LCDR2 |
| SEQ ID NO: 158 | Polypeptide sequence of Clone 3.3 LCDR3 |
| SEQ ID NO: 159 | Polypeptide sequence of Clone 1.1 |
| SEQ ID NO: 160 | Polypeptide sequence of Clone 1.2 |
| SEQ ID NO: 161 | Polypeptide sequence of Clone 1.3 |
| SEQ ID NO: 162 | Polypeptide sequence of Clone 1.4 |
| SEQ ID NO: 163 | Polypeptide sequence of Clone 1.1 HCDR1 |
| SEQ ID NO: 164 | Polypeptide sequence of Clone 1.1 HCDR2 |
| SEQ ID NO: 165 | Polypeptide sequence of Clone 1.1 HCDR3 |
| SEQ ID NO: 166 | Polypeptide sequence of Clone 1.2 HCDR1 |
| SEQ ID NO: 167 | Polypeptide sequence of Clone 1.2 HCDR2 |
| SEQ ID NO: 168 | Polypeptide sequence of Clone 1.2 HCDR3 |
| SEQ ID NO: 169 | Polypeptide sequence of Clone 1.3 HCDR1 |
| SEQ ID NO: 170 | Polypeptide sequence of Clone 1.3 HCDR2 |
| SEQ ID NO: 171 | Polypeptide sequence of Clone 1.3 HCDR3 |
| SEQ ID NO: 172 | Polypeptide sequence of Clone 1.4 HCDR1 |
| SEQ ID NO: 173 | Polypeptide sequence of Clone 1.4 HCDR2 |
| SEQ ID NO: 174 | Polypeptide sequence of Clone 1.4 HCDR3 |
| SEQ ID NO: 175 | Polypeptide sequence of Clone 4.1 HC1 |
| SEQ ID NO: 176 | Polypeptide sequence of Clone 4.1 HC2 |
| SEQ ID NO: 177 | Polypeptide sequence of Clone 4.1 LC |
| SEQ ID NO: 178 | Polypeptide sequence of Clone 4.2 HC1 |
| SEQ ID NO: 179 | Polypeptide sequence of Clone 4.2 HC2 |
| SEQ ID NO: 180 | Polypeptide sequence of Clone 4.2 LC |
| SEQ ID NO: 181 | Polypeptide sequence of Clone 4.3 HC1 |
| SEQ ID NO: 182 | Polypeptide sequence of Clone 4.3 HC2 |
| SEQ ID NO: 183 | Polypeptide sequence of Clone 4.3 LC |
| SEQ ID NO: 184 | Polypeptide sequence of Clone 4.4 HC1 |
| SEQ ID NO: 185 | Polypeptide sequence of Clone 4.4 HC2 |
| SEQ ID NO: 186 | Polypeptide sequence of Clone 4.4 LC |
| SEQ ID NO: 187 | Polypeptide sequence of Clone 4.5 HC1 |
| SEQ ID NO: 188 | Polypeptide sequence of Clone 4.5 HC2 |
| SEQ ID NO: 189 | Polypeptide sequence of Clone 4.5 LC |
| SEQ ID NO: 190 | Polypeptide sequence of Clone 4.6 HC1 |
| SEQ ID NO: 191 | Polypeptide sequence of Clone 4.6 HC2 |
| SEQ ID NO: 192 | Polypeptide sequence of Clone 4.6 LC |
| SEQ ID NO: 193 | Polypeptide sequence of Clone 4.7 HC1 |
| SEQ ID NO: 194 | Polypeptide sequence of Clone 4.7 HC2 |
| SEQ ID NO: 195 | Polypeptide sequence of Clone 4.7 LC |
| SEQ ID NO: 196 | Polypeptide sequence of Clone 4.8 HC |
| SEQ ID NO: 197 | Polypeptide sequence of Clone 4.8 LC |
| SEQ ID NO: 198 | Polypeptide sequence of Clone 4.9 HC |
| SEQ ID NO: 199 | Polypeptide sequence of Clone 4.9 LC |
| SEQ ID NO: 200 | Polypeptide sequence of Clone 4.10 HC1 |
| SEQ ID NO: 201 | Polypeptide sequence of Clone 4.10 HC2 |
| SEQ ID NO: 202 | Polypeptide sequence of Clone 4.10 LC |
| SEQ ID NO: 203 | Polypeptide sequence of truncated hinge region |
| SEQ ID NO: 204 | Polypeptide sequence of native hinge region |
| SEQ ID NO: 205 | Polypeptide sequence combining SEQ ID NOs 58 and 167 |
| SEQ ID NO: 206 | Polypeptide sequence combining SEQ ID NOs 67 and 170 |
| SEQ ID NO: 207 | Polypeptide sequence combining SEQ ID NOs 65 and 165 |
| SEQ ID NO: 208 | Polypeptide sequence combining SEQ ID NOs 68 and 171 |
| SEQ ID NO: 209 | Polypeptide sequence of E22 peptide |

### SUMMARY OF THE FIGURES

The patent or application file contains at least one drawing executed in color. Copies of this patent or patent application publication with color drawing(s) will be provided by the Office upon request and payment of the necessary fee.

FIG. 1A Screening of anti-LTBR VHHs: HepG2 reporter assay in the absence of anti-His Tag cross-linking antibody

FIG. 1B Screening of anti-LTBR VHHs: HepG2 reporter assay in the presence of anti-His Tag cross-linking antibody

FIG. 2 In vitro VHH Fc fusion protein activity in chemokine induction assay using tumour cell line HCC1187

FIG. 3A Ligand receptor inhibition assay: Anti-LTBR VHH-Fc fusion proteins binding to A549 cell line

FIG. 3B Ligand receptor inhibition assay: Ligand Receptor Inhibition

FIG. 4 FACS Binding assay: Binding of anti-LTBR Abs to LTBR positive cell line A375

FIG. 5A NFKB activation of HepG2 reporter cells expressing human LTBR: HepG2 reporter assay

FIG. 5B NFKB activation of HepG2 reporter cells expressing human LTBR: HepG2 reporter assay in the presence of cross-linking Ab

FIG. 6 FACS Binding assay: Binding of anti-FAP Abs to FAP-overexpressing HEK293 cell line

FIG. 7A Production of human and mouse-specific FAP/LTBR bispecific antibodies: schematic diagram of a specific 1:1 format

FIG. 7B Production of human and mouse-specific FAP/LTBR bispecific antibodies: schematic diagram of a specific 2:1 format

FIG. 7C Production of human and mouse-specific FAP/LTBR bispecific antibodies: schematic diagram of a specific 2:2 format

FIG. 7D Production of human and mouse-specific FAP/LTBR bispecific antibodies: schematic diagram of a specific 2:2 format

FIG. 8A In vitro FAP/LTBR bispecific activity in chemokine induction assay using tumour cell line HCC1187 cultured with FAP positive primary human breast CAFs: Activity in HCC1187 monoculture

FIG. 8B In vitro FAP/LTBR bispecific activity in chemokine induction assay using tumour cell line HCC1187 cultured with FAP positive primary human breast CAFs: Activity in HCC1187:CHO-FAP co-culture

FIG. 8C In vitro FAP/LTBR bispecific activity in chemokine induction assay using tumour cell line HCC1187 cultured with FAP positive primary human breast CAFs: Activity in HCC1187 monoculture

FIG. 8D In vitro FAP/LTBR bispecific activity in chemokine induction assay using tumour cell line HCC1187 cultured with FAP positive primary human breast CAFs: Activity in HCC1187:CHO-FAP co-culture

FIG. 8E In vitro FAP/LTBR bispecific activity in chemokine induction assay using tumour cell line HCC1187 cultured with FAP positive primary human breast CAFs: Activity in HCC1187 monoculture

FIG. 8F In vitro FAP/LTBR bispecific activity in chemokine induction assay using tumour cell line HCC1187 cultured with FAP positive primary human breast CAFs: Activity in HCC1187:CHO-FAP co-culture

FIG. 9A In vitro FAP/LTBR bispecific activity in ICAM-1 upregulation assay on human breast CAFs: ICAM-1 upregulation on primary human breast fibroblast CAFs post-FAP/LTBR bsAb stimulation

FIG. 9B In vitro FAP/LTBR bispecific activity in ICAM-1 upregulation assay on human breast CAFs: ICAM-1 upregulation on primary human breast fibroblast CAFs post-FAP/LTBR bsAb stimulation

FIG. 10A In vitro FAP/LTBR bispecific activity in chemokine induction assay using tumour cell line RPMI-7951: CCL5 secretion post-FAP/LTBR bsAb stimulation in RPMI-7951 cells

FIG. 10B In vitro FAP/LTBR bispecific activity in chemokine induction assay using tumour cell line RPMI-7951: CCL5 secretion post-FAP/LTBR bsAb stimulation in RPMI-7951 cells

FIG. 11A HCC-1187:RPMI-7951 CCL-19 read out: Trans-activation in the presence of low FAP expressing line

FIG. 11B HCC-1187:RPMI-7951 CCL-5 read out: Cis-activation in the presence of double-positive cells

FIG. 12A In vitro surrogate FAP/LTBR bispecific activity in chemokine induction assay using tumour cell line 4T1 cultured with FAP positive CHO-overexpressing line (monoculture)

FIG. 12B In vitro surrogate FAP/LTBR bispecific activity in chemokine induction assay using tumour cell line 4T1 cultured with FAP positive CHO-overexpressing line (co-culture)

FIG. 12C In vitro surrogate FAP/LTBR bispecific activity in chemokine induction assay using tumour cell line 4T1 cultured with FAP positive CHO-overexpressing line

FIG. 12D In vitro surrogate FAP/LTBR bispecific activity in chemokine induction assay using tumour cell line 4T1 cultured with FAP positive CHO-overexpressing line

FIG. 12E In vitro surrogate FAP/LTBR bispecific activity in chemokine induction assay using tumour cell line 4T1 cultured with FAP positive CHO-overexpressing line

FIG. 13 In vitro activity of mouse FAP/LTBR agonist antibody in primary mouse lung fibroblasts (graph I, repeat experiment I)

FIG. 14 In vitro activity of mouse FAP/LTBR agonist antibody in primary mouse lung fibroblasts (graph II, repeat experiment II)

FIG. 15 In vivo efficacy with FAP/LTBR bispecific molecules in orthotopic breast cancer models: Tumour growth inhibition post-treatment with a FAP/LTBR bispecific

FIG. 16 Treatment schedule for MMTV-PyMT mBR9071 tumour model radiotherapy study

FIG. 17 In vivo efficacy with FAP/LTBR bispecific surrogate molecules in orthotopic breast cancer models: HEV induction post-treatment

FIG. 18 In vivo efficacy with FAP/LTBR bispecific surrogate molecules in orthotopic breast cancer models: Treatment response in orthotopic EMT6 model

FIG. 19A In vivo efficacy with FAP/LTBR bispecific surrogate molecules in orthotopic breast cancer models: B cell increase post-treatment

FIG. 19B In vivo efficacy with FAP/LTBR bispecific surrogate molecules in orthotopic breast cancer models: HEV induction post-treatment

FIG. 20 In vivo efficacy with FAP/LTBR bispecific surrogate molecules in subcutaneous lung cancer model: Treatment response in subcutaneous lung cancer model

FIG. 21A In vivo efficacy with FAP/LTBR bispecific surrogate molecules in subcutaneous lung cancer model: HEV induction post-treatment

FIG. 21B In vivo efficacy with FAP/LTBR bispecific surrogate molecules in subcutaneous lung cancer model: CD4+ increase post-treatment

FIG. 21C In vivo efficacy with FAP/LTBR bispecific surrogate molecules in subcutaneous lung cancer model: CD8+ increase post-treatment

FIG. 22A Development of TLS structure in subcutaneous lung cancer model post combination treatment of PD-L1 with surrogate FAP/LTBR BsAb. Representative image of T cell aggregates (CD8 staining)

FIG. 22B Development of TLS structure in subcutaneous lung cancer model post combination treatment of PD-L1 with surrogate FAP/LTBR BsAb. Representative image of B cell aggregates (CD19 staining)

FIG. 23A Histology quantification of changes in tumor microenvironment, increase of CD8 post combination treatment of PD-L1 with surrogate FAP/LTBR BsAb

FIG. 23B Histology quantification of changes in the tumour microenvironment, increase of CD19 post combination treatment of PD-L1 with surrogate FAP/LTBR BsAb

FIG. 24 In vivo efficacy with LTBR monospecific surrogate molecules in combination with radiotherapy in subcutaneous colon cancer model: Tumour growth inhibition of primary (irradiated) tumour post-treatment

FIG. 25 In vivo efficacy with LTBR monospecific surrogate molecules in combination with radiotherapy in subcutaneous colon cancer model: Tumour growth inhibition of abscopal (non-irradiated) tumour post-treatment

FIG. 26A In vivo efficacy with LTBR monospecific surrogate molecules in combination with radiotherapy in subcutaneous colon cancer model: Changes in tumor microenvironment, increase of CD4 post combination treatment of fractionated radiotherapy with surrogate FAP/LTBR BsAb

FIG. 26B In vivo efficacy with LTBR monospecific surrogate molecules in combination with radiotherapy in subcutaneous colon cancer model: Changes in tumor microenvironment, increase of CD8 post combination treatment of fractionated radiotherapy with surrogate FAP/LTBR BsAb

FIG. 27A HepG2 NFKB activation-induced luciferase assay results illustrating activation of LTBR by FAP/LTBR bispecific antibodies in the presence of FAP (monoculture)

FIG. 27B HepG2 NFKB activation-induced luciferase assay results illustrating activation of LTBR by FAP/LTBR bispecific antibodies in the presence of FAP (co-culture)

FIG. 27C HepG2 NFKB activation-induced luciferase assay results illustrating activation of LTBR by FAP/LTBR bispecific antibodies in the presence of FAP (monoculture)

FIG. 27D HepG2 NFKB activation-induced luciferase assay results illustrating activation of LTBR by FAP/LTBR bispecific antibodies in the presence of FAP (co-culture)

FIG. 28A In vivo efficacy with FAP/LTBR bispecific surrogate molecules in orthotopic breast cancer models: Treatment response in orthotopic EMT6 model (tumor volume) in combination with cancer vaccine (peptide antigen+adjuvant)

FIG. 28B In vivo efficacy with FAP/LTBR bispecific surrogate molecules in orthotopic breast cancer models: CD4+ increase post-treatment

FIG. 28C In vivo efficacy with FAP/LTBR bispecific surrogate molecules in orthotopic breast cancer models: CD8+ increase post-treatment

FIG. 29 In vivo efficacy with FAP/LTBR bispecific surrogate molecules in orthotopic breast cancer models: Treatment response in orthotopic EMT6 model (probability of survival) in combination with cancer vaccine (peptide antigen+adjuvant)

FIG. 30 In vivo efficacy with FAP/LTBR bispecific surrogate molecules in orthotopic breast cancer models: Rechallenge response in orthotopic EMT6 model (tumour volume) in combination with cancer vaccine (peptide antigen+adjuvant)

### DETAILED DESCRIPTION OF THE INVENTION

### Binding Agents

Binding agents are capable of binding to a target with an affinity (suitably expressed as a KD value, a Ka value, a kₒₙ-rate and/or a k_{off}-rate, as further described herein). Suitably the binding agent agonises, inversely agonises, antagonises or neutralises a specified target. In one embodiment the binding agent is a small molecule. Suitably, the binding agent comprises or consists of a binding polypeptide (also referred to as a “binding domain”). Polypeptides are said to be binding polypeptides when they comprise a domain wherein the domain comprises one or more stretches of amino acid residues which form an antigen-binding site, capable of binding to an epitope on a target with an affinity. ‘Binding polypeptide’ and ‘antigen-binding polypeptide’ are used synonymously herein. A polypeptide which binds to LTBR is synonymous with a polypeptide which is anti-LTBR and a polypeptide which binds to FAP is synonymous with a polypeptide which is anti-FAP. Binding polypeptides may include antibodies and fragments thereof, such as variable domains (all of which are further described below), antibodies modified to comprise additional binding regions and antibody mimetics. Further binding polypeptides may include, for example, DARPins (Binz et al. 2003), Affimers™, Fynomers™, Centyrins, Nanofitins® and cyclic peptides.

A number of binding agents (including monospecific binding domains) described in the examples below are referred to as ‘clones’ and are assigned specific clone numbers or IDs.

### Antibodies and Fragments Thereof

The binding agents are preferably antibodies or binding fragments thereof. Such fragments may be comprised in a non-related antibody (or antigen-binding fragment thereof) such that the construct comprising the binding agents is a chimeric antibody (or chimeric fragment thereof). A conventional antibody or immunoglobulin (Ig) is a protein comprising four polypeptide chains: two heavy (H, or HC) chains and two light (L, or LC) chains. Each chain is divided into a constant region and a variable region. The variable region comprises a heavy chain variable region paired with a light chain variable region. The heavy (H) chain variable region is abbreviated herein as VH region, and the light (L) chain variable region is abbreviated herein as VL region. These domains, domains related thereto and domains derived therefrom, are referred to herein as variable domains. The VH and VL regions (or ‘domains’) can be further subdivided into regions of hypervariability, termed “complementarity determining regions” (“CDRs”), interspersed with regions that are more conserved, termed “framework regions” (“FRs”). The framework and complementarity determining regions have been precisely defined (Kabat et al 1991). As the invention relates to binding agents or polypeptides comprising complementarity determining regions (CDRs) and, optionally framework regions (FRs), some explanation is first provided on how such CDRs/FRs are determined. The determination of the CDR regions in a variable region sequence generally depends on the algorithm/methodology applied, such as Kabat-, Chothia-, Martin (enhanced Chothia)-, IMGT (ImMunoGeneTics information system)-, or AbM-numbering schemes. See, e.g. Kabat et al. 1991; Chothia and Lesk 1987; the AbM definition is a compromise between Kabat and Chothia as used by Oxford Molecular's AbM antibody modelling software, see also Martin and Allen 2007; Strohl and Strohl 2012, chapter 3: Antibody structure-function relationships, pp. 37-56; www.imgt.org/IMGTScientificChart/Numbering/IMGTnumbering.html.

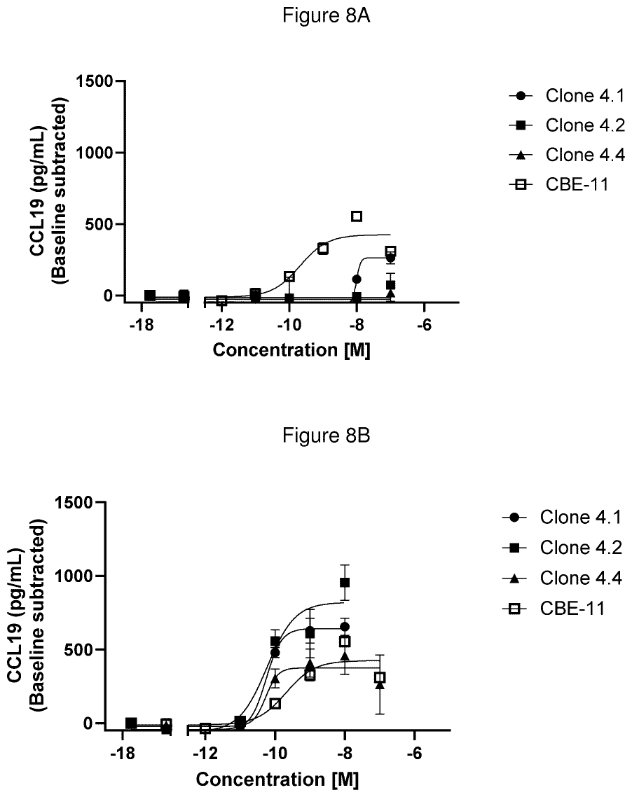

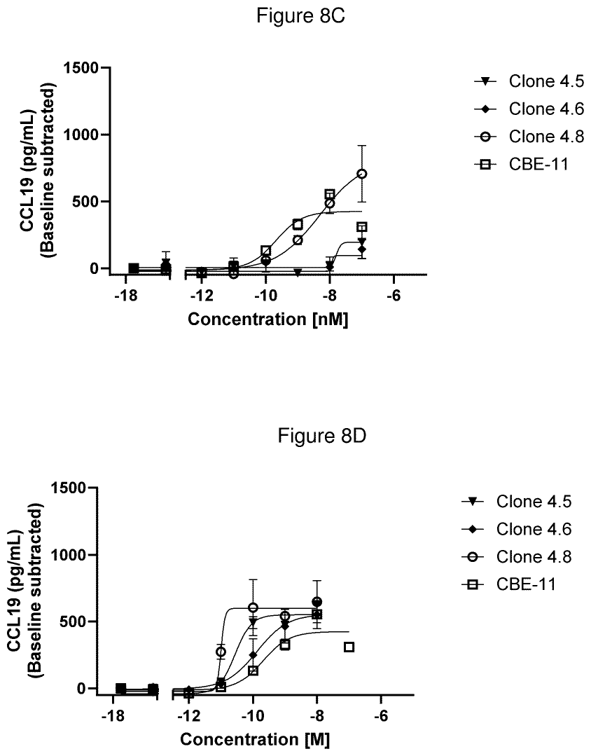

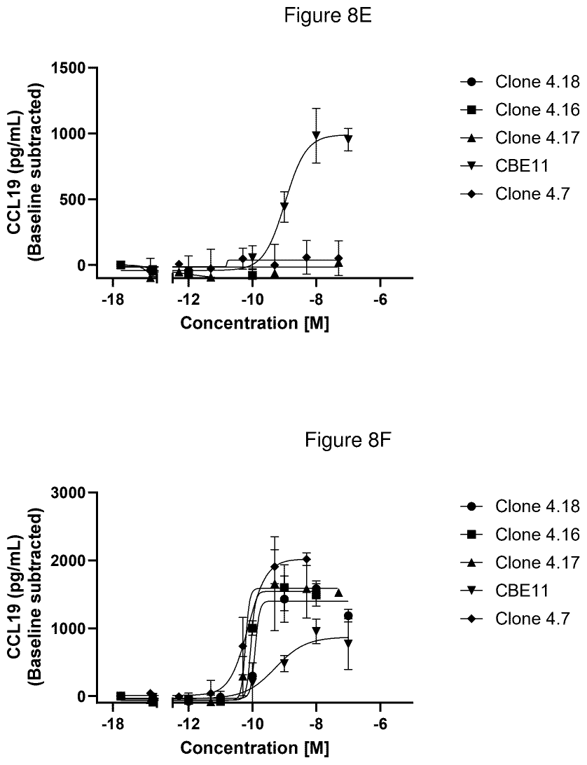

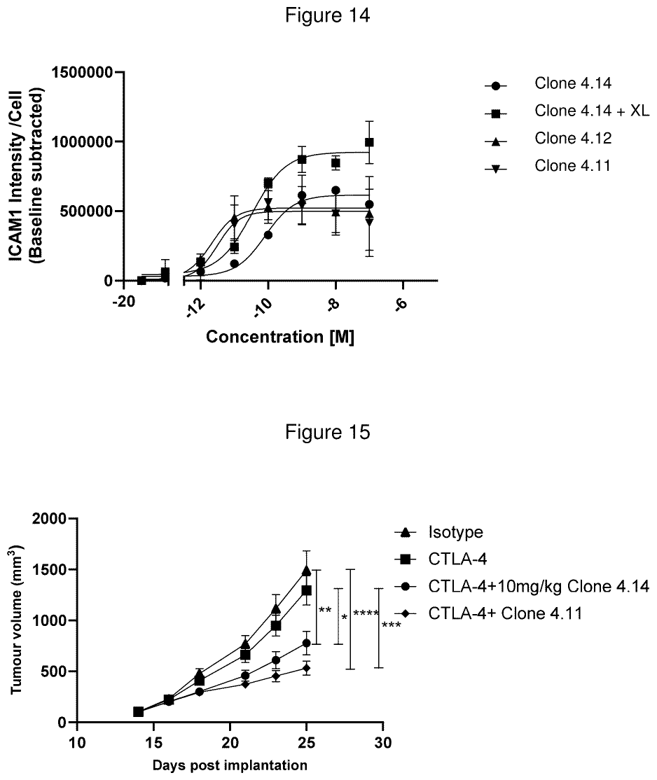

Applying different methods to the same variable region sequence may give rise to different CDR amino acid sequences wherein the differences may reside in CDR sequence length and/or -delineation within the variable region sequence, see FIG. 14 of WO2022136649A1 and FIG. 8 of WO2022136647A1 which illustrate the IMGT and Kabat systems applied in parallel on the same sequence and FIG. 64 of WO2022175532A1 which illustrates the Kabat, MacCallum, IMGT, AbM and Chothia systems applied in parallel on the same sequence. The CDRs comprised in the binding agents or polypeptides of the invention can therefore be described as the CDR sequences as present in a variable region sequence as characterized herein (more particularly an anti-LTBR or anti-FAP antibody/immunoglobulin/VHH sequence as characterized herein), or alternatively as determined or delineated according to a well-known methodology such as according to the Kabat-, Chothia-, Martin (enhanced Chothia), IMGT-, or AbM-numbering scheme or -method.

The delineation of the CDRs determines the delineation of the framework regions (FRs). As used in the claims and clauses herein, in the absence of an explicitly stated definition system, the Kabat CDR definition system is intended to be applied.

In a conventional antibody, each VH and VL region is composed of three CDRs and four FRs, arranged from amino-terminus to carboxy-terminus in the following order: FR1, CDR1, FR2, CDR2, FR3, CDR3, FR4. The VH region CDRs and FRs are denoted HCDR1, HCDR2, HCDR3, HFR1, HFR2, HFR3 and HFR4. The VL region CDRs and FRs are denoted LCDR1, LCDR2, LCDR3, LFR1, LFR2, LFR3 and LFR4. The conventional antibody tetramer of two heavy immunoglobulin chains and two light immunoglobulin chains is formed with the heavy and the light immunoglobulin chains inter-connected by e.g. disulfide bonds, and the heavy chains similarly connected. The heavy chain constant region includes three domains, CH1, CH2 and CH3. The light chain constant region is comprised of one domain, CL. The variable domain of the heavy chains and the variable domain of the light chains are binding domains that interact with an antigen. The constant regions of the antibodies typically mediate the binding of the antibody to host tissues or factors, including various cells of the immune system (e.g. effector cells) and the first component (C1q) of the classical complement system.

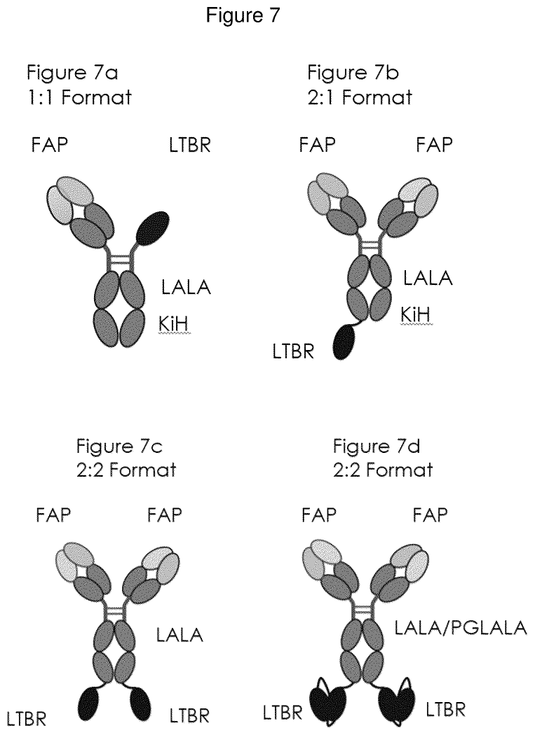

A conventional antibody comprises a homodimer of two identical heavy chains (HC) and two identical light chains (LC) (e.g. as shown in FIGS. 7c and 7d). Antibodies can also be produced which comprise a heterodimer of two different heavy chains (HC1 and HC2) and two identical light chains (LC) (e.g. as shown in FIGS. 7a and 7b).

A further exception to conventional antibody structure is found in sera of Camelidae. In addition to conventional antibodies, these sera possess special IgG antibodies. These IgG antibodies, known as heavy-chain antibodies (HCAbs), are devoid of the L chain polypeptide and lack the first constant domain (CH1). At its N-terminal region, the H chain of the homodimeric protein contains a dedicated immunoglobulin chain variable domain, referred to as the VHH, which serves to associate with its cognate antigen (Muyldermans 2013; Hamers-Casterman et al 1993).

The term “antibody” includes any substantially complete antibody protein comprising at least one antibody variable domain comprising at least one antigen binding site (ABS). Antibodies include, but are not limited to, immunoglobulins of types IgA, IgG, IgE, IgD, IgM (as well as subtypes thereof, e.g. IgG1). The overall structure of Immunoglobulin G (IgG) antibodies assembled from two identical heavy (H)-chain and two identical light (L)-chain polypeptides is well established and highly conserved in mammals (Padlan 1994).

A fragment of an antibody (which may also be referred to as “antibody fragment”, “antibody binding fragment”, “immunoglobulin fragment”, “antigen-binding fragment” or “antigen-binding polypeptide”) as used herein refers to a portion of an antibody that binds to the target (e.g. a molecule in which one or more immunoglobulin chains is not full length, but which binds to the target). Examples of binding fragments encompassed within the term antibody fragment include:
(i) a Fab fragment (a monovalent fragment consisting of the VL, VH, CL and CH1 domains);
(ii) a F(ab′)2 fragment (a bivalent fragment consisting of two Fab fragments linked by a disulphide bridge at the hinge region);
(iii) a Fd fragment (consisting of the VH and CH1 domains);
(iv) a Fv fragment (a variable domain or variable region, consisting of the VL and VH domains of a single arm of an antibody wherein the VL and VH domains together form an antigen binding site);
(v) a single chain variable fragment, scFv (consisting of VL and VH domains joined, possibly using recombinant methods, by a synthetic linker (e.g. a peptide) that enables them to be made as a single protein chain in which the VL and VH regions pair to form monovalent molecules);
(vi) a VH (a heavy chain variable domain);
(vii) a VL (a light chain variable domain);
(viii) a domain antibody (dAb, consisting of either the VH or VL domain);
(ix) a minibody (consisting of a pair of scFv fragments which are linked via CH3 domains); and
(x) a diabody (consisting of a noncovalent dimer of scFv fragments that consist of a VH domain from one antibody connected by a peptide linker a VL domain from another antibody);
(xi) a VHH (a heavy chain variable domain as described in detail above).

The antibody or fragment thereof may be a human antibody or derived from a human antibody. “Human antibody” refers to antibodies having variable and constant regions derived from human germline immunoglobulin sequences. Human subjects administered with said human antibodies do not generate cross-species antibody responses (for example termed HAMA responses—human-anti-mouse antibody) to the primary amino acids contained within said antibodies. Said human antibodies may include amino acid residues not encoded by human germline immunoglobulin sequences (e.g. mutations introduced by random or site-specific mutagenesis or by somatic mutation), for example in the CDRs and in particular CDR3. However, the term is not intended to include antibodies in which CDR sequences derived from the germline of another mammalian species, such as a mouse, have been grafted onto human framework sequences. Human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes or antibodies prepared, expressed, created or isolated by any other means that involves splicing of human immunoglobulin gene sequences to other DNA sequences, may also be referred to as “recombinant human antibodies”. Substituting at least one amino acid residue in the framework region of a non human immunoglobulin variable domain with the corresponding residue from a human variable domain is referred to as “humanisation”. Humanisation of a variable domain may reduce immunogenicity in humans.

Antibodies comprise a hinge region, as do certain constructs comprising antibody domains and antibody fragments. A naturally occurring hinge region is a flexible amino acid stretch in the central part of the heavy chains of the IgG and IgA immunoglobulin classes, which links these 2 chains by disulfide bonds. As used herein, a hinge region encompasses naturally occurring hinge regions and also variants thereof wherein suitably the variants maintain adequate flexibility and rigidity to maintain integrity of the antibody, fragment or construct, while maintaining binding ability. A native hinge region may for example comprise or consist of the polypeptide sequence SEQ ID NO: 204. The variant may be a truncated version (a ‘truncated hinge region’) of a naturally occurring hinge region, such as a hinge region truncated by 5 amino acids relative to a naturally occurring hinge region, or most suitably a truncated hinge region comprising or consisting of SEQ ID NO: 203. In one embodiment a hinge region consists of 10-80 amino acids, such as 20-70 amino acids, such as 30-60 amino acids, such as 40-50 amino acids.

Suitably the construct comprises an antibody, or an antibody fragment. In one embodiment, the antibody fragment comprises an scFv, Fv, Fab, Fab′, F(ab′)2, variable domain (e.g. VH, VL, VNAR or VHH), diabody or minibody. If the construct is an antibody or antibody fragment, then the binding regions of the antibody or antibody fragment (e.g. the Fab regions of the antibody or antibody fragment, or synthetically introduced further binding domains such as VHHs) provide the FAP binding agent(s) and the LTBR binding agent(s) of the construct of the invention.

Most suitably the construct is an antibody (i.e. a full length antibody). Antibodies of the invention can be of any class, e.g. IgG, IgA, IgM, IgE, IgD, or subclass thereof, and can comprise a kappa or lambda light chain. In one embodiment, the antibody is an IgG antibody, for example, at least one of subclasses, IgG1, IgG2, IgG3 or IgG4. In one embodiment, the antibody is an IgG1, most suitably human IgG1. In a further embodiment, the antibody may be in a format, such as an IgG format, that has been modified to confer desired properties, such as having the Fc mutated to reduce effector function, extend half-life, alter ADCC, or improve hinge stability. Such modifications are well known in the art and exemplary embodiments are described herein. Suitably, the antibody comprises PGLALA substitutions (substitutions Pro329Gly, Leu234Ala, and Leu235Ala, Schlothauer et al. 2016) in the Fc region. Suitably the antibody Fc region comprises 329Gly, 234Ala and 235Ala. Alternatively, the antibody comprises LALA substitutions (substitutions Leu234Ala, and Leu235Ala, Schlothauer et al. 2016) in the Fc region. Suitably the antibody Fc region comprises 234Ala and 235Ala. In a further embodiment the antibody comprises an STR-silenced Fc region (i.e. G236R in combination with the LALA substitutions described above, see Wilkinson et al. 2021). Therefore, in one embodiment, the Fc region comprises 234Ala, 235Ala and 236Arg. In a yet further embodiment, the Fc region is aglycosylated. The same Fc modifications may be applied to any construct comprising an Fc.

In one embodiment the antibody is multispecific, such as bispecific and multivalent, such as bivalent. Suitable constructs of these formats and other formats are detailed further below.

In a particular embodiment, the construct comprises, essentially consists of or consists of an antibody or a pair of scFvs. Most suitably the construct comprises an antibody.

In a further embodiment, the construct comprises or consists of an antibody and a VHH, wherein the VHH replaces one of the Fab regions (such as illustrated in FIG. 7a), the VHH is a binding polypeptide which binds to LTBR and the paired VH and VL of the remaining Fab region is a binding polypeptide which binds to FAP.

In an alternative embodiment, the construct comprises or consists of an antibody and a VHH, wherein the VHH is fused to the Fc region (e.g. a CH3 domain) of the antibody (such as illustrated in FIG. 7b), the VHH is a binding polypeptide which binds to LTBR and the paired VH and VL of each of the two Fab regions are binding polypeptides which bind to FAP. Suitably a further VHH is fused to the Fc region (e.g. the second CH3 domain), wherein the further VHH is a binding polypeptide which binds to LTBR (such as illustrated in FIG. 7c).

In a particular embodiment, the construct comprises or consists of an antibody and an scFv, wherein the scFv is fused to the Fc region (e.g. a CH3 domain) of the antibody, the scFv is a binding polypeptide which binds to LTBR and the paired VH and VL of each of the two Fab regions are binding polypeptides which bind to FAP. Suitably a further scFv is fused to the Fc region (e.g. the second CH3 domain), wherein the further scFv is a binding polypeptide which binds to LTBR (such as illustrated in FIG. 7d).

In one embodiment, an scFv may be fused to the C-terminus of an Fc. Suitably, a peptide linker such as a linker comprising Glycine and Serine residues, such as a G₄S linker (SEQ ID NO: 3) or a (G₄S)₄ linker (SEQ ID NO: 4) may be used. Suitably a disulfide bond may be situated in the VH44:VL100 position (Weatherill et al. 2012). In one embodiment, the linker does not comprise an Ig domain. In one embodiment, the linker is a linear peptide. In one embodiment, the linker is a hinge region. Most suitably, the scFv is fused to the CH3 domain by a linear peptide linker.

### Specificity, Affinity, Avidity and Potency

“Specificity” refers to the number of different types of antigens or antigenic determinants to which a particular antibody or fragment thereof can bind. The specificity of an antibody is the ability of the antibody to recognise a particular antigen as a unique molecular entity and distinguish it from another. An antibody that “specifically binds” to an antigen or an epitope is a term well understood in the art. A molecule is said to exhibit “specific binding” if it reacts more frequently, more rapidly, with greater duration and/or with greater affinity with a particular target antigen or epitope, than it does with alternative targets. An antibody “specifically binds” to a target antigen or epitope if it binds with greater affinity, avidity, more readily, and/or with greater duration than it binds to other substances. An antibody (or fragment thereof) may be considered to specifically bind to a target if the binding is statistically significant compared to a non-relevant binder. Specific binding of an antibody, or fragment thereof, to an antigen or antigenic determinant can be determined in any suitable known manner, including, for example, Scatchard analysis and/or competitive binding assays, such as radioimmunoassays (RIA), enzyme immunoassays (EIA) and sandwich competition assays, equilibrium dialysis, equilibrium binding, gel filtration, ELISA, or spectroscopy (e.g. using a fluorescence assay) and the different variants thereof known in the art.

In one embodiment by ‘binding specifically’ it is meant that the target is bound to with a KD of 10⁻⁸ M or less, such as 10⁻⁹ M or less whereas any other entity target is bound to with a KD of 10⁻⁷ M or more such as 10⁻⁶ M or more. More suitably, binding to any other entity cannot be measured.

“Affinity”, represented by the equilibrium constant for the dissociation of an antigen with an antigen-binding polypeptide (K_{D}), is a measure of the binding strength between an antigenic determinant and an antigen-binding site on the antibody (or fragment thereof): the lesser the value of the K_{D}, the stronger the binding strength between an antigenic determinant and the antigen-binding polypeptide. Alternatively, the affinity can also be expressed as the affinity constant (KA), which is 1/K_{D}. Affinity can be determined by known methods, depending on the specific antigen of interest. Any K_{D} value less than 10⁻⁶ M is considered to indicate binding. Suitably, constructs of the invention will bind targets with a dissociation constant of 10⁻⁶ M or less, more suitably 10⁻⁷ M or less, more suitably 10⁻⁸ M or less and more suitably 10⁻⁹ M or less.

Suitably, the K_{D} of a polypeptide of the invention is determined using surface plasmon resonance (SPR) or biolayer interferometry (“BLI”, such as by using an Octet instrument or such as by the method described in Example 4).

“Avidity” is the measure of the strength of binding between a polypeptide, an antibody or fragment thereof, and the pertinent antigen. Avidity is related to both the affinity between an antigenic determinant and its antigen binding site on the antibody and the number of pertinent binding sites present on the antibody.

“Potency” is a measure of the activity of a therapeutic agent expressed in terms of the amount required to produce an effect of given intensity. A highly potent agent evokes a greater response at low concentrations compared to an agent of lower potency that evokes a smaller response at low concentrations. Potency is a function of affinity and efficacy. Efficacy refers to the ability of therapeutic agent to produce a biological response upon binding to a target and the quantitative magnitude of this response. The term half maximal effective concentration (EC50) refers to the concentration of a therapeutic agent which causes a response halfway between the baseline and maximum after a specified exposure time. The therapeutic agent may cause inhibition or stimulation. It is commonly used, and is used herein, as a measure of potency.

### LTBR

Lymphotoxin beta receptor (LTBR), also known as tumour necrosis factor receptor superfamily member 3 (TNFRSF3), is a cell surface receptor for lymphotoxin involved in apoptosis and cytokine release. It is a member of the tumour necrosis factor receptor superfamily.

The construct of the invention comprises an LTBR binding agent. LTBR binding agents are disclosed in the prior art, for example anti-LTBR binding polypeptide BHA10 (WO2004002431, comprising heavy and light chain variable regions of SEQ ID NO: 1 and 2, respectively) and huCBE11 (WO2004002431).

Suitably the one or all of the LTBR binding agents comprise or consist of a variable domain which binds to LTBR, such as a VH, VL, VNAR or VHH, and more suitably a heavy chain variable domain such as a VHH.

The LTBR is suitably situated on the surface of stromal cells, fibroblasts, endothelial cells and/or cancer cells. Suitably the LTBR is situated on the surface of a cell wherein the cell also comprises FAP on its surface.

The LTBR binding agent suitably binds to the extracellular domain of LTBR.

In one embodiment, LTBR is a polypeptide sharing at least 50% identity, such as at least 60% identity, such as at least 70% identity, such as at least 80% identity, such as at least 90% identity, such as at least 95% identity with SEQ ID NO: 9 (full length human LTBR). Suitably, LTBR comprises such as consists of SEQ ID NO: 9 (full length human LTBR).

Suitably, the LTBR is the extracellular domain of human LTBR, i.e. the LTBR binding agent binds to the extracellular domain of human LTBR. In one embodiment, LTBR is a polypeptide sharing at least 50% identity, such as at least 60% identity, such as at least 70% identity, such as at least 80% identity, such as at least 90% identity, such as at least 95% identity with SEQ ID NO: 10 (human LTBR extracellular domain). Suitably, LTBR is a polypeptide comprising or consisting of SEQ ID NO: 10 (human LTBR extracellular domain).

In one embodiment, LTBR is a polypeptide sharing at least 50% identity, such as at least 60% identity, such as at least 70% identity, such as at least 80% identity, such as at least 90% identity, such as at least 95% identity with SEQ ID NO: 11 (full length Macaca fascicularis LTBR). Suitably, LTBR comprises such as consists of SEQ ID NO: 11 (full length Macaca fascicularis LTBR).

Suitably, the LTBR is the extracellular domain of Macaca fascicularis LTBR, i.e. the LTBR binding agent binds to the extracellular domain of Macaca fascicularis LTBR. In one embodiment, LTBR is a polypeptide sharing at least 50% identity, such as at least 60% identity, such as at least 70% identity, such as at least 80% identity, such as at least 90% identity, such as at least 95% identity with SEQ ID NO: 12 (Macaca fascicularis LTBR extracellular domain). Suitably, LTBR is a polypeptide comprising or consisting of SEQ ID NO: 12 (Macaca fascicularis LTBR extracellular domain).

In one embodiment, LTBR is a polypeptide sharing at least 50% identity, such as at least 60% identity, such as at least 70% identity, such as at least 80% identity, such as at least 90% identity, such as at least 95% identity with SEQ ID NO: 13 (full length mouse LTBR). Suitably, LTBR comprises such as consists of SEQ ID NO: 13 (full length mouse LTBR).

Suitably, the LTBR is the extracellular domain of mouse LTBR, i.e. the LTBR binding agent binds to the extracellular domain of mouse LTBR. In one embodiment, LTBR is a polypeptide sharing at least 50% identity, such as at least 60% identity, such as at least 70% identity, such as at least 80% identity, such as at least 90% identity, such as at least 95% identity with SEQ ID NO: 14 (mouse LTBR extracellular domain). In one embodiment, LTBR is a polypeptide comprising or consisting of SEQ ID NO: 14 (mouse LTBR extracellular domain).

Suitably one or all of the LTBR binding agents have an EC50 of 9.0E-09 M or less, such as 8.0E-09 M or less, such as 7.0E-09 M or less, such as 6.0E-09 M or less, such as 5.0E-09 M or less, such as 4.0E-09 M or less, such as 3.0E-09 M or less, such as 2.0E-09 M or less, such as 1.0E-09 M or less, such as 9.0E-10 M or less, such as 8.0E-10 M or less, such as 7.0E-10 M or less, such as 6.0E-10 M or less, such as 5.0E-10 M or less, such as 4.0E-10 M or less, such as 3.0E-10 M or less, such as 2.0E-10 M or less, such as 1.0E-10 M or less, such as 9.0E-11 M or less, such as 8.0E-11 M or less, such as 7.0E-11 M or less, such as 6.0E-11 M or less, such as 5.0E-11 M or less, such as 4.0E-11 M or less, such as 3.0E-11 M or less, such as 2.0E-11 M or less, such as 1.0E-11 M or less, such as 9.0E-12 M or less, such as 8.0E-12 M or less, such as 7.0E-12 M or less, such as 6.0E-12 M or less, such as 5.0E-12 M or less, such as 4.0E-12 M or less, such as 3.0E-12 M or less, such as 2.0E-12 M or less, such as 1.0E-12 M or less. Suitably the EC50 is established using a chemokine induction assay, for example assayed using an ELISA. More suitably the EC50 is established using an assay set out in any one of the Examples.

Suitably one or all of the LTBR binding agents have an EC50 as recited for any of the LTBR binding agents specified in the tables of the Examples or less.

Suitably the LTBR binding agent binds to LTBR specifically, i.e. the binding agent does not bind significantly to targets other than human LTBR, such as does not bind significantly to targets other than human, Macaca fascicularis or mouse LTBR, such as does not bind significantly to targets other than LTBR.

In one embodiment, the LTBR binding agent is the LTBR ligand, LIGHT, more suitably human LIGHT.

In one embodiment, the LTBR binding agent is not the LTBR ligand, human LIGHT, or more suitably the LTBR binding agent is not LIGHT.

Suitably LIGHT is a polypeptide comprising, consisting essentially of, or consisting of a sequence sharing at least 70%, such as at least 80%, such as at least 90%, such as at least 99%, such as 100% identity, with SEQ ID NO: 21 or SEQ ID NO: 22.

The construct of the invention may bind to LTBR with an avidity of 1.0E-08 M or less, such as 9.0E-09 M or less, such as 7.0E-09 M or less, such as 6.7E-09 M or less, such as 5.0E-09 M or less, such as 3.0E-09 M or less, such as 2.0E-09 M or less, such as 1.0E-09 M or less.

Suitably one or all of the LTBR binding agents bind to LTBR with an affinity (KD) of 20 nM or less, such as 15 nM or less, such as 10 nM or less, such as 9 nM or less, such as 8 nM or less, such as 7 nM or less, such as 6 nM or less, such as 5 nM or less, such as 4 nM or less, such as 3 nM or less, such as 2 nM or less, such as 1 nM or less. Suitably the KD is established using the assay set out in Example 4.

Suitably the LTBR binding agent agonises (i.e. activates), inversely agonises, antagonises or neutralises LTBR. In one embodiment the LTBR binding agent is an LTBR agonist. Suitably the agonist activates such NFKB signalling pathways. If the LTBR binding agent is an LTBR agonist, then suitably the LTBR binding agent (a) induces clustering of LTBR and more suitably (b) activates the classical NFKB pathway (suitably leading to expression of adhesion molecules such as ICAMs, such as ICAM-1, VCAM-1 and/or MAdCAM-1) and/or activates the alternative NFKB pathway (suitably leading to expression of chemokines, such as CCL5, CCL19, CCL21 and/or CXCL13).

Suitably the LTBR binding agent is an LTBR agonist in that the LTBR binding agent has an EC50 of 9.0E-09 M or less, such as 1.0E-08 M or less, such as 8.0E-09 M or less, such as 7.0E-09 M or less, such as 6.0E-09 M or less, such as 5.0E-09 M or less, such as 4.0E-09 M or less, such as 3.0E-09 M or less, such as 2.0E-09 M or less, such as 1.0E-09 M or less, such as 9.0E-10 M or less, such as 8.0E-10 M or less, such as 7.0E-10 M or less, such as 6.0E-10 M or less, such as 5.0E-10 M or less, such as 4.0E-10 M or less, such as 3.0E-10 M or less, such as 2.0E-10 M or less, such as 1.0E-10 M or less, such as 9.0E-11 M or less, such as 8.0E-11 M or less, such as 7.0E-11 M or less, such as 6.0E-11 M or less, such as 5.0E-11 M or less, such as 4.0E-11 M or less, such as 3.0E-11 M or less, such as 2.0E-11 M or less, such as 1.0E-11 M or less, such as 9.0E-12 M or less, such as 8.0E-12 M or less, such as 7.0E-12 M or less, such as 6.0E-12 M or less, such as 5.0E-12 M or less, such as 4.0E-12 M or less, such as 3.0E-12 M or less, such as 2.0E-12 M or less, such as 1.0E-12 M or less. Alternatively, the LTBR binding agent may have an EC50 as recited for any of the LTBR binding agents specified in the tables of the Examples or less. Suitably the EC50 is established using a chemokine induction assay, for example assayed using an ELISA. More suitably the EC50 is established using an assay set out in any one of the Examples.

In one embodiment, the construct binds to mouse LTBR. In an alternative embodiment, the construct binds to Macaca fascicularis LTBR. In an alternative embodiment, the construct binds to human LTBR.

One, a plurality, or all of the LTBR binding agents may be a variable domain (e.g. a heavy chain variable domain, e.g. a VHH) which binds to LTBR, suitably as defined further below.

In one embodiment, the LTBR-binding agent or polypeptide comprises at least one CDR, wherein the CDR is a HCDR1, HCDR2 or HCDR3 comprised in a heavy chain variable domain of SEQ ID NOs: 23-26 or 159-162. Suitably, such agent or polypeptide comprises at least such HCDR1 and HCDR2, such HCDR1 and HCDR3, or such HCDR2 and HCDR3. More suitably, such agent or polypeptide comprises such HCDR1, HCDR2 and HCDR3. More suitably, the HCDR1, HCDR2 and HCDR3 are delineated according to the Kabat-, Chothia-, Martin-, IMGT-, or AbM-method. More suitably the HCDR1 is chosen from SEQ ID NOs: 57, 60, 63, 66, 163, 166, 169 or 172 as defined by the Kabat-method; the HCDR2 is chosen from SEQ ID NOs: 58, 61, 64, 67, 164, 167, 170, 173, 205 or 206 as defined by the Kabat-method; and/or the HCDR3 is chosen from SEQ ID NOs: 59, 62, 65, 68, 165, 168, 171, 174, 207 or 208 as defined by the Kabat-method.

In one embodiment the variable domain which binds to LTBR comprises or consists of three heavy chain complementarity determining regions (HCDR1-HCDR3) and four framework regions (FR1-FR4), wherein HCDR1 comprises or consists of a polypeptide sequence sharing 70% or greater, 75% or greater, 80% or greater, 85% or greater, 90% or greater or 95% or greater sequence identity with SEQ ID NO: 57, 60, 63, 66, 163, 166, 169 or 172 (e.g. 57, 60, 63 or 66) or wherein HCDR1 comprises or consists of a polypeptide sequence having 3, 2 or 1 amino acid sequence difference with SEQ ID NO: 57, 60, 63, 66, 163, 166, 169 or 172 (e.g. 57, 60, 63 or 66); HCDR2 comprises or consists of a polypeptide sequence sharing 70% or greater, 75% or greater, 80% or greater, 85% or greater, 90% or greater or 95% or greater sequence identity with SEQ ID NO: 58, 61, 64, 67, 164, 167, 170, 173, 205 or 206 (e.g. 58, 61, 64 or 67) or wherein HCDR2 comprises or consists of a polypeptide sequence having 3, 2 or 1 amino acid sequence difference with SEQ ID NO: 58, 61, 64, 67, 164, 167, 170, 173, 205 or 206 (e.g. 58, 61, 64 or 67); and HCDR3 comprises or consists of a polypeptide sequence sharing 70% or greater, 75% or greater, 80% or greater, 85% or greater, 90% or greater or 95% or greater sequence identity with SEQ ID NO: 59, 62, 65, 68, 165, 168, 171, 174, 207 or 208 (e.g. 59, 62, 65 or 68) or wherein HCDR3 comprises or consists of a polypeptide sequence having 3, 2 or 1 amino acid sequence difference with SEQ ID NO: 59, 62, 65, 68, 165, 168, 171, 174, 207 or 208 (e.g. 59, 62, 65 or 68). More suitably HCDR1 comprises or consists of a polypeptide sequence of SEQ ID NO: 57, 60, 63, 66, 163, 166, 169 or 172 (e.g. 57, 60, 63 or 66), HCDR2 comprises or consists of a polypeptide sequence of SEQ ID NO: 58, 61, 64, 67, 164, 167, 170, 173, 205 or 206 (e.g. 58, 61, 64 or 67) and HCDR3 comprises or consists of a polypeptide sequence of SEQ ID NO: 59, 62, 65, 68, 165, 168, 171, 174, 207 or 208 (e.g. 59, 62, 65 or 68). In particular, these CDR polypeptide sequences were determined according to or with the Kabat-method.

In one embodiment the variable domain which binds to LTBR comprises the three heavy chain complementarity determining region amino acid sequences (HCDR1-HCDR3) of the variable domain amino acid sequence of SEQ ID NO: 23, 24, 25, 26, 159, 160, 161 or 162 (e.g. 23, 24, 25 or 26).

In one embodiment the variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 23, 24, 25, 26, 159, 160, 161 or 162 (e.g. 23, 24, 25 or 26). More suitably the variable domain which binds to LTBR comprises or consists of a polypeptide sequence of SEQ ID NO: 23, 24, 25, 26, 159, 160, 161 or 162 (e.g. 23, 24, 25 or 26). In one further embodiment thereto, the polypeptide sequence variation is in one or more the framework regions FR1-FR4 comprised in SEQ ID NO: 23, 24, 25, 26, 159, 160, 161 or 162 (e.g. 23, 24, 25 or 26). Alternatively, the polypeptide sequence variation is one or more of the framework regions FR1-FR4 comprised in SEQ ID NO: 23, 24, 25, 26, 159, 160, 161 or 162 (e.g. 23, 24, 25 or 26) and the CDR polypeptide sequences comprised within SEQ ID NO: 23, 24, 25, 26, 159, 160, 161 or 162 (e.g. 23, 24, 25 or 26) are the HCDR1-HCDR3 polypeptide sequences as outlined above. In one embodiment one, a plurality, or all of the LTBR binding agents comprise or consist of a paired heavy chain variable domain and light chain variable domain which bind to LTBR (e.g. an scFv), suitably as defined further below.

In one embodiment the heavy chain variable domain which binds to LTBR comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 69, 75, 81, 87, 93, 99, 105 or 111, HCDR2 comprises or consists of a polypeptide sequence sharing 70% or greater, 75% or greater, 80% or greater, 85% or greater, 90% or greater or 95% or greater sequence identity with SEQ ID NO: 70, 76, 82, 88, 94, 100, 106 or 112 and HCDR3 comprises or consists of a polypeptide sequence sharing 70% or greater, 75% or greater, 80% or greater, 85% or greater, 90% or greater or 95% or greater sequence identity with SEQ ID NO: 71, 77, 83, 89, 95, 101, 107 or 113 and the light chain variable domain which binds to LTBR comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 72, 78, 84, 90, 96, 102, 108 or 114, LCDR2 comprises or consists of a polypeptide sequence sharing 70% or greater, 75% or greater, 80% or greater, 85% or greater, 90% or greater or 95% or greater sequence identity with SEQ ID NO: 73, 79, 85, 91, 97, 103, 109 or 115 and LCDR3 comprises or consists of a polypeptide sequence sharing 70% or greater, 75% or greater, 80% or greater, 85% or greater, 90% or greater or 95% or greater sequence identity with SEQ ID NO: 74, 80, 86, 92, 98, 104, 110 or 116. More suitably HCDR1 comprises or consists of a polypeptide sequence of SEQ ID NO: 69, 75, 81, 87, 93, 99, 105 or 111, HCDR2 comprises or consists of a polypeptide sequence of SEQ ID NO: 70, 76, 82, 88, 94, 100, 106 or 112 and HCDR3 comprises or consists of a polypeptide sequence of SEQ ID NO: 71, 77, 83, 89, 95, 101, 107 or 113 and LCDR1 comprises or consists of a polypeptide sequence of SEQ ID NO: 72, 78, 84, 90, 96, 102, 108 or 114, LCDR2 comprises or consists of a polypeptide sequence of SEQ ID NO: 73, 79, 85, 91, 97, 103, 109 or 115 and LCDR3 comprises or consists of a polypeptide sequence of SEQ ID NO: 74, 80, 86, 92, 98, 104, 110 or 116.

In one embodiment the heavy chain variable domain which binds to LTBR comprises the three heavy chain complementarity determining region amino acid sequences (HCDR1-HCDR3) of the heavy variable domain amino acid sequence of SEQ ID NO: 27, 29, 31, 33, 35, 37, 39 or 41 and the light chain variable domain which binds to LTBR comprises the three light chain complementarity determining region amino acid sequences (LCDR1-LCDR3) of the light variable domain amino acid sequence of SEQ ID NO: 28, 30, 32, 34, 36, 38, 40 or 42.

In one embodiment the heavy chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 27, 29, 31, 33, 35, 37, 39 or 41 and the light chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 28, 30, 32, 34, 36, 38, 40 or 42. More suitably the heavy chain variable domain which binds to LTBR comprises or consists of a polypeptide sequence of SEQ ID NO: 27, 29, 31, 33, 35, 37, 39 or 41 and the light chain variable domain which binds to LTBR comprises or consists of a polypeptide sequence of SEQ ID NO: 28, 30, 32, 34, 36, 38, 40 or 42.

Preferably the HCDRs (and LCDRs) are provided in the heavy chain variable domain which binds to LTBR (and light chain variable domain which binds to LTBR) in the same combinations as set out in A/B. For example, it is preferable that HCDRs1-3 of SEQ ID NOs: 57, 58 and 59 are deployed in combination in e.g. a VHH (as set out for Clone 1.5), and it is preferable that (a) HCDRs1-3 of SEQ ID NOs: 69, 70 and 71 and (b) LCDRs1-3 of SEQ ID NOs: 72, 73 and 74 are deployed in combination (as set out for Clone 2.1) e.g. in an scFv. The same principle applies for combinations of heavy and light chain variable regions as set out in.

When multiple LTBR binding agents are deployed in a construct of the invention, the LTBR binding agents may be (a) identical or (b) different LTBR binding agents. If different, the LTBR binding agents are suitably independently selected from the LTBR binding agents described herein, such as those detailed above.

If the construct is an antibody, then in one embodiment one or all of the LTBR binding agents are a binding domain (e.g. a VHH or scFv, more suitably a VHH), linked to the constant region (e.g. the CH3 region).

### FAP

Fibroblast activation protein alpha (FAP-alpha, FAP) also known as prolyl endopeptidase FAP is an enzyme that in humans is encoded by the FAP gene. FAP is a 170 kDa membrane-bound gelatinase. FAP is highly expressed in cancer tissue across a wide range of cancer types and is particularly expressed on the surface of cancer associated fibroblasts (CAFs) present in the cancer stroma and on cancer cells (Zboralski et al 2022, Zhao et al 2022).

The construct of the invention comprises a FAP binding agent. FAP binding agents are disclosed in the prior art, for example anti-FAP binding polypeptide 28H1 (U.S. Ser. No. 10/577,429B2, comprising heavy and light chain variable regions of SEQ ID NO: 7 and 8, respectively).

Suitably the FAP binding agent achieves localisation (e.g. targeting of a tumour), co-location with the LTBR binding agent or clustering of LTBR in a bispecific; i.e. conditional activation. This conditional activation phenomenon should allow for reduced toxicity due to activation of LTBR-positive cells in the presence of FAP in the tumour (and not in tissues where FAP is not expressed). The FAP is suitably situated on the surface of stromal and/or cancer cells. Suitably the FAP is situated on the surface of a cell wherein the cell also comprises LTBR on its surface.

The FAP binding agent suitably binds to the extracellular domain of FAP.

In one embodiment, FAP is a polypeptide sharing at least 50% identity, such as at least 60% identity, such as at least 70% identity, such as at least 80% identity, such as at least 90% identity, such as at least 95% identity with SEQ ID NO: 15 (full length human FAP). Suitably, FAP comprises such as consists of SEQ ID NO: 15 (full length human FAP).

Suitably, the FAP is the extracellular domain of human FAP, i.e. the FAP binding agent binds to the extracellular domain of human FAP. In one embodiment, FAP is a polypeptide sharing at least 50% identity, such as at least 60% identity, such as at least 70% identity, such as at least 80% identity, such as at least 90% identity, such as at least 95% identity with SEQ ID NO: 16 (human FAP extracellular domain). Suitably, FAP is a polypeptide comprising or consisting of SEQ ID NO: 16.

In one embodiment, FAP is a polypeptide sharing at least 50% identity, such as at least 60% identity, such as at least 70% identity, such as at least 80% identity, such as at least 90% identity, such as at least 95% identity with SEQ ID NO: 17 (full length Macaca fascicularis FAP). Suitably, FAP comprises such as consists of SEQ ID NO: 17 (full length Macaca fascicularis FAP).

Suitably, the FAP is the extracellular domain of Macaca fascicularis FAP, i.e. the FAP binding agent binds to the extracellular domain of Macaca fascicularis FAP. In one embodiment, FAP is a polypeptide sharing at least 50% identity, such as at least 60% identity, such as at least 70% identity, such as at least 80% identity, such as at least 90% identity, such as at least 95% identity with SEQ ID NO: 18 (Macaca fascicularis FAP extracellular domain). Suitably, FAP is a polypeptide comprising or consisting of SEQ ID NO: 18.

In one embodiment, FAP is a polypeptide sharing at least 50% identity, such as at least 60% identity, such as at least 70% identity, such as at least 80% identity, such as at least 90% identity, such as at least 95% identity with SEQ ID NO: 19 (full length mouse FAP). Suitably, FAP comprises such as consists of SEQ ID NO: 19 (full length mouse FAP).

Suitably, the FAP is the extracellular domain of mouse FAP, i.e. the FAP binding agent binds to the extracellular domain of mouse FAP. In one embodiment, FAP is a polypeptide sharing at least 50% identity, such as at least 60% identity, such as at least 70% identity, such as at least 80% identity, such as at least 90% identity, such as at least 95% identity with SEQ ID NO: 20 (mouse FAP extracellular domain). Suitably, FAP is a polypeptide comprising or consisting of SEQ ID NO: 20 (mouse FAP extracellular domain).

Suitably one or all of the FAP binding agents have an EC50 of 9.0E-09 M or less, such as 8.0E-09 M or less, such as 7.0E-09 M or less, such as 6.0E-09 M or less, such as 5.0E-09 M or less, such as 4.0E-09 M or less, such as 3.0E-09 M or less, such as 2.0E-09 M or less, such as 1.0E-09 M or less, such as 9.0E-10 M or less, such as 8.0E-10 M or less, such as 7.0E-10 M or less, such as 6.0E-10 M or less, such as 5.0E-10 M or less, such as 4.0E-10 M or less, such as 3.0E-10 M or less, such as 2.0E-10 M or less, such as 1.0E-10 M or less, such as 9.0E-11 M or less, such as 8.0E-11 M or less, such as 7.0E-11 M or less, such as 6.0E-11 M or less, such as 5.0E-11 M or less, such as 4.0E-11 M or less, such as 3.0E-11 M or less, such as 2.0E-11 M or less, such as 1.0E-11 M or less, such as 9.0E-12 M or less, such as 8.0E-12 M or less, such as 7.0E-12 M or less, such as 6.0E-12 M or less, such as 5.0E-12 M or less, such as 4.0E-12 M or less, such as 3.0E-12 M or less, such as 2.0E-12 M or less, such as 1.0E-12 M or less.

Suitably the EC50 is established using one of the assays set out in the examples. Suitably one or all of the FAP binding agents have an EC50 as recited for any of the clones specified in the tables of the Examples or less.

Suitably the FAP binding agent binds to FAP specifically, i.e. the binding agent does not bind significantly to targets other than human FAP, such as does not bind significantly to targets other than human, Macaca fascicularis or mouse FAP, such as does not bind significantly to targets other than FAP.

The construct of the invention may bind to FAP with an avidity of 9.0E-10 M or less, such as 8.0E-10, such as 7.2E-10, such as 5.0E-10 M or less, such as 1.0E-10 M or less, such as 9.0E-11 M or less, such as 7.0E-11 M or less, such as 5.0E-11 M or less, such as 1.0E-11 M or less.

Suitably one or all of the FAP binding agents bind to FAP with an affinity (K_{D}) of 400 pM or less, such as 300 pM or less, such as 200 pM or less, such as 150 pM or less, such as 130 pM or less, such as 110 pM or less, such as 100 pM or less, such as 50 pM or less.

Suitably the KD is established using the assay set out in Example 4.

In one embodiment, the construct binds to mouse FAP. In an alternative embodiment, the construct binds to Macaca fascicularis FAP. In an alternative embodiment, the construct binds to human FAP.

In one embodiment one, a plurality, or all of the FAP binding agents comprise or consist of a paired heavy chain variable domain and light chain variable domain which bind to FAP (e.g. a Fab), suitably as defined further below.

In one embodiment the heavy chain variable domain which binds to FAP comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 117, 123, 129, 135, 141, 147 or 153, HCDR2 comprises or consists of a polypeptide sequence sharing 70% or greater, 75% or greater, 80% or greater, 85% or greater, 90% or greater or 95% or greater sequence identity with SEQ ID NO: 118, 124, 130, 136, 142, 148 or 154 and HCDR3 comprises or consists of a polypeptide sequence sharing 70% or greater, 75% or greater, 80% or greater, 85% or greater, 90% or greater or 95% or greater sequence identity with SEQ ID NO: 119, 125, 131, 137, 143, 149 or 155 and the light chain variable domain which binds to FAP comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 120, 126, 132, 138, 144, 150 or 156, LCDR2 comprises or consists of a polypeptide sequence sharing 70% or greater, 75% or greater, 80% or greater, 85% or greater, 90% or greater or 95% or greater sequence identity with SEQ ID NO: 121, 127, 133, 139, 145, 151 or 157 and LCDR3 comprises or consists of a polypeptide sequence sharing 70% or greater, 75% or greater, 80% or greater, 85% or greater, 90% or greater or 95% or greater sequence identity with SEQ ID NO: 122, 128, 134, 140, 146, 152 or 158. More suitably HCDR1 comprises or consists of a polypeptide sequence of SEQ ID NO: 117, 123, 129, 135, 141, 147 or 153, HCDR2 comprises or consists of a polypeptide sequence of SEQ ID NO: 118, 124, 130, 136, 142, 148 or 154 and HCDR3 comprises or consists of a polypeptide sequence of SEQ ID NO: 119, 125, 131, 137, 143, 149 or 155 and LCDR1 comprises or consists of a polypeptide sequence of SEQ ID NO: 120, 126, 132, 138, 144, 150 or 156, LCDR2 comprises or consists of a polypeptide sequence of SEQ ID NO: 121, 127, 133, 139, 145, 151 or 157 and LCDR3 comprises or consists of a polypeptide sequence of SEQ ID NO: 122, 128, 134, 140, 146, 152 or 158.

In one embodiment the heavy chain variable domain which binds to FAP comprises the three heavy chain complementarity determining region amino acid sequences (HCDR1-HCDR3) of the heavy variable domain amino acid sequence of SEQ ID NO: 43, 45, 47, 49, 51, 53 or 55 and the light chain variable domain which binds to FAP comprises the three light chain complementarity determining region amino acid sequences (LCDR1-LCDR3) of the light variable domain amino acid sequence of SEQ ID NO: 44, 46, 48, 50, 52, 54 or 56.

In one embodiment the heavy chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 43, 45, 47, 49, 51, 53 or 55 and the light chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 44, 46, 48, 50, 52, 54 or 56. More suitably the heavy chain variable domain comprises or consists of a polypeptide sequence of SEQ ID NO: 43, 45, 47, 49, 51, 53 or 55 and the light chain variable domain comprises or consists of a polypeptide sequence of SEQ ID NO: 44, 46, 48, 50, 52, 54 or 56.

Preferably the HCDRs and LCDRs are provided in the heavy chain variable domain which binds to FAP and light chain variable domain which binds to FAP in the same combinations as set out in A. For example, it is preferable that (a) HCDRs1-3 of SEQ ID NOs: 117, 118 and 119 and (b) LCDRs1-3 of SEQ ID NOs: 120, 121 and 122 are deployed in combination (as set out for Clone 3.5) e.g. in an Fab. The same principle applies for combinations of variable regions as set out in.

When multiple FAP binding agents are deployed in a construct of the invention, the FAP binding agents may be (a) identical or (b) different FAP binding agents. If different, the FAP binding agents are suitably independently selected from the FAP binding agents described herein, such as those detailed above.

If the construct is an antibody, then in one embodiment one or all of the FAP binding agents are a Fab (linked to the CH2 region).

### Polypeptide and Polynucleotide Sequences

For the purposes of comparing two closely-related polypeptide sequences, the “% sequence identity” between a first polypeptide sequence and a second polypeptide sequence may be calculated using NCBI BLAST v2.0, using standard settings for polypeptide sequences (BLASTP). For the purposes of comparing two closely-related polynucleotide sequences, the “% sequence identity” between a first nucleotide sequence and a second nucleotide sequence may be calculated using NCBI BLAST v2.0, using standard settings for nucleotide sequences (BLASTN).

Polypeptide or polynucleotide sequences are said to be the same as or “identical” to other polypeptide or polynucleotide sequences if they share 100% sequence identity over their entire length. Residues in sequences are numbered from left to right, i.e. from N- to C-terminus for polypeptides; from 5′ to 3′ terminus for polynucleotides.

A “difference” between polypeptide sequences refers to an insertion, deletion or substitution of a single amino acid residue in a position of the second sequence, compared to the first sequence. Two polypeptide sequences can contain one, two or more such amino acid differences. Insertions, deletions or substitutions in a second sequence which is otherwise identical (100% sequence identity) to a first sequence result in reduced % sequence identity. For example, if the identical sequences are 9 amino acid residues long, one substitution in the second sequence results in a sequence identity of 88.9%. If first and second polypeptide sequences are 9 amino acid residues long and share 6 identical residues, the first and second polypeptide sequences share greater than 66% identity (the first and second polypeptide sequences share 66.7% identity).

As used in the clauses and claims herein, numbering of polypeptide sequences and definitions of CDRs and FRs (i.e. HCDR1, HCDR2, HCDR3, HFR1, HFR2, HFR3, HFR4, LCDR1, LCDR2, LCDR3, LFR1, LFR2, LFR3 and LFR4) are as defined according to the Kabat system (Kabat et al., 1991, herein incorporated by reference for the purpose of CDR and FR definitions), unless mentioned otherwise. Alternative to the Kabat system, any of the Chothia-, Martin (enhanced Chothia)-, IMGT (ImMunoGeneTics information system)-, or AbM-numbering schemes can be applied. A “corresponding” amino acid residue between a first and second polypeptide sequence is an amino acid residue in a first sequence which shares the same position according to the Kabat system with an amino acid residue in a second sequence, whilst the amino acid residue in the second sequence may differ in identity from the first. Suitably corresponding residues will share the same number (and letter) if the framework and CDRs are the same length according to Kabat definition (or according to the Chothia-, Martin (enhanced Chothia)-, IMGT (ImMunoGeneTics information system)-, or AbM-definition or delineation). Alignment can be achieved manually or by using, for example, a known computer algorithm for sequence alignment such as NCBI BLAST v2.0 (BLASTP or BLASTN) using standard settings.

Residues recited in the polypeptide sequences listed herein may be unmodified or modified. If modified, they may be for example isomerized, deaminated or oxidated.

### Constructs

Constructs of the invention in at least some embodiments benefit from certain properties such as the ability to cluster LTBR, bind to LTBR and FAP on the same cell, potently agonise LTBR (e.g. LTBR NFKB pathways, such as classical and alternative NFKB pathways), bind to LTBR and FAP with desired affinity, and achieve favourable biodistribution when administered to a subject (e.g. tumour-targeting), due to FAP-dependent conditional activation. The properties specified in respect of the constituent monospecific LTBR binding agents and FAP binding agents may suitably equally apply to the construct of the invention.

Suitably the construct of the invention is conditionally activated at a target site. By conditionally activated it is meant that the construct clusters LTBR at a target site in a FAP-dependent manner (due to the anti-FAP specificity of the construct) such that the construct activates LTBR, resulting in activation of the classical and/or alternative NFKB pathway (as detailed further herein). The target site is suitably a tumour.

Suitably the LTBR binding agent is capable of binding LTBR on a cell and the FAP binding agent is capable of binding FAP on the same cell simultaneously and/or the LTBR binding agent is capable of binding LTBR on a first cell and the FAP binding agent is capable of binding FAP on a second cell simultaneously. Suitably the LTBR binding agent is capable of activating LTBR on cells which express both LTBR and FAP.

A construct according to the invention may comprise multiple binding agents and is therefore multivalent (e.g. bivalent). These multiple binding agents bind to different targets and therefore the construct is multispecific (e.g. bispecific). Such a construct may comprise (a) no more than one LTBR binding agent and no more than one FAP binding agent, (b) no more than one LTBR binding agent and more than one FAP binding agent (c) more than one LTBR binding agent and no more than one FAP binding agent or (d) more than one LTBR binding agent and more than one FAP binding agent. If the construct comprises more than one LTBR binding agent and/or FAP binding agent, then the LTBR binding agents may be the same or different and the FAP binding agents may be the same or different. Suitably the LTBR binding agents are the same. Suitably the FAP binding agents are the same. Most suitably, the LTBR binding agents are the same and the FAP binding agents are the same. If the LTBR binding agents are different, suitably they are independently selected from the LTBR binding agents defined herein. If the FAP binding agents are different, suitably they are independently selected from the FAP binding agents defined herein.

In one embodiment, the construct comprises only one FAP binding agent. Alternatively, the construct comprises more than one FAP binding agent. In one embodiment, the construct comprises only two FAP binding agents.

In one embodiment, the construct comprises only one LTBR binding agent. Alternatively, the construct comprises more than one LTBR binding agent. In one embodiment, the construct comprises only two LTBR binding agents.

The valency and specificity of a construct of the invention may be expressed as a ratio of FAP-binding to LTBR-binding valencies e.g. 1:1, 2:1, etc. A bispecific construct which is monovalent for FAP and monovalent for LTBR may be described as 1:1. A bispecific construct which is bivalent for FAP and monovalent for LTBR may be described as 2:1. A bispecific construct which is bivalent for FAP and bivalent for LTBR may be described as 2:2, and so on.

A FAP-monovalent construct is a construct with a single anti-FAP valency. A FAP-bivalent construct is a construct with two anti-FAP valencies, and so on. An LTBR-monovalent construct is a construct with a single anti-LTBR valency. An LTBR-bivalent construct is a construct with two anti-LTBR valencies, and so on.

In one embodiment the construct comprises or consists of an antibody, such as a bispecific antibody and/or bivalent antibody.

A construct according to the invention may comprise at least one further binding agent (such as an antibody or antigen-binding fragment thereof), which is neither an LTBR binding agent nor a FAP binding agent. Alternatively, a construct according to the invention may comprise no further binding agents.

A suitable construct may comprise or consist of an antibody, wherein in one embodiment the first variable region binds to LTBR and the second variable region binds to FAP.

A further suitable construct may comprise multiple antibodies.

Other suitable constructs may comprise or consist of one or a plurality of antibody fragments as described above, wherein each antibody fragment may contribute one or more binding domains. Suitable antibody fragments include for example an scFv, Fv, Fab, Fab′, F(ab′)2, variable domain (e.g. VH, VL, VNAR or VHH), diabody or minibody. In one embodiment, the construct comprises or consists of two scFvs.

The binding agents can be linked (or ‘fused’) to each other directly (i.e. without use of a linker) or indirectly via a linker or via further components such as immunoglobulin domains. The binding agents can be linked (or ‘fused’) to components of the construct, such as constant domains, directly (i.e. without use of a linker) or indirectly via a linker. Suitably, the linker is a peptide. Suitably the linker is selected so as to allow binding of the binding agents to their targets, while maintaining structural integrity. If intended for administration to a subject, the linker is suitably non-immunogenic in the subject to which the binding agents are administered.

Suitably the peptide linker is no longer than 50 amino acids, such as no longer than 40 amino acids, such as no longer than 30 amino acids, such as no longer than 20 amino acids, such as no longer than 10 amino acids, such as no longer than 9 amino acids, such as no longer than 8 amino acids, such as no longer than 7 amino acids, such as no longer than 6 amino acids, such as no longer than 5 amino acids.

Suitably the peptide linker is longer than 6 amino acids, such as longer than 7 amino acids, such as longer than 8 amino acids, such as longer than 9 amino acids, such as longer than 10 amino acids, such as longer than 20 amino acids, such as longer than 30 amino acids, such as longer than 40 amino acids, such as longer than 50 amino acids.

Suitably the peptide linker comprises or consists of glycine and serine residues, such as comprising or consisting of a G₄S linker (SEQ ID NO: 3).

In one embodiment the binding agents are linked to each other in a construct in the form of a bispecific, multivalent (e.g. bivalent) antibody. A bispecific antibody may be referred to as a “bsAb”.

A particularly suitable bispecific antibody format is that generated via the knobs-into-holes approach (Merchant et al. 1998). Alternatively, or in addition, one or more (most suitably, all) Golay mutations in the CH1/CL regions may be introduced to obtain efficient light chain pairing (Golay et al. 2016).

In one embodiment, bispecific constructs of the invention having more than one valency for LTBR and/or FAP may comprise multiple scFvs, wherein each scFv is fused to the C-terminal of an Fc by a peptide linker, such as a linker comprising glycine and serine residues, such as a G₄S linker (SEQ ID NO: 3).

If an Fc is present in the construct (e.g. if the construct is an antibody), then suitably the LTBR binding agent is linked directly or indirectly to the Fc (e.g. to one of the CH3 domains). If two LTBR binding agents are present, each LTBR binding agent may be linked to one of the CH3 domains of the Fc, i.e. the first LTBR binding agent is linked to the first CH3 domain and the second LTBR binding agent is linked to the second CH3 domain.

In one embodiment the linker does not comprise an Ig domain. In one embodiment, the linker is a linear peptide. In one embodiment, the linker is a hinge region. Most suitably, the LTBR binding agent is linked indirectly to the CH3 domain by a linear peptide linker.

A number of constructs (including bispecific constructs) described in the examples below are referred to as ‘clones’ and are assigned specific clone numbers or IDs.

### Cancer

In one embodiment the construct of the invention is for use in the treatment of cancer. In one embodiment, the cancer may be a tumour. In one embodiment, the cancer may be a non-haematological cancer. In this context, the LTBR binding agent is most suitably an LTBR agonist (as discussed in more detail above).

### Administration

The constructs and polypeptides of the invention are typically intended for use with mammalian subjects, in particular human subjects. The constructs and polypeptides will typically be administered to a subject in need thereof, in particular a mammalian subject in need thereof, in particular a human subject in need thereof.

The construct or polypeptide may be administered by any suitable route, which may depend on the nature of the specific agents. Exemplary routes include oral, parenteral, buccal, sublingual, nasal or rectal administration. Suitably, the construct is administered parenterally, such as intravenously (i.v. or IV) or intraperitoneally (i.p. or IP).

The cancer vaccine and/or adjuvant, if present, may be administered by any suitable route, which may depend on the nature of the specific agents. Exemplary routes include oral, parenteral, buccal, sublingual, nasal or rectal administration. Suitably, the vaccine and/or adjuvant are administered parenterally, such as interperitoneally.

The construct or polypeptide may be provided in the form of a pharmaceutical composition comprising the construct or polypeptide and a pharmaceutically acceptable carrier or excipient.

If delivered orally, the construct or polypeptide may suitably be delivered in a solid pharmaceutical composition (such as a tablet, capsule or lozenge) or in a liquid pharmaceutical composition (such as a suspension, emulsion or solution).

A liquid formulation will generally consist of a suspension or solution of the construct in a suitable liquid carrier e.g. an aqueous solvent such as water, ethanol or glycerine, or a non-aqueous solvent, such as polyethylene glycol or an oil. The formulation may also contain a suspending agent, preservative, flavouring and/or colouring agent.

A tablet formulation can be prepared using any suitable pharmaceutical carrier(s) routinely used for preparing solid formulations, such as magnesium stearate, starch, lactose, sucrose and cellulose.

Suitably, the pharmaceutical composition is in unit dose form, such as a tablet, capsule, vial or prefilled syringe. Suitably the unit dose form is for intravenous administration.

The pharmaceutical composition may for example contain from 0.1% to 99.99% by weight, for example from 10 to 60% by weight, of the active material, depending on the method of administration. The pharmaceutical composition may contain from 0.01% to 99% by weight, for example 40% to 90% by weight, of the carrier, depending on the method of administration. The pharmaceutical composition may contain from 0.05 mg to 2000 mg of the active material, for example from 1.0 mg to 500 mg, depending on the method of administration. The pharmaceutical composition may contain from 50 mg to 1000 mg of the carrier, for example from 100 mg to 400 mg, depending on the method of administration.

The dose of the pharmaceutical composition used will vary in the usual way with the seriousness of the cancer, the weight of the sufferer, and other similar factors. However, as a general guide, suitable unit doses may be 0.05 mg to 1000 mg, more suitably 1.0 mg to 500 mg, and such unit doses may be administered more than once a day, for example two or three a day. Such therapy may extend for a number of weeks, months or longer.

The dose provided to a subject will typically be a safe and effective dose, i.e. an amount providing an acceptable balance of desired benefits and undesired side effects. A “safe and effective amount” is intended to include an amount of a compound that is effective to achieve a desirable effect in treatment of a disease-state. A desirable effect is typically clinically significant and/or measurable, for instance in the context of (a) inhibiting the disease-state, i.e., slowing or arresting its development; and/or (b) relieving the disease-state, i.e., causing regression of the disease state or a reduction in associated symptoms. The safe and effective amount is one that is sufficient to achieve the desirable effect.

For avoidance of doubt, a “safe and effective amount” as recited herein can be achieved by any suitable dosage regimen. Hence, for example, references herein to administering a safe and effective amount of a composition, such as by a particular administration route, include achieving the safe and effective amount via a single dose or by plural doses, such as administered by the specified administration route. For instance, intravenously administering a safe and effective amount includes both intravenously administering a single dose and intravenously administering any plural number of doses, provided that a safe and effective amount is thereby achieved by intravenous administration.

### Polynucleotides, Vectors and Cells

In one embodiment there is provided a set of one or more polynucleotides encoding the construct of the invention. Also provided is a set of one or more expression vectors collectively comprising these polynucleotides. Also provided is a cell comprising the expression vectors or polynucleotides. Further is provided a method of producing a construct of the invention, the method comprising culturing the cell under suitable conditions such that the polynucleotide is expressed, and the construct is produced.

### Combinations

In certain embodiments, the construct or polypeptide of the invention may be used in combination with further agents. For example, in one embodiment there is provided a composition comprising a construct or polypeptide of the invention and a checkpoint inhibitor (such as an anti-CTLA-4 antibody, an anti-PD-L1 antibody and/or an anti-PD-1 antibody). Suitably these compositions may be for use in the treatment of cancer (e.g. a tumour).

The constructs, compositions or polypeptides of the invention may be for use in the treatment of cancer (e.g. a tumour), optionally in combination with radiotherapy (e.g. hypo-fractionated radiotherapy), chemotherapy or antibody-drug conjugates (ADCs). The construct of the invention may be for use in the treatment of cancer with one of the above agents, or one of the above agents may be for use in the treatment of cancer with a construct of the invention. Administration of (a) the construct or polypeptide of the invention and (b) the checkpoint inhibitor and/or radiotherapy and/or chemotherapy and/or ADCs may be sequential, separate or simultaneous.

### FURTHER SPECIFIC EMBODIMENTS

Specific constructs of the invention are characterised in the examples.

In one embodiment the construct of the invention comprises, essentially consists of or consists of a format as described in for clone 4.1, 4.2, 4.3, 4.4, 4.5, 4.6, 4.7, 4.8, 4.9, 4.10, 4.11 or 4.12.

The construct of the invention may comprise, essentially consist of or consist of a format as depicted in FIGS. 7a-7d. The construct of the invention may comprise or consist of of the polypeptide sequence(s) of clone 4.1, 4.2, 4.3, 4.4, 4.5, 4.6, 4.7, 4.8, 4.9, 4.10, 4.11 or 4.12.

The construct of the invention may in some embodiments be provided in the following particular formats.

The construct may comprise, essentially consist or consist of a bispecific, FAP-monovalent, LTBR-monovalent antibody comprising a heterodimer of two polypeptide chains, the first chain comprising or consisting of from N to C terminus a Fab region, a hinge region, a CH2 domain and a CH3 domain; and the second chain comprising or consisting of from N to C terminus a VHH, a hinge region and a CH2 domain and a CH3 domain, wherein the hinge regions are linked via a disulphide bond, the CH2 and CH3 domains are associated to form an Fc region, the Fab region comprises a paired VH and VL at its N-terminus which form a FAP binding site and the VHH forms an LTBR binding site (e.g. essentially as set out in FIG. 7a). This is one embodiment of a 1:1 format construct.

The construct may comprise, essentially consist or consist of a first heavy chain, a light chain and a second heavy chain, wherein the first heavy chain comprises from N to C terminus a VH, a CH1, a CH2 and a CH3, the second heavy chain comprises from N to C terminus a VHH, a CH2 and a CH3, the light chain comprises from N to C terminus a VL and a CL and associates with the VH and CH1 of the first heavy chain to form a Fab region and the CH2 and CH3 domains of the first and second heavy chains associate to form an Fc region, wherein the VH and VL bind to FAP and the VHH binds to LTBR (e.g. essentially as set out in FIG. 7a). This is one embodiment of a 1:1 format construct.

The construct may comprise, essentially consist or consist of a bispecific, FAP-bivalent, LTBR-monovalent antibody comprising a heterodimer of two polypeptide chains, the first chain comprising or consisting of from N to C terminus a Fab region, a hinge region, a CH2 domain, a CH3 domain and a VHH; and the second chain comprising or consisting of from N to C terminus a Fab region, a hinge region, a CH2 domain and a CH3 domain, wherein the hinge regions are linked via a disulphide bond, the CH2 and CH3 domains are associated to form an Fc region, the Fab regions each comprise a paired VH and VL at their N-terminus which each form a FAP binding site and the VHH forms an LTBR binding site at the C-terminus (e.g. essentially as set out in FIG. 7b). This is one embodiment of a 2:1 format construct.

The construct may comprise, essentially consist of a first heavy chain, a first light chain, a second heavy chain and a second light chain wherein the first heavy chain comprises from N to C terminus a VH, a CH1, a CH2, a CH3 and a VHH, the second heavy chain comprises from N to C terminus a VH, a CH1, a CH2 and a CH3, the first light chain comprises from N to C terminus a VL and a CL and associates with the VH and CH1 of the first heavy chain to form a Fab region, the second light chain comprises from N to C terminus a VL and a CL and associates with the VH and CH1 of the second heavy chain to form a Fab region and the CH2 and CH3 domains of the first and second heavy chains associate to form an Fc region, wherein the VH and VL of each Fab region bind to FAP and the VHH binds to LTBR (e.g. essentially as set out in FIG. 7b). This is one embodiment of a 2:1 format construct.

The construct may comprise, essentially consist or consist of a bispecific, FAP-bivalent, LTBR-bivalent antibody comprising a homodimer of two polypeptide chains, the first chain comprising or consisting of from N to C terminus a Fab region, a hinge region, a CH2 domain, a CH3 domain and a VHH; and the second chain comprising or consisting of from N to C terminus a Fab region, a hinge region, a CH2 domain, a CH3 domain and a VHH wherein the hinge regions are linked via a disulphide bond, the CH2 and CH3 domains are associated to form an Fc region, the Fab regions each comprise a paired VH and VL at their N-terminus which each form a FAP binding site and the VHHs each form an LTBR binding site at the C-terminus (e.g. essentially as set out in FIG. 7c). This is one embodiment of a 2:2 format construct.

The construct may comprise, essentially consist or consist of a first heavy chain, a first light chain, a second heavy chain and a second light chain wherein the first heavy chain comprises from N to C terminus a VH, a CH1, a CH2, a CH3 and a VHH, the second heavy chain comprises from N to C terminus a VH, a CH1, a CH2, a CH3 and a VHH, the first light chain comprises from N to C terminus a VL and a CL and associates with the VH and CH1 of the first heavy chain to form a Fab region, the second light chain comprises from N to C terminus a VL and a CL and associates with the VH and CH1 of the second heavy chain to form a Fab region and the CH2 and CH3 domains of the first and second heavy chains associate to form an Fc region, wherein the VH and VL of each Fab region bind to FAP and the VHHs bind to LTBR (e.g. essentially as set out in FIG. 7c). This is one embodiment of a 2:2 format construct.

The construct may comprise, essentially consist or consist of a bispecific, FAP-bivalent, LTBR-bivalent antibody comprising a homodimer of two polypeptide chains, the first chain comprising or consisting of from N to C terminus a Fab region, a hinge region, a CH2 domain, a CH3 domain and an scFv; and the second chain comprising or consisting of from N to C terminus a Fab region, a hinge region, a CH2 domain, a CH3 domain and an scFv wherein the hinge regions are linked via a disulphide bond, the CH2 and CH3 domains are associated to form an Fc region, the Fab regions each comprise a paired VH and VL at their N-terminus which each form a FAP binding site and the scFvs each form an LTBR binding site at the C-terminus (e.g. essentially as set out in FIG. 7d). This is one embodiment of a 2:2 format construct.

The construct may comprise, essentially consist or consist of a first heavy chain, a first light chain, a second heavy chain and a second light chain wherein the first heavy chain comprises from N to C terminus a VH, a CH1, a CH2, a CH3 and a paired VH and VL, the second heavy chain comprises from N to C terminus a VH, a CH1, a CH2, a CH3 and a paired VH and VL, the first light chain comprises from N to C terminus a VL and a CL and associates with the VH and CH1 of the first heavy chain to form a Fab region, the second light chain comprises from N to C terminus a VL and a CL and associates with the VH and CH1 of the second heavy chain to form a Fab region and the CH2 and CH3 domains of the first and second heavy chains associate to form an Fc region, wherein the VH and VL of each Fab region bind to FAP and the paired VH and VLs bind to LTBR (e.g. essentially as set out in FIG. 7d). This is one embodiment of a 2:2 format construct.

Also provided are (a) an antibody which binds to the epitopes to which the polypeptide or construct of the invention binds, (b) an antibody which competes for binding to LTBR with the construct of the invention and (c) an antibody which competes for binding to FAP with the construct of the invention.

Also provided is an embodiment wherein the construct is an antibody comprising
(a) one or more Fab domains, each comprising a paired heavy chain variable domain and light chain variable domain which bind to FAP; wherein
   (i) the heavy chain variable domain comprises or consists of a polypeptide sequence of SEQ ID NO: 43 and the light chain variable domain comprises or consists of a polypeptide sequence of SEQ ID NO: 44;
   (ii) the heavy chain variable domain comprises or consists of a polypeptide sequence of SEQ ID NO: 45 and the light chain variable domain comprises or consists of a polypeptide sequence of SEQ ID NO: 46; or
   (iii) the heavy chain variable domain comprises or consists of a polypeptide sequence of SEQ ID NO: 47 and the light chain variable domain comprises or consists of a polypeptide sequence of SEQ ID NO: 48,
and
(b) one or more paired heavy chain variable domain and light chain variable domain which bind to LTBR;
   wherein the heavy chain variable domain comprises or consists of a polypeptide sequence of SEQ ID NO: 31 and the light chain variable domain comprises or consists of a polypeptide sequence of SEQ ID NO: 32
or one or more variable domains which bind to LTBR; wherein
   (i) wherein the heavy chain variable domain comprises or consists of a polypeptide sequence of SEQ ID NO: 24; or
   (ii) wherein the heavy chain variable domain comprises or consists of a polypeptide sequence of SEQ ID NO: 25.

In a further embodiment the construct is an antibody comprising:
(i) a heterodimer of two different heavy chains (HC1 and HC2) and two identical light chains (LC) wherein:
   (a) the HC1 polypeptide sequence comprises or consists of SEQ ID NO: 175, the HC2 polypeptide sequence comprises or consists of SEQ ID NO: 176 and the LC polypeptide sequence comprises or consists of SEQ ID NO: 177;
   (b) the HC1 polypeptide sequence comprises or consists of SEQ ID NO: 178, the HC2 polypeptide sequence comprises or consists of SEQ ID NO: 179 and the LC polypeptide sequence comprises or consists of SEQ ID NO: 180;
   (c) the HC1 polypeptide sequence comprises or consists of SEQ ID NO: 181, the HC2 polypeptide sequence comprises or consists of SEQ ID NO: 182 and the LC polypeptide sequence comprises or consists of SEQ ID NO: 183;
   (d) the HC1 polypeptide sequence comprises or consists of SEQ ID NO: 184, the HC2 polypeptide sequence comprises or consists of SEQ ID NO: 185 and the LC polypeptide sequence comprises or consists of SEQ ID NO: 186;
   (e) the HC1 polypeptide sequence comprises or consists of SEQ ID NO: 187, the HC2 polypeptide sequence comprises or consists of SEQ ID NO: 188 and the LC polypeptide sequence comprises or consists of SEQ ID NO: 189;
   (f) the HC1 polypeptide sequence comprises or consists of SEQ ID NO: 190, the HC2 polypeptide sequence comprises or consists of SEQ ID NO: 191 and the LC polypeptide sequence comprises or consists of SEQ ID NO: 192;
   (g) the HC1 polypeptide sequence comprises or consists of SEQ ID NO: 193, the HC2 polypeptide sequence comprises or consists of SEQ ID NO: 194 and the LC polypeptide sequence comprises or consists of SEQ ID NO: 195; or
   (h) the HC1 polypeptide sequence comprises or consists of SEQ ID NO: 200, the HC2 polypeptide sequence comprises or consists of SEQ ID NO: 201 and the LC polypeptide sequence comprises or consists of SEQ ID NO: 202 or
(ii) a homodimer of two identical heavy chains (HC) and two identical light chains (LC) wherein:
   (a) the HC polypeptide sequence comprises or consists of SEQ ID NO: 196 and the LC polypeptide sequence comprises or consists of SEQ ID NO: 197 or
   (b) the HC polypeptide sequence comprises or consists of SEQ ID NO: 198 and the LC polypeptide sequence comprises or consists of SEQ ID NO: 199.

Also provided is a construct comprising an antibody or antibody fragment comprising a FAP binding domain and an LTBR binding domain, wherein
(i) the LTBR binding domain is a variable domain which binds to LTBR which comprises or consists of three heavy chain complementarity determining regions (HCDR1-HCDR3) and four framework regions (FR1-FR4), wherein:
(a) HCDR1 comprises or consists of a polypeptide sequence of SEQ ID NO: 60 or 172, HCDR2 comprises or consists of a polypeptide sequence of SEQ ID NO: 61 or 173 and HCDR3 comprises or consists of a polypeptide sequence of SEQ ID NO: 62 or 174 or
(b) HCDR1 comprises or consists of a polypeptide sequence of SEQ ID NO: 57 or 166, HCDR2 comprises or consists of a polypeptide sequence of SEQ ID NO: 58 or 167 and HCDR3 comprises or consists of a polypeptide sequence of SEQ ID NO: 59 or 168 or
(c) HCDR1 comprises or consists of a polypeptide sequence of SEQ ID NO: 63 or 163, HCDR2 comprises or consists of a polypeptide sequence of SEQ ID NO: 64 or 164 and HCDR3 comprises or consists of a polypeptide sequence of SEQ ID NO: 65 or 165 or
(d) HCDR1 comprises or consists of a polypeptide sequence of SEQ ID NO: 66 or 169, HCDR2 comprises or consists of a polypeptide sequence of SEQ ID NO: 67 or 170 and HCDR3 comprises or consists of a polypeptide sequence of SEQ ID NO: 68 or 171;
or
(ii) the LTBR binding domain is a paired heavy chain variable domain and light chain variable domain wherein the heavy chain variable domain comprises or consists of three complementarity determining regions (HCDR1-HCDR3) and four framework regions (HFR1-HFR4) and the light chain variable domain comprises or consists of three complementarity determining regions (LCDR1-LCDR3) and four framework regions (LFR1-LFR4), wherein:
(a) HCDR1 comprises or consists of a polypeptide sequence of SEQ ID NO: 69, HCDR2 comprises or consists of a polypeptide sequence of SEQ ID NO: 70 and HCDR3 comprises or consists of a polypeptide sequence of SEQ ID NO: 71 and wherein LCDR1 comprises or consists of a polypeptide sequence of SEQ ID NO: 72, LCDR2 comprises or consists of a polypeptide sequence of SEQ ID NO: 73 and LCDR3 comprises or consists of a polypeptide sequence of SEQ ID NO: 74 or
(b) HCDR1 comprises or consists of a polypeptide sequence of SEQ ID NO: 75, HCDR2 comprises or consists of a polypeptide sequence of SEQ ID NO: 76 and HCDR3 comprises or consists of a polypeptide sequence of SEQ ID NO: 77 and wherein LCDR1 comprises or consists of a polypeptide sequence of SEQ ID NO: 78, LCDR2 comprises or consists of a polypeptide sequence of SEQ ID NO: 79 and LCDR3 comprises or consists of a polypeptide sequence of SEQ ID NO: 80 or
(c) HCDR1 comprises or consists of a polypeptide sequence of SEQ ID NO: 81, HCDR2 comprises or consists of a polypeptide sequence of SEQ ID NO: 82 and HCDR3 comprises or consists of a polypeptide sequence of SEQ ID NO: 83 and wherein LCDR1 comprises or consists of a polypeptide sequence of SEQ ID NO: 84, LCDR2 comprises or consists of a polypeptide sequence of SEQ ID NO: 85 and LCDR3 comprises or consists of a polypeptide sequence of SEQ ID NO: 86 or
(d) HCDR1 comprises or consists of a polypeptide sequence of SEQ ID NO: 87, HCDR2 comprises or consists of a polypeptide sequence of SEQ ID NO: 88 and HCDR3 comprises or consists of a polypeptide sequence of SEQ ID NO: 89 and wherein LCDR1 comprises or consists of a polypeptide sequence of SEQ ID NO: 90, LCDR2 comprises or consists of a polypeptide sequence of SEQ ID NO: 91 and LCDR3 comprises or consists of a polypeptide sequence of SEQ ID NO: 92 or
(e) HCDR1 comprises or consists of a polypeptide sequence of SEQ ID NO: 93, HCDR2 comprises or consists of a polypeptide sequence of SEQ ID NO: 94 and HCDR3 comprises or consists of a polypeptide sequence of SEQ ID NO: 95 and wherein LCDR1 comprises or consists of a polypeptide sequence of SEQ ID NO: 96, LCDR2 comprises or consists of a polypeptide sequence of SEQ ID NO: 97 and LCDR3 comprises or consists of a polypeptide sequence of SEQ ID NO: 98 or
(f) HCDR1 comprises or consists of a polypeptide sequence of SEQ ID NO: 99, HCDR2 comprises or consists of a polypeptide sequence of SEQ ID NO: 100 and HCDR3 comprises or consists of a polypeptide sequence of SEQ ID NO: 101 and wherein LCDR1 comprises or consists of a polypeptide sequence of SEQ ID NO: 102, LCDR2 comprises or consists of a polypeptide sequence of SEQ ID NO: 103 and LCDR3 comprises or consists of a polypeptide sequence of SEQ ID NO: 104 or
(g) HCDR1 comprises or consists of a polypeptide sequence of SEQ ID NO: 105, HCDR2 comprises or consists of a polypeptide sequence of SEQ ID NO: 106 and HCDR3 comprises or consists of a polypeptide sequence of SEQ ID NO: 107 and wherein LCDR1 comprises or consists of a polypeptide sequence of SEQ ID NO: 108, LCDR2 comprises or consists of a polypeptide sequence of SEQ ID NO: 109 and LCDR3 comprises or consists of a polypeptide sequence of SEQ ID NO: 110 or
(h) HCDR1 comprises or consists of a polypeptide sequence of SEQ ID NO: 111, HCDR2 comprises or consists of a polypeptide sequence of SEQ ID NO: 112 and HCDR3 comprises or consists of a polypeptide sequence of SEQ ID NO: 113 and wherein LCDR1 comprises or consists of a polypeptide sequence of SEQ ID NO: 114, LCDR2 comprises or consists of a polypeptide sequence of SEQ ID NO: 115 and LCDR3 comprises or consists of a polypeptide sequence of SEQ ID NO: 116;
and
the FAP binding domain is a paired heavy chain variable domain and light chain variable domain wherein the heavy chain variable domain comprises or consists of three complementarity determining regions (HCDR1-HCDR3) and four framework regions (HFR1-HFR4) and the light chain variable domain comprises or consists of three complementarity determining regions (LCDR1-LCDR3) and four framework regions (LFR1-LFR4), wherein:
(a) HCDR1 comprises or consists of a polypeptide sequence of SEQ ID NO: 117, HCDR2 comprises or consists of a polypeptide sequence of SEQ ID NO: 118 and HCDR3 comprises or consists of a polypeptide sequence of SEQ ID NO: 119 and wherein LCDR1 comprises or consists of a polypeptide sequence of SEQ ID NO: 120, LCDR2 comprises or consists of a polypeptide sequence of SEQ ID NO: 121 and LCDR3 comprises or consists of a polypeptide sequence of SEQ ID NO: 122 or
(b) HCDR1 comprises or consists of a polypeptide sequence of SEQ ID NO: 123, HCDR2 comprises or consists of a polypeptide sequence of SEQ ID NO: 124 and HCDR3 comprises or consists of a polypeptide sequence of SEQ ID NO: 125 and wherein LCDR1 comprises or consists of a polypeptide sequence of SEQ ID NO: 126, LCDR2 comprises or consists of a polypeptide sequence of SEQ ID NO: 127 and LCDR3 comprises or consists of a polypeptide sequence of SEQ ID NO: 128 or
(c) HCDR1 comprises or consists of a polypeptide sequence of SEQ ID NO: 129, HCDR2 comprises or consists of a polypeptide sequence of SEQ ID NO: 130 and HCDR3 comprises or consists of a polypeptide sequence of SEQ ID NO: 131 and wherein LCDR1 comprises or consists of a polypeptide sequence of SEQ ID NO: 132, LCDR2 comprises or consists of a polypeptide sequence of SEQ ID NO: 133 and LCDR3 comprises or consists of a polypeptide sequence of SEQ ID NO: 134 or
(d) HCDR1 comprises or consists of a polypeptide sequence of SEQ ID NO: 135, HCDR2 comprises or consists of a polypeptide sequence of SEQ ID NO: 136 and HCDR3 comprises or consists of a polypeptide sequence of SEQ ID NO: 137 and wherein LCDR1 comprises or consists of a polypeptide sequence of SEQ ID NO: 138, LCDR2 comprises or consists of a polypeptide sequence of SEQ ID NO: 139 and LCDR3 comprises or consists of a polypeptide sequence of SEQ ID NO: 140 or
(e) HCDR1 comprises or consists of a polypeptide sequence of SEQ ID NO: 141, HCDR2 comprises or consists of a polypeptide sequence of SEQ ID NO: 142 and HCDR3 comprises or consists of a polypeptide sequence of SEQ ID NO: 143 and wherein LCDR1 comprises or consists of a polypeptide sequence of SEQ ID NO: 144, LCDR2 comprises or consists of a polypeptide sequence of SEQ ID NO: 145 and LCDR3 comprises or consists of a polypeptide sequence of SEQ ID NO: 146 or
(f) HCDR1 comprises or consists of a polypeptide sequence of SEQ ID NO: 147, HCDR2 comprises or consists of a polypeptide sequence of SEQ ID NO: 148 and HCDR3 comprises or consists of a polypeptide sequence of SEQ ID NO: 149 and wherein LCDR1 comprises or consists of a polypeptide sequence of SEQ ID NO: 150, LCDR2 comprises or consists of a polypeptide sequence of SEQ ID NO: 151 and LCDR3 comprises or consists of a polypeptide sequence of SEQ ID NO: 152 or
(g) HCDR1 comprises or consists of a polypeptide sequence of SEQ ID NO: 153, HCDR2 comprises or consists of a polypeptide sequence of SEQ ID NO: 154 and HCDR3 comprises or consists of a polypeptide sequence of SEQ ID NO: 155 and wherein LCDR1 comprises or consists of a polypeptide sequence of SEQ ID NO: 156, LCDR2 comprises or consists of a polypeptide sequence of SEQ ID NO: 157 and LCDR3 comprises or consists of a polypeptide sequence of SEQ ID NO: 158.

### Polypeptides Binding to Either LTBR or FAP

In addition to constructs comprising a FAP binding agent and an LTBR binding agent, the invention provides (a) constructs which bind to FAP and (b) constructs which bind to LTBR. Such constructs may, for example, find utility in the bispecific constructs of the invention. Monospecific polypeptides (“clones”) used in the examples of the invention are summarised as follows.

| Clone ID | Target | Format |
|---|---|---|
| 1.1 (parental | LTBR | VHH (monovalent) |
| clone for 1.7) | | |
| 1.2 (parental | LTBR | VHH (monovalent) |
| clone for 1.5) | | |
| 1.3 (parental | LTBR | VHH (monovalent) |
| clone for 1.8) | | |
| 1.4 (parental | LTBR | VHH (monovalent) |
| clone for 1.6) | | |
| 1.5 | LTBR | VHH (monovalent) |
| 1.6 | LTBR | VHH (monovalent) |
| 1.7 | LTBR | VHH (monovalent) |
| 1.8 | LTBR | VHH (monovalent) |
| 1.9 | LTBR | VHH-Fc (bivalent) |
| 1.10 | LTBR | VHH-Fc (bivalent) |
| 1.11 | LTBR | VHH-Fc (bivalent) |
| 1.12 | LTBR | VHH-Fc (bivalent) |
| 2.1 | LTBR | mAb (bivalent) |
| 2.2 | LTBR | mAb (bivalent) |
| 2.3 | LTBR | mAb (bivalent) |
| 2.4 | LTBR | mAb (bivalent) |
| 2.5 | LTBR | mAb (bivalent) |
| 2.6 | LTBR | mAb (bivalent) |
| 2.7 | LTBR | mAb (bivalent) |
| 2.8 | LTBR | mAb (bivalent) |
| 3.1 | FAP | mAb (bivalent) |
| 3.2 | FAP | mAb (bivalent) |
| 3.3 | FAP | mAb (bivalent) |
| 3.4 | FAP | mAb (bivalent) |
| 3.5 | FAP | mAb (bivalent) |
| 3.6 | FAP | mAb (bivalent) |
| 3.7 | FAP | mAb (bivalent) |

### Polypeptides Binding to LTBR

The LTBR binding polypeptide may comprise or consist of a variable domain which binds to LTBR (e.g. a heavy chain variable domain, such as a VHH), suitably as defined further below.

In one embodiment, the LTBR-binding polypeptide comprises at least one CDR, wherein the CDR is a HCDR1, HCDR2 or HCDR3 comprised in a heavy chain variable domain of SEQ ID NOs: 23-26 or 159-162. Suitably, such polypeptide comprises at least such HCDR1 and HCDR2, such HCDR1 and HCDR3, or such HCDR2 and HCDR3. More suitably, such polypeptide comprises such HCDR1, HCDR2 and HCDR3. More suitably, the HCDR1, HCDR2 and HCDR3 are delineated according to the Kabat-, Chothia-, Martin-, IMGT-, or AbM-method. More suitably the HCDR1 is chosen from SEQ ID NOs: 57, 60, 63, 66, 163, 166, 169 or 172 as defined by the Kabat-method; the HCDR2 is chosen from SEQ ID NOs: 58, 61, 64, 67, 164, 167, 170, 173, 205 or 206 as defined by the Kabat-method; and/or the HCDR3 is chosen from SEQ ID NOs: 59, 62, 65, 68, 165, 168, 171, 174, 207 or 208 as defined by the Kabat-method.

In one embodiment the variable domain which binds to LTBR comprises or consists of three heavy chain complementarity determining regions (HCDR1-HCDR3) and four framework regions (FR1-FR4), wherein HCDR1 comprises or consists of a polypeptide sequence sharing 70% or greater, 75% or greater, 80% or greater, 85% or greater, 90% or greater or 95% or greater sequence identity with SEQ ID NO: 57, 60, 63, 66, 163, 166, 169 or 172 (e.g. 57, 60, 63 or 66) or wherein HCDR1 comprises or consists of a polypeptide sequence having 3, 2 or 1 amino acid sequence difference with SEQ ID NO: 57, 60, 63, 66, 163, 166, 169 or 172 (e.g. 57, 60, 63 or 66); HCDR2 comprises or consists of a polypeptide sequence sharing 70% or greater, 75% or greater, 80% or greater, 85% or greater, 90% or greater or 95% or greater sequence identity with SEQ ID NO: 58, 61, 64, 67, 164, 167, 170, 173, 205 or 206 (e.g. 58, 61, 64 or 67) or wherein HCDR2 comprises or consists of a polypeptide sequence having 3, 2 or 1 amino acid sequence difference with SEQ ID NO: 58, 61, 64, 67, 164, 167, 170, 173, 205 or 206 (e.g. 58, 61, 64 or 67); and HCDR3 comprises or consists of a polypeptide sequence sharing 70% or greater, 75% or greater, 80% or greater, 85% or greater, 90% or greater or 95% or greater sequence identity with SEQ ID NO: 59, 62, 65, 68, 165, 168, 171, 174, 207 or 208 (e.g. 59, 62, 65 or 68) or wherein HCDR3 comprises or consists of a polypeptide sequence having 3, 2 or 1 amino acid sequence difference with SEQ ID NO: 59, 62, 65, 68, 165, 168, 171, 174, 207 or 208 (e.g. 59, 62, 65 or 68). More suitably HCDR1 comprises or consists of a polypeptide sequence of SEQ ID NO: 57, 60, 63, 66, 163, 166, 169 or 172 (e.g. 57, 60, 63 or 66), HCDR2 comprises or consists of a polypeptide sequence of SEQ ID NO: 58, 61, 64, 67, 164, 167, 170, 173, 205 or 206 (e.g. 58, 61, 64 or 67) and HCDR3 comprises or consists of a polypeptide sequence of SEQ ID NO: 59, 62, 65, 68, 165, 168, 171, 174, 207 or 208 (e.g. 59, 62, 65 or 68). In particular, these CDR polypeptide sequences were determined or delineated according to or with the Kabat-method.

In one embodiment the variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 23, 24, 25, 26, 159, 160, 161 or 162 (e.g. 23, 24, 25 or 26). More suitably the variable domain which binds to LTBR comprises or consists of a polypeptide sequence of SEQ ID NO: 23, 24, 25, 26, 159, 160, 161 or 162 (e.g. 23, 24, 25 or 26). In one further embodiment thereto, the polypeptide sequence variation is in one or more the framework regions FR1-FR4 comprised in SEQ ID NO: 23, 24, 25, 26, 159, 160, 161 or 162 (e.g. 23, 24, 25 or 26). Alternatively, the polypeptide sequence variation is one or more of the framework regions FR1-FR4 comprised in SEQ ID NO: 23, 24, 25, 26, 159, 160, 161 or 162 (e.g. 23, 24, 25 or 26) and the CDR polypeptide sequences comprised within SEQ ID NO: 23, 24, 25, 26, 159, 160, 161 or 162 (e.g. 23, 24, 25 or 26) are the HCDR1-HCDR3 polypeptide sequences as outlined above.

In one embodiment the LTBR binding polypeptide comprises or consists of a paired heavy chain variable domain and light chain variable domain which bind to LTBR (e.g. an scFv), suitably as defined further below.

In one embodiment the heavy chain variable domain which binds to LTBR comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 69, 75, 81, 87, 93, 99, 105 or 111, HCDR2 comprises or consists of a polypeptide sequence sharing 70% or greater, 75% or greater, 80% or greater, 85% or greater, 90% or greater or 95% or greater sequence identity with SEQ ID NO: 70, 76, 82, 88, 94, 100, 106 or 112 and HCDR3 comprises or consists of a polypeptide sequence sharing 70% or greater, 75% or greater, 80% or greater, 85% or greater, 90% or greater or 95% or greater sequence identity with SEQ ID NO: 71, 77, 83, 89, 95, 101, 107 or 113 and the light chain variable domain which binds to LTBR comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 72, 78, 84, 90, 96, 102, 108 or 114, LCDR2 comprises or consists of a polypeptide sequence sharing 70% or greater, 75% or greater, 80% or greater, 85% or greater, 90% or greater or 95% or greater sequence identity with SEQ ID NO: 73, 79, 85, 91, 97, 103, 109 or 115 and LCDR3 comprises or consists of a polypeptide sequence sharing 70% or greater, 75% or greater, 80% or greater, 85% or greater, 90% or greater or 95% or greater sequence identity with SEQ ID NO: 74, 80, 86, 92, 98, 104, 110 or 116. More suitably HCDR1 comprises or consists of a polypeptide sequence of SEQ ID NO: 69, 75, 81, 87, 93, 99, 105 or 111, HCDR2 comprises or consists of a polypeptide sequence of SEQ ID NO: 70, 76, 82, 88, 94, 100, 106 or 112 and HCDR3 comprises or consists of a polypeptide sequence of SEQ ID NO: 71, 77, 83, 89, 95, 101, 107 or 113 and LCDR1 comprises or consists of a polypeptide sequence of SEQ ID NO: 72, 78, 84, 90, 96, 102, 108 or 114, LCDR2 comprises or consists of a polypeptide sequence of SEQ ID NO: 73, 79, 85, 91, 97, 103, 109 or 115 and LCDR3 comprises or consists of a polypeptide sequence of SEQ ID NO: 74, 80, 86, 92, 98, 104, 110 or 116.

In one embodiment the heavy chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 27, 29, 31, 33, 35, 37, 39 or 41 and the light chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 28, 30, 32, 34, 36, 38, 40 or 42. More suitably the heavy chain variable domain which binds to LTBR comprises or consists of a polypeptide sequence of SEQ ID NO: 27, 29, 31, 33, 35, 37, 39 or 41 and the light chain variable domain which binds to LTBR comprises or consists of a polypeptide sequence of SEQ ID NO: 28, 30, 32, 34, 36, 38, 40 or 42.

In one embodiment the variable domain which binds to LTBR comprises or consists of three heavy chain complementarity determining regions (HCDR1-HCDR3) and four framework regions (FR1-FR4), wherein HCDR1 comprises or consists of a polypeptide sequence sharing 70% or greater, 75% or greater, 80% or greater, 85% or greater, 90% or greater or 95% or greater sequence identity with SEQ ID NO: 163, 166, 169 or 172, HCDR2 comprises or consists of a polypeptide sequence sharing 70% or greater, 75% or greater, 80% or greater, 85% or greater, 90% or greater or 95% or greater sequence identity with SEQ ID NO: 164, 167, 170 or 173 and HCDR3 comprises or consists of a polypeptide sequence sharing 70% or greater, 75% or greater, 80% or greater, 85% or greater, 90% or greater or 95% or greater sequence identity with SEQ ID NO: 165, 168, 171 or 174. More suitably HCDR1 comprises or consists of a polypeptide sequence of SEQ ID NO: 163, 166, 169 or 172, HCDR2 comprises or consists of a polypeptide sequence of SEQ ID NO: 164, 167, 170 or 173 and HCDR3 comprises or consists of a polypeptide sequence of SEQ ID NO: 165, 168, 171 or 174.

In one embodiment the variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 159, 160, 161 or 162. More suitably the variable domain which binds to LTBR comprises or consists of a polypeptide sequence of SEQ ID NO: 159, 160, 161 or 162.

Preferably the HCDRs (and LCDRs) are provided in the heavy chain variable domain which binds to LTBR (and paired light chain variable domain which binds to LTBR) in the same combinations as set out in A/B. For example, it is preferable that HCDRs1-3 of SEQ ID Nos: 57, 58 and 59 are deployed in combination (as set out for Clone 1.5) e.g. in a VHH, and it is preferable that (a) HCDRs1-3 of SEQ ID Nos: 69, 70 and 71 and (b) LCDRs1-3 of SEQ ID Nos: 72, 73 and 74 are deployed in combination (as set out for Clone 2.1) e.g. in an scFv. The same principle applies for combinations of heavy chain variable regions and light chain variable regions as set out in.

Also provided is a polypeptide which binds to LTBR comprising or consisting of the polypeptide sequence(s) of clone 1.1, 1.2, 1.3, 1.4, 1.5, 1.6, 1.7, 1.8, 1.9, 1.10, 1.11, 1.12, 2.1, 2.2, 2.3, 2.4, 2.5, 2.6, 2.7 or 2.8; or an antibody which competes for binding to LTBR with an anti-LTBR polypeptide of the invention.

Suitably the polypeptide specifically binds to LTBR, for example in that any other entity is bound to with a K_{D} of 10⁻⁷ M or more, such as 10⁻⁶ M or more. Suitably the polypeptide specifically binds to the extracellular domain of LTBR. In some embodiments, the LTBR is on the surface of stromal cells. In some embodiments, the LTBR is on the surface of endothelial cells. In some embodiments, the LTBR is on the surface of fibroblasts. In further embodiments, the LTBR is situated on the surface of cancer cells, myeloid cells and/or macrophages (e.g. present in the TME).

In some embodiments the LTBR binding polypeptide is an LTBR agonist, such that binding to LTBR leads to activation of LTBR NFKB pathways, such as the canonical and/or non-canonical NFKB pathways. If the LTBR binding agent is an LTBR agonist, then suitably the LTBR binding agent (a) induces clustering of LTBR and more suitably (b) activates the classical NFKB pathway (suitably leading to expression of adhesion molecules such as ICAMs, such as ICAM-1, VCAM-1 and/or MAdCAM-1) and/or activates the alternative NFKB pathway (suitably leading to expression of chemokines, such as CCL5, CCL19, CCL21 and/or CXCL13).

The LTBR binding polypeptide may bind to LTBR with an affinity (K_{D}) of 20 nM or less, such as 15 nM or less, such as 10 nM or less, such as 9 nM or less, such as 8 nM or less, such as 7 nM or less, such as 6 nM or less, such as 5 nM or less, such as 4 nM or less, such as 3 nM or less, such as 2 nM or less, such as 1 nM or less. In some embodiments, the LTBR binding polypeptide may bind to LTBR with an affinity (K_{D}) as recited in or less. The affinity may be established as set out in Example 2.

Suitably the LTBR binding polypeptide has an EC50 of 9.0E-09 M or less, such as 8.0E-09 M or less, such as 7.0E-09 M or less, such as 6.0E-09 M or less, such as 5.0E-09 M or less, such as 4.0E-09 M or less, such as 3.0E-09 M or less, such as 2.0E-09 M or less, such as 1.0E-09 M or less, such as 9.0E-10 M or less, such as 8.0E-10 M or less, such as 7.0E-10 M or less, such as 6.0E-10 M or less, such as 5.0E-10 M or less, such as 4.0E-10 M or less, such as 3.0E-10 M or less, such as 2.0E-10 M or less, such as 1.0E-10 M or less, such as 9.0E-11 M or less, such as 8.0E-11 M or less, such as 7.0E-11 M or less, such as 6.0E-11 M or less, such as 5.0E-11 M or less, such as 4.0E-11 M or less, such as 3.0E-11 M or less, such as 2.0E-11 M or less, such as 1.0E-11 M or less, such as 9.0E-12 M or less, such as 8.0E-12 M or less, such as 7.0E-12 M or less, such as 6.0E-12 M or less, such as 5.0E-12 M or less, such as 4.0E-12 M or less, such as 3.0E-12 M or less, such as 2.0E-12 M or less, such as 1.0E-12 M or less.

Alternatively, the LTBR binding polypeptide may have an EC50 as recited for any of the clones specified in the tables of the Examples or less.

Features described above in respect of multispecific constructs of the invention may also apply mutatis mutandis to certain embodiments of the polypeptides of the invention which bind to LTBR.

Also provided is an anti-LTBR polypeptide or construct of the invention wherein the polypeptide or construct is for use in the treatment of cancer in combination with a cancer vaccine. Suitably the polypeptide or construct and cancer vaccine are for use in combination with an adjuvant. Suitably the polypeptide, construct cancer vaccine and/or adjuvant are administered sequentially, separately or simultaneously. In some embodiments, the cancer vaccine may be an antigenic peptide or a polynucleotide encoding an antigen (such as RNA (e.g. mRNA) or DNA), optionally comprised in a pharmaceutically acceptable excipient. The cancer vaccine may be a tumour associated antigen or an encoded tumour associated antigen. Suitably the cancer vaccine is a tumour associated antigen comprising or consisting of the polypeptide sequence KCLQDNNWDYTRYAQAFTLLKAKG (SEQ ID NO: 209). In one embodiment the adjuvant is a TLR agonist, such as a TLR-9 agonist, such as a CpG oligonucleotide.

### Polypeptides Binding to FAP

In one embodiment the FAP binding polypeptide comprises or consists of a paired heavy chain variable domain and light chain variable domain which bind to FAP (e.g. a Fab), suitably as defined further below.

In one embodiment the heavy chain variable domain which binds to FAP comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 117, 123, 129, 135, 141, 147 or 153, HCDR2 comprises or consists of a polypeptide sequence sharing 70% or greater, 75% or greater, 80% or greater, 85% or greater, 90% or greater or 95% or greater sequence identity with SEQ ID NO: 118, 124, 130, 136, 142, 148 or 154 and HCDR3 comprises or consists of a polypeptide sequence sharing 70% or greater, 75% or greater, 80% or greater, 85% or greater, 90% or greater or 95% or greater sequence identity with SEQ ID NO: 119, 125, 131, 137, 143, 149 or 155 and the light chain variable domain which binds to FAP comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 120, 126, 132, 138, 144, 150 or 156, LCDR2 comprises or consists of a polypeptide sequence sharing 70% or greater, 75% or greater, 80% or greater, 85% or greater, 90% or greater or 95% or greater sequence identity with SEQ ID NO: 121, 127, 133, 139, 145, 151 or 157 and LCDR3 comprises or consists of a polypeptide sequence sharing 70% or greater, 75% or greater, 80% or greater, 85% or greater, 90% or greater or 95% or greater sequence identity with SEQ ID NO: 122, 128, 134, 140, 146, 152 or 158. More suitably HCDR1 comprises or consists of a polypeptide sequence of SEQ ID NO: 117, 123, 129, 135, 141, 147 or 153, HCDR2 comprises or consists of a polypeptide sequence of SEQ ID NO: 118, 124, 130, 136, 142, 148 or 154 and HCDR3 comprises or consists of a polypeptide sequence of SEQ ID NO: 119, 125, 131, 137, 143, 149 or 155 and LCDR1 comprises or consists of a polypeptide sequence of SEQ ID NO: 120, 126, 132, 138, 144, 150 or 156, LCDR2 comprises or consists of a polypeptide sequence of SEQ ID NO: 121, 127, 133, 139, 145, 151 or 157 and LCDR3 comprises or consists of a polypeptide sequence of SEQ ID NO: 122, 128, 134, 140, 146, 152 or 158.

In one embodiment the heavy chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 43, 45, 47, 49, 51, 53 or 55 and the light chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 44, 46, 48, 50, 52, 54 or 56. More suitably the heavy chain variable domain comprises or consists of a polypeptide sequence of SEQ ID NO: 43, 45, 47, 49, 51, 53 or 55 and the light chain variable domain comprises or consists of a polypeptide sequence of SEQ ID NO: 44, 46, 48, 50, 52, 54 or 56.

Preferably the HCDRs and LCDRs are provided in the heavy chain variable domain which binds to FAP and light chain variable domain which binds to FAP in the same combinations as set out in A. For example, it is preferable that (a) HCDRs1-3 of SEQ ID Nos: 117, 118 and 119 and (b) LCDRs1-3 of SEQ ID Nos: 120, 121 and 122 are deployed in combination (as set out for Clone 3.5) e.g. in a Fab. The same principle applies for combinations of variable regions as set out in.

Also provided is a polypeptide which binds to FAP comprising or consisting of the polypeptide sequence(s) of clone 3.1, 3.2, 3.3, 3.4, 3.5, 3.6 or 3.7; or, an antibody which competes for binding to FAP with the anti-FAP polypeptides of the invention.

Suitably the FAP binding polypeptide specifically binds to FAP, for example such that any other entity is bound to with a KD of 10⁻⁷ M or more, such as 10⁻⁶ M or more. In some embodiments, the FAP is on the surface of stromal cells (e.g. CAFs) and/or on the surface of cancer cells. Suitably the FAP binding polypeptide specifically binds to the extracellular domain of FAP.

Suitably the FAP binding polypeptide binds to FAP with an affinity (K_{D}) of 400 pM or less, such as 300 pM or less, such as 200 pM or less, such as 150 pM or less, such as 130 pM or less, such as 110 pM or less, such as 100 pM or less, such as 50 pM or less. Alternatively the FAP binding polypeptide binds to FAP with an affinity (K_{D}) as recited in or less. Suitably the affinity is established as set out in Example 3.

Suitably the FAP binding polypeptide has an EC50 of 9.0E-09 M or less, such as 8.0E-09 M or less, such as 7.0E-09 M or less, such as 6.0E-09 M or less, such as 5.0E-09 M or less, such as 4.0E-09 M or less, such as 3.0E-09 M or less, such as 2.0E-09 M or less, such as 1.0E-09 M or less, such as 9.0E-10 M or less, such as 8.0E-10 M or less, such as 7.0E-10 M or less, such as 6.0E-10 M or less, such as 5.0E-10 M or less, such as 4.0E-10 M or less, such as 3.0E-10 M or less, such as 2.0E-10 M or less, such as 1.0E-10 M or less, such as 9.0E-11 M or less, such as 8.0E-11 M or less, such as 7.0E-11 M or less, such as 6.0E-11 M or less, such as 5.0E-11 M or less, such as 4.0E-11 M or less, such as 3.0E-11 M or less, such as 2.0E-11 M or less, such as 1.0E-11 M or less, such as 9.0E-12 M or less, such as 8.0E-12 M or less, such as 7.0E-12 M or less, such as 6.0E-12 M or less, such as 5.0E-12 M or less, such as 4.0E-12 M or less, such as 3.0E-12 M or less, such as 2.0E-12 M or less, such as 1.0E-12 M or less. Alternatively the FAP binding polypeptide has an EC50 as recited for any of the clones specified in the tables of the Examples or less.

Features described above in respect of multispecific constructs of the invention may also apply mutatis mutandis to certain embodiments of the polypeptides of the invention which bind to FAP.

### Polypeptides Binding to LTBR or FAP

The polypeptides described above which bind to LTBR or FAP may comprise an antibody, such as consist of an antibody, or may comprise or consist of one or more antibody fragments. A suitable fragment may include for example an scFv, Fv, Fab, Fab′, F(ab′)2, variable domain (e.g. VH, VL, VNAR or VHH), diabody or minibody.

The polypeptides may comprise a further binding agent which is neither an LTBR binding agent nor a FAP binding agent. In some embodiments, the polypeptides may comprise no further binding agents.

Also provided is an antibody which binds to the epitope of any one of the polypeptide of the anti-LTBR or anti-FAP polypeptides of the invention.

The anti-LTBR or anti-FAP polypeptides of the invention may be for use as a medicament, such as for use in the treatment of cancer; or may be for use in the manufacture of a medicament, such as for use in the treatment of cancer. There is provided a method of treating cancer comprising administering to a subject in need thereof an anti-FAP or anti-LTBR polypeptide of the invention. The cancer may be a tumour or a non-haematological cancer.

Also provided is a set of one or more polynucleotides encoding the anti-FAP or anti-LTBR polypeptides of the invention, a set of one or more expression vectors collectively comprising such polynucleotides, and a cell comprising the expression vectors or polynucleotides. Also provided is a method of producing an anti-FAP or anti-LTBR polypeptide of the invention, the method comprising culturing the cell under suitable conditions such that the polynucleotide is expressed, and the polypeptide is produced.

The invention is further exemplified by the following non-limiting examples.

### EXAMPLES

### 1. Example 1. Generation of Anti-LTBR Nanobodies (VHHs)

### 1.1. Immunisation and Library Constructions

VHHs were generated by immunising llamas and alpacas with recombinant protein, essentially as described elsewhere (Pardon et al., 2014) (Henry & MacKenzie, 2018). Briefly, animals were immunised with human LTBR human IgG1 Fc chimera (Acro Biosystems, cat. #LTR-H5251).

Phage display libraries derived from peripheral blood mononuclear cells (PBMCs) were prepared and used as previously described (Pardon et al., 2014) (Henry & MacKenzie, 2018). The VHH fragments were cloned into a M13 phagemid vector containing Myc and His6 tags. The library was rescued by infecting exponentially growing Escherichia coli TG1 cells and superinfection with VCSM13 helper phage.

The human LTBR immunised phage libraries were subjected to two consecutive selection rounds on human LTBR human IgG1 Fc chimera protein (Sino Biological, cat. #10581-H02H). 1.2. Screening of anti-LTBR VHHs

In a binding ELISA, 830 VHH clones from the immunisation and selection campaigns were screened as crude periplasmic extracts. Plates were coated with 5 μg/ml of NeutrAvidin in PBS (overnight at 4° C.), followed by blocking with 1% casein in PBS (2 h at RT). Next, biotinylated hLTBR (OriGene, cat. #AR51717PU-S) was captured at 10 nM in 0.1% casein in PBS (1 h at RT). Detection was done with 5 μg/ml anti-c-myc antibody 9E10 (Merck, cat. #11667203001) and 0.16 μg/ml anti-mouse IgG-HRP (Jackson Immuno Research, cat. #715-035-150), both in 0.1% casein in PBS (1 h at RT). Sequence analysis of 808 hits revealed that these belonged to 149 different clusters of related sequences. 270 unique sequences were then confirmed for binding utilising off-rate analysis on human and cynomolgus LTBR (as described below).

To confirm species cross-reactivity, either human or cynomolgus recombinant LTBR-fused on human Fc (SinoBiological, cat. #10581-H02H and 90101-C02H) were immobilised on CM5 sensor chips (cat. #CFJB 334). 1:5 dilutions of crude periplasmic extracts of clones expressing VHHs were injected at 30 μl/min for 2 min in HBS-EP pH 7.4 buffer to allow for binding to chip-bound antigen. Next, an HBS-EP pH7.4 buffer without VHHs was injected for 5 minutes at the same flow rate to enable spontaneous dissociation of bound VHHs. Surface regeneration was done with 10 ul of 1 mM Glycine pH 1.5/1 M NaCl injected twice between samples. From the sensorgrams obtained on a BiacoreT200 machine, koff values were calculated by fitting a 1:1 interaction model (Langmuir model) using Insight Evaluation Software (Biacore). Based on this analysis, four clones were selected for further evaluation, and the results are included in. The result confirmed that the selected clone recognised human and cynomolgus LTBR with comparable binding strength.

TABLE 1
Off-rate analysis on human and cynomolgus
LTBR on selected leads by Biacore.

| Clone | Human LTBR | Cynomologus LTBR |
|---|---|---|
| ID | koff (1/S) | koff (1/s) |
| 1.1 | 8.0 × 10−04 | 1.4 × 10−03 |
| 1.2 | 6.9 × 10−04 | 2.6 × 10−03 |
| 1.3 | 3.8 × 10−03 | 3.1 × 10−03 |
| 1.4 | 1.8 × 10−03 | 9.7 × 10−04 |

Synthetic DNA fragments encoding the VHHs were subcloned into an E. coli expression vector under the control of an IPTG-inducible lac promoter in a frame with N-terminal PeIB signal peptide (which directs the recombinant proteins to the periplasmic compartment) and C-terminal FLAG3 and His6 tags. VHH proteins were purified from these clones employing IMAC chromatography followed by desalting according to well-established procedures (Pardon et al., 2014).

The purified proteins were tested for binding to endogenously expressed human LTBR in HepG2 cells. The expression level was confirmed with anti-LTBR clone CBE-11 (“huCBE11” as disclosed in WO2004002431). The HepG2 cells were resuspended to a final concentration of 1.0×10⁶ cells/ml in FACS buffer. A series dilution of purified VHHs was incubated with mouse anti-FLAG biotinylated antibody (Sigma Aldrich, cat. #F9291-1MG) at 5 μg/ml in FACS buffer for 30 min at RT with shaking. Cell suspensions were distributed into a 96-well v-bottom plate and incubated with the above-described VHH/antibody mixture for 1 hour on ice while shaking. VHH binding to cells was detected with streptavidin R-PE (Invitrogen, cat. #.SA10044) at 1:400 dilution (0.18 μg/ml) in FACS buffer, incubated for 30 minutes on ice with shaking and protection from light. The cells were acquired by flow cytometry. The data was analysed using FloJo V10 and GraphPad Prism. The EC50 values can be found in.

TABLE 2
EC50 values for binding curves to HepG2 cell line

| | HepG2 Binding |
|---|---|
| Clone ID | EC50 [M] |
| 1.1 | 4.1E−10 |
| 1.2 | 1.6E−09 |
| 1.3 | 1.2E−09 |
| 1.4 | 2.3E−09 |

Activation of LTBR by its ligands LTα1β2 or LIGHT occurs via clustering of multiple LTBR receptors leading to activation of an intracellular signal transduction cascade. Therefore, activation of the receptor with a monovalent molecule is not expected. The activation of human LTBR was evaluated in NFKB Luciferase Reporter HepG2 cells (endogenous LTBR expression Signosis, cat. #SL-0017) in the presence and absence of cross-linking anti-His antibody.

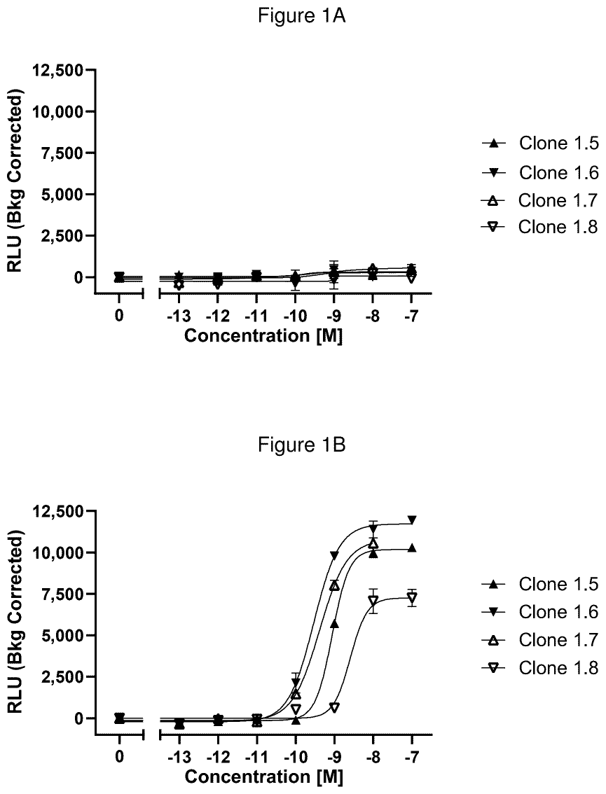

For the reporter assay, cells were plated in 96-well plates (Greiner) at a density of 3×10⁴ cells/well and cultured overnight at 37° C. and 5% CO2 in Eagle's Minimum Essential Medium (EMEM, Gibco) supplemented with 10% heat-inactivated FBS and 100 U/mL penicillin and streptomycin (Gibco). Cells were then incubated with monovalent His Tag containing VHHs cross-linked with anti-His tag mAb (Jackson, cat. #300-005-240) for 6 hours. After 6 h, a ONE-Glo Ex Solution (Promega, cat. no E8120) was added to the well and incubated for 10 min before being read on a BMG Pherastar FSX plate reader. Background-corrected RLU values were plotted against stimulant (antibody or antibody fragment) concentration for all conditions tested in GraphPad Prism, and the EC50 values can be found in. FIGS. 1a and 1b demonstrate that, as expected, the monovalent VHHs do not lead to activation of the LTBR unless cross-linked with anti-His Tag antibodies.

TABLE 3
EC50 values from the HepG2 reporter assay

| | | Cross-linked VHH |
|---|---|---|
| | | with anti-His tag |
| | Monovalent | Ab |
| Clone ID | VHH | EC50 [M] |
| 1.5 | Inactive | 8.7E−010 |
| 1.6 | Inactive | 2.9E−010 |
| 1.7 | Inactive | 4.1E−010 |
| 1.8 | Inactive | 2.5E−009 |

### 1.3. Sequence Optimisation of Monovalent VHHs

The protein sequences of four selected VHHs were modified to improve their humanisation towards human IGHV3 and JH germline consensus sequences as well as their chemical and biophysical stability while minimising the impact on target binding. To this end, different mutant variants were generated for each lead, which were then compared for their capacity to compete for binding to human LTBR endogenously expressed on HepG2 cells versus their respective FLAG3-tagged parental sequences using competition flow cytometry. Melting (Tm) temperatures of the different variants were determined. The variants were also subjected to temperature stress (1 week @40° C.) followed by analytical size exclusion in combination with multiangle laser light scattering analysis (aSEC-MALLS) to assess the oligomerisation propensity. In addition, the VHHs were subjected to long-term temperature stress (4 weeks @40° C.) and forced oxidative stress (10 mM H2O2 for 3 hours @37° C.), followed by detailed peptide mapping mass spectrometry to assess amino acid stability. Amino acid residues with poor chemical stability (>5% modification after relevant stress) were replaced by suitable alternatives. The sequence-optimised mutants with desired properties were selected for further assessment.

1.4. Formatting into VHH-Fc Fusions and Functional Testing

VHH-Fc fusions were generated by combining sequence-optimised anti-LTBR VHHs with human IgG1 Fc domain, separated by flexible Glycine Serine linkers. The constructs were cloned into an expression vector and expressed by transient CHO suspension cell transfection. VHH-Fc fusion proteins were purified from cell culture supernatants by affinity chromatography using protein A resin and followed by one polishing chromatography step: preparative size exclusion (SEC).

SEC-HPLC and mass spectrometry assessed the purity and heterogeneity of the samples. All samples were confirmed to have a monomer content of >95% and contain <10% impurities before functional read-out. The list of constructs can be found in.

TABLE 4
List of VHH-Fc fusion constructs

| Clone ID | Specificity | Format | Isotype |
|---|---|---|---|
| 1.9 | LTBR | VHH-Fc | Human |
| | | | IgG1 |
| 1.10 | LTBR | VHH-Fc | Human |
| | | | IgG1 |
| 1.11 | LTBR | VHH-Fc | Human |
| | | | IgG1 |
| 1.12 | LTBR | VHH-Fc | Human |
| | | | IgG1 |

### 1.4.1. NFKB Activation of HepG2 Reporter Cells Expressing Human LTBR

The ability of anti-LTBR VHH-Fc fusion proteins to activate LTBR was tested in NFKB luciferase reporter HepG2 (Signosis, cat. no SL0017-FP). Clustering endogenous LTBR on HepG2 cells leads to activation of endogenous NFKB and translocation from the cytoplasm and nucleus, which binds to the promoter region and induces luciferase expression. After that, a luciferase substrate/lysis reagent mix (ONE-GLO EX Promega) is added to cells, which allows quantification in the bioluminescence reaction, where enzymatic activity is proportional to luciferase expression.

A monospecific anti-LTBR agonist (BHA10, as disclosed in WO2004002431) was used as a positive control. In short, HepG2 NFKB luciferase (Lc) (30000 cells/well) were seeded, and the 1:10 series dilution of VHH-Fc fusion proteins and controls was added to the cells and incubated for 6 h at 37° C., 5% CO2, prior to the addition of detection reagents. After 6 h, a ONE-Glo Ex Solution (Promega, cat. no E8120) was added to the well and incubated for 10 min before being read on a BMG Pherastar FSX plate reader. Background-corrected RLU values were plotted against stimulant concentration for all conditions tested in GraphPad Prism, and the EC50 value can be found in. The results confirmed that formatting monomeric VHHs into bivalent molecules leads to LTBR activation with comparable or higher potency than the control monoclonal antibody (BHA10).

TABLE 5
EC50 values of HepG2 NFKB activation-
induced luciferase activity

| | HepG2 Reporter assay |
|---|---|
| Clone ID | EC50 [M] |
| 1.9 | 3.2e−12 |
| 1.10 | 9.5e−12 |
| 1.11 | 1.2e−10 |
| 1.12 | 2.9e−11 |
| BHA10 | 1.2e−11 |

### 1.4.2. In Vitro VHH Fc Fusion Protein Activity in a Chemokine Induction Assay Using Tumor Cell Line HCC1187

An activation assay measuring chemokine secretion was used to investigate the ability of anti-LTBR VHH-Fc fusion proteins to activate LTBR endogenously expressed on tumor cells (HCC1187). In short, HCC1187 (20000/well) were seeded in their growth medium. A 1:10, 7-point serial dilution series in duplicates of antibodies and controls was prepared in assay medium as 2-fold concentration stocks, added to the cells, and incubated for 24 h at 37° C., 5% CO2. After incubation, the supernatants were collected and cleared by centrifugation, and the chemokine level was determined by CCL19 DuoSet ELISA (R&D system, cat. no DY361) following the manufacturer's instructions. Absorbance was read on the BMG Pherastar FSX plate reader. OD corrected values (450-540 nm) for the standard curve were analysed in Prism, according to the manufacturer's recommendation, to interpolate cytokine concentrations for the test wells. Dose-response curves were plotted using GraphPad Prism Version 9, applying non-linear fits (log(agonist) vs response (variable slope—four parameters). EC50 values can be found in.

TABLE 6
EC50 values of upregulation of CCL19
in HCC1187 chemokine induction assay

| | HCC1187 chemokine |
|---|---|
| | induction assay |
| Clone ID | EC50 [M] |
| 1.9 | 3.5E−010 |
| 1.10 | 6.0E−011 |
| 1.11 | 2.5E−010 |
| 1.12 | 9.1E−009 |
| BHA10 | 5.3E−010 |

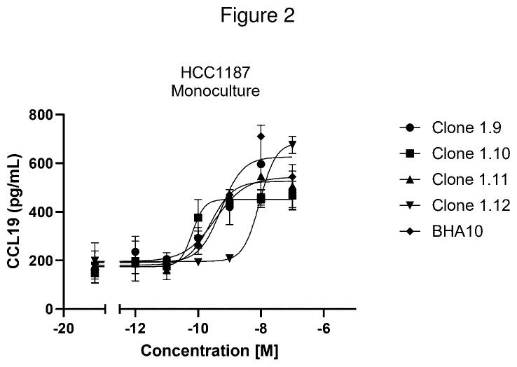

FIG. 2 shows that anti-LTBR VHH-Fc fusion proteins induced dose-dependent CCL19 production in HCC1887 cells.

### 1.4.3. Ligand Receptor Inhibition Assay

The ability of VHH-Fc fusion proteins to bind to endogenously expressed LTBR on the A549 tumor cell and to inhibit human LIGHT (Acro, cat. no LIT-H5242) binding to A549 cells was assessed. BHA10 was employed in the assay as a positive control. Recombinant human LIGHT was labelled with PE fluorochrome, using PE/R-Phycoerythrin Conjugation Kit—Lightning-Link® (Abcam, cat no ab102918), following the manufacturer's instructions. 1×10⁶ cells were resuspended in the FACS buffer (1% BSA, 0.1% Sodium Azide in PBS), and 50 ul was added to each well (5×10⁵ cells/well) of a 96-U bottom plate. The cells were washed and resuspended in 25 ul of antibody dilution (2× final concentration) followed by 0.5 h incubation at 4° C., 25 ul of PE labelled LIGHT at 48 nM (2× final concentration) in FACS buffer was subsequently added to the cells and additionally incubated for 1 h at 4° C. The cells were washed, and cells were further stained with a secondary detection antibody, Alexa Fluor®647-conjugated AffiniPure Fab Fragment Goat Anti-Human IgG (H+L) (Stratech, cat. no 109-607-003), for 1 h incubation at 4° C. Next, cells were extensively washed and fixed with Paraformaldehyde Solution, 4% in PBS (Thermo Scientific, cat no J19943.K2) for 20 mins, following the manufacturer's instructions. The cells were then washed, resuspended in PBS, and read on Beckman Coulter CytoFLEX. The data were analysed using FloJo V10 and GraphPad Prism v9. The IC50 and EC50 values can be found in.

TABLE 7
IC50 values for ligand-receptor inhibition and EC50 values
of binding curves to LTBR positive cell line (A549)

| | Ligand Receptor Inhibition | A549 |
|---|---|---|
| Clone ID | IC50 [M] | EC50 [M] |
| 1.9 | 2.9E−10 | 3.9E−10 |
| 1.10 | 9.9E−11 | 1.6E−10 |
| 1.11 | 1.6E−10 | 2.4E−10 |
| 1.12 | 4.0E−10 | 5.3E−10 |
| BHA10 | 6.7E−10 | 7.8E−10 |

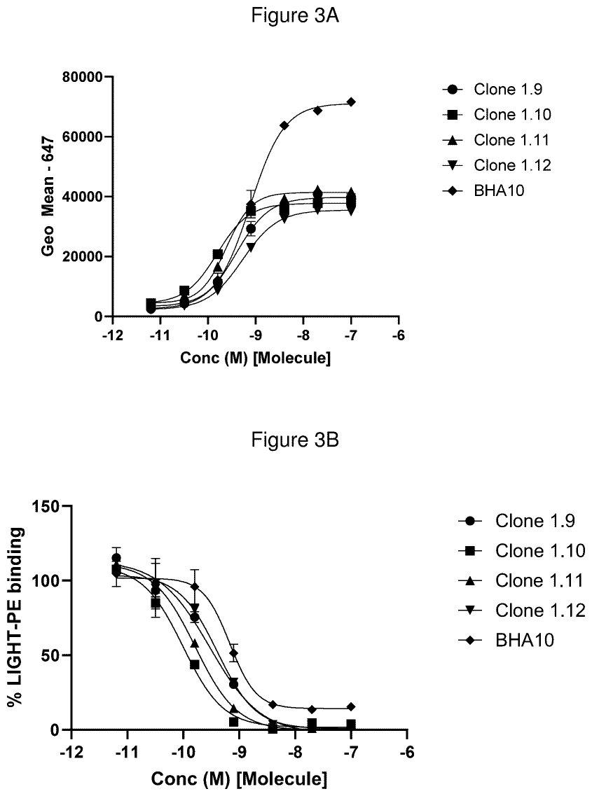

FIG. 3a confirmed that all tested antibodies bind strongly to endogenously express LTBR on A549 tumor cell line.

FIG. 3b confirms that all tested clones demonstrated an ability to inhibit LTBR-LIGHT interaction with IC50 values ranging from 0.16 nM to 0.09 nM. The positive control antibody, BHA10, showed partial inhibition.

### 1.4.4. Epitope Competition Assay by Octet

An epitope binning assay on Octet Red96 was employed to confirm whether the selected anti-LTBR VHHs compete for the same epitope. In short, 5 ug/ml of recombinant human LTBR was amine coupled into AR2G biosensors at pH 5.0, following manufacturer instructions. The first VHH was loaded onto pre-coated LTBR biosensors until saturation was reached, followed by the sensor's exposure to the competitor/second VHH. Detection of any additional binding on the biosensor by the second antibody indicates an unoccupied epitope (non-competitor). In contrast, no second antibody binding on the biosensor indicates a blocked epitope (competitor). The results suggest that only Clone 1.10 binds to a distinct epitope, whereas all the other clones bind to an overlapping epitope.

### 2. Example 2 Generation of Anti-LTBR Monoclonal Antibodies

### 2.1. Immunisation

Two cohorts, 5 mice each, of transgenic humanised mice, ATX-GK Cross, were immunised with human LTBR recombinant protein or plasmid DNA encoded to express human LTBR cDNA using standard 5-week RIMMS protocol for proteins (10 pg subcutaneous dosing of antigen emulsified in complete Freund's adjuvant followed by 5 weekly subcutaneous dosing of antigen emulsified in incomplete Freund's adjuvant) or an extended protocol for DNA (25 pg co-injection with pBoost4 adjuvant vector injected ID every week for six weeks with a final pre-fusion boost with 10 μg of recombinant protein injected IP).

Sample bleeds were taken at week four and tested for antigen-positive serum titer and purification tag-negative serum titer by ELISA. ELISA plates were coated with either 1 μg/ml of human LTBR with human Fc (Acro H5259), cyno LTBR with human Fc (R&D 10759-LR), human LTBR with mouse Fc (M-300-15, produced in-house), mouse LTBR with mouse Fc (R&D 1008-LR), or internal mouse Fc control (P-110). Antigen-coated plates were incubated with 8-point 10-fold serial dilutions of sera starting at 1:100. Antibodies bound to antigen were detected by anti-mouse IgG HRP secondary antibody and one-step TMB solution. The absorbance signal at 450 nm was measured with an ELISA microplate reader.

### 2.2. Hybridomas

Immune tissues from high-titer mice were harvested and preserved for antibody discovery. Hybridoma cell lines producing LTBR antibodies were produced by the fusion of single B Cells from the spleen and lymph nodes of titer-positive mice with myeloma cells. Twenty 96 well plates of hybridoma fusions were generated and expanded. Hybridomas expressing LTBR-specific antibodies were detected by antigen binding by ELISA. The affinity of antibodies in the hybridoma supernatants was measured by Bio-Layer Interferometry (BLI) using the Octet instrument. LTBR antibodies in hybridoma supernatant were loaded on a biosensor. The response was measured as a nm shift in the interference pattern and was proportional to the number of antibodies bound to the biosensor's surface. The binding interaction of LTBR to the immobilised antibodies was measured as an association (kon). Following analyte association, the biosensor was dipped into PBS without LTBR, and the bound antigen was allowed to dissociate from the antibody (kdis), KD (M), or affinity of the antibodies for LTBR was measured as kdis/kon.

Heavy and light chains from validated hybridomas were sequenced. RNA was isolated from LTBR antibody-secreting hybridomas, and heavy and light chain variable regions were cloned by reverse transcription using gene-specific primers followed by PCR amplification with variable chain gene-specific primers. PCR products were sequenced by standard Sanger sequencing methods.

### 2.3. Phage Display

Variable heavy and light chains were amplified from the spleen of high titer immunised mice by reverse transcription using gene-specific primers followed by PCR amplification with variable chain gene-specific primers. Both variable regions were cloned into a phage display vector designed to express Fabs on phage g3p protein for the diverse library. For the common light chain library, only the heavy chain variable regions were cloned into the phage display vector as before. Libraries of phage expressing unique Fabs were amplified and purified. Phage were allowed to bind to biotinylated LTBR antigens (human, mouse or cyno) captured on streptavidin magnetic beads. Phage remaining bound to antigen beads after several stringent washes was eluted using a basic triethylamine solution and neutralised with Tris buffer pH 8.0. Eluted phages were reinfected into TG1 bacterial cells, amplified by co-infection with M13 helper phage, and purified by PEG precipitation. Purified phages expressing Fabs were selected for antigen binding as described. Phage from the second round was diluted and infected into TG1 cells. Polyclonal pools of phage output from two rounds of panning were tested by ELISA to confirm that the pools contained LTBR-specific phage. Variable heavy and light chain regions were sequenced from single infected bacterial colonies using a rolling circle amplification and standard Sanger sequencing.

### 2.4. Antibody Expression

Unique variable heavy and light chain pairs from all discovery techniques were cloned into vectors designed to express full-length antibodies as IgGs containing LALA silencing mutations in HEK293 cells under the control of a CMV promoter. Antibody expression vectors were complexed with polyethylenimine and transfected into HEK293 cultures. After 5 days of shaking at 37° C. in HEK293 cell culture media, antibodies were captured on agarose-based protein A resin. After several stringent washes, antibodies were eluted in glycine solution, pH 3, neutralised with HEPES, pH 9, and buffer exchanged into PBS. The purity of purified proteins was confirmed by SEC-HPLC and CE-SDS analysis. Each antibody was assigned a unique clone number.

### 2.5. Binding of Anti-LTBR Antibodies to A375 Tumor Cell Line

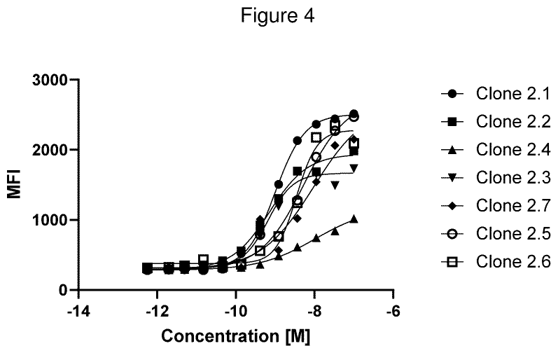

For binding to LTBR-positive cells, the A375 tumor cell line was used. At the same time, binding to LTBR-negative HEK293 cells was tested to confirm specificity. In short, the cells were resuspended in the FACS binding buffer, and live/dead efluro450 viability dye was added at a 1:1000 dilution to the cell suspension, followed by incubation at room temperature in the dark for 30 minutes. The cells were then diluted to 2 e5/ml, and 100 ul was added to wells of a 96-well U-bottom plate as required. For generating an EC50, a 100 nM stock was prepared and then diluted 3 fold serially across 12 points. The cells were pelleted by centrifugation, and the supernatant was discarded, followed by the addition of a series of dilutions of anti-LTBR antibodies and incubation for 45 minutes at 4 C in the dark. Cells were washed two times with PBS with 3% (w/v) BSA before adding 60 ul of fluorescently labelled secondary antibody (goat anti-human IgG) diluted to 1:200 in PBS. Incubation continued at 4 C for at least 30 minutes, followed by two washes in PBS pH 7.4. Cells were either fixed with 2% PFA (30 minutes at 4 C) followed by washout and storage in 100 ul of PBS or immediately analysed on a Flow Cytometer. The data were analysed using FloJo V10 and GraphPad Prism. No binding to the HEK cell line was detected for any tested clones, confirming the specific interaction between anti-LTBR antibodies and LTBR receptor. The results are summarised in. FIG. 4 demonstrates dose-dependent binding to the native form of LTBR, expressed on the A375 tumor cell line.

TABLE 8
EC50 value for binding of anti-LTBR antibodies to A375 cell line

| Clone ID | A375 EC50 [M] | HEK293 cell line |
|---|---|---|
| 2.1 | 1.0E−009 | No binding detected |
| 2.2 | 7.2E−010 | No binding detected |
| 2.3 | 6.6E−010 | No binding detected |
| 2.4 | NP | No binding detected |
| 2.5 | 5.4E−009 | No binding detected |
| 2.6 | 3.5E−009 | No binding detected |
| 2.7 | 9.5E−009 | No binding detected |
| 2.8 | Not Tested | No binding detected |

NP Not reaching a plateau, EC50 cannot be evaluated

### 2.6. Binding Kinetics of Anti-LTBR Antibodies to Human and Cynomolgus LTBR

The binding of anti-LTBR antibodies to human and cynomolgus LTBR was investigated by Surface Plasmon Resonance (SPR) on Carterra LSA. An EC30M chip was used to capture the test antibodies. Activation with EDC and sNHS occurred in MES pH 5.5 to prepare the chip for amine coupling. Goat anti-human IgG Fc antibody was then captured to the chip via incubation for 10 minutes. Excess conjugation sites were quenched with 1M Ethanolamine pH 8. To measure monovalent binding kinetics, antibodies were printed/spotted onto the chip and allowed to bind to the goat antibody, followed by subsequent washing. After the preparation, the printed chip was exposed to repeated rounds of association and dissociation with increasing analyte concentrations. In this work, recombinant proteins (Human LTBR (Acro) or cyno LTBR His (R&D)) were serially diluted across a range starting at 1500 nM with 3-fold dilutions and 10 points. The curves were fitted to global fit in a 1:1 binding model to obtain K_{D} values. The binding affinity to both species is reported. Tested antibodies demonstrated comparable affinities to human and cynomolgus LTBR.

TABLE 9
KD values for binding kinetics to human and cynomolgus LTBR

| Clone ID | huLTBR KD [M] | CyLTBR KD [M] |
|---|---|---|
| 2.1 | 2.5E−10 | 2.6E−10 |
| 2.2 | 5.6E−10 | 7.1E−10 |
| 2.3 | 5.8E−10 | 3.9E−10 |
| 2.4 | 3.6E−08 | 5.3E−08 |
| 2.5 | 2.1E−09 | 1.6E−09 |
| 2.6 | 1.8E−10 | 2.4E−11 |
| 2.7 | 4.3E−09 | 1.3E−09 |
| 2.8 | 2.8E−10 | 2.6E−09 |

### 2.7. Epitope Competition Assay by Carterra LSA

A high-throughput epitope binning experiment was done on real-time label-free biosensors (Carterra LSA) to sort a panel of mAbs into bins based on their ability to block one another for binding to the antigen. In a pairwise epitope binning analysis, antigen and antibody 2 (analyte antibody) are sequentially applied to the sensor chip (HC200M) covalently pre-loaded with antibody 1 (ligand antibody). An increase in response upon exposure to the analyte antibody indicates non-competition between the two antibodies, whereas a lack of change in the signal indicates competition. Antibodies in the test set that have the same blocking profiles towards others are grouped into one bin. Community network plots are used to explore the clustering of mAbs that share similar but not necessarily identical competition profiles. Rather than relying strictly on the sandwiching/blocking assignments in the heat map, as the Bin network plots do, hierarchical clustering is applied to the sorted heat map to generate dendrograms, which progressively group mAbs. Community bins for tested anti-LTBR antibodies are shown in.

TABLE 10
Community bins of anti-LTBR antibodies defined
by epitope competition assay on Carterra

| Clone ID | Community bin |
|---|---|
| 2.1 | 4 |
| 2.2 | 4 |
| 2.3 | 4 |
| 2.4 | 7 |
| 2.5 | 7 |
| 2.6 | 7 |
| 2.7 | 7 |
| 2.8 | 2 |

### 2.8. NFKB Activation of HepG2 Reporter Cells Expressing Human LTBR

To investigate the agonistic function of anti-LTBR antibodies, the NFKB luciferase reporter HepG2 cell line (Signosis, cat. no SL0017-FP) was employed. Clustering endogenous LTBR on HepG2 cells leads to activation of endogenous NfKb and translocation from cytoplasm to the nucleus, where it binds to the promoter region and induces luciferase expression. After that, a luciferase substrate/lysis reagent mix (ONE-GLO EX Promega) is added to cells, which allows quantification in the bioluminescence reaction, where enzymatic activity is proportional to luciferase expression. Antibodies were tested in the presence and absence of cross-linking antibody (anti-human Fc, Jackson, cat no 109-005-098) in a 2:1 ratio. BHA10 was employed as a positive control in this assay.

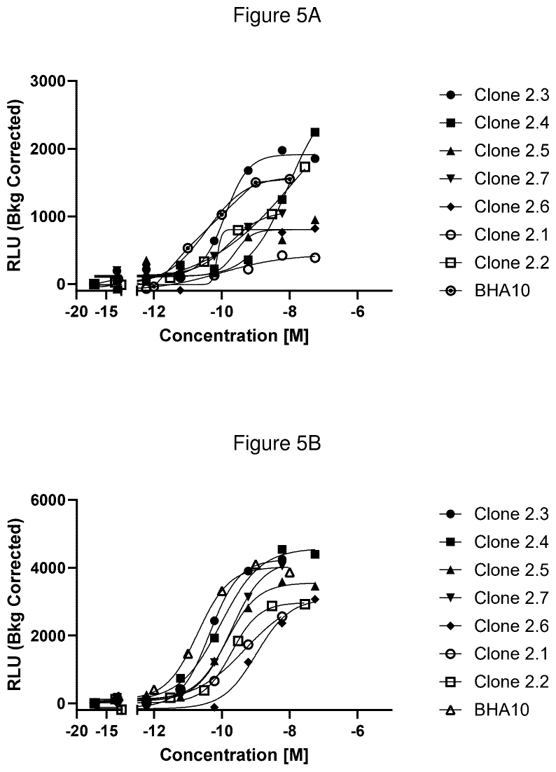

HepG2 NF-Kb Lc (30000 cells/well) were seeded alone in their growth media. The 1:10 series dilution of monospecific antibodies, in the presence or absence of cross-linking agent was added to the cells and incubated for 6 h at 37° C., 5% CO2, prior to the addition of detection reagents. After 6 h, a ONE-Glo Ex Solution (Promega, cat. no E8120) was added to the well and incubated for 10 min before being read on a BMG Pherastar FSX plate reader. Background corrected RLU values were plotted against stimulant concentration for all conditions tested in GraphPad Prism and shown in FIGS. 5a (in the absence of cross-linking antibody) and 5b (in the presence of cross-linking antibody) respectively. The EC50 value can be found in.

TABLE 11
EC50 values for anti-LTBR antibodies agonistic activity in HepG2
reporter assay in the presence or absence of cross-linking antibody
HepG2 reporter assay

| | EC50 (w/o | EC50 (with |
|---|---|---|
| | cross-linking | cross-linking |
| Clone ID | antibody) [M] | antibody) [M] |
| 2.1 | Low level | 1.6E−10 |
| 2.2 | 1.7E−8 | 1.9E−10 |
| 2.3 | 1.3E−10 | 4.3E−11 |
| 2.4 | Low level | 9.0E−11 |
| 2.5 | 2.4E−10 | 1.4E−10 |
| 2.6 | 7.3E−11 | 1.0E−10 |
| 2.7 | 1.7E−10 | 1.8E−10 |
| 2.8 | Not Tested | Not Tested |
| BHA10 | 5.2E−11 | 2.9E−11 |

A broad panel of anti-LTBR antibodies with varied agonistic activity was selected. In most cases, the presence of cross-linking antibody led to a higher potency.

### 3. Example 3 Generation of Anti-FAP Antibodies

### 3.1. Immunisation

Two cohorts, 5 mice each, of transgenic humanised mice, ATX-GK Cross, were immunised with human FAP (Acro Bioscience, cat no FAP-H5244) recombinant protein or plasmid DNA encoded to express human FAP cDNA using standard 5-week RIMMS protocol for proteins (10 pg subcutaneous dosing of antigen emulsified in complete Freund's adjuvant followed by 5 weekly subcutaneous dosing of antigen emulsified in incomplete Freund's adjuvant) or an extended protocol for DNA (25 pg co-injection with pBoost4 adjuvant vector injected ID every week for six weeks with a final pre-fusion boost with 10 μg of recombinant protein injected IP). Sample bleeds were taken at week four and tested for antigen-positive serum titer and purification tag-negative serum titer by ELISA. ELISA plates were coated with 1 μg/ml of human FAP-His (Acro) or internal His tag control (CD22-His). Antigen-coated plates were incubated with 8-point 10-fold serial dilutions of sera starting at 1:100. Antibodies bound to antigen were detected by anti-mouse IgG HRP secondary antibody and one-step TMB solution. The absorbance signal at 450 nm was measured with an ELISA microplate reader. Strong titers from mice dosed with either recombinant protein or cDNA human FAP detected on Day 21 were observed with no reaction to the His Tag.

### 3.2. Hybridoma

Immune tissues from high-titer mice were harvested and preserved for antibody discovery. Hybridoma cell lines producing FAP antibodies were produced by the fusion of single B Cells from the spleen and lymph nodes of titer-positive mice with myeloma cells. Twenty 96 well plates of hybridoma fusions were generated and expanded. Hybridomas expressing FAP-specific antibodies were detected by antigen binding by ELISA. The affinity of antibodies in the hybridoma supernatants was measured by Bio-Layer Interferometry (BLI) using the Octet instrument. FAP antibodies in the hybridoma supernatant were loaded on a biosensor. The response was measured as a nm shift in the interference pattern and was proportional to the number of antibodies bound to the biosensor's surface. The binding interaction of FAP to the immobilised antibodies was measured as an association (kon). Following analyte association, the biosensor was dipped into PBS without FAP, and the bound antigen was allowed to dissociate from the antibody (kdis), KD (M), or affinity of the antibodies for FAP was measured as kdis/kon.

Heavy and light chains from validated hybridomas were sequenced. RNA was isolated from FAP antibody-secreting hybridomas, and heavy and light chain variable regions were cloned by reverse transcription using gene-specific primers followed by PCR amplification with variable chain gene-specific primers. PCR products were sequenced by standard Sanger sequencing methods.

### 3.3. Phage Display

Variable heavy and light chains were amplified from the spleen of high titer immunised mice by reverse transcription using gene-specific primers followed by PCR amplification with variable chain gene-specific primers. Both variable regions were cloned into a phage display vector designed to express Fabs on phage g3p protein for the diverse library. For the common light chain library, only the heavy chain variable region were cloned into the phage display vector as before. Libraries of phage expressing unique Fabs were amplified and purified. Phage were allowed to bind to biotinylated FAP antigens (human, mouse or cyno) captured on streptavidin magnetic beads. After several stringent washes, the phage remaining bound to antigen beads was eluted using a basic triethylamine solution and neutralised with Tris buffer pH 8.0. Eluted phages were reinfected into TG1 bacterial cells, amplified by co-infection with M13 helper phage, and purified by PEG precipitation. Purified phages expressing Fabs were selected for antigen binding as described. Phage from the second round was diluted and infected into TG1 cells. Polyclonal pools of phage output from two rounds of panning were tested by ELISA to confirm that the pools contained FAP-specific phage. Variable heavy and light chain regions were sequenced from single infected bacterial colonies using a rolling circle amplification and standard Sanger sequencing.

### 3.4. Antibody Expression

Unique variable heavy and light chain pairs from all discovery techniques were cloned into vectors designed to express full-length antibodies as IgGs containing LALA silencing mutations in HEK293 cells under the control of a CMV promoter. Antibody expression vectors were complexed with polyethylenimine and transfected into HEK293 cultures. After 5 days of shaking at 37° C. in 293 cell culture media, antibodies were captured on agarose-based protein A resin. After several stringent washes, antibodies were eluted in glycine solution, pH 3, neutralised with HEPES, pH 9, and buffer exchanged into PBS. The purity of purified proteins was confirmed by SEC-HPLC and CE-SDS analysis. Each antibody was assigned a unique clone number.

### 3.5. Binding of Anti-FAP Antibodies to FAP-Overexpressing HEK293 Cell Line

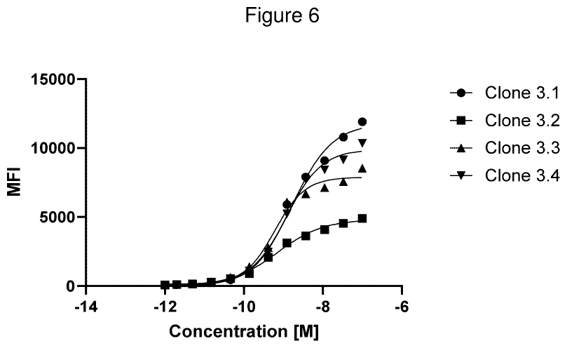

The binding of antibodies to human FAP expressed on transfected HEK293 cells was measured by FACS. In addition, binding to parental HEK293 was tested to confirm antibody specificity. In short, the cells were resuspended in the FACS binding buffer and live/dead efluro450 viability dye was added at a 1:1000 dilution to the cell suspension, followed by incubation at room temperature in the dark for 30 minutes. The cells were then diluted to 2 e5/ml, and 100 ul was added to wells of a 96-well U-bottom plate as required. For generating an EC50, a 100 nM stock was prepared and then diluted 3 fold serially across 12 points. The cells were pelleted by centrifugation, and the supernatant was discarded, followed by the addition of a series of dilutions of anti-FAP antibodies and incubation for 45 minutes at 4° C. in the dark. Cells were washed two times with PBS with 3% (w/v) BSA before adding 60 ul of fluorescently labelled secondary antibody (goat anti-human IgG) diluted to 1:200 in PBS. Incubation continued at 4 C for at least 30 minutes, followed by two washes in PBS pH 7.4. Cells were either fixed with 2% PFA (30 minutes at 4° C.) followed by washout and storage in 100 ul of PBS or immediately analysed on a Flow Cytometer. The data was analysed using FloJo V10 and GraphPad Prism. No binding to the HEK parental cell line was detected for any tested clones, confirming the specific interaction between anti-FAP antibodies and the FAP receptor. The results are summarised in. FIG. 6 demonstrates dose-dependent binding to FAP overexpressed on the HEK293 cell line.

TABLE 12
EC50 values for binding of anti-FAP
antibodies to FAP-HEK293 cell line

| | HuFAP-HEK293 | Hek293 parental |
|---|---|---|
| Clone ID | EC50 [M] | cell line |
| 3.1 | 1.7E−009 | No binding |
| 3.2 | 7.0E−010 | No binding |
| 3.3 | 6.0E−010 | No binding |
| 3.4 | 1.1E−009 | No binding |

### 3.6. Binding Kinetics of Anti-FAP Antibodies to Human and Cynomolgus FAP

Binding of anti-FAP antibodies to human and cynomolgus FAP was investigated by Surface Plasmon Resonance (SPR) on Carterra LSA. An EC30M chip was used to capture the test antibodies. The chip was activated for bioconjugation using EDC and sNHS in MES pH 5.5 to prepare the chip for amine coupling. Goat anti-human IgG Fc antibody was then captured to the chip via incubation for 10 minutes. Excess conjugation sites were quenched with 1M Ethanolamine pH 8. To measure monovalent binding kinetics, antibodies were printed/spotted onto the chip and allowed to bind to the goat antibody, followed by subsequent washing. After the preparation, the printed chip was exposed to repeated rounds of association and dissociation with increasing analyte concentrations. In this work, recombinant proteins (Human FAP (Acro) or cyno FAP His (Acro)) were serially diluted across a range starting at 1500 nM with 3-fold dilutions and 10 points. The curves were fitted to global fit in a 1:1 binding model to obtain KD values. The binding affinity to both human and cynomolgus FAP is reported in. Tested antibodies demonstrated comparable affinities to human and cynomolgus FAP.

TABLE 13
Binding affinity of anti-FAP antibodies
to human and cynomolgus FAP

| | HuFAP | cyFAP |
|---|---|---|
| Clone ID | KD[M] | KD[M] |
| 3.1 | 4.0E−11 | 5.1E−11 |
| 3.2 | 4.7E−11 | 2.8E−11 |
| 3.3 | 2.3E−11 | 2.1E−11 |
| 3.4 | 3.3E−11 | 3.0E−11 |

### 3.7. Epitope Competition Assay

A high-throughput epitope binning experiment was done on real-time label-free biosensors (Carterra LSA) to sort a panel of mAbs into bins based on their ability to block one another for binding to the antigen. In a pairwise epitope binning analysis, antigen and antibody 2 (analyte antibody) were sequentially applied to the sensor chip (HC200M) covalently pre-loaded with antibody 1 (ligand antibody). An increase in response upon exposure to the analyte antibody indicates non-competition between the two antibodies, whereas a lack of change in the signal indicates competition. Antibodies in the test set that have the same blocking profiles towards others are grouped into one bin. Community network plots were used to explore the clustering of mAbs that share similar but not necessarily identical competition profiles. Rather than relying strictly on the sandwiching/blocking assignments in the heat map, as the Bin network plots do, hierarchical clustering was applied to the sorted heat map to generate dendrograms, which progressively group mAbs. Community bins for tested anti-FAP antibodies are shown in. The results confirmed a diverse panel of anti-FAP antibodies, which were taken forward for further evaluation.

TABLE 14
Community bin defined by epitope competition assay on Carterra

| Clone ID | Community bin |
|---|---|
| 3.1 | 1 |
| 3.2 | 5 |
| 3.3 | 8 |
| 3.4 | 2 |

### 3.8. Sequence Optimisation of Anti-FAP Antibodies

The variable region of an antibody that has undergone somatic mutation may contain framework residues that differ from the germline sequence from which the antibody is derived. Therefore, to decrease the immunogenicity of the variable regions of antibody (VH and VL), the framework residues were reverted to corresponding human germline sequences. In short, different mutant variants were generated for each clone, which were then compared for their capacity to bind recombinant FAP on Octet. In each case the clone which was closest to human germline sequences was selected for further evaluation.

### 4. Example 4 Generation of Bispecific Antibodies Targeting FAP and LTBR

### 4.1. Production of Human and Mouse-Specific FAP/LTBR Bispecific Antibodies

The anti-LTBR VHHs identified in Example 1 were shown to have agonistic activity in an NFKB reporter assay when cross-linked by an external cross-linking agent (e.g. an anti-His antibody or when formatted as a bivalent molecule on a human IgG1 Fc region). These clones presented the most favourable functional properties for bispecific antibody evaluation.

A highly diverse panel of anti-FAP antibodies was identified in the previous examples with a range of affinities that bind to different huFAP regions.

The human cynomolgus cross-reactive anti-LTBR agonist VHHs (as described in Example 1) or anti-LTBR agonist antibodies (as described in Example 2) and anti-FAP antibodies (as described in Example 3) were used to generate constructs comprising a FAP binding agent and an LTBR binding agent. These constructs were FAP/LTBR bispecific agonistic antibodies. Different formats of bispecific antibodies were prepared with either bivalent or monovalent binding for LTBR and monovalent or bivalent binding for FAP, i.e. FAP:LTBR 1:1 format, 2:1 format, or 2:2 format. Schematic illustrations of the different bispecific formats produced are represented in FIGS. 7a-7d. These FAP/LTBR bispecific agonistic antibodies comprised one or more FAP binding agents and one or more LTBR binding agents variously in the form of (a) one or two anti-FAP Fab region(s) and (b) one or two anti-LTBR VHH(s) or one or two anti-LTBR scFv(s). FIG. 7a includes an anti-LTBR VHH replacing one Fab, FIG. 7b includes one anti-LTBR VHH linked via a synthetic linker to a CH3 domain, FIG. 7c includes one anti-LTBR VHH linked via a synthetic linker to each of the two CH3 domains and FIG. 7d includes one anti-LTBR scFv linked via a synthetic linker to each of the two CH3 domains. FIG. 7a includes an anti-FAP Fab and FIGS. 7b-d include two anti-FAP Fabs. Each construct was assigned a unique clone number.

The variable regions of human-specific constructs were cloned into expression vectors containing the constant region of human IgG1. In some cases, to generate either 2:1 or 2:2 formats, scFvs or VHHs containing sequences of variable regions of anti-LTBR antibodies (Examples 1 and 2) were fused to the C-terminus of an Fc by a G₄S linker (SEQ ID NO: 3). As scFv fragments could have stability issues, an additional disulfide bond was introduced in the VH44:VL100 position (Weatherill et al., 2012). A well-established knob-into-holes strategy was employed to ensure the correct assembly of heavy chains (Merchant et al., 1998). In addition, to reduce the binding to FcyRs, LALA (Leu234Ala and Leu235Ala) or PGLALA (Pro329Gly, Leu234Ala, and Leu235Ala), Schlothauer et al. 2016, were introduced in the Fc region.

To produce murine surrogate antibodies, the variable regions of mouse-specific constructs were cloned into expression vectors containing the constant region of mouse IgG2a or mouse IgG1. To ensure the correct assembly of light and heavy chains, published mutations in CH1/CL to promote sufficient light chain pairing and electrostatic steering mutations into the CH3 domain were introduced to ensure specific heavy chain pairing (Wang et al., 2020) were introduced. Like human constructs to prevent the binding to FcyRs, PGLALA (Pro329Gly, Leu234Ala, and Leu235Ala, Schlothauer et al. 2016) were introduced in the Fc region. In some cases, to generate either 2:1 or 2:2 formats, scFv containing sequences of variable regions 5G11 were fused to the C-terminus of an Fc by a G₄S linker (SEQ ID NO: 3). As scFv fragments could have stability issues, an additional disulfide bond was introduced in the VH44:VL100 position (Weatherill et al., 2012).

In parallel, (i) a monospecific control antibody, (ii) anti-human LTBR control clones BHA10, and CBE-11 (as disclosed in WO2004002431), (iii) anti-FAP clone 28H1 (as disclosed in U.S. Ser. No. 10/577,429B2) and (iv) anti-mouse LTBR control (clone 5G11) were cloned into expression vectors containing either the constant region of human IgG1, rat IgG2a or mouse IgG1. PGLALA mutations (Pro329Gly, Leu234Ala, and Leu235Ala, (Schlothauer et al., 2016)) were introduced in the constant region of the human IgG1/mouse IgG2a heavy chains to abrogate binding to Fc gamma receptors.

In addition, FAP/LTBR bispecific comparative example antibodies were produced, clones: P1AH5886, P1AG5459 and PCAG5461 (as disclosed in WO2023/117834A1).

The bispecific and monospecific controls were expressed by transient transfection of CHO suspension cells. Antibodies were purified from cell culture supernatants by affinity chromatography using protein A resin and followed by one or two chromatography steps: preparative size exclusion (SEC) and/or ion exchange to enrich the heterodimer.

The purity and heterogeneity of the samples were assessed by SEC-H PLC and mass spectrometry. All samples were confirmed to have a monomer content of 95% and contain <10% impurities prior to functional read-out.

The resulting bispecific and monospecific antibodies are listed in, and the sequences of listed clone variable regions can be found in

TABLE 15
List of bispecific antibodies and monospecific controls

| | | LTBR | FAP | |
|---|---|---|---|---|
| Clone ID | Format | Clone | Clone | Isotype |
| 4.1 | 1:1 Heterodimer consisting | 1.6 | 3.5 | Human |
| | of LTBR VHH paired with | | | IgG1 |
| | FAP Fab | | | LALA |
| 4.2 | 1:1 Heterodimer consisting | 1.7 | 3.5 | Human |
| | of LTBR VHH paired with | | | IgG1 |
| | FAP Fab | | | LALA |
| 4.3 | 1:1 Heterodimer consisting | 1.6 | 3.6 | Human |
| | of LTBR VHH paired with | | | IgG1 |
| | FAP Fab | | | LALA |
| 4.4 | 2:1 Heterodimer consisting | 1.6 | 3.6 | Human |
| | of FAP mAb with C terminal | | | IgG1 |
| | fused LTBR VHH | | | LALA |
| 4.5 | 2:1 Heterodimer consisting | 1.6 | 3.5 | Human |
| | of FAP mAb with C terminal | | | IgG1 |
| | fused LTBR VHH | | | LALA |
| 4.6 | 2:1 Heterodimer consisting | 1.7 | 3.6 | Human |
| | of FAP mAb with C terminal | | | IgG1 |
| | fused LTBR VHH | | | LALA |
| 4.7 | 2:1 Heterodimer consisting | 2.3 | 3.7 | Human |
| | of FAP mAb with C terminal | | | IgG1 |
| | fused LTBR scFv | | | LALA |
| 4.8 | 2:2 Homodimer IgG | 1.7 | 3.5 | Human |
| | consisting of FAP mAb with | | | IgG1 |
| | C-terminal fused anti-LTBR | | | LALA |
| | VHHs | | | |
| 4.9 | 2:2 Homodimer IgG | 1.6 | 3.5 | Human |
| | consisting of FAP mAb with | | | IgG1 |
| | C-terminal fused anti-LTBR | | | LALA |
| | VHHs | | | |
| 4.10 | 1:1 Heterodimer consisting | 1.6 | 3.4 | Human |
| | of LTBR VHH paired with | | | IgG1 |
| | FAP Fab | | | LALA |
| 4.11 | 2:2 Homodimer IgG | 5G11 | 28H1 | Mouse |
| | consisting of FAP mAb with | | | IgG2a |
| | C-terminal fused anti-LTBR | | | LALAPG |
| | scFvs | | | |
| 4.12 | 2:2 Homodimer IgG | 5G11 | 28H1 | Mouse |
| | consisting of FAP mAb with | | | IgG2a |
| | C-terminal fused anti-LTBR | | | LALA |
| | scFvs | | | |
| 4.13 | 2:2 Homodimer IgG | Herceptin | 28H1 | Mouse |
| | consisting of huHER2 mAb | | | IgG2a |
| | with C-terminal fused anti- | | | LALA |
| | LTBR scFvs | | | |
| 4.14 | A monoclonal antibody | 5G11 | NA | Rat |
| | specific to mouse LTBR | | | IgG2a |
| 4.15 | A monoclonal antibody | 5G11 | NA | Mouse |
| | specific to mouse LTBR | | | IgG1 |
| 4.16 | 2:1 Heterodimer consisting | 2.2 | 3.3 | Human |
| | of FAP mAb with C terminal | | | IgG1 |
| | fused LTBR scFv | | | LALA |
| 4.17 | 2:1 Heterodimer consisting | 2.3 | 3.1 | Human |
| | of FAP mAb with C terminal | | | IgG1 |
| | fused LTBR scFv | | | LALA |
| 4.18 | 2:1 Heterodimer consisting | 2.2 | 3.1 | Human |
| | of FAP mAb with C terminal | | | IgG1 |
| | fused LTBR scFv | | | LALA |
| BHA10 | A monoclonal antibody | BHA10 | NA | Human |
| | specific to human LTBR | | | IgG1 |
| CBE-11 | A monoclonal antibody | CBE-11 | NA | Human |
| | specific to human LTBR | | | IgG1 |
| P1AH5886 | 2:1 Heterodimer consisting | P1AE9459 | 212 | Human |
| Comparative | of LTBR mAb with C | | | IgG1 |
| example 1 | terminal fused FAP in Fab | | | |
| | format | | | |
| P1AG5459 | 2:1 Heterodimer consisting | P1AE9459 | 28H1 | Human |
| Comparative | of LTBR mAb with C | | | IgG1 |
| example 2 | terminal fused FAP in Fab | | | |
| | format | | | |
| P1AG5461 | 2:1 Heterodimer consisting | P1AE9459 | DP47 | Human |
| Comparative | of LTBR mAb with C | | (WO2023117834) | IgG1 |
| example 3 | terminal fused DP47 in Fab | | | |
| | format | | | |

All 1:1 format constructs detailed in the table above included a truncated hinge region in HC1 (DKTHTCPPCP, SEQ ID NO: 203) and a native hinge region in HC2 (EPKSCDKTHTCP, SEQ ID NO: 204). The constructs of other formats included native hinge regions in the HC (EPKSCDKTHTCP, SEQ ID NO: 204).

TABLE 16
Amino acid sequences of variable regions of FAP and LTBR clones

| | Heavy chain variable region | SEQ | Light chain variable region | SEQ ID |
|---|---|---|---|---|
| | polypeptide sequence | ID NO | polypeptide sequence | NO |
| BHA10 | QVQLVQSGAEVKKPGSSVKV | 1 | DIQMTQSPSSLSASVGDR | 2 |
| (anti- | SCKASGYTFTTYYLHWVRQA | | VTITCKASQNVGINVAWY | |
| LTBR) | PGQGLEWMGWIYPGNVHAQ | | QQKPGKAPKSLISSASYR | |
| | YNEKFKGRVTITADKSTSTAY | | YSGVPSRFSGSGSGTDFT | |
| | MELSSLRSEDTAVYYCARSW | | LTISSLQPEDFATYFCQQY | |
| | EGFPYWGQGTTVTVSS | | DTYPFTFGQGTKVEIK | |
| 5G11 | QVQLKESGPGLVQPSQTLSL | 5 | DVQMTQSPSYLAASPGES | 6 |
| (anti- | TCTVSGFSLTTYSVHWVRQH | | VSIRCKASKSISNNLAWYQ | |
| LTBR) | SGKSLEWMGRMWTDGDTSY | | EKPGKANKLLIHSGSTLQS | |
| | NSAFTSRLSISRDTSKSQVFL | | GTPSRFSGSGSGTDFTLTI | |
| | KMNSLQTEDTGTYYCARGY | | RSLEFEDFAVYYCQQYNE | |
| | WYFDFWGPGTMVTVSS | | YPYTFGAGTKLELK | |
| 28H1 | EVQLLESGGGLVQPGGSLRL | 7 | EIVLTQSPGTLSLSPGERA | 8 |
| (anti- | SCAASGFTFSSHAMSWVRQ | | TLSCRASQSVSRSYLAWY | |
| FAP) | APGKGLEWVSAIWASGEQYY | | QQKPGQAPRLLIIGASTRA | |
| | ADSVKGRFTISRDNSKNTLYL | | TGIPDRFSGSGSGTDFTLT | |
| | QMNSLRAEDTAVYYCAKGW | | ISRLEPEDFAVYYCQQGQ | |
| | LGNFDYWGQGTLVTVSS | | VIPPTFGQGTKVEIK | |
| 1.5 | DVQLVESGGGLVQPGGSLRL | 23 | NA | NA |
| (anti- | SCAASGSSIFSFEIMAWYRQ | | | |
| LTBR) | APGKQRELVAVINKGGEANY | | | |
| | PDSVKGRFTISRDNAKNTVYL | | | |
| | QMNSLRPEDTAVYYCNADAV | | | |
| | PHGSYWGQGTLVTVSS | | | |
| 1.6 | DVQLVESGGGLVQPGGSLRL | 24 | NA | NA |
| (anti- | SCKASGTTFSDRAFGWYRQ | | | |
| LTBR) | APGKQRELVATISTAGLTWY | | | |
| | DVSVKGRFTISRDNAKNTVYL | | | |
| | QMNSLRPEDTAVYYCNTFRG | | | |
| | NWGQGTLVTVSS | | | |
| 1.7 | DVQLVESGGGLVQPGGSLRL | 25 | NA | NA |
| (anti- | SCAASGRFFGIYDMYWYRQA | | | |
| LTBR) | PGKQRELVAISTSGGHTNYA | | | |
| | DSVKGRFTISRDNAKNTVYL | | | |
| | QMNSLRPEDTAVYYCNIQRV | | | |
| | DAPGFYWGQGTLVTVSS | | | |
| 1.8 | DVQLVESGGGLVQPGGSLRL | 26 | NA | NA |
| (anti- | SCAASGRTFTNYRMAWFRQ | | | |
| LTBR) | APGKEREAVAAINWGGGGTY | | | |
| | YADSVKGRFTISRDNAKNTV | | | |
| | YLQMNSLRPEDTAVYYCAAD | | | |
| | FTGWGSYFDYWGQGTLVTV | | | |
| | SS | | | |
| 2.1 | EVQLVESGGGLVKPGGSLRL | 27 | DIQMTQSPSSLSASIGDRV | 28 |
| (anti- | SCAASGFTFSSYCMNWVRQ | | TITCQASQDISNFLSWYQQ | |
| LTBR) | APGKGLEWVSSISTGNTYIYY | | KPGKAPNLLIFDASNLETG | |
| | SDSVKGRFTISRDNAKNSLYL | | VPSRFSESGSGTDFSFTIT | |
| | QMNSLRAEDTAVYYCARARY | | SLQPEDIATYYCQQYYNV | |
| | NWNYDAFDIWGQGTMVTVS | | PLTFGGGTKVEIK | |
| | S | | | |
| 2.2 | EVQLVESGGGLVKPGGSLRL | 29 | DIQMTQSPSTLSASVGDR | 30 |
| (anti- | SCAASRFTFSSYSMNWVRQ | | VTITCRASQSISSWLAWY | |
| LTBR) | APGKGLEWVSSISPSSSYIYY | | QQKPGKAPKFLIYKASSLE | |
| | ADSVKGRFTISRDNAKNSLYL | | SGVPSRFSGSGSGTEFTL | |
| | QMNSLRAEDTAVYYCARDHY | | TISSLQPDDFATYYCQQY | |
| | NWNFDALDIWGQGTMVTVS | | NSFSRTFGQGTKVEIK | |
| | S | | | |
| 2.3 | EVQLVESGGGLVQPGGSLRL | 31 | DIVMTQSPDSLAVSLGER | 32 |
| (anti- | SCAASGFTFSSYAMSWVRQ | | ATINCKSSQNVLYSSNNK | |
| LTBR) | APGKGLEWVSGISGSGGRTN | | NYLTWYQQKPGQPPKLIIY | |
| | YADSVKGRFTISRDNSKNTLY | | WASTRESGVPDRFSGSG | |
| | LQMNSLRAEDTAVYYCAKSD | | SGTDFTLTISSLQAEDVAV | |
| | NWHYLDAFDIWGQGTMVTV | | YYCQQYFNTPPTFGQGTK | |
| | SS | | VEIK | |
| 2.4 | QVQLVESGGGVVQPGRSLR | 33 | DIVMTQSPDSLAVSLGER | 34 |
| (anti- | LSCAASGFTFSSYGIHWVRQ | | ATINCKSSQSVFYSSNNK | |
| LTBR) | APGKGLEWVAVIWYDGSNKY | | NYLVWYQQKPGQPPKLLI | |
| | YADSVKGRFTISRDNSKNTLY | | YWASTRASGVPDRFSGS | |
| | LQMNSLRAEDTAVYYCARDG | | GSGTDFTLTISSLQAEDVA | |
| | GTGNYYYMDVWGKGTTVTV | | VYYCQQYYNTPLTFGGGT | |
| | SS | | KVEIK | |
| 2.5 | EVQLVESGGGLVQPGRSLRL | 35 | DIVMTQSPDSLAVSLGER | 36 |
| (anti- | SCAASGFTFDDYDIHWVRQA | | ATINCKSSQSVLYSSNNKN | |
| LTBR) | PGKGLEWVSGINWNSITIGYA | | YLAWYQQKPGQPPKLLIY | |
| | DSVKGRFTISRDNAKNSLYLQ | | WASTRESGVPDRFSGSG | |
| | MNSLRAEDTALYYCAKDRGS | | SGTDFTLTISSLQAEDVAV | |
| | GWRIFDYWGQGTLVTVSS | | YYCQQYYSTPYTFGQGTK | |
| | | | LEIK | |
| 2.6 | QVQLVQSGTEMKKPGASVK | 37 | DIVMTQSPDSLAVSLGER | 38 |
| (anti- | VSCKASGYTFSSYDISWVRQ | | ATINCKSSQSVLYSSNNKN | |
| LTBR) | APGQGLEWMGWFSAYNGN | | YLAWYQQKPGQPPNLLIY | |
| | SNYAQKFQGRVTMTTDTSTN | | WASIRESGVPERFSGSGS | |
| | TAYMELRSLRSDDTAVYYCA | | GTDFTLTISSLQAEDVAVY | |
| | REGYYGNSGFFDYWGQGTL | | YCQQYYTSPYTFGQGTKL | |
| | VTVSS | | EIK | |
| 2.7 | EVQLVESGGGLVQPGGSLRL | 39 | DIVMTQSPDSLAVSLGER | 40 |
| (anti- | SCAASGFIFDDYDMHWVRQA | | ATINCKSSQSVLYSSNNKN | |
| LTBR) | PGKGLEWVSGINWNSNRVD | | YLAWYQQKPGQPPKLLIY | |
| | YADSVKGRFTISRDNARNSL | | WASTRESGVSDRFSGSG | |
| | YLQMNSLRPEDTAFYYCVKD | | SGTDFTLTISSLQAEDVAV | |
| | KTSSWYVFDSWGQGTPVTV | | YYCQQYYSIPYTFGQGTK | |
| | SS | | LEIK | |
| 2.8 | QVQLQESGPGLVKPSGTLSL | 41 | DIQMTQSPSSLSASVGDR | 42 |
| (anti- | TCAVSGGSISSSNWWSWVR | | VNITCRASQSISSYLNWYQ | |
| LTBR) | QPPGKGLEWIGDIYHDGTTY | | QKPGKAPKLLIYAPSSLQS | |
| | YNPSLKSRVTVSVDKSKNQF | | GVPSRFSGGGSGTDFTLT | |
| | SLKLSSVTAADTAVYYCARSP | | ISSLQPEDFATYFCQQSYS | |
| | SGTTDYFDYWGQGTLVTVSS | | TPPTFGPGTKVDIK | |
| 3.5 | QLQLQESGPGLVKPSETLSL | 43 | DIQMTQSPSSLSASVGDR | 44 |
| (anti- | TCTVSGGSISSSTYYWGWIR | | VTITCRASQSISKYLNWYQ | |
| FAP) | QPPGKGLEWIGSIFYSGNTY | | QKPGKAPKLLIYAASSLQS | |
| | YNPSLKSRVTISVDTSKNHFS | | GVPSRFSGSGSGTDFTLTI | |
| | LKLSSVTAADTAVYYCAREIIE | | SSLQPEDFATYYCQQSNS | |
| | PRPGYFDYWGQGTLVTVSS | | IPRTFGQGTKVEIK | |
| 3.6 | QVQLVQSGAEVKKPGASVKV | 45 | DIQMTQSPSTLSASVGDR | 46 |
| (anti- | SCKASGYTFTGYYMHWVRQ | | VTITCRASQSIGSWLAWY | |
| FAP) | APGQGLEWMGWIYPNTGGT | | QQKPGKAPKLLIYKASSLE | |
| | NYAQNFQGRVTMTRDTSIST | | SGVPSRFSGSGSGTEFTL | |
| | AYMELSRLRSDDTAVYYCAR | | TISSLQPDDFATYYCQQY | |
| | DDYNKNLDYWGQGTLVTVS | | NSYFRTFGQGTKVEIK | |
| | S | | | |
| 3.7 | QVQLVQSGAEVKKPGASVKV | 47 | DIQMTQSPSSLSASVGDR | 48 |
| (anti- | SCKASGYTFTGYYMNWVRQ | | VTITCRASQNIGSYLNWYQ | |
| FAP) | APGQGLEWMGWINPQSGDV | | QKPGKAPKLLIYAASSLQS | |
| | NFAQKFQGRVTMTRDTSIST | | GVPSRFSGSGSGTDFTLTI | |
| | AYMELSRLRSDDTAVYYCAR | | SSLQPEDFATYYCQQANS | |
| | QFRGYSYGYGMDVWGQGTT | | FPFTFGPGTKVDIKRTV | |
| | VTVSS | | | |
| 3.4 | EVQLVESGGGLVKPGGSLRL | 49 | DIQMTQSPSSLSASVGDR | 50 |
| (anti- | SCVASGFTFSLAWMNWVRQ | | VTITCRASQSINSYFNWYQ | |
| FAP) | APGKGLEWVGHIKSRADGGS | | QKPGKAPKLLIYAASGLQS | |
| | TDYAAPVKGRFTISRDDSKNT | | GVPSRFSGSGSGTDFTLTI | |
| | LYLHMNSLKTEDTAVYFCSR | | SSLQPEDFATYYCQQSYS | |
| | FYWNYVLDYWGQGTLVTVS | | PPYTFGQGTNLEIK | |
| | S | | | |
| 3.1 | QLQLQESGPGLVKPSETLSL | 51 | DIQMTQSPSSLSASVGDR | 52 |
| (anti- | TCTVSGGSISSSTYYWGWIR | | VTITCRASQSISKYLNWYQ | |
| FAP) | QPPGKGLEWIGSIFYSGNTY | | QKPGKAPKFLIYAASSLQS | |
| | YNPSLKSRVTISVDTSKNQFS | | GVPSRFSGSGSGTDFTLTI | |
| | LRLNSVTAADTAVYYCAREIIE | | SSLQPEDFATYYCQQSNS | |
| | PRPGYFDYWGQGTLVTVSS | | IPRTFGQGTKVEIK | |
| 3.2 | QVQLVQSGAEVKKPGASVKV | 53 | DIQMTQSPSTLSASVGDR | 54 |
| (anti- | SCKASGYTFTGYYMHWVRQ | | GTITCRASQSIGSWLAWY | |
| FAP) | APGQGLEWMGWIYPNTGGT | | QQKPGKTPKLLIYKASSLE | |
| | NYAQNFQGRVTMTRDTSIST | | SGVPSRFSGSGSGTEFTL | |
| | AYMELSSLRSDDTAVYYCAR | | TISSLQPDDFATYYCQQY | |
| | DDYNKNLDYWGQGTLVTVS | | NSYFRTFGQGTKVEIK | |
| | S | | | |
| 3.3 | QVQLVQSGTEVKKPGASVKV | 55 | DIQMTQSPSSLSASVGDR | 56 |
| (anti- | SCKASGYTFTGYYMNWVRQ | | VTITCRASQNIGSYLNWYQ | |
| FAP) | APGQGLEWVGWINPNSGDT | | QKPGKAPKLLIYAASSLQS | |
| | NFAQKFQGRVTMTRDSSIST | | GVPSRFSGSGSGTDFTLTI | |
| | AYMELSRLRSDDTAVYYCAR | | SSLQPEDFATYYCQQANS | |
| | QFRGYSYGYGMDVWGKGTT | | FPFTFGPGTKVDIK | |
| | VTVSS | | | |
| 1.1 (anti- | EVQLVESGGGLVQPGGSLRL | 159 | NA | |
| LTBR) | SCAASGRFFGIYDMYWYRQP | | | |
| | PGKQRELVAISTSGGHTNYA | | | |
| | DSVKGRFTISRDNAKNTVYL | | | |
| | QMNSLRPEDTAVYYCNIQRV | | | |
| | DAPGMYWGQGTLVTVSS | | | |
| 1.2 (anti- | EVQLVESGGGLVQPGGSLRL | 160 | NA | |
| LTBR) | SCAASGSSIFSFEIMAWYRQ | | | |
| | APGKQRELVAVINKGGEANY | | | |
| | PDSVEGRFTISRDNAKNTVYL | | | |
| | QMNSLRPEDTAVYYCNADAV | | | |
| | PHGSYWGQGTLVTVSS | | | |
| 1.3 (Anti- | EVQLVESGGGLVQPGGSLRL | 161 | NA | |
| LTBR) | SCAASGRTFTNYRMAWFRQ | | | |
| | APGKEREAVAAINWNGGGTY | | | |
| | YADSVKGRFTISRDNAKNTV | | | |
| | YLQMNSLRPEDTAVYYCAAD | | | |
| | STGWGSYFDYWGQGTLVTV | | | |
| | SS | | | |
| 1.4 (Anti- | EVQLVESGGGLVQPGGSLRL | 162 | NA | |
| LTBR) | SCKASGTTFSDRAFGWYRQ | | | |
| | APGKQRELVATISTAGLTWY | | | |
| | DVSVKGRFIISRDNPENTVYL | | | |
| | QMNSLRPEDTAVYYCNTFRG | | | |
| | NWGQGTLVTVSS | | | |

TABLE 17A
Amino acid sequences of CDRs of FAP and LTBR clones. For VHHs, HCDR sequences
are provided. For paired VH/VLs clones HCDR sequences are provided in the first
row for each clone, followed by LCDR sequences in the second row for the same clone.

| Clone | | SEQ ID | | SEQ ID | | SEQ ID |
|---|---|---|---|---|---|---|
| ID | CDR1 | NO | CDR2 | NO | CDR3 | NO |
| 1.1 | IYDMY | 163 | ISTSGGHTNYADS | 164 | QRVDAPGMY | 165 |
| | | | VKG | | | |
| 1.2 | SFEIMA | 166 | VINKGGEANYPDS | 167 | DAVPHGSY | 168 |
| | | | VEG | | | |
| 1.3 | NYRMA | 169 | AINWNGGGTYYAD | 170 | DSTGWGSYFDY | 171 |
| | | | SVKG | | | |
| 1.4 | DRAFG | 172 | TISTAGLTWYDVSV | 173 | FRGN | 174 |
| | | | KG | | | |
| 1.5 | SFEIMA | 57 | VINKGGEAN | 58 | DAVPHGSY | 59 |
| | | | YPDSVKG | | | |
| 1.6 | DRAFG | 60 | TISTAGLTW | 61 | FRGN | 62 |
| | | | YDVSVKG | | | |
| 1.7 | IYDMY | 63 | ISTSGGHTN | 64 | QRVDAPGFY | 65 |
| | | | YADSVKG | | | |
| 1.8 | NYRMA | 66 | AINWGGGGTYYAD | 67 | DFTGWGSYFDY | 68 |
| | | | SVKG | | | |
| 2.1 | SYCMN | 69 | SISTGNTYIY | 70 | ARYNWNYDA | 71 |
| | | | YSDSVKG | | FDI | |
| 2.1 | QASQDISN | 72 | DASNLET | 73 | QQYYNVPLT | 74 |
| | FLS | | | | | |
| 2.2 | SYSMN | 75 | SISPSSSYIY | 76 | DHYNWNFDA | 77 |
| | | | YADSVKG | | LDI | |
| 2.2 | RASQSISS | 78 | KASSLES | 79 | QQYNSFSRT | 80 |
| | WLA | | | | | |
| 2.3 | SYAMS | 81 | GISGSGGRT | 82 | SDNWHYLDA | 83 |
| | | | NYADSVKG | | FDI | |
| 2.3 | KSSQNVL | 84 | WASTRES | 85 | QQYFNTPPT | 86 |
| | YSSN | | | | | |
| | NKNYLT | | | | | |
| 2.4 | SYGIH | 87 | VIWYDGSNK | 88 | DGGTGNYYY | 89 |
| | | | YYADSVKG | | MDV | |
| 2.4 | KSSQSVF | 90 | WASTRAS | 91 | QQYYNTPLT | 92 |
| | YSSN | | | | | |
| | NKNYLV | | | | | |
| 2.5 | DYDIH | 93 | GINWNSITIG | 94 | DRGSGWRIF | 95 |
| | | | YADSVKG | | DY | |
| 2.5 | KSSQSVL | 96 | WASTRES | 97 | QQYYSTPYT | 98 |
| | YSSN | | | | | |
| | NKNYLA | | | | | |
| 2.6 | SYDIS | 99 | WFSAYNGNS | 100 | EGYYGNSGF | 101 |
| | | | NYAQKFQG | | FDY | |
| 2.6 | KSSQSVL | 102 | WASIRES | 103 | QQYYTSPYT | 104 |
| | YSSN | | | | | |
| | NKNYLA | | | | | |
| 2.7 | DYDMH | 105 | GINWNSNR | 106 | DKTSSWYVF | 107 |
| | | | VDYADSVKG | | DS | |
| 2.7 | KSSQSVL | 108 | WASTRES | 109 | QQYYSIPYT | 110 |
| | YSSN | | | | | |
| | NKNYLA | | | | | |
| 2.8 | SSNWWS | 111 | DIYHDGTTY | 112 | SPSGTTDYF | 113 |
| | | | YNPSLKS | | DY | |
| 2.8 | RASQSISS | 114 | APSSLQS | 115 | QQSYSTPPT | 116 |
| | YLN | | | | | |
| 3.5 | SSTYYWG | 117 | SIFYSGNTY | 118 | EIIEPRPGYF | 119 |
| | | | YNPSLKS | | DY | |
| 3.5 | RASQSISK | 120 | AASSLQS | 121 | QQSNSIPRT | 122 |
| | YLN | | | | | |
| 3.6 | GYYMH | 123 | WIYPNTGG | 124 | DDYNKNLDY | 125 |
| | | | TNYAQNFQG | | | |
| 3.6 | RASQSIGS | 126 | KASSLES | 127 | QQYNSYFRT | 128 |
| | WLA | | | | | |
| 3.7 | GYYMN | 129 | WINPQSGDVNFAQ | 130 | QFRGYSYGY | 131 |
| | | | KFQG | | GMDV | |
| 3.7 | RASQNIGS | 132 | AASSLQS | 133 | QQANSFPFT | 134 |
| | YLN | | | | | |
| 3.4 | LAWMN | 135 | HIKSRADGG | 136 | FYWNYVLDY | 137 |
| | | | STDYAAPVKG | | | |
| 3.4 | RASQSINS | 138 | AASGLQS | 139 | QQSYSPPYT | 140 |
| | YFN | | | | | |
| 3.1 | SSTYYWG | 141 | SIFYSGNTYYN | 142 | EIIEPRPGYF | 143 |
| | | | PSLKS | | DY | |
| 3.1 | RASQSISK | 144 | AASSLQS | 145 | QQSNSIPRT | 146 |
| | YLN | | | | | |
| 3.2 | GYYMH | 147 | WIYPNTGGTN | 148 | DDYNKNLDY | 149 |
| | | | YAQNFQG | | | |
| 3.2 | RASQSIGS | 150 | KASSLES | 151 | QQYNSYFRT | 152 |
| | WLA | | | | | |
| 3.3 | GYYMN | 153 | WINPNSGDTN | 154 | QFRGYSYGY | 155 |
| | | | FAQKFQG | | GMDV | |
| 3.3 | RASQNIGS | 156 | AASSLQS | 157 | QQANSFPFT | 158 |
| | YLN | | | | | |

The heavy chain and light chain polypeptide sequences for Clones 4.1 to 4.10 are as follows, wherein for homodimers, HC=heavy chain, LC=light chain. In the case of heterodimers, polypeptide sequences for the first (HC1) and second (HC2) heavy chains are provided in addition to the LC polypeptide sequence.
Clone 4.1 HC1: SEQ ID NO: 175, HC2: SEQ ID NO: 176, LC: SEQ ID NO: 177
Clone 4.2 HC1: SEQ ID NO: 178, HC2: SEQ ID NO: 179, LC: SEQ ID NO: 180
Clone 4.3 HC1: SEQ ID NO: 181, HC2: SEQ ID NO: 182, LC: SEQ ID NO: 183
Clone 4.4 HC1: SEQ ID NO: 184, HC2: SEQ ID NO: 185, LC: SEQ ID NO: 186
Clone 4.5 HC1: SEQ ID NO: 187, HC2: SEQ ID NO: 188, LC: SEQ ID NO: 189
Clone 4.6 HC1: SEQ ID NO: 190, HC2: SEQ ID NO: 191, LC: SEQ ID NO: 192
Clone 4.7 HC1: SEQ ID NO: 193, HC2: SEQ ID NO: 194, LC: SEQ ID NO: 195
Clone 4.8 HC: SEQ ID NO: 196, LC: SEQ ID NO: 197
Clone 4.9 HC: SEQ ID NO: 198, LC: SEQ ID NO: 199
Clone 4.10 HC1: SEQ ID NO: 200, HC2: SEQ ID NO: 201, LC: SEQ ID NO: 202

TABLE 17B
Alternative amino acid sequences of CDRs or LTBR VHHs. Alternative CDRs
resulting from combination of nearly identical individual CDRs are provided
with their amino acid sequence and SEQ ID NO:.

| | CDR | CDR | Alternative CDR |
|---|---|---|---|
| # | (SEQ ID NO:) | (SEQ ID NO:) | (sequence and SEQ ID NO:) |
| 1 | 58 | 167 | VINKGGEANYPDSVXG wherein X at |
| | | | position 15 is Lys (K) or Glu (E) |
| | | | (SEQ ID NO: 205) |
| 2 | 67 | 170 | AINWXGGGTYYADSVKG wherein X |
| | | | at position 5 is Gly (G) or Asn (N) |
| | | | (SEQ ID NO: 206) |
| 3 | 65 | 165 | QRVDAPGXY wherein X at position 8 |
| | | | is Phe (F) or Met (M) |
| | | | (SEQ ID NO: 207) |
| 4 | 68 | 171 | DXTGWGSYFDY wherein X at |
| | | | position 2 is Phe (F) or Ser (S) |
| | | | (SEQ ID NO: 208) |

### 4.2. Binding of FAP/LTBR Bispecific Antibodies to Human FAP and LTBR-Expressing Cells

To test the binding of FAP/LTBR bispecific antibodies to human FAP on cells, a binding assay using a FAP-over expressing CHO-K1 recombinant cell line (BPS, cat no. 79947) or a FAP overexpressing transient HEK293 cell line was used. To test the binding of FAP/LTBR bispecific antibodies to human LTBR on cells, a binding assay using the endogenously LTBR-expressing A549 (ATCC, cat. no CCL-185) or A375 tumor cell line was used. In this experiment, an LTBR monospecific antibody (BHA10) was used as a control.

In some cases, the secondary antibody interfered with detection; therefore, the bispecific antibodies were labelled directly with Alexa Fluor®647 Conjugation Kit (Fast)—Lightning-Link®(Abcam, cat no ab269823), following manufacturer instructions. In that case, the staining protocol was as described below, apart from adding a secondary antibody.

In short, 1×10⁶ cells were resuspended in the FACS buffer (1% BSA, 0.1% Sodium Azide in PBS), and 50 μl was added to each well (5×10⁴ cells/well) of a 96-U bottom plate. The cells were washed once and resuspended in 50 μl/well of FACS buffer containing a 1:3, 11-point series dilutions of constructs 4.1-4.6 and clones 4.8-4.9 followed by 1 h incubation at 4° C. After extensive washing, cells were further stained with a secondary detection antibody, R-Phycoerythrin-conjugated AffiniPure Goat Anti-Human IgG, Fcγ Fragment Specific (Stratech, cat. no 109-115-098-JIR), for 1 h incubation at 4° C. Next, cells were washed and resuspended in FACS buffer and read on Beckman Coulter CytoFLEX. The data were analysed using FloJo V10 and GraphPad Prism v9. Clone 4.7 was tested for binding to A375 (LTBR positive cell line) and the HEK293-FAP overexpressing cell line, as described in examples 2.4 and 3.5. The EC50 values can be found in.

TABLE 18
EC50 values for binding of FAP/LTBR bispecific
antibodies to LTBR and FAP positive cell lines

| Clone ID | A549 | A375 | CHO-FAP | Hek-FAP |
|---|---|---|---|---|
| 4.1 | 2.9E−08 | No tested | 2.8E−010 | No tested |
| 4.2 | 3.2E−09 | No tested | 4.7E−010 | No tested |
| 4.3 | 6.9E−09 | No tested | 8.0E−010 | No tested |
| 4.4 | 4.5E−08 | No tested | 2.0E−010 | No tested |
| 4.5 | 2.9E−08 | No tested | 1.1E−010 | No tested |
| 4.6 | NP | No tested | 3.3E−010 | No tested |
| 4.7 | No tested | 6.0E−009 | No tested | 3.4E−010 |
| 4.16 | No tested | NP | No tested | 4.0E−010 |
| 4.17 | No tested | NP | No tested | 8.0E−010 |
| 4.18 | No tested | NP | No tested | 1.0E−09 |
| BHA10 | 5.5E−011 | No tested | No tested | No tested |

NP = Not reaching a plateau, EC50 cannot be evaluated.

As demonstrated in the table above, all the FAP/LTBR bispecific antibodies bind to LTBR-positive cells with a lower potency than control BHA10, which has an EC50 value in the pM range. As discussed above, a lower binding affinity may be beneficial in the context of an agonistic antibody.

4.3. NFKB Activation of HepG2 Reporter Cells Expressing Human LTBR Co-Cultured with a FAP-Overexpressing CHO Cell Line

For FAP-mediated cross-linking, a FAP-CHO-K1 recombinant cell line (BPS, cat no. 79947) was used.

To assess the ability of the FAP/LTBR bispecific antibodies to activate LTBR conditionally, the molecules were tested in a NFKB luciferase reporter HEPG2 assay (Signosis, cat. no SL0017-FP) in the presence or absence of a CHO-overexpressing FAP cell line (FAP-CHO-K1). Clustering endogenous LTBR on HepG2 cells leads to activation of endogenous NFKB and translocation from the cytoplasm and nucleus, where it binds to the promoter region and induces luciferase expression. After that, a luciferase substrate/lysis reagent mix (ONE-GLO EX Promega) is added to cells, which allows quantification in the bioluminescence reaction, where enzymatic activity is proportional to luciferase expression.

The monospecific anti-LTBR agonist CBE-11 was used as a positive control. HepG2 NFKB Lc (30000 cells/well) were either co-seeded with FAP overexpressing CHO cells (15000 cells/well) or seeded alone in their growth media. The 1:10 series dilution of bispecific antibodies and controls was added to the cells and incubated for 6 h at 37° C., 5% CO2, prior to the addition of detection reagents. After 6 h ONE-Glo Ex Solution (Promega, cat. no E8120) was added to the well and incubated for 10 min before being read on a BMG Pherastar FSX plate reader. Background-corrected RLU values were plotted against stimulant concentration for all conditions tested in GraphPad Prism, and the EC50 value can be found in.

TABLE 19
EC50 values of HepG2 NFKB activation-
induced luciferase activity

HepG2 Reporter assay

| | HepG2 | HepG2:FAP-CHO- |
|---|---|---|
| | Monoculture | K1 Co-culture |
| Clone ID | EC50[M] | EC50[M] |
| 4.1 | Inactive | 7.0E−11 |
| 4.2 | Inactive | 3.0E−11 |
| 4.3 | Low-level activity | 6.0E−11 |
| 4.4 | Inactive | 3.1E−11 |
| 4.5 | Inactive | 4.0E−11 |
| 4.6 | Inactive | 5.3E−11 |
| 4.7 | Inactive | 2.3E−11 |
| 4.8 | 2.2E−10 | 2.3E−11 |
| 4.9 | 7.0E−11 | 6.0E−12 |
| 4.10 | Inactive | 3.6E−11 |
| 4.16 | Inactive | 1.6E−11 |
| 4.17 | Inactive | 1.7E−11 |
| 4.18 | Inactive | 2.3E−11 |
| CBE-11 | 3.3E−11 | 4.6E−11 |
| P1AH5886 | 1.6E−10 | 2.3E−11 |
| Comparative | | |
| example 1 | | |

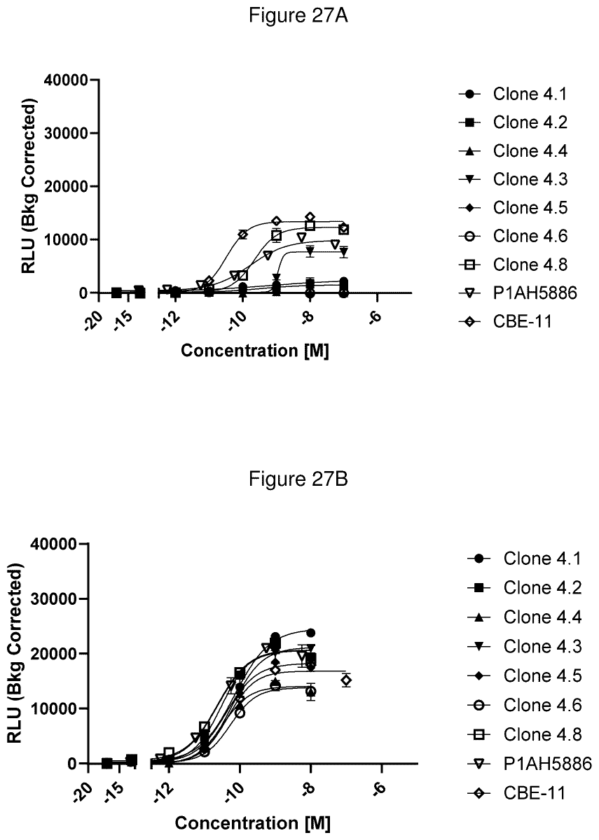

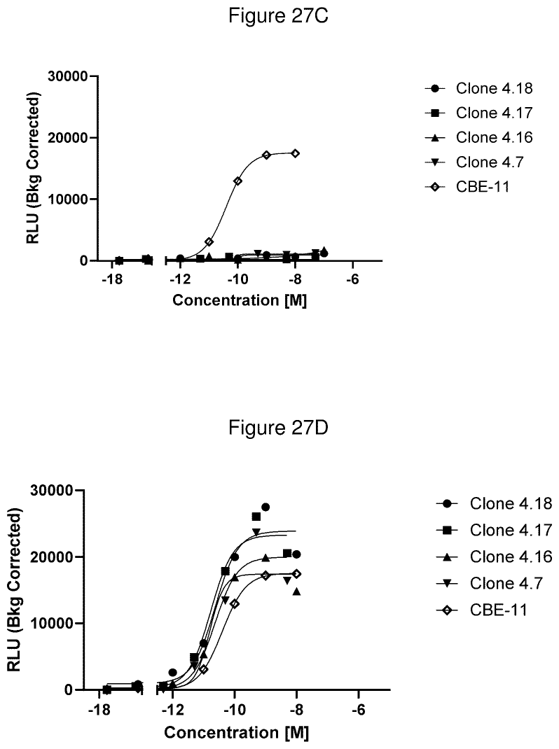

The results confirmed that the FAP/LTBR bispecific antibodies containing a monovalent LTBR arm (1:1 and 2:1 format) activate LTBR in a FAP-dependent way, with minimal or no activity in the absence of FAP, as demonstrated by representative data in FIGS. 27a,c (monoculture) and 27b, d (co-culture) performed in the HEPG2 Reporter assay. This is in contrast to the control monospecific antibody CBE-11, which activates HepG2 cells to a similar extent in the presence or absence of FAP-positive cells.

The FAP/LTBR bispecific antibodies (clones 4.8 and 4.9) and comparative example antibody clone P1AH5886 (WO2023117834) containing two LTBR binding sites retained more LTBR activity in the absence of FAP. In contrast to monospecific control, the activity is further increased in the presence of FAP-positive cells. As discussed above, this conditional activation phenomenon should allow for reduced toxicity due to activation of LTBR-positive cells in the presence of FAP in the tumour (and not in tissues where FAP is not expressed).

Due to the complexity of the bispecific, the minimal activity in the monoculture could be due to the presence of some residual impurities in the sample.

4.4. In Vitro FAP/LTBR Bispecific Antibody Activity in a Chemokine Induction Assay Using Tumor Cell Line HCC1187 Cultured with FAP-Positive Primary Human Breast CAFs

An activation assay measuring chemokine secretion was used to evaluate the ability of the LTBR/FAP bispecific antibodies to conditionally cluster and activate LTBR endogenously expressed on cells (tumor cell line HCC1187).

The primary human breast CAFs were used as a source of FAP to determine the biological activities of the LTBR/FAP bispecific antibodies in a physiologically relevant setting.

The expression of LTBR on HCC1187 and primary human breast CAFs was confirmed by flow cytometry.

HCC1187 (20000/well) were co-seeded either with primary human breast CAFs, isolated from cancer patients (20000/well) or alone in their growth medium. A 1:10, 7-point serial dilution series in duplicates of bispecific antibodies and controls were prepared in assay medium as 2-fold concentration stocks, added to the cells, and incubated for 24 h at 37° C., 5% CO2.

After incubation, the supernatants were collected and cleared by centrifugation, and the chemokine level was determined by CCL19 DuoSet ELISA (R&D system, cat. no DY361) following the manufacturer's instructions. Absorbance was read on the BMG Pherastar FSX plate reader. OD corrected values (450-540 nm) for the standard curves were analysed in Prism, according to the manufacturer's recommendation, in order to interpolate cytokine concentrations for the test wells. Dose-response curves were plotted using GraphPad Prism Version 9, applying non-linear fits (log(agonist) vs response (variable slope—four parameters).

The FAP/LTBR bispecific antibodies induce secretion of CCL19 on HCC1187 cells in a dose-dependent manner (FIGS. 8a-80. These data show that LTBR activation occurs only upon clustering LTBR in the presence of FAP-positive cells (primary human breast CAFs, in that case) for molecules with a single LTBR binding site. Similarly, as shown in example 4.3, bispecific constructs with two binding sites for LTBR retained some function in the monoculture. EC50 values can be found in.

Surprisingly, the FAP/LTBR bispecific antibodies (clones 4.1-4.6) showed much greater potency in the co-culture compared to the control antibody CBE-11 which could be explained by lower binding affinity to LTBR-positive cells. As reported in the literature, low-affinity anti-4-1 BB, CD40 monoclonal antibodies mediate higher agonistic activity, suggesting that low-affinity antibody-induced agonism is a conserved feature among TNFRs (Yu et al., Nature 2023).

TABLE 20
EC50 values of upregulation of CCL19 on HCC1187 in the presence
and absence of FAP positive primary human breast CAFs

HCC1187:CAF co-culture assay

| | HCC1187 | HCC1187:CAF |
|---|---|---|
| | Monoculture | Co-culture |
| Clone ID | EC50[M] | EC50[M] |
| 4.1 | Inactive | 5.5E−011 |
| 4.2 | Inactive | 6.0E−011 |
| 4.3 | Low-level activity | 9.0E−011 |
| 4.4 | Inactive | 5.4E−011 |
| 4.5 | Inactive | 2.3E−011 |
| 4.6 | Inactive | 1.3E−011 |
| 4.7 | Inactive | 7.3E−011 |
| 4.16 | Inactive | 9.0E−011 |
| 4.17 | Inactive | 6.0E−011 |
| 4.18 | Inactive | 1.2E−010 |
| 4.8 | 4.8Ee−009 | 2.5E−011 |
| 4.9 | 3.8E−010 | 1.9E−011 |
| 4.10 | Inactive | 4.1E−010 |
| CBE-11 | 2.3E−010 | 2.6E−010 |

### 4.5. In Vitro FAP/LTBR Bispecific Antibody Activity in an ICAM-1 Upregulation Assay on Human Breast CAFs

It has been reported that stimulation of LTBR leads to the activation of canonical NF-KB1-RelA and the alternative NF-KB2-RelB pathways (Benezech et al. 2012), which result in the induction of chemokines CXCL13, CCl21, and CCL19 and adhesion molecules ICAM1 and VCAM1.

A monoculture assay with primary human breast CAFs was performed to verify if activation of LTBR with the FAP/LTBR bispecific antibodies leads to upregulation of adhesion molecules. ICAM-1 expression was evaluated by immunofluorescence staining.

The expression of both targets on primary human breast CAFs was confirmed by flow cytometry using control antibodies.

Human breast CAFs co-expressing both receptors (FAP and LTBR) were seeded at 20k/well in a 96-well plate and left to adhere for 24h. Following this, a 7-point, 10-fold dilution series of bsAb and control LTBR agonist were prepared as ×10 stocks in culture medium, added to cells and incubated at 37° C., 5% CO2 for 24 h. After stimulation, the cells were fixed by the addition of 100 ul 8% formaldehyde (4% final) for 20 minutes and washed with PBS. Cells were incubated with blocking buffer (3% BSA/0.3% Triton X-100 in PBS (WNN)) for 1 h at RT to prevent non-specific binding. For immunostaining, cells were incubated with rabbit monoclonal affinity purified antibody to ICAM-1 (Abcam, cat. no ab282575 diln 1/500 in 3% BSA in PBS (WN)) O/N at 4° C. Cells were then washed three times with 100 ul wash buffer (10 mM sodium phosphate, 0.15 M NaCl, 0.05% Tween-20 buffer) followed by incubation with secondary antibody AF546 conjugated anti-Rabbit IgG (H+L) polyclonal antibody donkey IgG for 1 h at RT (Invitrogen, cat. no A10040, 1/500 diln in 3% BSA in PBS (WN)). In parallel, nuclei were counterstained with the nuclear stain Hoechst 33342 (Invitrogen, H3570, 1/10000 diln). Cells were washed a further three times with wash buffer before being transferred into PBS for image acquisition. Fluorescence images were captured on Cell Insight CX7 HCS platform. Image acquisition and analysis were performed with Thermo Scientific HCS studio: cellomics scan V6.6.2 software using the Spot Detector Bioapplication V4. This bioapplication measures fluorescence intensities on a single cell level, allowing rapid protein expression analysis. Data are presented as average fluorescence intensity per cell (ICAM1 expression) minus intensity from untreated conditions (baseline subtracted) and plotted with GraphPad Prism Version 9.

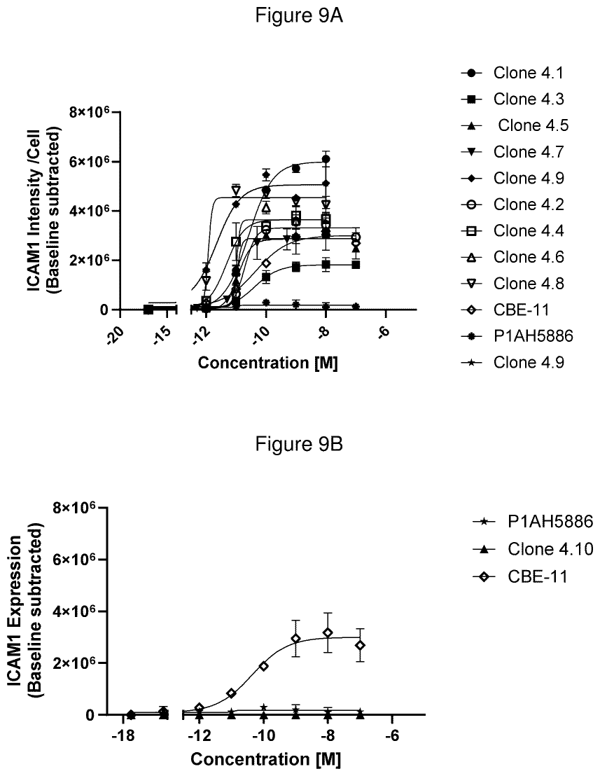

FIGS. 9a and 9b show that the FAP/LTBR bispecific antibodies, apart from comparative example P1AH5886 (WO2023117834) and clone 4.10, achieved potent LTBR activation measured by upregulation of ICAM-1 on primary fibroblasts. Comparative example P1AH5886 did not achieve potent LTBR activation in this assay.

This led to the conclusion that the anti-FAP/LTBR bispecific antibodies can function in cis by co-engaging FAP and LTBR on the same cells, as well as in trans by targeting LTBR expressed on one cell (e.g. stroma) and FAP on adjacent cells, as demonstrated in FIG. 8a-8f. The data is summarised in.

The FAP/LTBR bispecific antibodies in 2:2 format (clones 4.8 and 4.9) have the lowest EC50 suggesting a higher potency than the FAP/LTBR bispecific antibodies in 2:1 or 1:1 format. However, the activity of 2:2 formats, as shown in the previous examples, could be FAP-dependent and FAP-independent, as the bivalent LTBR format retained some agonistic function (Examples 4.3 and 4.4).

Surprisingly, the FAP/LTBR bispecific antibody clone 4.10 is unable to upregulate ICAM-1 in circumstances where FAP and LTBR are expressed on the same cell, even though it contains the same functional LTBR binding agent as clones 4.1, 4.3, 4.4 and 4.5. However, clone 4.10 contains a different FAP binding agent (binding to a distinct epitope). This distinct epitope appears to be instrumental in achieving ICAM-1 upregulation in a FAP/LTBR bispecific antibody.

Without wishing to be bound by theory, this observation may indicate that ICAM-1 upregulation can be achieved by binding to both FAP and LTBR simultaneously on the same cell (‘cis’) (while binding to FAP on a first cell and binding to LTBR on a second cell simultaneously (‘trans’) may not give rise to ICAM-1 upregulation). This FAP epitope may facilitate binding to both FAP and LTBR simultaneously on the same cell.

TABLE 21
EC50 values for ICAM1 upregulation in CAFs monoculture assay

| Clone ID | EC50 [M] |
|---|---|
| 4.1 | 2.6E−011 |
| 4.2 | 1.9E−011 |
| 4.3 | 4.0E−011 |
| 4.4 | 4.8E−012 |
| 4.5 | 1.1E−011 |
| 4.6 | 1.0E−011 |
| 4.7 | 1.2E−011 |
| 4.8 | 1.2E−012 |
| 4.9 | 2.2E−012 |
| 4.10 | Inactive |
| CBE-11 | 4.4E−011 |
| P1AH5886 | Inactive |
| Comparative | |
| example 1 | |

### 4.6. In Vitro FAP/LTBR Bispecific Antibody Activity in a Chemokine Induction Assay Using Tumor Cell Line RPMI-7951

To investigate if the FAP/LTBR bispecific antibodies can activate LTBR efficiently on double-positive cells in cis (such as Fibroblast Reticular cells which are essential for forming and maintaining TLS), a monoculture assay was performed with tumor cell line RPMI-7951. The expression of FAP and LTBR was confirmed by flow cytometry. Briefly, RPMI-7951 were seeded at 50 K/well into 96-V bottom plates in culture media. A 7-point, 10-fold dilution series for the FAP/LTBR bispecific antibodies and control LTBR agonist antibody CBE11 were prepared as ×10 stocks in culture medium, added to cells and incubated at 37° C., 5% CO2 for 24 h. After 24h, plates were centrifuged, and the supernatant was collected. Following the manufacturer's instructions, the chemokine CCL5 level in the supernatant was determined by human CCL5 DuoSet ELISA (R&D system, cat. no DY278). Absorbance was read on the BMG Pherastar FSX plate reader. OD corrected values (450-540 nm) for the standard curve were analysed in GraphPad Prism, according to the manufacturer's recommendation, in order to interpolate cytokine concentrations for the test wells. Dose-response curves were plotted using GraphPad Prism Version 9, applying non-linear fits (log(agonist) vs response (variable slope-four parameters). The data is summarised in.

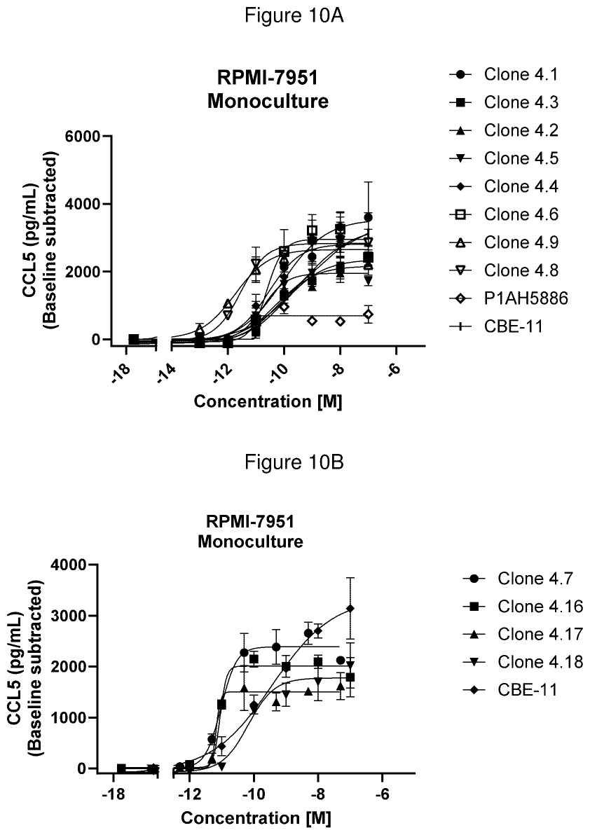

FIGS. 10a and 10b show potent LTBR activation with the FAP/LTBR bispecific antibodies and the LTBR agonist control antibody, resulting in pro-inflammatory CCL5 chemokine upregulation. This is in contrast to the comparative example bispecific antibody P1AH5886, which does not lead to the secretion of CCL5 on RPMI-7951. From the results obtained, it can be concluded that the FAP/LTBR bispecific antibodies can sufficiently cluster LTBR even if both receptors are co-expressed. Similarly, as was described in Example 4.5, clones 4.8 and 4.9 induced significantly higher levels of CCL5 compared to the FAP/LTBR bispecifics in 1:1 or 2:1 format.

Taken together, these data confirm that the FAP/LTBR bispecific antibodies can activate LTBR in cis sufficiently (when both receptors co-expressed) and in trans by neighbouring cells in a FAP-dependent manner, leading to the induction of chemokines, such as CCL19 and CCL5 and upregulation of adhesion molecules, such as ICAM-1.

TABLE 22
EC50 values for chemokine induction in RPMI-7951

| Clone ID | EC50 [M] |
|---|---|
| 4.1 | 9.0E−011 |
| 4.2 | 6.3E−011 |
| 4.3 | 1.0E−010 |
| 4.4 | 2.8E−011 |
| 4.5 | 1.8E−011 |
| 4.6 | 2.4E−011 |
| 4.7 | 9.6E−012 |
| 4.16 | 7.8E−012 |
| 4.17 | 1.0E−011 |
| 4.18 | 6.7E−011 |
| 4.8 | 2.9E−012 |
| 4.9 | 1.8E−012 |
| 4.10 | Not Tested |
| CBE-11 | 3.8E−010 |
| P1AH5886 | Low level |
| Comparative | of activity |
| example 1 | |

4.7. In Vitro FAP/LTBR Bispecific Antibody Trans Activity in a Chemokine Induction Assay Using Low-FAP Expressing Cells (RPMI-7951) when in Co-Culture with HCC-1187

The potency of the FAP/LTBR bispecific antibodies was determined in the presence of a low-FAP expressing cell line (RPMI-7951). The expression level was confirmed by flow cytometry, and FAP expression was around 4-fold lower than on previously used primary human breast CAFs (Example 4.4). Since RPMI-7951 and HCC1187 secrete unique chemokines upon LTBR stimulation, as described in examples 4.4 and 4.6, it was possible to evaluate cis and trans activation simultaneously.

Since HCC1187 are single positive cells (LTBR is present on the cell surface, but not FAP), for FAP/LTBR bispecific antibodies to cluster LTBR and subsequently activate downstream signalling pathways, these bispecific antibodies must co-engage FAP expressed on RPMI-7951 cells and LTBR expressed on HCC1187 cells. If CCL19 secretion is observed in this assay upon adding FAP/LTBR bispecific antibodies, this suggests trans activation of LTBR occurs. Cis activation was characterised in parallel as RPMI-7951 cells express both FAP and LTBR and secrete CCL5 upon LTBR stimulation.

In short, RPMI-7951 cells (20,000 cells/well) were seeded in 96-well plates for RPMI-7951 monoculture and co-culture plates alongside media-only plates for HCC1187 monoculture. Plates were then incubated for 24 hours at 37° C. and 5% CO2 before adding HCC1187 cells and compounds. The next day, HCC1187 cells (20,000 cells/well) were seeded into media-only plates, and RPMI-7951 co-culture plates were prepared on day 1. A 7-point, 10-fold dilution series for the FAP/LTBR bispecific antibodies and control LTBR agonist antibody (CBE-11) were prepared as ×10 stocks in culture medium and added to the cell plates. After 24h, plates were centrifuged, and the supernatant was collected. Following the manufacturer's instructions, the chemokine levels of CCL5 and CCL19 were determined in the supernatant by human CCL5 DuoSet ELISA (R&D system, cat. no DY278) and human CCL19 DuoSet ELISA (R&D system, cat. no DY361). Absorbance was read on the BMG Pherastar FSX plate reader. OD corrected values (450-540 nm) for the standard curve were analysed in GraphPad Prism, according to the manufacturer's recommendation, in order to interpolate cytokine concentrations for the test wells. Dose-response curves were plotted using GraphPad Prism Version 9, applying non-linear fits (log(agonist) vs response (variable slope—four parameters). The data are provided in.

TABLE 23
EC50 values for cis and trans activation
in the HCC1187:RPMI-7951 co-culture assay

| | | HCC- | |
|---|---|---|---|
| | | 1187:RPMI7951 | HCC- |
| | HCC1187 | (cis-activation) | 1187:RPMI7951 |
| | monoculture | co-culture | (trans-activation) |
| | assay CCL19 | assay | co-culture assay, |
| | read-out | CCL5 read-out | CCL19 Read-out |
| Clone ID | EC50 [M] | EC50 [M] | EC50 [M] |
| 4.1 | Inactive | 5.2E−011 | 8.4E−11 |
| 4.2 | Low level | 1.1E−010 | 1.6E−10 |
| 4.3 | Low level | 1.2E−010 | 1.1E−10 |
| 4.4 | Inactive | 1.2E−011 | Inactive |
| 4.5 | Inactive | 1.2E−011 | Inactive |
| 4.6 | Inactive | 1.3E−011 | Inactive |
| 4.7 | Inactive | 1.0E−011 | Inactive |
| 4.8 | 1.4E−009 | 1.3E−012 | 1.0E−011 |
| 4.9 | 4.7E−011 | 1.3E−012 | 1.1E−011 |
| CBE-11 | 2.389e−010 | 1.3E−010 | 8.4E−11 |

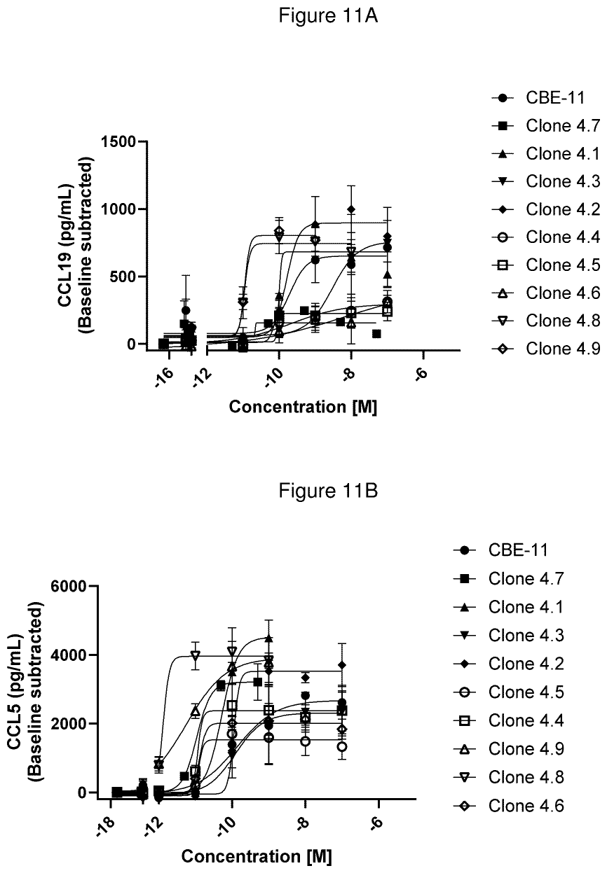

FIG. 11a suggests that the FAP/LTBR bispecific antibodies in 1:1 format (see bispecific antibodies 4.1, 4.2 and 4.3) can induce the secretion of CCL19 as a result of FAP-mediated activation of LTBR, even in the presence of low levels of FAP.

FIG. 11b confirms the previous result described in Example 4.6 that the FAP/LTBR bispecific antibodies can cluster LTBR sufficiently on double-positive cells (FAP and LTBR positive), which leads to dose-dependent CCL5 secretion.

Taking together this data and Example 4.4 suggests that the potency of the FAP/LTBR bispecific antibodies correlates with FAP expression density on cells.

### 4.8. The Binding Affinity of LTBR/FAP Bispecific Antibodies to Human and Cynomolgus Recombinant LTBR and FAP by Octet

To confirm species cross-reactivity, binding affinity to human and cynomolgus recombinant proteins for both targets was measured by Octet. Such cross-reactivity is advantageous, as it allows dosing and safety testing of the antibody molecules to be performed in cynomolgus monkeys during preclinical development.

Briefly, Biotinylated hLTBR-His-Avitag (Acro, cat no LTR-H82E9-25 μg) was loaded on SA Streptavidin biosensors (Fortebio) in kinetics buffer (ForteBio) followed by a 2-fold dilution series starting at 50 nM of FAP/LTBR bispecific antibodies. Binding kinetics were studied in a 1× Kinetics buffer where the association was allowed for 100 seconds, followed by dissociation for 300 seconds. Data generated were referenced by subtracting a parallel buffer blank, the baseline was aligned with the y-axis, inter-step correlation by alignment against dissociation was performed, and the data were smoothed by a Savitzky-Golay filter in the data analysis software. The processed data were fitted using a 1:1 Langmuir binding model with R\hat{ }2 as a measurement of fitting accuracy. The K_{D} and R\hat{ }2 values can be found in.

A 2-fold series dilution starting at 50 nM of cynomologus recombinant LTBR was immobilised on the surface of ARG biosensors (Fortebio), followed by FAP/LTBR bispecific at 25 nM. Binding kinetics were studied in a 1× Kinetics buffer where the association was allowed for 180 seconds, followed by dissociation for 100 seconds. Sensor tips were regenerated using ethylene glycol. Data generated were referenced by subtracting a parallel buffer blank, the baseline was aligned with the y-axis, inter-step correlation by alignment against dissociation was performed, and the data were smoothed by a Savitzky-Golay filter in the data analysis software. The processed data were fitted using a 1:1 Langmuir binding model with R\hat{ }2 as a measurement of fitting accuracy. The KD values can be found in.

Binding kinetics to both targets for clone 4.7 was carried out as described in Example 2.6 and 3.6, and the KD value can be found in.

Binding to recombinant FAP proteins was investigated by Bio-Layer Interferometry (BLI) using the Octet instrument. Using standard amine coupling chemistry, an anti-Fc antibody was immobilised on the surface of ARG biosensors (Fortebio). The FAP/LTBR bispecific antibodies were loaded on the previously coated surface with anti-Fc capture antibody, followed by a 2-fold series dilution starting at 50 nM of either cyno FAP (Acro, cat no FAP-C82Q5) or human FAP (Acro, cat no FAP-H82Q6). Binding kinetics were studied in a 1× Kinetics buffer where the association was allowed for 200 seconds, followed by dissociation for 400 seconds. Sensor tips were regenerated using 10 mM Glycine pH 2.0. Data generated were referenced by subtracting a parallel buffer blank, the baseline was aligned with the y-axis, inter-step correlation by alignment against dissociation was performed, and the data were smoothed by a Savitzky-Golay filter in the data analysis software. The processed data were fitted using a 1:1 Langmuir binding model with R\hat{ }2 as a measurement of fitting accuracy. The Results are summarised in. Avidity was observed as FAP is naturally a dimeric antigen, and the limited detection by Octet with these molecules has been reached. Species cross-reactivity has been confirmed for all test samples.

TABLE 24
Kinetics parameters for FAP/LTBR bispecific antibody binding
to human and cyno LTBR and FAP measured by Octet

| | huLTBR | cyLTBR | HuFAP | CyFAP |
|---|---|---|---|---|
| Clone ID | KD [nM] | KD[nM] | KD [pM] | KD [pM] |
| 4.1 | 4.0 | 7.7 | 182* | Low pM* |
| 4.2 | 3.2 | 6.75 | 325* | Low pM* |
| 4.3 | 3.1 | 5.78 | 192* | Low pM* |
| 4.4 | 4.6 | 8.30 | 231* | Low pM* |
| 4.5 | 6.5 | 5.22 | 100* | Low pM* |
| 4.6 | 4.3 | 17.1 | 44.3* | Low pM* |
| 4.7 | 1.8 | 0.4 | 160* | 150* |
| 4.9 | 0.17 | 8.65 | Low pM* | Low pM* |

*values outside instrument specifications

4.9. In Vitro Surrogate FAP/LTBR Bispecific Antibody Activity in a Chemokine Induction Assay Using Tumor Cell Line 4T1 Cultured with FAP Positive CHO-Overexpressing Line

Since the anti-FAP antibody (clone 28H1) selectively binds to both human and murine antigens, it is possible to use the CHO-human FAP-K1 line as a source of FAP in the following assay.

To assess the ability of surrogate mouse FAP/LTBR bispecific antibodies to activate LTBR conditionally, FAP/LTBR bispecific antibodies and monoclonal LTBR agonist antibodies were tested in 4T1 (ATCC, cat no. CRL-2539) in the presence or absence of FAP-CHO-K1 cell line. In addition, the monospecific monoclonal anti-LTBR antibodies were tested in the presence or absence of a cross-linking agent to induce sufficient LTBR clustering. Anti-Fc antibody was used as a cross-linking agent.

Briefly, 4T1 (20000/well) were co-seeded either with FAP-CHO-K1 (20000/well) or alone in their growth medium. A 1:10, 7-point serial dilution series in duplicates of bispecific antibodies and controls were prepared in assay medium as 10-fold concentration stocks, added to the cells, and incubated for 24 h at 37° C., 5% CO2.

After incubation, the supernatants were collected and cleared by centrifugation, and the chemokine level was determined by CCL5 DuoSet ELISA (DY478-05) following the manufacturer's instructions. Absorbance was read on the BMG Pherastar FSX plate reader. OD corrected values (450-540 nm) for the standard curves were analysed in Prism, according to the manufacturer's recommendation, in order to interpolate cytokine concentrations for the test wells. Dose-response curves were plotted using GraphPad Prism Version 9, applying non-linear fits (log(agonist) vs response (variable slope—four parameters). EC50 values can be found in.

TABLE 25
EC50 values for chemokine induction in 4T1

| | | 4T1:CHO-FAP |
|---|---|---|
| | 4T1 monoculture | co-culture |
| | CCL5 read-out | CCL5 read-out |
| Clone ID | EC50[M] | EC50[M] |
| 4.11 | Low level | 4.8E−012 |
| 4.12 | Low level | 4.9E−012 |
| 4.13 | Low level | Low level |
| 4.14 | 4.7E−010 | 2.8E−010 |
| 4.14 + cross-linking | 1.4E−010 | 9.6E−011 |
| antibody | | |
| 4.15 | 8.4E−010 | NA |
| 4.15 + cross-linking | 3.6E−011 | NA |
| antibody | | |
| P1AG5459 | 1.2E−009 | 2.5E−011 |
| Comparative | | |
| example 2 | | |
| P1AG5461 | 7.9E−010 | 8.6E−010 |
| Comparative | | |
| example 3 | | |

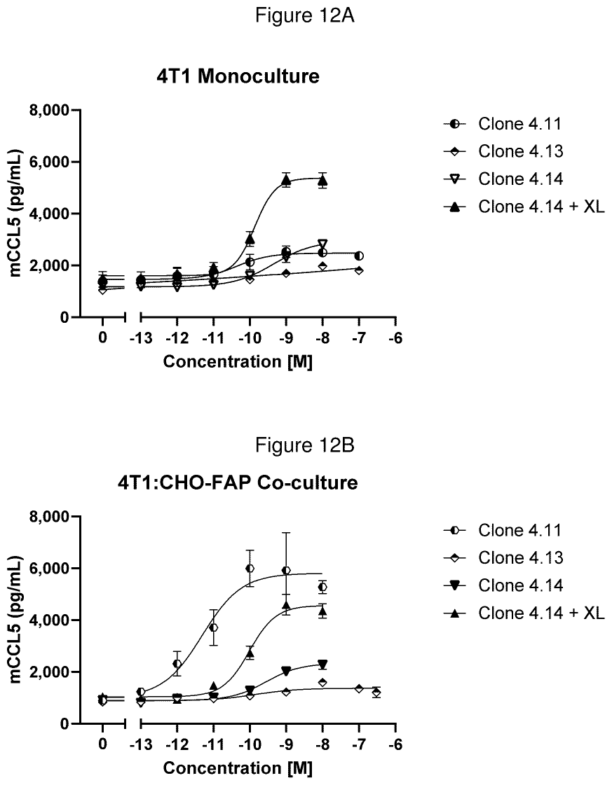

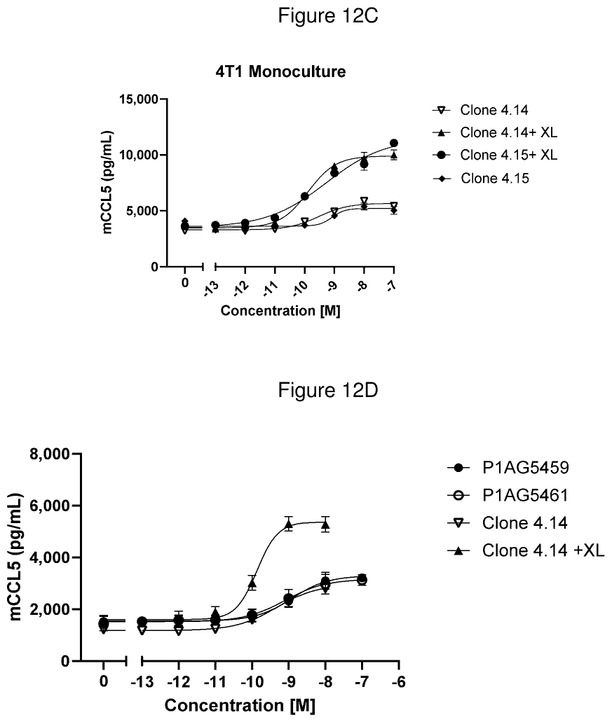

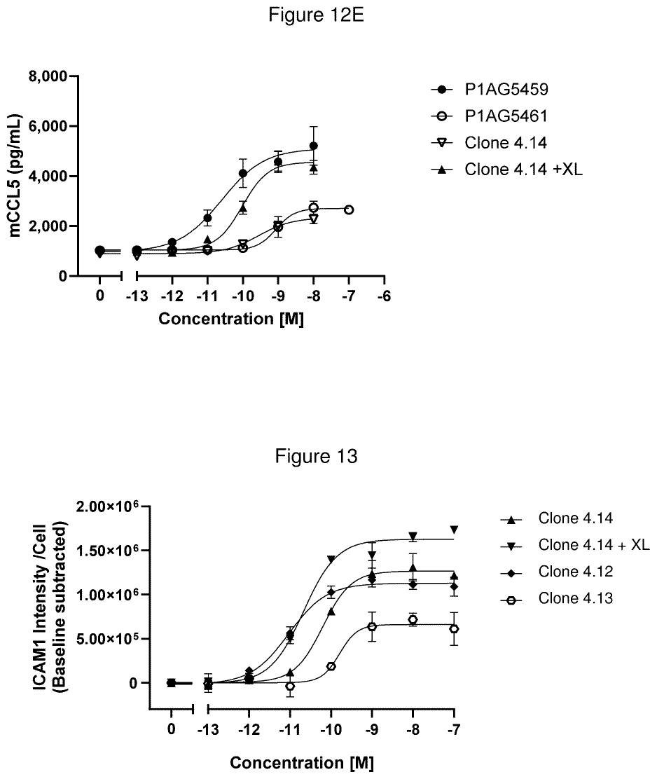

FIGS. 12a and 12b show that the surrogate FAP/LTBR bispecific antibodies, in contrast to the non-targeted LTBR bispecific (clone 4.13), conditionally activate LTBR, with limited activity in the absence of FAP. The results show that the FAP-dependent activity of clone 4.11 is more potent, demonstrated by lower EC50 values and increased max activity compared to cross-linked monospecific LTBR agonist antibody (clone 4.14).

FIG. 12c shows that monospecific LTBR agonist antibody 5G11, regardless of the Fc backbone, demonstrates the same potency in the 4T1 monoculture assay, resulting in dose-dependent secretion of CCL5 (results with clone 4.14 and 4.15).

Activation of LTBR using the monospecific LTBR agonist antibody was achieved in the absence of FAP, and it could potentially lead to toxicity due to the broad expression of LTBR in normal tissue. A higher activation level of LTBR was achieved when the monospecific antibody was cross-linked by anti-Fc antibody.

In contrast to surrogate FAP/LTBR bispecific antibody (clone 4.12) FIG. 12d shows that comparative surrogate bispecific molecules (FAP targeted P1AG5459 and non-targeted P1AG55461) activate LTBR to the same extent as the monospecific LTBR agonist control antibody (clone 4.14) in 4T1 monoculture assay without crosslinking. The potency of FAP targeted comparative molecules is being enhanced upon co-culture with FAP positive cell line (FIG. 12e) in contrast to non-targeted control, which demonstrated comparable activity in the mono and co-culture.

### 4.10. In Vitro Activity of Mouse LTBR/FAP Bispecific Agonist Antibody in Primary Mouse Lung Fibroblasts

A monoculture assay with primary mouse lung fibroblasts (PMLF) was employed to understand if stimulation with mouse FAP/LTBR bispecific antibody agonists of fibroblasts results in the upregulation of adhesion molecules, such as ICAM-1.

Primary mouse lung fibroblasts (PMLF) were derived from naive (untreated) mice. PMLF were seeded (20k/well) in 96 well plates and left to adhere for 24h. Following this, a 7-point, 10-fold dilution series of FAP/LTBR bsAb and control LTBR agonist antibody were prepared as ×10 stocks in culture medium, added to cells and incubated at 37° C., 5% CO2 for 48 h. After stimulation, the cells were fixed by the addition of 100 ul 8% formaldehyde (4% final) for 20 minutes and washed with PBS. Cells were incubated with blocking buffer (3% BSA/0.3% Triton X-100 in PBS (W/V/V)) for 1 h at RT to prevent non-specific binding. For immunostaining, cells were incubated with rat monoclonal affinity purified antibody to ICAM-1 (Biolegend, cat no 116102) diln 1/100 in 3% BSA in PBS (W/V)) O/N at 4° C. Cells were then washed three times with 100 ul wash buffer (10 mM sodium phosphate, 0.15 M NaCl, 0.05% Tween-20 buffer) followed by incubation with secondary antibody anti-Rat IgG (H+L) AF488 pAb, Donkey IgG for 1 h at RT (Invitrogen, cat. no A-21208, 1/500 diln in 3% BSA in PBS (W/V)). In parallel, nuclei were counterstained with the nuclear stain Hoechst 33342 (Invitrogen, H3570, 1/10000 diln). Cells were washed a further three times with wash buffer before being transferred into PBS for image acquisition. Fluorescence images were captured on Cell Insight CX7 HCS platform. Image acquisition and analysis were performed with Thermo Scientific HCS studio: cellomics scan V6.6.2 software using the Spot Detector Bioapplication V4. This bioapplication measures fluorescence intensities on a single cell level, allowing rapid protein expression analysis. Data are presented as average fluorescence intensity per cell (ICAM1 expression) minus intensity from untreated condition (baseline subtracted) and plotted with GraphPad Prism Version 9. Stimulation with LTBR/FAP bispecific agonist antibodies results in the concentration-dependent upregulation of adhesion molecules, as represented in FIGS. 13 and 14.

These data show that the surrogate FAP/LTBR bispecific antibodies are capable of inducing clustering and signalling of LTBR in a FAP-dependent manner (due to FAP binding) resulting in chemokine induction and adhesion molecule upregulation on cells, similar to human constructs, as opposed to irrelevant-target control (clone 4.13, which does not bind to FAP) which demonstrated reduced adhesion molecule upregulation (FIGS. 13 and 14) and no chemokine induction (FIGS. 12a and 12b).

TABLE 26
EC50 ICAM1 induction in PMLF

| | PMLF |
|---|---|
| Clone ID | EC50[M] |
| 4.11 | 3.1E−012 |
| 4.12 | 2.1E−012 |
| 4.13 | 1.6E−010 |
| 4.14 | 6.3E−011 |
| 4.14 + cross-linking | 2.1E−011 |
| antibody | |

4.11. In Vivo Efficacy with FAP/LTBR Surrogate Bispecific Antibodies in Orthotopic Breast Cancer Models

Having shown that the surrogate FAP/LTBR bispecific antibodies had the ability to activate LTBR receptor in a FAP-dependent manner, resulting in dose-dependent chemokine induction and adhesion upregulation, it was desirable to test the function of the FAP/LTBR bispecific antibodies in vivo in syngenic immunocompetent tumor models.

The efficacy of the mouse surrogate FAP/LTBR bispecific antibodies in combination with anti-CTLA-4 checkpoint inhibition was tested in the mouse mammary MMTV-PyMT mBR9071 tumor model (Crown Biosciences). Briefly, the mBR9071 tumor model was derived by harvesting tumor fragments from MMTV-PyMT mice on a C57BL/6 background. The tumor fragments were passaged in vivo in stock C57BL/6 mice, where tumor fragments were harvested and used for inoculation into the final recipient C57BL/6 mice. Each recipient mouse was inoculated orthotopically in the mammary fat pad with tumor fragments 2-3 mm in diameter. Mice were randomised into treatment groups when the mean tumor size reached approximately 80-120 mm³. Mice were treated twice weekly with isotype control Ab, anti-CTLA-4 Ab, anti-CTLA-4 Ab+LTBR antibody clone 4.14, or anti-CTLA-4+FAP/LTBR bispecific antibody clone 4.11 ( ). All antibodies were dosed at 10 mg/kg, with intraperitoneal (IP) administration. The anti-CTLA-4 Ab was clone 9D9, Cat. no. BP0164 (BioXell). The study was terminated when the mice in the control group reached humane endpoints. On the day of termination, tumor from all groups were dissociated and digested enzymatically for flow cytometry. The cells were stained for the presence of HEV (CD31+, MECA 79+), T cells (CD3+, CD4+, CD8+) and B cells (CD19+). The cells were fixed and acquired on a BD LSR Fortessa, and data was analysed by Kaluza.

TABLE 27
Treatment schedule in orthotopic breast cancer MMTV-PyMT

| | No of | | | |
|---|---|---|---|---|
| Group | animals | Agents | Dose | Schedule |
| 1 | 10 | Anti-CTLA-4 (clone | 10 | Biwk × 2 (4 |
| | | 9D9) + Clone 4.14 | mg/kg | doses in total) |
| 2 | 10 | Anti- CTLA-4 (clone | 10 | Biwk × 2 (4 |
| | | 9D9) + Clone 4.11 | mg/kg | doses in total) |
| 3 | 10 | Isotype (mouse IgG1 + | 10 | Biwk × 2 (4 |
| | | mouse IgG2b) | mg/kg | doses in total) |
| 4 | 10 | Anti-CTLA-4 (clone 9D9) | 10 | Biwk × 2 (4 |
| | | | mg/kg | doses in total) |

Treatment with anti-CTLA-4 alone had no significant effect on tumor growth compared to isotype control. Mice that received the combination of anti-CTLA-4 and clone 4.14 or 4.11 had highly significant tumor growth inhibition compared to isotype or anti-CTLA-4 treatment alone (FIG. 15). There was no significant body weight loss or changes in liver enzymes ALT and AST in any of the treatment groups (data not shown). The statistical analysis was done with two-way anova.

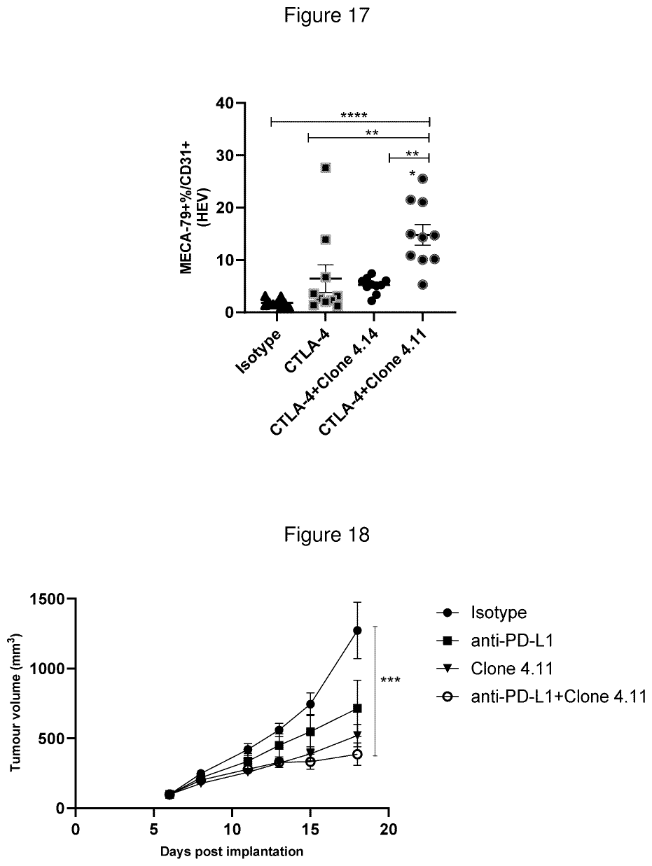

The combination treatments also resulted in an increase in CD8+ T cells in the tumor compared to isotype or anti-CTLA-4 treatment alone, as measured by flow cytometry on the day of study termination (data not shown). Induction of high endothelial venules (HEVs-CD31+MECA-79+ cells) was observed to be significantly increased only in mice treated with the anti-CTLA-4 plus FAP×LTBR bispecific antibody (clone 4.11) combination compared to all other treatment groups, measured by flow cytometry (FIG. 17). The tumor growth curves and flow cytometry results were plotted using GraphPad Prism Version 9. The statistical analysis was done with one-way ANOVA (*p<0.05, **p<0.01, ***p<0.001 ****p<0.0001).

The efficacy of clone 4.11 in combination with anti-PD-L1 checkpoint inhibition was also measured in the syngeneic EMT6 breast cancer model. Briefly, BALB/c mice were inoculated orthotopically in the mammary gland with EMT6 tumor cells (5×10⁵ cells) for tumor development. Mice were randomised into treatment groups when the mean tumor size reached approximately 100 mm³. Mice were treated with isotype control, anti-PD-L1 (clone 10F.9G2, Cat. no. BP0101, BioXell), FAP/LTBR bispecific antibody clone 4.11, or a combination of anti-PD-L1+FAP/LTBR bispecific antibody clone 4.11. All antibodies were dosed twice weekly at 10 mg/kg, with IP administration 0.

TABLE 28
Treatment schedule in orthotopic EMT6 mouse in vivo model

| | No of | | | |
|---|---|---|---|---|
| Group | animals | Compounds name | Dose | Schedule |
| 1 | 10 | Isotype Control | 10 mg/kg | Biwk (5 doses |
| | | | | in total) |
| 2 | 10 | Anti-PD-L1 (10F.9G2, | 10 mg/kg | Biwk (5 doses |
| | | rat IgG2a) | | in total) |
| 3 | 10 | FAP × LTBR bsAb | 10 mg/kg | Biwk (5 doses |
| | | clone 4.11 | | in total) |
| 4 | 10 | anti-PD-L1 + FAP × | 10 mg/kg | Biwk (5 doses |
| | | LTBR bsAb clone 4.11 | | in total) |

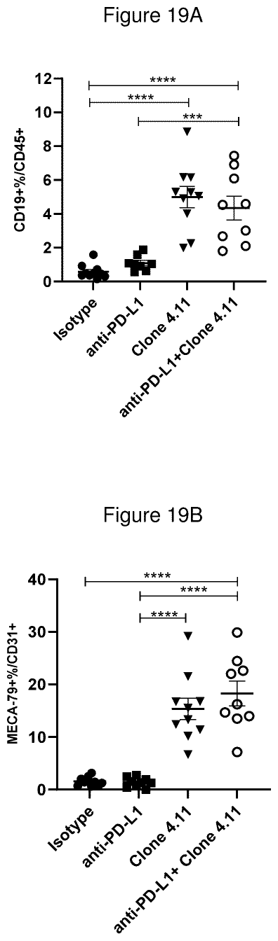

The combination of anti-PD-L1+FAP/LTBR bispecific antibody clone 4.11 resulted in highly significant tumor growth inhibition compared to isotype control (FIG. 18), leading to tumor regressions in 4/10 mice. There was no significant body weight loss in any of the treatment groups. The monotherapy clone 4.11 and combination treatment of anti-PD-L1+ clone 4.11 also resulted in a significant increase in B cells in the tumor compared to isotype or anti-PD-L1 treatment alone, as measured by flow cytometry (FIG. 19a). Induction of HEVs was observed to be significantly increased in mice treated with clone 4.11 monotherapy and combination treatment of anti-PD-L1+ clone 4.11, measured by flow cytometry (FIG. 19b).

4.12. In Vivo Efficacy with a FAP/LTBR Bispecific Surrogate Antibody in a Subcutaneous Lung Cancer Model

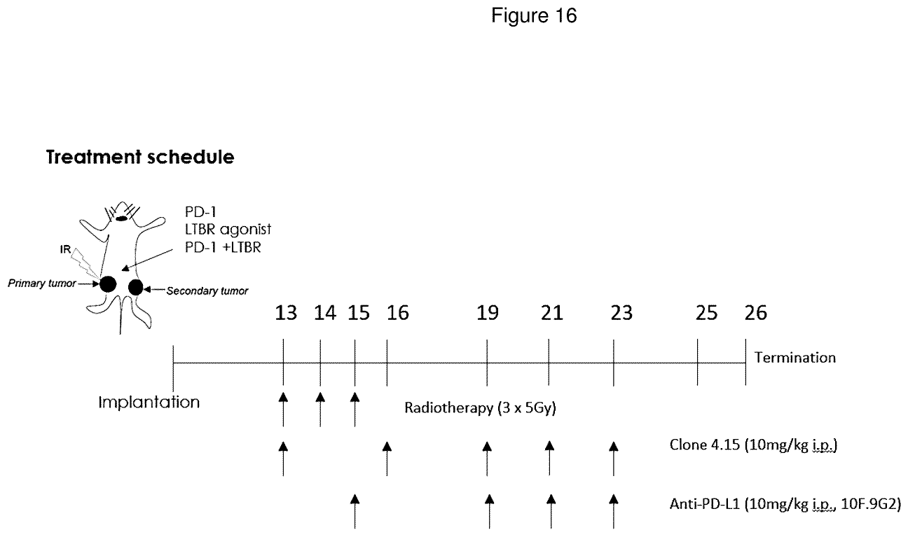

In vivo efficacy of the FAP/LTBR bispecific antibody clone 4.11 with anti-PD-L1 was tested in the mLU6054 mBR9071 NSCLC mouse model (Crown Biosciences). Tumor fragments from the mLU6054 mouse lung tumor model were harvested from stock C57BL/6 mice and used for inoculation into final recipient mice. Each mouse was inoculated subcutaneously in the right front flank with a tumor fragment (2-3 mm in diameter). Recipient mice were randomised into treatment groups when the mean tumor size reached approximately 80-120 mm3. Mice were treated with isotype control, anti-PD-L1, or anti-PD-L1+FAP/LTBR bispecific antibody clone 4.11, twice weekly at 10 mg/kg, with IP administration, as demonstrated in FIG. 16 and. The study was terminated when the mice in the control group reached humane endpoints. On the day of termination, tumor from all groups were dissociated and digested enzymatically for flow cytometry. The cells were stained for the presence of HEV (CD31+, MECA 79+) and T cells (CD3+, CD4+, CD8+). The cells were fixed and acquired on a BD LSR Fortessa, and data was analysed by Kaluza. IHC analysis, tumor were fixed in 10% neutral buffered formalin for 24 hours and then transferred into 70% ethanol before paraffin embedding.

TABLE 29
Treatment schedule in mLU6054 subcutaneous
mouse in vivo model

| | No of | Compounds | | |
|---|---|---|---|---|
| Group | animals | name | Dose | Schedule |
| 1 | 10 | Isotype | 10 mg/kg | Biwk × 2 (4 |
| | | | | doses in total) |
| 2 | 10 | PDL-1 | 10 mg/kg | Biwk × 2 (4 |
| | | (10F.9G2) | | doses in total) |
| 3 | 10 | PDL-1 | 10 mg/kg | Biwk × 2 |
| | | (10F.9G2) + | | (FAP × LTBR bs |
| | | FAP × LTBR | | Ab Clone 4.11 |
| | | BsAb Clone | | (5 doses in |
| | | 4.11 | | total, PDL-1 |
| | | | | (10F.9G2) 4 |
| | | | | doses in total) |

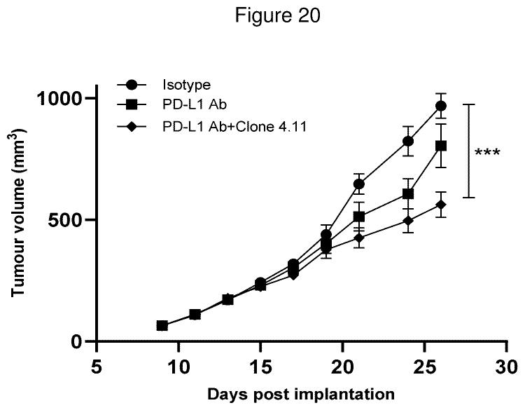

The combination of anti-PD-L1+FAP/LTBR bispecific antibody clone 4.11 resulted in significant tumor growth inhibition compared to isotype control (FIG. 20). There was no significant body weight loss in an of the treatment groups (data not shown). The tumor growth curves and flow cytometry results were plotted using GraphPad Prism Version 9. The statistical analysis of tumor growth curves were done with two two-way ANOVA (*p<0.05, **p<0.01, ****p<0.0001).

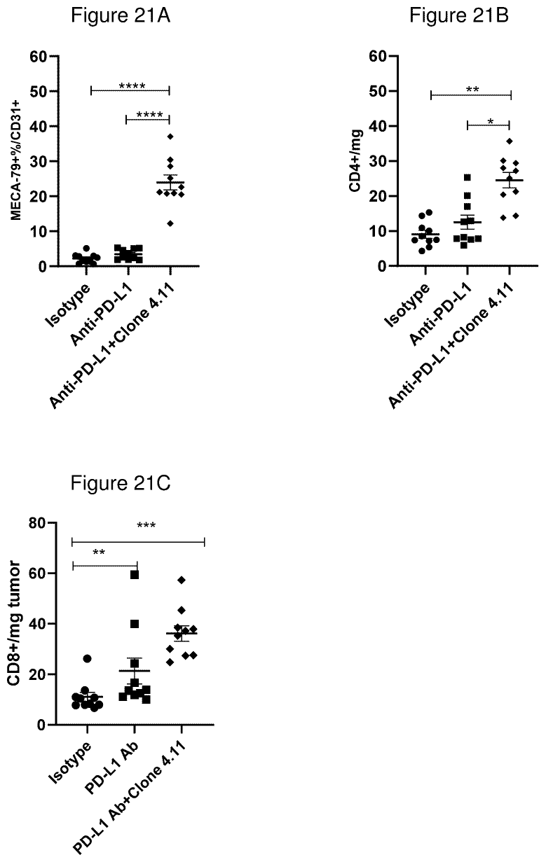

The combination treatment of anti-PD-L1+ clone 4.11 resulted in a significant increase in HEV (CD31+MECA79+ cells) formation in the tumor and increased CD4+ and CD8+ T cells compared to isotype or anti-PD-L1 monotherapy treatment, measured by flow cytometry (FIG. 21a, b, and c). The statistical analysis was done with one-way ANOVA (*p<0.05, ** p<0.01, ***p<0.001 ****p<0.0001).

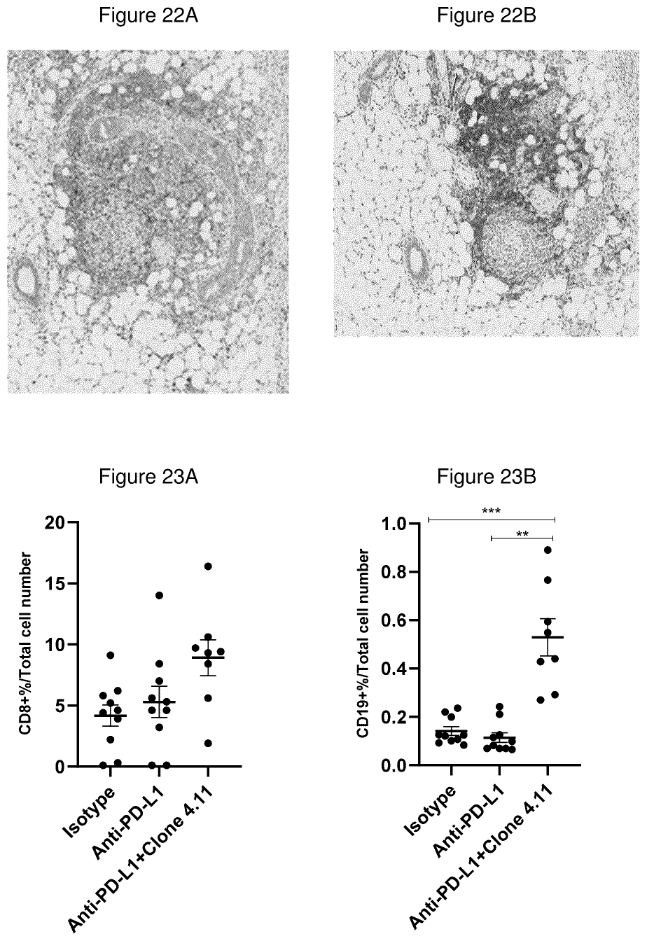

For IHC analysis, tumor were fixed in 10% neutral buffered formalin for 24 hours and then transferred into 70% ethanol before paraffin embedding. FFPE tumor samples were stained by IHC for CD8 and CD19 to look at the location of CD8+ T cells and CD19+ B cells. mLU6054 tumors that were treated with the anti-PD-L1+ clone 4.11 combination showed organised infiltration of CD8+ T cells (FIG. 22a) and CD19+ B cells (FIG. 22b) typical of Tertiary Lymphoid Structure development. These structures were not observed in isotype or anti-PD-L1 monotherapy-treated mice.

In addition, digital quantification of the number of CD8+ and CD19+ cells in tumors by IHC showed an increase of CD8+ T cells and a significant increase of CD19+ B cells in the tumor microenvironment in tumors treated with combination therapy compared to isotype or anti-PD-L1 monotherapy treatment alone (FIGS. 23a and 23b).

In conclusion, the combination treatment of anti-PD-L1 and the FAP/LTBR bispecific antibody led to changes in the tumor microenvironment represented by an increase of immune cells (T and B cells), and the formation of high endothelial venules (HEV), which are specialised blood vessels mediating lymphocyte trafficking to lymph nodes (LNs) and other secondary lymphoid organs. In addition, highly organised aggregates of immune cells with clear T and B cell zones that form TLS were detected.
4.13. In Vivo Efficacy with Anti-LTBR Surrogate Clones in Combination with Radiotherapy in Subcutaneous Colon Cancer Model

Radiation therapy (RT) is a highly effective tool for cancer treatment designed to control or eliminate a solid tumor to provide clinical benefit. In addition, evidence in the literature suggests that certain radiation doses and fraction schemes contribute to inducing immunological cell death, which can result in a systematic anticancer response known as an abscopal effect. To understand if hypo-fractionated radiotherapy provides an additive or synergistic effect with checkpoint inhibition or LTBR agonism to induce a systemic antitumor immune response that can act at a distant non-irritated tumour (abscopal effect), the MC38 tumor model was used.

MC38 tumor fragments were harvested from subcutaneously implanted donor C56bl/6 mice. Tumor fragments (20-30 mg) were subcutaneously implanted into both flanks of C57bl/6 recipient mice. Animals were randomized based on their individual tumor volume in the right flank. Randomization was performed when the tumor on right flank reached approximately 80-100 mm3. Mice that were treated with radiotherapy only to the right flank tumors only, irradiated at 5 Gray once daily for three consecutive days. The left flank tumors were not irradiated during a study. All antibody treatments were dosed IP at 10 mg/kg. Mice were treated with isotype control, anti-PD-L1, radiation only, radiation+anti-PD-L1, radiation+LTBR agonist clone 4.15, or LTBR agonist clone 4.15 monotherapy ( ). The study was terminated when the mice in the control group reached humane endpoints (mean value of primary and secondary tumor). On the day of termination, the primary tumor from all groups were dissociated and digested enzymatically for flow cytometry. The cells were stained for the presence of T cells (CD3+, CD4+, CD8+). The cells were fixed and acquired on a BD LSR Fortessa, and data was analysed by Kaluza. The tumor growth curves and flow cytometry results were plotted using GraphPad Prism Version 9. The statistical analysis of tumor growth curves were done with two two-way ANOVA (*p<0.05, **p<0.01, ****p<0.0001).

TABLE 30
Treatment schedule in MC38 mouse in vivo model

| Group No | Compound name | Dose | Ab dosing schedule |
|---|---|---|---|
| 1 | Isotype Control | 10 mg/kg | Biwk from Day 13 |
| | | | (randomisation) |
| 2 | Anti-PD-L1 | 10 mg/kg | Biwk from Day 15 |
| | (clone 10F.9G2) | | |
| 3 | Radiation | 3 × 5Gy | 5Gy on day 13, 14, 15 |
| 4 | Radiation + | 3 × 5Gy + | 5Gy on days 13, 14, 15 |
| | anti-PD-L1 | 10 mg/kg | Biwk from Day 15 |
| | (clone 10F.9G2) | | |
| 5 | Radiation + | 3 × 5Gy + | 5Gy on days 13, 14, 15 |
| | LTBR agonist | 10 mg/kg | Biwk from Day 13 |
| | (Clone 4.15) | | |
| 6 | LTBR agonist | 10 mg/kg | Biwk from Day 13 |
| | (Clone 4.15) | | |

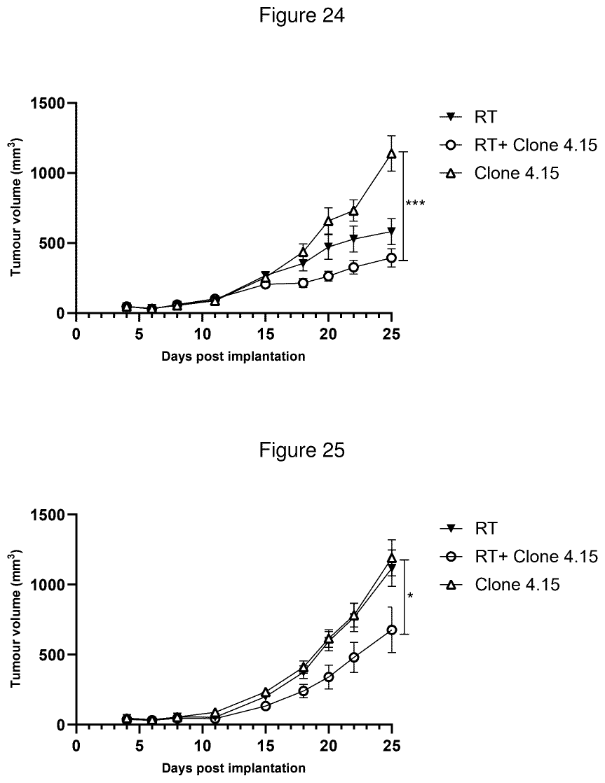

In the right (irradiated) tumor, combining anti-LTBR clone 4.15 with radiation significantly inhibited tumor growth compared to treatment with antibody alone (FIG. 24). In the left (non-irradiated) tumor, the mice that were treated with radiotherapy+anti-LTBR clone 4.15 showed significant inhibition of tumor growth compared to radiation alone or anti-LTBR treatment alone, indicating the combination of anti-LTBR with radiation results in a systemic anti-tumor response (FIG. 25). The treatment with anti-PD-L1 did not result in an abscopal effect in the non-irradiated tumour (data not shown). The isotype control group had rapid tumor growth, with animals reaching humane endpoint quickly and being removed from study, therefore data is not shown.

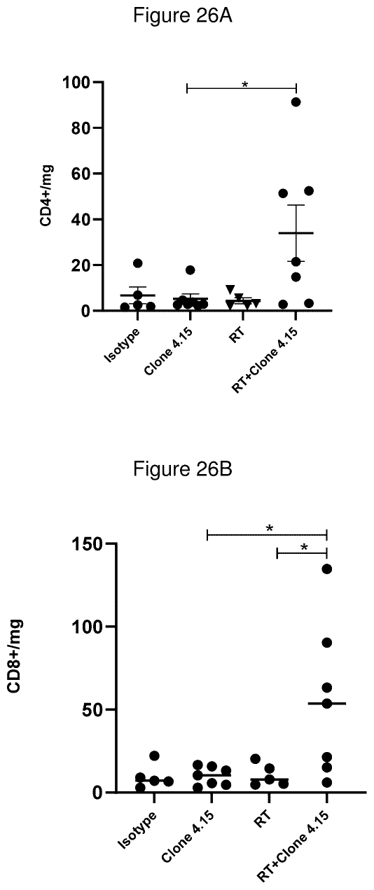

In the right (irradiated) tumors, the combination of radiation with anti-LTBR resulted in a significant increase is CD4+ and CD8+ T cells compared to monotherapy treatment as measured by flow cytometry (FIGS. 26a and 26b), demonstrating changes in the tumor immune microenvironment and synergy between the treatments. The statistical analysis was done with one way anova (*p<0.05, **p<0.01, ***p<0.001 ****p<0.0001).

4.14. In Vivo Efficacy with FAP-LTBR Surrogate Molecules in Combination with E22 Peptide Antigen in Orthotopic Breast Cancer Models

Cancer vaccines typically constitute a tumor specific peptide(s) component administered together with an adjuvant eg a TLR agonist. It is reported in the literature that EMT6 tumors express tumour-associated antigens, including E22 peptide—KCLQDNNWDYTRYAQAFTLLKAKG (Castiglioni et al. 2023, SEQ ID NO: 209). To show the benefit of an LTBR agonist in combination with a cancer vaccine, a combination study was performed using a model cancer vaccine consisting of the E22 peptide together with CpG-ODN 1826 (a well described TLR9 agonist). The activity of this model cancer vaccine was tested adjuvant either on its own or in combination with FAP/LTBR agonist bispecific antibody. LTBR agonism in combination with the model cancer vaccine, FAP/LTBR agonist bispecific antibody induces a potent anti-tumor immune response, inhibiting tumour growth in an EMT6 breast cancer model.

The efficacy of clone 4.11 in combination with an E22 peptide antigen was measured in the syngenic EMT6 breast cancer model. Briefly, BALB/c mice were inoculated orthotopically in the mammary fat pad with EMT6 tumor cells (5×10⁵ cells) for tumor development. Mice were randomised into treatment groups when the mean tumor size reached approximately 100 mm3. Mice were treated with isotype control, adjuvant (TLR-9 agonist, InvivoGen, cat. no. vac-1826-1)+E22 peptide as a control, FAP/LTBR bispecific antibody clone 4.11, or a combination of adjuvant+E22 peptide together with FAP/LTBR bispecific antibody clone 4.11. FAP/LTBR bispecific antibody was dosed three times per week at 10 mg/kg by IP administration (Table 31). An adjuvant only control was also included in the experiment, however in the absence of the peptide it's activity was increased, likely suggesting a different mechanism of this adjuvant in the presence or absence of peptide. Accordingly, we compared FAP/LTBR bsAb+peptide+adjuvant to a control group dosed with the peptide+adjuvant only. The study was terminated when the mice in the control group reached humane endpoints. On the day of termination, tumors from all groups were dissociated and digested enzymatically for flow cytometry. Where tumors from a given group were too small to process (around 100 mm³) multiple tumors were pooled together for flow cytometry analysis. The cells were stained for T cell markers (CD45+, CD3+, CD4+, CD8+). The cells were fixed and acquired on a BD LSR Fortessa, and data was analysed by Kaluza.

TABLE 31
Treatment schedule in orthotopic EMT6 mouse in vivo model

| Group | Animals | Compounds | Dose | Schedule |
|---|---|---|---|---|
| 1 | 10 | Isotype Control | 10 mg/kg | Three times per week |
| | | | | (6 doses in total) |
| 2 | 10 | FAP × LTBR | 10 mg/kg | Three times per week |
| | | bsAb clone 4.11 | | (6 doses in total) |
| 3 | 10 | Peptide + | 90 μg, | D 0 and D 7 |
| | | adjuvant | 40 μg | |
| 4 | 10 | Peptide + | 90 μg, | D 0 and D 7 |
| | | adjuvant + | 40 μg | Three times per week |
| | | FAP × LTBR | 10 mg/kg | (6 doses in total) |
| | | bsAb clone 4.11 | | |

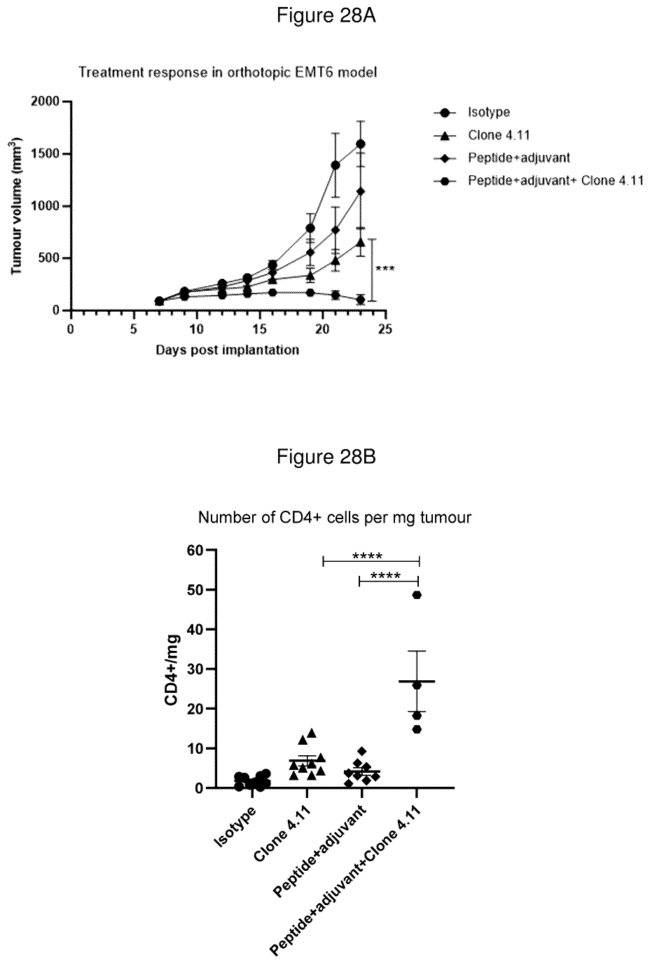

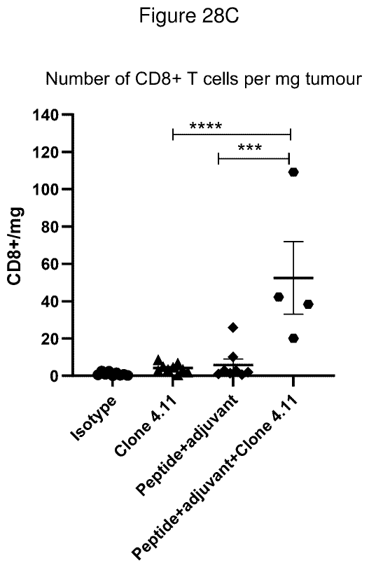

The combination of FAP/LTBR bispecific antibody, clone 4.11+adjuvant+peptide, resulted in highly significant tumor growth inhibition compared to a control group (adjuvant+peptide), leading to tumor growth regressions in 8/10 mice, in contrast to 3/10 in peptide+adjuvant, and 1/10 in clone 4.11 control groups (FIG. 28a). The tumor growth curves and flow cytometry results were plotted using GraphPad Prism Version 9. The statistical analysis was done with one-way ANOVA (*p<0.05, **p<0.01, ***p<0.001 ****p<0.0001).

The combination of FAP/LTBR bispecific antibody clone 4.11+adjuvant+peptide, resulted in a significant increase in CD4+ and CD8+ T cells in the tumor compared to isotype or adjuvant+peptide groups (FIGS. 28b and 28c). The statistical analysis was done with one-way ANOVA (*p<0.05, **p<0.01, ***p<0.001 ****p<0.0001).

4.15. In Vivo Efficacy with FAP-LTBR Surrogate Molecules in Combination with E22 Peptide Antigen in Orthotopic Breast Cancer Models (Rechallenge Study)

It is desirable to induce durable anti-tumour immunity for successful cancer immunotherapy, encompassing both the elimination of primary tumours and the prevention of recurrence. This rechallenge study evaluated the robustness of the immune response by reintroducing tumour cells following initial treatment-induced complete regression.

To elucidate the durability of the response elicited by clone 4.11 in combination with the E22 peptide (Castiglioni et al., Nature 2023), additional in vivo studies were conducted utilizing a syngeneic EMT6 breast cancer model. Mice were randomised into treatment cohorts when the mean tumour volume reached approximately 100 mm³. Treatment groups consisted of: (1) isotype control, (2) adjuvant (TLR-9 agonist, InvivoGen, cat. no. vac-1826-1)+E22 peptide as a control, (3) FAP/LTBR bispecific antibody clone 4.11, and (4) a combination of adjuvant+E22 peptide+FAP/LTBR bispecific antibody clone 4.11. The FAP/LTBR bispecific antibody was administered intraperitoneally at a dosage of 10 mg/kg, three times weekly. Mice were euthanised individually upon tumours reaching a predetermined volume threshold. Details of the treatment schedule are provided in Table 32.

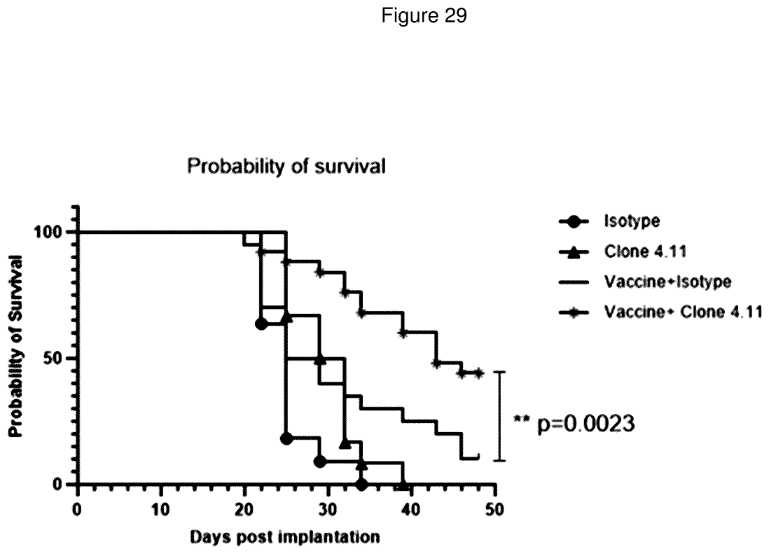

Combination therapy with the FAP-LTBR bispecific antibody (clone 4.11) and peptide vaccination (E22 peptide) resulted in durable complete regressions (CRs) in 28% of the subjects, as demonstrated in FIG. 29. Survival curves were generated using the Kaplan-Meier method, and the Log-rank test was employed to analyse the mouse survival data using GraphPad Prism Version 9. Statistical analysis was performed using one-way ANOVA (*p<0.05, **p<0.01, *** p<0.001, ****p<0.0001). Subsequently, two mice from the vaccine monotherapy group and seven mice from the vaccine+clone 4.11 group exhibiting complete regression were rechallenged 91 days after treatment cessation. The rechallenge consisted of a second implantation of EMT6 cells into the contralateral (left) mammary fat pad. Age-matched naive mice, unexposed to primary tumour implantation, received an equivalent number of rechallenge cells and served as controls.

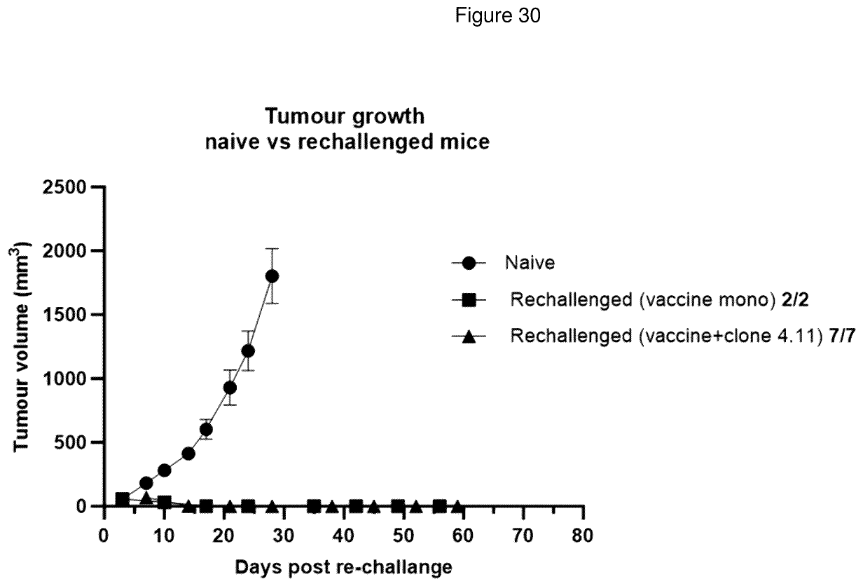

Notably, all rechallenged mice rejected the tumours, in contrast to the age-matched naive controls, suggesting the development of robust, long-term immune memory, as shown in FIG. 30. Tumour growth curves were plotted using GraphPad Prism Version 9.

TABLE 32
Treatment schedule in orthotopic
EMT6-huHER2 mouse in vivo model

| Group | Animals | Compounds | Dose | Schedule |
|---|---|---|---|---|
| 1 | 20 | Isotype Control | 10 mg/kg | Three times per week |
| 2 | 20 | FAP × LTBR | 10 mg/kg | Three times per week |
| | | bsAb clone 4.11 | | |
| 3 | 20 | Peptide + | 100 ug, | D 0 and D 7 |
| | | adjuvant | 20 ug | |
| 4 | 25 | Peptide + | 100 ug, | D 0 and D 7 |
| | | adjuvant + | 20 ug | Three times per week |
| | | FAP × LTBR | 10 mg/kg | |
| | | bsAb clone 4.11 | | |

The higher frequency of complete regressions observed in the combination group suggests possible synergistic effects between the peptide vaccine and the bispecific antibody.

The successful rejection of rechallenged tumours may indicate the generation of functional memory T cells capable of recognising and eliminating tumour cells. This outcome supports the hypothesis that the combination therapy induces a robust and durable adaptive immune response against tumour-associated antigens.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

Concentration percentages stated herein are w/v unless specified otherwise. The unit prefixes μ and u are used interchangeably herein for ‘micro’.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the claims which follow.

All publications, including but not limited to patents and patent applications, cited in this specification are herein incorporated by reference as if each individual publication were specifically and individually indicated to be incorporated by reference herein as though fully set forth.

### CLAUSES OF THE INVENTION

A series of clauses setting out embodiments of the invention are as follows. These embodiments generally relate to constructs comprising both a FAP binding agent and an LTBR binding agent.
Clause 1. A construct comprising a FAP binding agent and an LTBR binding agent.
Clause 2. The construct of clause 1 wherein the construct comprises only one FAP binding agent.
Clause 3. The construct of clause 1 wherein the construct comprises more than one FAP binding agent.
Clause 4. The construct of clause 3 wherein the construct comprises only two FAP binding agents.
Clause 5. The construct of any one of clauses 1 to 4 wherein the construct comprises only one LTBR binding agent.
Clause 6. The construct of any one of clauses 1 to 4 wherein the construct comprises more than one LTBR binding agent.
Clause 7. The construct of clause 6 wherein the construct comprises only two LTBR binding agents.
Clause 8. The construct of any one of clauses 1 to 7 wherein one or all of the LTBR binding agents comprise or consist of a binding polypeptide which binds to LTBR.
Clause 9. The construct of any one of clauses 1 to 8 wherein one or all of the FAP binding agents comprise or consist of a binding polypeptide which binds to FAP.
Clause 10. The construct of any one of clauses 1 to 9, wherein the construct comprises an antibody.
Clause 11. The construct of any one of clauses 1 to 9, wherein the construct consists of an antibody.
Clause 12. The construct of either clause 10 or 11 wherein the antibody is a bispecific antibody.
Clause 13. The construct of any one of clauses 10 to 12 wherein the antibody is a bivalent antibody.
Clause 14. The construct of clause 13 wherein the antibody consists essentially of a bispecific, bivalent antibody.
Clause 15. The construct of clause 14 wherein the antibody consists of a bispecific, bivalent antibody.
Clause 16. The construct of any one of clauses 10 to 15, wherein the antibody is an IgG1 antibody.
Clause 17. The construct of any one of clauses 1 to 10, wherein the construct comprises one or more antibody fragments.
Clause 18. The construct of any one of clauses 1 to 9, wherein the construct consists of one or more antibody fragments.
Clause 19. The construct of either clause 17 or 18, wherein the one or more antibody fragments are independently selected from an scFv, Fv, Fab, Fab′, F(ab′)2, variable domain (e.g. VH, VL, VNAR or VHH), diabody or minibody.
Clause 20. The construct of clause 19 wherein the construct comprises or consists of two scFvs.
Clause 21. The construct of either clause 19 or 20 wherein the scFvs comprise a disulfide bond in the VH44:VL100 position.
Clause 22. The construct of any one of clauses 1, 10, 11 or 16 to 19 wherein the construct has a 1:1 format.
Clause 23. The construct of any one of clauses 1, 10, 11 or 16 to 19 wherein the construct has a 2:1 format.
Clause 24. The construct of any one of clauses 1, 10, 11 or 16 to 19 wherein the construct has a 2:2 format.
Clause 25. The construct of any one of clauses 1 to 24 wherein one or all of the LTBR binding agents comprise or consist of a variable domain which binds to LTBR.
Clause 26. The construct of clause 25, wherein the variable domain which binds to LTBR is selected from a VH, VL, VNAR or VHH.
Clause 27. The construct of clause 26 wherein the variable domain which binds to LTBR is a heavy chain variable domain.
Clause 28. The construct of clause 27 wherein the heavy chain variable domain which binds to LTBR is a VHH.
Clause 29. The construct of any one of clauses 1 to 28 wherein the construct comprises a further binding agent which is neither an LTBR binding agent nor a FAP binding agent.
Clause 30. The construct of any one of clauses 1 to 28 wherein the construct comprises no further binding agents.
Clause 31. The construct of any one of clauses 25 to 30 wherein the variable domain which binds to LTBR comprises or consists of three heavy chain complementarity determining regions (HCDR1-HCDR3) and four framework regions (FR1-FR4), wherein HCDR1 comprises or consists of a polypeptide sequence sharing 70% or greater, 75% or greater, 80% or greater, 85% or greater, 90% or greater or 95% or greater sequence identity with SEQ ID NO: 57 or 166 or wherein HCDR1 comprises or consists of a polypeptide sequence having 3, 2 or 1 amino acid sequence difference with SEQ ID NO: 57 or 166, HCDR2 comprises or consists of a polypeptide sequence sharing 70% or greater, 75% or greater, 80% or greater, 85% or greater, 90% or greater or 95% or greater sequence identity with SEQ ID NO: 205, 58 or 167 or wherein HCDR2 comprises or consists of a polypeptide sequence having 3, 2 or 1 amino acid sequence difference with SEQ ID NO: 205, 58 or 167, and HCDR3 comprises or consists of a polypeptide sequence sharing 70% or greater, 75% or greater, 80% or greater, 85% or greater, 90% or greater or 95% or greater sequence identity with SEQ ID NO: 59 or 168 or wherein HCDR3 comprises or consists of a polypeptide sequence having 3, 2 or 1 amino acid sequence difference with SEQ ID NO: 59 or 168.
Clause 32. The construct of clause 31 wherein HCDR1 comprises or consists of a polypeptide sequence of SEQ ID NO: 57 or 166, HCDR2 comprises or consists of a polypeptide sequence of SEQ ID NO: 205, 58 or 167 and HCDR3 comprises or consists of a polypeptide sequence of SEQ ID NO: 59 or 168.
Clause 33. The construct of any one of clauses 25 to 30 wherein the variable domain which binds to LTBR comprises or consists of three heavy chain complementarity determining regions (HCDR1-HCDR3) and four framework regions (FR1-FR4), wherein HCDR1 comprises or consists of a polypeptide sequence sharing 70% or greater, 75% or greater, 80% or greater, 85% or greater, 90% or greater or 95% or greater sequence identity with SEQ ID NO: 60 or 172 or wherein HCDR1 comprises or consists of a polypeptide sequence having 3, 2 or 1 amino acid sequence difference with SEQ ID NO: 60 or 172, HCDR2 comprises or consists of a polypeptide sequence sharing 70% or greater, 75% or greater, 80% or greater, 85% or greater, 90% or greater or 95% or greater sequence identity with SEQ ID NO: 61 or 173 or wherein HCDR2 comprises or consists of a polypeptide sequence having 3, 2 or 1 amino acid sequence difference with SEQ ID NO: 61 or 173, and HCDR3 comprises or consists of a polypeptide sequence sharing 70% or greater, 75% or greater, 80% or greater, 85% or greater, 90% or greater or 95% or greater sequence identity with SEQ ID NO: 62 or 173 or wherein HCDR3 comprises or consists of a polypeptide sequence having 3, 2 or 1 amino acid sequence difference with SEQ ID NO: 62 or 173.
Clause 34. The construct of clause 33 wherein HCDR1 comprises or consists of a polypeptide sequence of SEQ ID NO: 60 or 172, HCDR2 comprises or consists of a polypeptide sequence of SEQ ID NO: 61 or 173 and HCDR3 comprises or consists of a polypeptide sequence of SEQ ID NO: 62 or 174.
Clause 35. The construct of any one of clauses 25 to 30 wherein the variable domain which binds to LTBR comprises or consists of three heavy chain complementarity determining regions (HCDR1-HCDR3) and four framework regions (FR1-FR4), wherein HCDR1 comprises or consists of a polypeptide sequence sharing 70% or greater, 75% or greater, 80% or greater, 85% or greater, 90% or greater or 95% or greater sequence identity with SEQ ID NO: 63 or 163 or wherein HCDR1 comprises or consists of a polypeptide sequence having 3, 2 or 1 amino acid sequence difference with SEQ ID NO: 63 or 163, HCDR2 comprises or consists of a polypeptide sequence sharing 70% or greater, 75% or greater, 80% or greater, 85% or greater, 90% or greater or 95% or greater sequence identity with SEQ ID NO: 64 or 164 or wherein HCDR2 comprises or consists of a polypeptide sequence having 3, 2 or 1 amino acid sequence difference with SEQ ID NO: 64 or 164, and HCDR3 comprises or consists of a polypeptide sequence sharing 70% or greater, 75% or greater, 80% or greater, 85% or greater, 90% or greater or 95% or greater sequence identity with SEQ ID NO: 207, 65 or 165 or wherein HCDR3 comprises or consists of a polypeptide sequence having 3, 2 or 1 amino acid sequence difference with SEQ ID NO: 207, 65 or 165.
Clause 36. The construct of clause 35 wherein HCDR1 comprises or consists of a polypeptide sequence of SEQ ID NO: 63 or 163, HCDR2 comprises or consists of a polypeptide sequence of SEQ ID NO: 64 or 164 and HCDR3 comprises or consists of a polypeptide sequence of SEQ ID NO: 207, 65 or 165.
Clause 37. The construct of any one of clauses 25 to 30 wherein the variable domain which binds to LTBR comprises or consists of three heavy chain complementarity determining regions (HCDR1-HCDR3) and four framework regions (FR1-FR4), wherein HCDR1 comprises or consists of a polypeptide sequence sharing 70% or greater, 75% or greater, 80% or greater, 85% or greater, 90% or greater or 95% or greater sequence identity with SEQ ID NO: 66 or 169 or wherein HCDR1 comprises or consists of a polypeptide sequence having 3, 2 or 1 amino acid sequence difference with SEQ ID NO: 66 or 169, HCDR2 comprises or consists of a polypeptide sequence sharing 70% or greater, 75% or greater, 80% or greater, 85% or greater, 90% or greater or 95% or greater sequence identity with SEQ ID NO: 206, 67 or 170 or wherein HCDR2 comprises or consists of a polypeptide sequence having 3, 2 or 1 amino acid sequence difference with SEQ ID NO: 206, 67 or 170, and HCDR3 comprises or consists of a polypeptide sequence sharing 70% or greater, 75% or greater, 80% or greater, 85% or greater, 90% or greater or 95% or greater sequence identity with SEQ ID NO: 208, 68 or 171 or wherein HCDR3 comprises or consists of a polypeptide sequence having 3, 2 or 1 amino acid sequence difference with SEQ ID NO: 208, 68 or 171.
Clause 38. The construct of clause 37 wherein HCDR1 comprises or consists of a polypeptide sequence of SEQ ID NO: 66 or 169, HCDR2 comprises or consists of a polypeptide sequence of SEQ ID NO: 206, 67 or 170 and HCDR3 comprises or consists of a polypeptide sequence of SEQ ID NO: 208, 68 or 171.
Clause 39. The construct of any one of clauses 25 to 30 wherein the variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 23 or 160.
Clause 40. The construct of clause 39 wherein the variable domain which binds to LTBR comprises or consists of a polypeptide sequence of SEQ ID NO: 23 or 160.
Clause 41. The construct of any one of clauses 25 to 30 wherein the variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 24 or 162.
Clause 42. The construct of clause 41 wherein the variable domain which binds to LTBR comprises or consists of a polypeptide sequence of SEQ ID NO: 24 or 162.
Clause 43. The construct of any one of clauses 25 to 30 wherein the variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 25 or 159.
Clause 44. The construct of clause 43 wherein the variable domain which binds to LTBR comprises or consists of a polypeptide sequence of SEQ ID NO: 25 or 159.
Clause 45. The construct of any one of clauses 25 to 30 wherein the variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 26 or 161.
Clause 46. The construct of clause 45 wherein the variable domain which binds to LTBR comprises or consists of a polypeptide sequence of SEQ ID NO: 26 or 161.
Clause 47. The construct of any one of clauses 1 to 24 wherein one or all of the LTBR binding agents comprise or consist of a paired heavy chain variable domain and light chain variable domain which bind to LTBR.
Clause 48. The construct of clause 47, wherein one or all of the LTBR binding agents is independently selected from an scFv, Fv, Fab, Fab′, F(ab′)2, diabody or minibody.
Clause 49. The construct of either clause 47 or 48 wherein the heavy chain variable domain which binds to LTBR comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 69, HCDR2 comprises or consists of a polypeptide sequence sharing 70% or greater, 75% or greater, 80% or greater, 85% or greater, 90% or greater or 95% or greater sequence identity with SEQ ID NO: 70 and HCDR3 comprises or consists of a polypeptide sequence sharing 70% or greater, 75% or greater, 80% or greater, 85% or greater, 90% or greater or 95% or greater sequence identity with SEQ ID NO: 71 and the light chain variable domain which binds to LTBR comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 72, LCDR2 comprises or consists of a polypeptide sequence sharing 70% or greater, 75% or greater, 80% or greater, 85% or greater, 90% or greater or 95% or greater sequence identity with SEQ ID NO: 73 and LCDR3 comprises or consists of a polypeptide sequence sharing 70% or greater, 75% or greater, 80% or greater, 85% or greater, 90% or greater or 95% or greater sequence identity with SEQ ID NO: 74.
Clause 50. The construct of clause 49 wherein HCDR1 comprises or consists of a polypeptide sequence of SEQ ID NO: 69, HCDR2 comprises or consists of a polypeptide sequence of SEQ ID NO: 70 and HCDR3 comprises or consists of a polypeptide sequence of SEQ ID NO: 71 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 72, LCDR2 comprises or consists of a polypeptide sequence of SEQ ID NO: 73 and LCDR3 comprises or consists of a polypeptide sequence of SEQ ID NO: 74.
Clause 51. The construct of either clause 47 or 48 wherein the heavy chain variable domain which binds to LTBR comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 75, HCDR2 comprises or consists of a polypeptide sequence sharing 70% or greater, 75% or greater, 80% or greater, 85% or greater, 90% or greater or 95% or greater sequence identity with SEQ ID NO: 76 and HCDR3 comprises or consists of a polypeptide sequence sharing 70% or greater, 75% or greater, 80% or greater, 85% or greater, 90% or greater or 95% or greater sequence identity with SEQ ID NO: 77 and the light chain variable domain which binds to LTBR comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 78, LCDR2 comprises or consists of a polypeptide sequence sharing 70% or greater, 75% or greater, 80% or greater, 85% or greater, 90% or greater or 95% or greater sequence identity with SEQ ID NO: 79 and LCDR3 comprises or consists of a polypeptide sequence sharing 70% or greater, 75% or greater, 80% or greater, 85% or greater, 90% or greater or 95% or greater sequence identity with SEQ ID NO: 80.
Clause 52. The construct of clause 51 wherein HCDR1 comprises or consists of a polypeptide sequence of SEQ ID NO: 75, HCDR2 comprises or consists of a polypeptide sequence of SEQ ID NO: 76 and HCDR3 comprises or consists of a polypeptide sequence of SEQ ID NO: 77 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 78, LCDR2 comprises or consists of a polypeptide sequence of SEQ ID NO: 79 and LCDR3 comprises or consists of a polypeptide sequence of SEQ ID NO: 80.
Clause 53. The construct of either clause 47 or 48 wherein the heavy chain variable domain which binds to LTBR comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 81, HCDR2 comprises or consists of a polypeptide sequence sharing 70% or greater, 75% or greater, 80% or greater, 85% or greater, 90% or greater or 95% or greater sequence identity with SEQ ID NO: 82 and HCDR3 comprises or consists of a polypeptide sequence sharing 70% or greater, 75% or greater, 80% or greater, 85% or greater, 90% or greater or 95% or greater sequence identity with SEQ ID NO: 83 and the light chain variable domain which binds to LTBR comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 84, LCDR2 comprises or consists of a polypeptide sequence sharing 70% or greater, 75% or greater, 80% or greater, 85% or greater, 90% or greater or 95% or greater sequence identity with SEQ ID NO: 85 and LCDR3 comprises or consists of a polypeptide sequence sharing 70% or greater, 75% or greater, 80% or greater, 85% or greater, 90% or greater or 95% or greater sequence identity with SEQ ID NO: 86.
Clause 54. The construct of clause 53 wherein HCDR1 comprises or consists of a polypeptide sequence of SEQ ID NO: 81, HCDR2 comprises or consists of a polypeptide sequence of SEQ ID NO: 82 and HCDR3 comprises or consists of a polypeptide sequence of SEQ ID NO: 83 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 84, LCDR2 comprises or consists of a polypeptide sequence of SEQ ID NO: 85 and LCDR3 comprises or consists of a polypeptide sequence of SEQ ID NO: 86.
Clause 55. The construct of either clause 47 or 48 wherein the heavy chain variable domain which binds to LTBR comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 87, HCDR2 comprises or consists of a polypeptide sequence sharing 70% or greater, 75% or greater, 80% or greater, 85% or greater, 90% or greater or 95% or greater sequence identity with SEQ ID NO: 88 and HCDR3 comprises or consists of a polypeptide sequence sharing 70% or greater, 75% or greater, 80% or greater, 85% or greater, 90% or greater or 95% or greater sequence identity with SEQ ID NO: 89 and the light chain variable domain which binds to LTBR comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 90, LCDR2 comprises or consists of a polypeptide sequence sharing 70% or greater, 75% or greater, 80% or greater, 85% or greater, 90% or greater or 95% or greater sequence identity with SEQ ID NO: 91 and LCDR3 comprises or consists of a polypeptide sequence sharing 70% or greater, 75% or greater, 80% or greater, 85% or greater, 90% or greater or 95% or greater sequence identity with SEQ ID NO: 92.
Clause 56. The construct of clause 55 wherein HCDR1 comprises or consists of a polypeptide sequence of SEQ ID NO: 87, HCDR2 comprises or consists of a polypeptide sequence of SEQ ID NO: 88 and HCDR3 comprises or consists of a polypeptide sequence of SEQ ID NO: 89 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 90, LCDR2 comprises or consists of a polypeptide sequence of SEQ ID NO: 91 and LCDR3 comprises or consists of a polypeptide sequence of SEQ ID NO: 92.
Clause 57. The construct of either clause 47 or 48 wherein the heavy chain variable domain which binds to LTBR comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 93, HCDR2 comprises or consists of a polypeptide sequence sharing 70% or greater, 75% or greater, 80% or greater, 85% or greater, 90% or greater or 95% or greater sequence identity with SEQ ID NO: 94 and HCDR3 comprises or consists of a polypeptide sequence sharing 70% or greater, 75% or greater, 80% or greater, 85% or greater, 90% or greater or 95% or greater sequence identity with SEQ ID NO: 95 and the light chain variable domain which binds to LTBR comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 96, LCDR2 comprises or consists of a polypeptide sequence sharing 70% or greater, 75% or greater, 80% or greater, 85% or greater, 90% or greater or 95% or greater sequence identity with SEQ ID NO: 97 and LCDR3 comprises or consists of a polypeptide sequence sharing 70% or greater, 75% or greater, 80% or greater, 85% or greater, 90% or greater or 95% or greater sequence identity with SEQ ID NO: 98.
Clause 58. The construct of clause 57 wherein HCDR1 comprises or consists of a polypeptide sequence of SEQ ID NO: 93, HCDR2 comprises or consists of a polypeptide sequence of SEQ ID NO: 94 and HCDR3 comprises or consists of a polypeptide sequence of SEQ ID NO: 95 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 96, LCDR2 comprises or consists of a polypeptide sequence of SEQ ID NO: 97 and LCDR3 comprises or consists of a polypeptide sequence of SEQ ID NO: 98.
Clause 59. The construct of either clause 47 or 48 wherein the heavy chain variable domain which binds to LTBR comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 99, HCDR2 comprises or consists of a polypeptide sequence sharing 70% or greater, 75% or greater, 80% or greater, 85% or greater, 90% or greater or 95% or greater sequence identity with SEQ ID NO: 100 and HCDR3 comprises or consists of a polypeptide sequence sharing 70% or greater, 75% or greater, 80% or greater, 85% or greater, 90% or greater or 95% or greater sequence identity with SEQ ID NO: 101 and the light chain variable domain which binds to LTBR comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 102, LCDR2 comprises or consists of a polypeptide sequence sharing 70% or greater, 75% or greater, 80% or greater, 85% or greater, 90% or greater or 95% or greater sequence identity with SEQ ID NO: 103 and LCDR3 comprises or consists of a polypeptide sequence sharing 70% or greater, 75% or greater, 80% or greater, 85% or greater, 90% or greater or 95% or greater sequence identity with SEQ ID NO: 104.
Clause 60. The construct of clause 59 wherein HCDR1 comprises or consists of a polypeptide sequence of SEQ ID NO: 99, HCDR2 comprises or consists of a polypeptide sequence of SEQ ID NO: 100 and HCDR3 comprises or consists of a polypeptide sequence of SEQ ID NO: 101 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 102, LCDR2 comprises or consists of a polypeptide sequence of SEQ ID NO: 103 and LCDR3 comprises or consists of a polypeptide sequence of SEQ ID NO: 104.
Clause 61. The construct of any one of either clause 47 or 48 wherein the heavy chain variable domain which binds to LTBR comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 105, HCDR2 comprises or consists of a polypeptide sequence sharing 70% or greater, 75% or greater, 80% or greater, 85% or greater, 90% or greater or 95% or greater sequence identity with SEQ ID NO: 106 and HCDR3 comprises or consists of a polypeptide sequence sharing 70% or greater, 75% or greater, 80% or greater, 85% or greater, 90% or greater or 95% or greater sequence identity with SEQ ID NO: 107 and the light chain variable domain which binds to LTBR comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 108, LCDR2 comprises or consists of a polypeptide sequence sharing 70% or greater, 75% or greater, 80% or greater, 85% or greater, 90% or greater or 95% or greater sequence identity with SEQ ID NO: 109 and LCDR3 comprises or consists of a polypeptide sequence sharing 70% or greater, 75% or greater, 80% or greater, 85% or greater, 90% or greater or 95% or greater sequence identity with SEQ ID NO: 110.
Clause 62. The construct of clause 61 wherein HCDR1 comprises or consists of a polypeptide sequence of SEQ ID NO: 105, HCDR2 comprises or consists of a polypeptide sequence of SEQ ID NO: 106 and HCDR3 comprises or consists of a polypeptide sequence of SEQ ID NO: 107 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 108, LCDR2 comprises or consists of a polypeptide sequence of SEQ ID NO: 109 and LCDR3 comprises or consists of a polypeptide sequence of SEQ ID NO: 110.
Clause 63. The construct of either clause 47 or 48 wherein the heavy chain variable domain which binds to LTBR comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 111, HCDR2 comprises or consists of a polypeptide sequence sharing 70% or greater, 75% or greater, 80% or greater, 85% or greater, 90% or greater or 95% or greater sequence identity with SEQ ID NO: 112 and HCDR3 comprises or consists of a polypeptide sequence sharing 70% or greater, 75% or greater, 80% or greater, 85% or greater, 90% or greater or 95% or greater sequence identity with SEQ ID NO: 113 and the light chain variable domain which binds to LTBR comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 114, LCDR2 comprises or consists of a polypeptide sequence sharing 70% or greater, 75% or greater, 80% or greater, 85% or greater, 90% or greater or 95% or greater sequence identity with SEQ ID NO: 115 and LCDR3 comprises or consists of a polypeptide sequence sharing 70% or greater, 75% or greater, 80% or greater, 85% or greater, 90% or greater or 95% or greater sequence identity with SEQ ID NO: 116.
Clause 64. The construct of clause 63 wherein HCDR1 comprises or consists of a polypeptide sequence of SEQ ID NO: 111, HCDR2 comprises or consists of a polypeptide sequence of SEQ ID NO: 112 and HCDR3 comprises or consists of a polypeptide sequence of SEQ ID NO: 113 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 114, LCDR2 comprises or consists of a polypeptide sequence of SEQ ID NO: 115 and LCDR3 comprises or consists of a polypeptide sequence of SEQ ID NO: 116.
Clause 65. The construct of either clause 47 or 48 wherein the heavy chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 27 and the light chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 28.
Clause 66. The construct of clause 65 wherein the heavy chain variable domain comprises or consists of a polypeptide sequence of SEQ ID NO: 27 and the light chain variable domain comprises or consists of a polypeptide sequence of SEQ ID NO: 28.
Clause 67. The construct of either clause 47 or 48 wherein the heavy chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 29 and the light chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 30.
Clause 68. The construct of clause 67 wherein the heavy chain variable domain comprises or consists of a polypeptide sequence of SEQ ID NO: 29 and the light chain variable domain comprises or consists of a polypeptide sequence of SEQ ID NO: 30.
Clause 69. The construct of either clause 47 or 48 wherein the heavy chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 31 and the light chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 32.
Clause 70. The construct of clause 69 wherein the heavy chain variable domain comprises or consists of a polypeptide sequence of SEQ ID NO: 31 and the light chain variable domain comprises or consists of a polypeptide sequence of SEQ ID NO: 32.
Clause 71. The construct of either clause 47 or 48 wherein the heavy chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 33 and the light chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 34.
Clause 72. The construct of clause 71 wherein the heavy chain variable domain comprises or consists of a polypeptide sequence of SEQ ID NO: 33 and the light chain variable domain comprises or consists of a polypeptide sequence of SEQ ID NO: 34.
Clause 73. The construct of either clause 47 or 48 wherein the heavy chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 35 and the light chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 36.
Clause 74. The construct of clause 73 wherein the heavy chain variable domain comprises or consists of a polypeptide sequence of SEQ ID NO: 35 and the light chain variable domain comprises or consists of a polypeptide sequence of SEQ ID NO: 36.
Clause 75. The construct of either clause 47 or 48 wherein the heavy chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 37 and the light chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 38.
Clause 76. The construct of clause 75 wherein the heavy chain variable domain comprises or consists of a polypeptide sequence of SEQ ID NO: 37 and the light chain variable domain comprises or consists of a polypeptide sequence of SEQ ID NO: 38.
Clause 77. The construct of either clause 47 or 48 wherein the heavy chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 39 and the light chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 40.
Clause 78. The construct of clause 77 wherein the heavy chain variable domain comprises or consists of a polypeptide sequence of SEQ ID NO: 39 and the light chain variable domain comprises or consists of a polypeptide sequence of SEQ ID NO: 40.
Clause 79. The construct of either clause 47 or 48 wherein the heavy chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 41 and the light chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 42.
Clause 80. The construct of clause 79 wherein the heavy chain variable domain comprises or consists of a polypeptide sequence of SEQ ID NO: 41 and the light chain variable domain comprises or consists of a polypeptide sequence of SEQ ID NO: 42.
Clause 81. The construct of any one of clauses 1 to 80 wherein one or all of the FAP binding agents comprise or consist of a paired heavy chain variable domain and light chain variable domain which binds to FAP.
Clause 82. The construct of clause 81, wherein one or all of the FAP binding agents are independently selected from an scFv, Fv, Fab, Fab′, F(ab′)2, diabody or minibody.
Clause 83. The construct of either clause 81 or 82 the heavy chain variable domain which binds to FAP comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 117, HCDR2 comprises or consists of a polypeptide sequence sharing 70% or greater, 75% or greater, 80% or greater, 85% or greater, 90% or greater or 95% or greater sequence identity with SEQ ID NO: 118 and HCDR3 comprises or consists of a polypeptide sequence sharing 70% or greater, 75% or greater, 80% or greater, 85% or greater, 90% or greater or 95% or greater sequence identity with SEQ ID NO: 119 and the light chain variable domain which binds to FAP comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 120, LCDR2 comprises or consists of a polypeptide sequence sharing 70% or greater, 75% or greater, 80% or greater, 85% or greater, 90% or greater or 95% or greater sequence identity with SEQ ID NO: 121 and LCDR3 comprises or consists of a polypeptide sequence sharing 70% or greater, 75% or greater, 80% or greater, 85% or greater, 90% or greater or 95% or greater sequence identity with SEQ ID NO: 122.
Clause 84. The construct of clause 83 wherein HCDR1 comprises or consists of a polypeptide sequence of SEQ ID NO: 117, HCDR2 comprises or consists of a polypeptide sequence of SEQ ID NO: 118 and HCDR3 comprises or consists of a polypeptide sequence of SEQ ID NO: 119 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 120, LCDR2 comprises or consists of a polypeptide sequence of SEQ ID NO: 121 and LCDR3 comprises or consists of a polypeptide sequence of SEQ ID NO: 122.
Clause 85. The construct of either clause 81 or 82 wherein the heavy chain variable domain which binds to FAP comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 123, HCDR2 comprises or consists of a polypeptide sequence sharing 70% or greater, 75% or greater, 80% or greater, 85% or greater, 90% or greater or 95% or greater sequence identity with SEQ ID NO: 124 and HCDR3 comprises or consists of a polypeptide sequence sharing 70% or greater, 75% or greater, 80% or greater, 85% or greater, 90% or greater or 95% or greater sequence identity with SEQ ID NO: 125 and the light chain variable domain which binds to FAP comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 126, LCDR2 comprises or consists of a polypeptide sequence sharing 70% or greater, 75% or greater, 80% or greater, 85% or greater, 90% or greater or 95% or greater sequence identity with SEQ ID NO: 127 and LCDR3 comprises or consists of a polypeptide sequence sharing 70% or greater, 75% or greater, 80% or greater, 85% or greater, 90% or greater or 95% or greater sequence identity with SEQ ID NO: 128.
Clause 86. The construct of clause 85 wherein HCDR1 comprises or consists of a polypeptide sequence of SEQ ID NO: 123, HCDR2 comprises or consists of a polypeptide sequence of SEQ ID NO: 124 and HCDR3 comprises or consists of a polypeptide sequence of SEQ ID NO: 125 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 126, LCDR2 comprises or consists of a polypeptide sequence of SEQ ID NO: 127 and LCDR3 comprises or consists of a polypeptide sequence of SEQ ID NO: 128.
Clause 87. The construct of either clause 81 or 82 wherein the heavy chain variable domain which binds to FAP comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 129, HCDR2 comprises or consists of a polypeptide sequence sharing 70% or greater, 75% or greater, 80% or greater, 85% or greater, 90% or greater or 95% or greater sequence identity with SEQ ID NO: 130 and HCDR3 comprises or consists of a polypeptide sequence sharing 70% or greater, 75% or greater, 80% or greater, 85% or greater, 90% or greater or 95% or greater sequence identity with SEQ ID NO: 131 and the light chain variable domain which binds to FAP comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 132, LCDR2 comprises or consists of a polypeptide sequence sharing 70% or greater, 75% or greater, 80% or greater, 85% or greater, 90% or greater or 95% or greater sequence identity with SEQ ID NO: 133 and LCDR3 comprises or consists of a polypeptide sequence sharing 70% or greater, 75% or greater, 80% or greater, 85% or greater, 90% or greater or 95% or greater sequence identity with SEQ ID NO: 134.
Clause 88. The construct of clause 87 wherein HCDR1 comprises or consists of a polypeptide sequence of SEQ ID NO: 129, HCDR2 comprises or consists of a polypeptide sequence of SEQ ID NO: 130 and HCDR3 comprises or consists of a polypeptide sequence of SEQ ID NO: 131 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 132, LCDR2 comprises or consists of a polypeptide sequence of SEQ ID NO: 133 and LCDR3 comprises or consists of a polypeptide sequence of SEQ ID NO: 134.
Clause 89. The construct of either clause 81 or 82 wherein the heavy chain variable domain which binds to FAP comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 135, HCDR2 comprises or consists of a polypeptide sequence sharing 70% or greater, 75% or greater, 80% or greater, 85% or greater, 90% or greater or 95% or greater sequence identity with SEQ ID NO: 136 and HCDR3 comprises or consists of a polypeptide sequence sharing 70% or greater, 75% or greater, 80% or greater, 85% or greater, 90% or greater or 95% or greater sequence identity with SEQ ID NO: 137 and the light chain variable domain which binds to FAP comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 138, LCDR2 comprises or consists of a polypeptide sequence sharing 70% or greater, 75% or greater, 80% or greater, 85% or greater, 90% or greater or 95% or greater sequence identity with SEQ ID NO: 139 and LCDR3 comprises or consists of a polypeptide sequence sharing 70% or greater, 75% or greater, 80% or greater, 85% or greater, 90% or greater or 95% or greater sequence identity with SEQ ID NO: 140.
Clause 90. The construct of clause 89 wherein HCDR1 comprises or consists of a polypeptide sequence of SEQ ID NO: 135, HCDR2 comprises or consists of a polypeptide sequence of SEQ ID NO: 136 and HCDR3 comprises or consists of a polypeptide sequence of SEQ ID NO: 137 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 138, LCDR2 comprises or consists of a polypeptide sequence of SEQ ID NO: 139 and LCDR3 comprises or consists of a polypeptide sequence of SEQ ID NO: 140.
Clause 91. The construct of any either clause 81 or 82 wherein the heavy chain variable domain which binds to FAP comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 141, HCDR2 comprises or consists of a polypeptide sequence sharing 70% or greater, 75% or greater, 80% or greater, 85% or greater, 90% or greater or 95% or greater sequence identity with SEQ ID NO: 142 and HCDR3 comprises or consists of a polypeptide sequence sharing 70% or greater, 75% or greater, 80% or greater, 85% or greater, 90% or greater or 95% or greater sequence identity with SEQ ID NO: 143 and the light chain variable domain which binds to FAP comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 144, LCDR2 comprises or consists of a polypeptide sequence sharing 70% or greater, 75% or greater, 80% or greater, 85% or greater, 90% or greater or 95% or greater sequence identity with SEQ ID NO: 145 and LCDR3 comprises or consists of a polypeptide sequence sharing 70% or greater, 75% or greater, 80% or greater, 85% or greater, 90% or greater or 95% or greater sequence identity with SEQ ID NO: 146.
Clause 92. The construct of clause 91 wherein HCDR1 comprises or consists of a polypeptide sequence of SEQ ID NO: 141, HCDR2 comprises or consists of a polypeptide sequence of SEQ ID NO: 142 and HCDR3 comprises or consists of a polypeptide sequence of SEQ ID NO: 143 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 144, LCDR2 comprises or consists of a polypeptide sequence of SEQ ID NO: 145 and LCDR3 comprises or consists of a polypeptide sequence of SEQ ID NO: 146.
Clause 93. The construct of either clause 81 or 82 wherein the heavy chain variable domain which binds to FAP comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 147, HCDR2 comprises or consists of a polypeptide sequence sharing 70% or greater, 75% or greater, 80% or greater, 85% or greater, 90% or greater or 95% or greater sequence identity with SEQ ID NO: 148 and HCDR3 comprises or consists of a polypeptide sequence sharing 70% or greater, 75% or greater, 80% or greater, 85% or greater, 90% or greater or 95% or greater sequence identity with SEQ ID NO: 149 and the light chain variable domain which binds to FAP comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 150, LCDR2 comprises or consists of a polypeptide sequence sharing 70% or greater, 75% or greater, 80% or greater, 85% or greater, 90% or greater or 95% or greater sequence identity with SEQ ID NO: 151 and LCDR3 comprises or consists of a polypeptide sequence sharing 70% or greater, 75% or greater, 80% or greater, 85% or greater, 90% or greater or 95% or greater sequence identity with SEQ ID NO: 152.
Clause 94. The construct of clause 93 wherein HCDR1 comprises or consists of a polypeptide sequence of SEQ ID NO: 147, HCDR2 comprises or consists of a polypeptide sequence of SEQ ID NO: 148 and HCDR3 comprises or consists of a polypeptide sequence of SEQ ID NO: 149 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 150, LCDR2 comprises or consists of a polypeptide sequence of SEQ ID NO: 151 and LCDR3 comprises or consists of a polypeptide sequence of SEQ ID NO: 152.
Clause 95. The construct of either clause 81 or 82 wherein the heavy chain variable domain which binds to FAP comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 153, HCDR2 comprises or consists of a polypeptide sequence sharing 70% or greater, 75% or greater, 80% or greater, 85% or greater, 90% or greater or 95% or greater sequence identity with SEQ ID NO: 154 and HCDR3 comprises or consists of a polypeptide sequence sharing 70% or greater, 75% or greater, 80% or greater, 85% or greater, 90% or greater or 95% or greater sequence identity with SEQ ID NO: 155 and the light chain variable domain which binds to FAP comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 156, LCDR2 comprises or consists of a polypeptide sequence sharing 70% or greater, 75% or greater, 80% or greater, 85% or greater, 90% or greater or 95% or greater sequence identity with SEQ ID NO: 157 and LCDR3 comprises or consists of a polypeptide sequence sharing 70% or greater, 75% or greater, 80% or greater, 85% or greater, 90% or greater or 95% or greater sequence identity with SEQ ID NO: 158.
Clause 96. The construct of clause 95 wherein HCDR1 comprises or consists of a polypeptide sequence of SEQ ID NO: 153, HCDR2 comprises or consists of a polypeptide sequence of SEQ ID NO: 154 and HCDR3 comprises or consists of a polypeptide sequence of SEQ ID NO: 155 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 156, LCDR2 comprises or consists of a polypeptide sequence of SEQ ID NO: 157 and LCDR3 comprises or consists of a polypeptide sequence of SEQ ID NO: 158.
Clause 97. The construct of either clause 81 or 82 wherein the heavy chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 7 and the light chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 8.
Clause 98. The construct of clause 97 wherein the heavy chain variable domain comprises or consists of a polypeptide sequence of SEQ ID NO: 7 and the light chain variable domain comprises or consists of a polypeptide sequence of SEQ ID NO: 8.
Clause 99. The construct of either clause 81 or 82 wherein the heavy chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 43 and the light chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 44.
Clause 100. The construct of clause 99 wherein the heavy chain variable domain comprises or consists of a polypeptide sequence of SEQ ID NO: 43 and the light chain variable domain comprises or consists of a polypeptide sequence of SEQ ID NO: 44.
Clause 101. The construct of either clause 81 or 82 wherein the heavy chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 45 and the light chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 46.
Clause 102. The construct of clause 101 wherein the heavy chain variable domain comprises or consists of a polypeptide sequence of SEQ ID NO: 45 and the light chain variable domain comprises or consists of a polypeptide sequence of SEQ ID NO: 46.
Clause 103. The construct of either clause 81 or 82 wherein the heavy chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 47 and the light chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 48.
Clause 104. The construct of clause 103 wherein the heavy chain variable domain comprises or consists of a polypeptide sequence of SEQ ID NO: 47 and the light chain variable domain comprises or consists of a polypeptide sequence of SEQ ID NO: 48.
Clause 105. The construct of either clause 81 or 82 wherein the heavy chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 49 and the light chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 50.
Clause 106. The construct of clause 105 wherein the heavy chain variable domain comprises or consists of a polypeptide sequence of SEQ ID NO: 49 and the light chain variable domain comprises or consists of a polypeptide sequence of SEQ ID NO: 50.
Clause 107. The construct of either clause 81 or 82 wherein the heavy chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 51 and the light chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 52.
Clause 108. The construct of clause 107 wherein the heavy chain variable domain comprises or consists of a polypeptide sequence of SEQ ID NO: 51 and the light chain variable domain comprises or consists of a polypeptide sequence of SEQ ID NO: 52.
Clause 109. The construct of either clause 81 or 82 wherein the heavy chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 53 and the light chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 54.
Clause 110. The construct of clause 109 wherein the heavy chain variable domain comprises or consists of a polypeptide sequence of SEQ ID NO: 53 and the light chain variable domain comprises or consists of a polypeptide sequence of SEQ ID NO: 54.
Clause 111. The construct of either clause 81 or 82 wherein the heavy chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 55 and the light chain variable domain which binds to FAP comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 56.
Clause 112. The construct of clause 111 wherein the heavy chain variable domain comprises or consists of a polypeptide sequence of SEQ ID NO: 55 and the light chain variable domain comprises or consists of a polypeptide sequence of SEQ ID NO: 56.
Clause 113. The construct of any one of clauses 1 to 112 wherein the construct comprises an Fc region, wherein optionally one or all of the LTBR binding agents are linked directly by a peptide linker to the Fc region, for example to the CH3 region.
Clause 114. The construct of clause 113 wherein the peptide linker is no longer than 50 amino acids, such as no longer than 40 amino acids, such as no longer than 30 amino acids, such as no longer than 20 amino acids, such as no longer than 10 amino acids, such as no longer than 9 amino acids, such as no longer than 8 amino acids, such as no longer than 7 amino acids, such as no longer than 6 amino acids, such as no longer than 5 amino acids.
Clause 115. The construct of either clause 113 or 114 wherein the peptide linker is longer than 6 amino acids, such as longer than 7 amino acids, such as longer than 8 amino acids, such as longer than 9 amino acids, such as longer than 10 amino acids, such as longer than 20 amino acids, such as longer than 30 amino acids, such as longer than 40 amino acids, such as longer than 50 amino acids.
Clause 116. The construct of any one of clauses 113 to 115 wherein the peptide linker comprises or consists of glycine and serine residues, such as comprising or consisting of a G₄S linker (SEQ ID NO: 3).
Clause 117. The construct of any one of clauses 1 to 116 wherein one or all of the LTBR binding agents specifically bind to LTBR.
Clause 118. The construct of clause 117 wherein one or all of the LTBR binding agents specifically bind to LTBR in that any other entity is bound to with a K_{D} of 10⁻⁷ M or more, such as 10⁻⁶ M or more.
Clause 119. The construct of any one of clauses 1 to 118 wherein one or all of the LTBR binding agents specifically bind to the extracellular domain of LTBR.
Clause 120. The construct of any one of clauses 1 to 119, wherein the LTBR is on the surface of stromal cells.
Clause 121. The construct of any one of clauses 1 to 120, wherein the LTBR is on the surface of cancer cells, myeloid cells and/or macrophages.
Clause 122. The construct of any one of any one of clauses 1 to 121 wherein one or all of the LTBR binding agents is an LTBR agonist.
Clause 123. The construct of any one of clause 122 wherein the LTBR agonist is an agonist of NFKB pathways.
Clause 124. The construct of any one of clause 123 wherein the LTBR agonist (a) induces clustering of LTBR and/or (b) activates the classical NFKB pathway (such as leading to expression of adhesion molecules such as ICAMs, such as ICAM-1, VCAM-1 and/or MAdCAM-1) and/or activates the alternative NFKB pathway (such as leading to expression of chemokines, such as CCL5, CCL19, CCL21 and/or CXCL13).
Clause 125. The construct of any one of clauses 1 to 124 wherein the LTBR is human, Macaca fascicularis or mouse LTBR.
Clause 126. The construct of clause 125 wherein the LTBR is human LTBR.
Clause 127. The construct of any one of clauses 1 to 126 wherein one or all of the FAP binding agents specifically bind to FAP.
Clause 128. The construct of clause 127 wherein one or all of the FAP binding agents specifically bind to FAP in that any other entity is bound to with a K_{D} of 10⁻⁷ M or more, such as 10⁻⁶ M or more.
Clause 129. The construct of any one of clauses 1 to 128, wherein the FAP is on the surface of stromal cells.
Clause 130. The construct of any one of clauses 1 to 129, wherein the FAP is on the surface of cancer cells.
Clause 131. The construct of any one of clauses 1 to 130 wherein one or all of the FAP binding agents specifically bind to the extracellular domain of FAP.
Clause 132. The construct of any one of clauses 1 to 131 wherein the FAP is human, Macaca fascicularis or mouse FAP.
Clause 133. The construct of clause 132 wherein the FAP is human FAP.
Clause 134. The construct of any one of clauses 1 to 133, wherein one or all of the LTBR binding agents are directly linked to one or all of the FAP binding agents.
Clause 135. The construct of any one of clauses 1 to 133, wherein one or all of the LTBR binding agents are indirectly linked to one or all of the FAP binding agents.
Clause 136. The construct of clause 135, wherein the indirect linkage is via a linker.
Clause 137. The construct of any one of clauses 1 to 136 wherein one or all of the LTBR binding agents are capable of binding LTBR on a first cell and one or all of the FAP binding agents are capable of binding FAP on a second cell simultaneously.
Clause 138. The construct of any one of clauses 1 to 137 wherein one or all of the LTBR binding agents are capable of binding LTBR on a cell and one or all of the FAP binding agents are capable of binding FAP on the same cell simultaneously.
Clause 139. The construct of any one of clauses 1 to 138 wherein the construct is multivalent for LTBR and binds to LTBR with an avidity of 1.0E-08 M or less, such as 9.0E-09 M or less, such as 7.0E-09 M or less, such as 6.7E-09 M or less, such as 5.0E-09 M or less, such as 3.0E-09 M or less.
Clause 140. The construct of clause 139 wherein the construct binds to LTBR with an avidity of 2.0E-09 M or less, such as 1.0E-09 M or less.
Clause 141. The construct of any one of clauses 1 to 140 wherein the construct is multivalent for FAP and binds to FAP with an avidity of 9.0E-10 M or less, such as 8.0E-10, such as 7.2E-10, such as 5.0E-10 M or less, such as 1.0E-10 M or less, such as 9.0E-11 M or less, such as 7.0E-11 M or less.
Clause 142. The construct of clause 141 wherein the construct binds to FAP with an avidity of 5.0E-11 M or less, such as 1.0E-11 M or less.
Clause 143. The construct of any one of clauses 1 to 142 wherein one or all of the LTBR binding agents bind to LTBR with an affinity (K_{D}) of 20 nM or less, such as 15 nM or less, such as 10 nM or less, such as 9 nM or less, such as 8 nM or less, such as 7 nM or less, such as 6 nM or less, such as 5 nM or less, such as 4 nM or less, such as 3 nM or less, such as 2 nM or less, such as 1 nM or less.
Clause 144. The construct of any one of clauses 1 to 143 wherein one or all of the FAP binding agents bind to FAP with an affinity (K_{D}) of 400 pM or less, such as 300 pM or less, such as 200 pM or less, such as 150 pM or less, such as 130 pM or less, such as 110 pM or less, such as 100 pM or less, such as 50 pM or less.
Clause 145. The construct of any one of clauses 1 to 142 wherein one or all of the LTBR binding agents bind to LTBR with an affinity (K_{D}) as recited in or less.
Clause 146. The construct of any one of clauses 1 to 142 or 145 wherein one or all of the FAP binding agents bind to FAP with an affinity (K_{D}) as recited in or less.
Clause 147. The construct of any one of clauses 139 to 146 wherein the LTBR binding affinity or avidity is established as set out in Example 2 or Example 4, and/or the FAP binding affinity is established as set out in Example 3 or Example 4.
Clause 148. The construct of any one of clauses 1 to 147 wherein one or all of the LTBR binding agents have an EC50 of 9.0E-09 M or less, such as 8.0E-09 M or less, such as 7.0E-09 M or less, such as 6.0E-09 M or less, such as 5.0E-09 M or less, such as 4.0E-09 M or less, such as 3.0E-09 M or less, such as 2.0E-09 M or less, such as 1.0E-09 M or less, such as 9.0E-10 M or less, such as 8.0E-10 M or less, such as 7.0E-10 M or less, such as 6.0E-10 M or less, such as 5.0E-10 M or less, such as 4.0E-10 M or less, such as 3.0E-10 M or less, such as 2.0E-10 M or less, such as 1.0E-10 M or less, such as 9.0E-11 M or less, such as 8.0E-11 M or less, such as 7.0E-11 M or less, such as 6.0E-11 M or less, such as 5.0E-11 M or less, such as 4.0E-11 M or less, such as 3.0E-11 M or less, such as 2.0E-11 M or less, such as 1.0E-11 M or less, such as 9.0E-12 M or less, such as 8.0E-12 M or less, such as 7.0E-12 M or less, such as 6.0E-12 M or less, such as 5.0E-12 M or less, such as 4.0E-12 M or less, such as 3.0E-12 M or less, such as 2.0E-12 M or less, such as 1.0E-12 M or less.
Clause 149. The construct of any one of clauses 1 to 148 wherein one or all of the FAP binding agents have an EC50 of 9.0E-09 M or less, such as 8.0E-09 M or less, such as 7.0E-09 M or less, such as 6.0E-09 M or less, such as 5.0E-09 M or less, such as 4.0E-09 M or less, such as 3.0E-09 M or less, such as 2.0E-09 M or less, such as 1.0E-09 M or less, such as 9.0E-10 M or less, such as 8.0E-10 M or less, such as 7.0E-10 M or less, such as 6.0E-10 M or less, such as 5.0E-10 M or less, such as 4.0E-10 M or less, such as 3.0E-10 M or less, such as 2.0E-10 M or less, such as 1.0E-10 M or less, such as 9.0E-11 M or less, such as 8.0E-11 M or less, such as 7.0E-11 M or less, such as 6.0E-11 M or less, such as 5.0E-11 M or less, such as 4.0E-11 M or less, such as 3.0E-11 M or less, such as 2.0E-11 M or less, such as 1.0E-11 M or less, such as 9.0E-12 M or less, such as 8.0E-12 M or less, such as 7.0E-12 M or less, such as 6.0E-12 M or less, such as 5.0E-12 M or less, such as 4.0E-12 M or less, such as 3.0E-12 M or less, such as 2.0E-12 M or less, such as 1.0E-12 M or less.
Clause 150. The construct of any one of clauses 1 to 147 wherein one or all of the LTBR binding agents have an EC50 as recited for any of the clones specified in the tables of the Examples or less.
Clause 151. The construct of any one of clauses 1 to 147 or 150 wherein one or all of the FAP binding agents have an EC50 as recited for any of the clones specified in the tables of the Examples or less.
Clause 152. The construct of any one of clauses 148 to 151 wherein the EC50 is established in an assay as set out in the Examples.
Clause 153. The construct of any one of clauses 148 to 152 wherein the EC50 is established in respect of binding to a HepG2 cell line.
Clause 154. The construct of clause 153 wherein the EC50 is established in a HepG2 reporter assay.
Clause 155. The construct of any one of clauses 148 to 152 wherein the EC50 is established in a HepG2 NF-KB activation-induced luciferase activity.
Clause 156. The construct of any one of clauses 148 to 152 wherein the EC50 is established in respect of upregulation of CCL19 on HCC1187 cells.
Clause 157. The construct of any one of clauses 148 to 152 wherein the EC50 is established in respect of binding to LTBR positive A549 cell line.
Clause 158. The construct of any one of clauses 148 to 152 wherein the EC50 is established in respect of binding to the A375 tumour cell line.
Clause 159. The construct of any one of clauses 148 to 152 wherein the EC50 is established in respect of binding to the FAP-HEK293 cell line.
Clause 160. The construct of any one of clauses 148 to 152 wherein the EC50 is established in respect of binding to an LTBR and FAP positive cell line (e.g. A549 or A375, or CHO-FAP or Hek-FAP).
Clause 161. The construct of any one of clauses 148 to 152 wherein the EC50 is established in respect of ICAM1 upregulation in a CAFs monoculture assay.
Clause 162. The construct of clause 161 wherein the EC50 is established in respect of ICAM1 induction in PMLF.
Clause 163. The construct of any one of clauses 148 to 152 wherein the EC50 is established in a HCC1187:RPMI-7951 co-culture assay.
Clause 164. The construct of any one of clauses 148 to 152 wherein the EC50 is established in a chemokine induction assay.
Clause 165. The construct of clause 164 wherein the chemokine induction is measured with an ELISA.
Clause 166. The construct of either clause 164 or 165 wherein the chemokine is selected from CCL2, CCL5 or CCL19.
Clause 167. The construct of clause 166 wherein the chemokine is CCL19 on HCC1187 cells in the presence or absence of CAFs.
Clause 168. The construct of any one of clauses 164 to 167 wherein the chemokine induction is measured in RPMI-7951.
Clause 169. The construct of any one of clauses 164 to 167 wherein the chemokine induction is measured in 4T1 monoculture or 4T1:CHO-FAP co-culture.
Clause 170. The construct of any one of clauses 1 to 169 wherein the construct induces clustering of LTBR.
Clause 171. The construct of any one of clauses 1 to 170 wherein the construct is an antibody comprising:
(i) a heterodimer of two different heavy chains (HC1 and HC2) and two identical light chains (LC) wherein:
   (a) the HC1 polypeptide sequence comprises or consists of SEQ ID NO: 175, the HC2 polypeptide sequence comprises or consists of SEQ ID NO: 176 and the LC polypeptide sequence comprises or consists of SEQ ID NO: 177;
   (b) the HC1 polypeptide sequence comprises or consists of SEQ ID NO: 178, the HC2 polypeptide sequence comprises or consists of SEQ ID NO: 179 and the LC polypeptide sequence comprises or consists of SEQ ID NO: 180;
   (c) the HC1 polypeptide sequence comprises or consists of SEQ ID NO: 181, the HC2 polypeptide sequence comprises or consists of SEQ ID NO: 182 and the LC polypeptide sequence comprises or consists of SEQ ID NO: 183;
   (d) the HC1 polypeptide sequence comprises or consists of SEQ ID NO: 184, the HC2 polypeptide sequence comprises or consists of SEQ ID NO: 185 and the LC polypeptide sequence comprises or consists of SEQ ID NO: 186;
   (e) the HC1 polypeptide sequence comprises or consists of SEQ ID NO: 187, the HC2 polypeptide sequence comprises or consists of SEQ ID NO: 188 and the LC polypeptide sequence comprises or consists of SEQ ID NO: 189;
   (f) the HC1 polypeptide sequence comprises or consists of SEQ ID NO: 190, the HC2 polypeptide sequence comprises or consists of SEQ ID NO: 191 and the LC polypeptide sequence comprises or consists of SEQ ID NO: 192;
   (g) the HC1 polypeptide sequence comprises or consists of SEQ ID NO: 193, the HC2 polypeptide sequence comprises or consists of SEQ ID NO: 194 and the LC polypeptide sequence comprises or consists of SEQ ID NO: 195; or
   (h) the HC1 polypeptide sequence comprises or consists of SEQ ID NO: 200, the HC2 polypeptide sequence comprises or consists of SEQ ID NO: 201 and the LC polypeptide sequence comprises or consists of SEQ ID NO: 202 or
(ii) a homodimer of two identical heavy chains (HC) and two identical light chains (LC) wherein:
   (a) the HC polypeptide sequence comprises or consists of SEQ ID NO: 196 and the LC polypeptide sequence comprises or consists of SEQ ID NO: 197 or
   (b) the HC polypeptide sequence comprises or consists of SEQ ID NO: 198 and the LC polypeptide sequence comprises or consists of SEQ ID NO: 199.
Clause 172. A method of activating LTBR in a tumour in a subject, the method comprising administering to the subject the construct of any one of clauses 1 to 171.
Clause 173. The construct of any one of clauses 1 to 172 wherein the construct is for use as a medicament.
Clause 174. A construct of clause 173 for use in the treatment of cancer.
Clause 175. A method of treating cancer comprising administering to a subject in need thereof the construct of any one of clauses 1 to 171.
Clause 176. Use of a construct of any one of clauses 1 to 171 in the manufacture of a medicament for the treatment of cancer.
Clause 177. The construct for use, method or use of any one of clauses 174 to 176 wherein the cancer is a tumour.
Clause 178. The construct for use, method or use of any one of clauses 174 to 176 wherein the cancer is a non-haematological cancer.
Clause 179. A set of one or more polynucleotides encoding the construct of any one of clauses 1 to 171.
Clause 180. A set of one or more expression vectors collectively comprising the polynucleotides of clause 179.
Clause 181. A cell comprising the expression vectors or polynucleotides of either clause 179 or 180.
Clause 182. A method of producing a construct of any one of clauses 1 to 171, the method comprising culturing the cell of clause 181 under suitable conditions such that the polynucleotide is expressed, and the construct is produced.
Clause 183. A composition comprising a construct of any one of clauses 1 to 171 and an anti-CTLA-4 antibody.
Clause 184. A composition comprising a construct of any one of clauses 1 to 171 and an anti-PD-L1 antibody.
Clause 185. A composition comprising a construct of any one of clauses 1 to 171 and an anti-PD-1 antibody.
Clause 186. The composition of any one of clauses 183 to 185 for use in the treatment of cancer (e.g. a tumour).
Clause 187. A construct of any one of clauses 1 to 171 for use in the treatment of cancer (e.g. a tumour) in combination with radiotherapy.
Clause 188. A construct for use of clause 187 wherein the radiotherapy is hypo-fractionated radiotherapy.
Clause 189. A construct for use or method of any one of clauses 173 to 178 for use in the treatment of cancer in combination with chemotherapy, ADCs or a cancer vaccine.
Clause 190. A construct of any one of clauses 1 to 171 for use in the treatment of cancer in combination with an anti-CTLA-4 antibody, optionally in combination with radiotherapy.
Clause 191. An anti-CTLA-4 antibody for use in the treatment of cancer in combination with a construct of any one of clauses 1 to 171, optionally in combination with radiotherapy.
Clause 192. The construct or anti-CTLA-4 antibody for use of clause 190 or 191 wherein the anti-CTLA-4 antibody is clone 9D9.
Clause 193. A construct of any one of clauses 1 to 171 for use in the treatment of cancer in combination with an anti-PD-L1 antibody, optionally in combination with radiotherapy.
Clause 194. An anti-PD-L1 antibody for use in the treatment of cancer in combination with a construct of any one of clauses 1 to 171, optionally in combination with radiotherapy.
Clause 195. A construct of any one of clauses 1 to 171 for use in the treatment of cancer in combination with an anti-PD-1 antibody, optionally in combination with radiotherapy.
Clause 196. An anti-PD-1 antibody for use in the treatment of cancer in combination with a construct of any one of clauses 1 to 171, optionally in combination with radiotherapy.
Clause 197. The construct for use or the anti-CTLA-4 antibody for use, the anti-PD-L1 antibody for use or the anti-PD-1 antibody for use of any one of clauses 187 to 196 wherein the anti-CTLA-4 antibody, the anti-PD-L1 antibody or the anti-PD-1 antibody are each administered sequentially, separately or simultaneously with the construct.
Clause 198. The construct for use or the anti-CTLA-4 antibody for use, the anti-PD-L1 antibody for use or the anti-PD-1 antibody for use of any one of clauses 189 to 196 wherein the construct, anti-CTLA-4 antibody, the anti-PD-L1 antibody, the anti-PD-1 antibody, the radiotherapy, the ADCs or the chemotherapy are each administered sequentially, separately or simultaneously.
Clause 199. The construct of any one of clauses 1 to 198 comprising, essentially consisting of or consisting of a format as described in for clone 4.1, 4.2, 4.3, 4.4, 4.5, 4.6, 4.7, 4.8, 4.9, 4.10, 4.11 or 4.12.
Clause 200. The construct of any one of clauses 1 to 198 comprising, essentially consisting of or consisting of a format as depicted in FIGS. 7a-7d.
Clause 201. The construct of any one of clauses 1 to 200 comprising or consisting of the polypeptide sequence(s) of clone 4.1, 4.2, 4.3, 4.4, 4.5, 4.6, 4.7, 4.8, 4.9, 4.10, 4.11 or 4.12 (such as clone 4.1, 4.2, 4.3, 4.4, 4.5, 4.6, 4.7, 4.8, 4.9 or 4.10).
Clause 202. The construct of any one of clauses 1 to 201 comprising, consisting essentially of or consisting of a bispecific, FAP-monovalent, LTBR-monovalent antibody comprising a heterodimer of two polypeptide chains, the first chain comprising or consisting of from N to C terminus a Fab region, a hinge region, a CH2 domain and a CH3 domain; and the second chain comprising or consisting of from N to C terminus a VHH, a hinge region and a CH2 domain and a CH3 domain, wherein the hinge regions are linked via a disulphide bond, the CH2 and CH3 domains are associated to form an Fc region, the Fab region comprises a paired VH and VL at its N-terminus which form a FAP binding site and the VHH forms an LTBR binding site (e.g. essentially as set out in FIG. 7a).
Clause 203. The construct of any one of clauses 1 to 201 comprising, essentially consisting or consisting of a first heavy chain, a light chain and a second heavy chain, wherein the first heavy chain comprises from N to C terminus a VH, a CH1, a CH2 and a CH3, the second heavy chain comprises from N to C terminus a VHH, a CH2 and a CH3, the light chain comprises from N to C terminus a VL and a CL and associates with the VH and CH1 of the first heavy chain to form a Fab region and the CH2 and CH3 domains of the first and second heavy chains associate to form an Fc region, wherein the VH and VL bind to FAP and the VHH binds to LTBR (e.g. essentially as set out in FIG. 7a).
Clause 204. The construct of any one of clauses 1 to 201 comprising, consisting essentially of or consisting of a bispecific, FAP-bivalent, LTBR-monovalent antibody comprising a heterodimer of two polypeptide chains, the first chain comprising or consisting of from N to C terminus a Fab region, a hinge region, a CH2 domain, a CH3 domain and a VHH; and the second chain comprising or consisting of from N to C terminus a Fab region, a hinge region, a CH2 domain and a CH3 domain, wherein the hinge regions are linked via a disulphide bond, the CH2 and CH3 domains are associated to form an Fc region, the Fab regions each comprise a paired VH and VL at their N-terminus which each form a FAP binding site and the VHH forms an LTBR binding site (e.g. essentially as set out in FIG. 7b).
Clause 205. The construct of any one of clauses 1 to 201 comprising, consisting essentially of or consisting of a first heavy chain, a first light chain, a second heavy chain and a second light chain wherein the first heavy chain comprises from N to C terminus a VH, a CH1, a CH2, a CH3 and a VHH, the second heavy chain comprises from N to C terminus a VH, a CH1, a CH2 and a CH3, the first light chain comprises from N to C terminus a VL and a CL and associates with the VH and CH1 of the first heavy chain to form a Fab region, the second light chain comprises from N to C terminus a VL and a CL and associates with the VH and CH1 of the second heavy chain to form a Fab region and the CH2 and CH3 domains of the first and second heavy chains associate to form an Fc region, wherein the VH and VL of each Fab region bind to FAP and the VHH binds to LTBR (e.g. essentially as set out in FIG. 7b).
Clause 206. The construct of any one of clauses 1 to 201 comprising, consisting essentially of or consisting of a bispecific, FAP-bivalent, LTBR-bivalent antibody comprising a homodimer of two polypeptide chains, the first chain comprising or consisting of from N to C terminus a Fab region, a hinge region, a CH2 domain, a CH3 domain and a VHH; and the second chain comprising or consisting of from N to C terminus a Fab region, a hinge region, a CH2 domain, a CH3 domain and a VHH wherein the hinge regions are linked via a disulphide bond, the CH2 and CH3 domains are associated to form an Fc region, the Fab regions each comprise a paired VH and VL at their N-terminus which each form a FAP binding site and the VHHs each form an LTBR binding site (e.g. essentially as set out in FIG. 7c).
Clause 207. The construct of any one of clauses 1 to 201 comprising, consisting essentially of or consisting of a first heavy chain, a first light chain, a second heavy chain and a second light chain wherein the first heavy chain comprises from N to C terminus a VH, a CH1, a CH2, a CH3 and a VHH, the second heavy chain comprises from N to C terminus a VH, a CH1, a CH2, a CH3 and a VHH, the first light chain comprises from N to C terminus a VL and a CL and associates with the VH and CH1 of the first heavy chain to form a Fab region, the second light chain comprises from N to C terminus a VL and a CL and associates with the VH and CH1 of the second heavy chain to form a Fab region and the CH2 and CH3 domains of the first and second heavy chains associate to form an Fc region, wherein the VH and VL of each Fab region bind to FAP and the VHHs bind to LTBR (e.g. essentially as set out in FIG. 7c).
Clause 208. The construct of any one of clauses 1 to 201 comprising, consisting essentially of or consisting of a bispecific, FAP-bivalent, LTBR-bivalent antibody comprising a homodimer of two polypeptide chains, the first chain comprising or consisting of from N to C terminus a Fab region, a hinge region, a CH2 domain, a CH3 domain and an scFv; and the second chain comprising or consisting of from N to C terminus a Fab region, a hinge region, a CH2 domain, a CH3 domain and an scFv wherein the hinge regions are linked via a disulphide bond, the CH2 and CH3 domains are associated to form an Fc region, the Fab regions each comprise a paired VH and VL at their N-terminus which each form a FAP binding site and the scFvs each form an LTBR binding site (e.g. essentially as set out in FIG. 7d).
Clause 209. The construct of any one of clauses 1 to 201 comprising, consisting essentially of or consisting of a first heavy chain, a first light chain, a second heavy chain and a second light chain wherein the first heavy chain comprises from N to C terminus a VH, a CH1, a CH2, a CH3 and a paired VH and VL, the second heavy chain comprises from N to C terminus a VH, a CH1, a CH2, a CH3 and a paired VH and VL, the first light chain comprises from N to C terminus a VL and a CL and associates with the VH and CH1 of the first heavy chain to form a Fab region, the second light chain comprises from N to C terminus a VL and a CL and associates with the VH and CH1 of the second heavy chain to form a Fab region and the CH2 and CH3 domains of the first and second heavy chains associate to form an Fc region, wherein the VH and VL of each Fab region bind to FAP and the paired VH and VLs bind to LTBR (e.g. essentially as set out in FIG. 7d).
Clause 210. The construct of any one of clauses 1 to 201 comprising, consisting essentially of or consisting of a heterodimer bispecific, FAP-monovalent, LTBR-monovalent antibody having an anti-LTBR VHH and an anti-FAP Fab wherein the anti-LTBR VHH comprises or consists of the heavy chain variable region polypeptide sequence of clone 1.6 and the anti-FAP Fab comprises the heavy chain variable region and light chain variable region polypeptide sequences of clone 3.5 (e.g. clone 4.1).
Clause 211. The construct of any one of clauses 1 to 201 comprising, consisting essentially of or consisting of a heterodimer bispecific, FAP-monovalent, LTBR-monovalent antibody having an anti-LTBR VHH and an anti-FAP Fab wherein the anti-LTBR VHH comprises or consists of the heavy chain variable region polypeptide sequence of clone 1.7 and the anti-FAP Fab comprises the heavy chain variable region and light chain variable region polypeptide sequences of clone 3.5 (e.g. clone 4.2).
Clause 212. The construct of any one of clauses 1 to 201 comprising, consisting essentially of or consisting of a heterodimer bispecific, FAP-monovalent, LTBR-monovalent antibody having an anti-LTBR VHH and an anti-FAP Fab wherein the anti-LTBR VHH comprises or consists of the heavy chain variable region polypeptide sequence of clone 1.6 and the anti-FAP Fab comprises the heavy chain variable region and light chain variable region polypeptide sequences of clone 3.6 (e.g. clone 4.3).
Clause 213. The construct of any one of clauses 1 to 201 comprising, consisting essentially of or consisting of a heterodimer bispecific, FAP-bivalent, LTBR-monovalent antibody having an anti-LTBR VHH and two anti-FAP Fabs wherein the anti-LTBR VHH comprises or consists of the heavy chain variable region polypeptide sequence of clone 1.6 and the anti-FAP Fabs comprise the heavy chain variable region and light chain variable region polypeptide sequences of clone 3.6 and wherein the LTBR VHH is fused to the Fc, such as a CH3 domain of the Fc (e.g. clone 4.4).
Clause 214. The construct of any one of clauses 1 to 201 comprising, consisting essentially of or consisting of a heterodimer bispecific, FAP-bivalent, LTBR-monovalent antibody having an anti-LTBR VHH and two anti-FAP Fabs wherein the anti-LTBR VHH comprises or consists of the heavy chain variable region polypeptide sequence of clone 1.6 and the anti-FAP Fabs comprise the heavy chain variable region and light chain variable region polypeptide sequences of clone 3.5 and wherein the LTBR VHH is fused to the Fc, such as a CH3 domain of the Fc (e.g. clone 4.5).
Clause 215. The construct of any one of clauses 1 to 201 comprising, consisting essentially of or consisting of a heterodimer bispecific, FAP-bivalent, LTBR-monovalent antibody having an anti-LTBR VHH and two anti-FAP Fabs wherein the anti-LTBR VHH comprises or consists of the polypeptide sequence of heavy chain variable region clone 1.7 and the anti-FAP Fabs comprise of the heavy chain variable region and light chain variable region polypeptide sequences of clone 3.6 and wherein the LTBR VHH is fused to the Fc, such as a CH3 domain of the Fc (e.g. clone 4.6).
Clause 216. The construct of any one of clauses 1 to 201 comprising, consisting essentially of or consisting of a heterodimer bispecific, FAP-bivalent, LTBR-monovalent antibody having an anti-LTBR scFv and two anti-FAP Fabs wherein the anti-LTBR scFv comprises or consists of the heavy chain variable region and light chain variable region polypeptide sequence of clone 2.3 and the anti-FAP Fabs comprise the heavy chain variable region and light chain variable region polypeptide sequences of clone 3.7 and wherein the LTBR scFv is fused to the Fc, such as a CH3 domain of the Fc (e.g. clone 4.7).
Clause 217. The construct of any one of clauses 1 to 201 comprising, consisting essentially of or consisting of a homodimer FAP-bivalent, LTBR-bivalent, bispecific, antibody having two anti-LTBR VHHs and two anti-FAP Fabs wherein the anti-LTBR VHHs comprise or consist of the heavy chain variable region polypeptide sequence of clone 1.7 and the anti-FAP Fabs comprise the heavy chain variable region and light chain variable region polypeptide sequences of clone 3.5 and wherein the LTBR VHHs are fused to the Fc, such as the CH3 domains of the Fc (e.g. clone 4.8).
Clause 218. The construct of any one of clauses 1 to 201 comprising, consisting essentially of or consisting of a homodimer FAP-bivalent, LTBR-bivalent, bispecific, antibody having two anti-LTBR VHHs and two anti-FAP Fabs wherein the anti-LTBR VHHs comprise or consist of the heavy chain variable region polypeptide sequence of clone 1.6 and the anti-FAP Fabs comprise the heavy chain variable region and light chain variable region polypeptide sequences of clone 3.5 and wherein the LTBR scFvs are fused to the Fc, such as the CH3 domains of the Fc (e.g. clone 4.9).
Clause 219. The construct of any one of clauses 1 to 201 comprising, consisting essentially of or consisting of a heterodimer bispecific, FAP-monovalent, LTBR-monovalent antibody having an anti-LTBR VHH and an anti-FAP Fab wherein the anti-LTBR VHH comprises or consists of the heavy chain variable region polypeptide sequence of clone 1.6 and the anti-FAP Fab comprises the heavy chain variable region and light chain variable region polypeptide sequences of clone 3.4 (e.g. clone 4.10).
Clause 220. An antibody which binds to the epitopes of the polypeptide or construct of any one of clauses 1 to 201.
Clause 221. An antibody which competes for binding to LTBR with the construct of any one of clauses 1 to 201.
Clause 222. An antibody which competes for binding to FAP with the construct of any one of clauses 1 to 201.
Clause 223. The construct of any one of clauses 1 to 28 wherein the LTBR-binding agent comprises at least one CDR, wherein the CDR is a HCDR1, HCDR2 or HCDR3 comprised in a heavy chain variable domain of SEQ ID NOs: 23-26 or 159-162.
Clause 224. The construct of clause 223 wherein the LTBR-binding agent comprises at least such HCDR1 and HCDR2, such HCDR1 and HCDR3, or such HCDR2 and HCDR3.
Clause 225. The construct of clause 223 wherein the LTBR-binding agent comprises such HCDR1, HCDR2 and HCDR3.
Clause 226. The construct according to any one of clauses 223 to 225 wherein the HCDR1, HCDR2 and HCDR3 are delineated according to the Kabat-, Chothia-, Martin-, IMGT- or AbM-method.
Clause 227. The construct according to any one of clauses 223 to 225 wherein the HCDR1 is chosen from SEQ ID NOs: 57, 60, 63, 66, 163, 166, 169 or 172 as defined by the Kabat-method; the HCDR2 is chosen from SEQ ID NOs: 58, 61, 64, 67, 164, 167, 170, 173, 205 or 206 as defined by the Kabat-method; and/or the HCDR3 is chosen from SEQ ID NOs: 59, 62, 65, 68, 165, 168, 171, 174, 207 or 208 as defined by the Kabat-method.
Clause 228. The construct, construct for use, antibody or method of any one of clauses 189 to 227 wherein the construct is for use in the treatment of cancer in combination with a cancer vaccine.
Clause 229. The construct for use or method of clause 228 wherein the construct and cancer vaccine are for use in combination with an adjuvant.
Clause 230. The construct for use or method of either clause 228 or 229 wherein the construct, cancer vaccine and/or adjuvant are administered sequentially, separately or simultaneously.
Clause 231. The construct for use or method of any one of clauses 228 to 230 wherein the cancer vaccine is an antigenic peptide or a polynucleotide encoding an antigen (such as RNA (e.g. mRNA) or DNA), optionally comprised in a pharmaceutically acceptable excipient.
Clause 232. The construct for use or method of any one of clauses 228 to 231 wherein the cancer vaccine is a tumour associated antigen or an encoded tumour associated antigen.
Clause 233. The construct for use or method of clause 232 wherein the cancer vaccine is a tumour associated antigen comprising or consisting of the polypeptide sequence KCLQDNNWDYTRYAQAFTLLKAKG (SEQ ID NO: 209).
Clause 234. The construct for use or method of any one of clauses 229 to 233 wherein the adjuvant is a TLR agonist.
Clause 235. The construct for use or method of clause 234 wherein the adjuvant is a TLR-9 agonist.
Clause 236. The construct for use or method of clause 235 wherein the adjuvant is a CpG oligonucleotide.

A series of clauses setting out further embodiments of the invention are as follows. These embodiments generally relate to (i) anti-LTBR binding polypeptides or (ii) anti-FAP binding polypeptides.
Clause 1 B. A binding polypeptide comprising or consisting of a heavy chain variable domain which binds to LTBR wherein the heavy chain variable domain comprises or consists of three heavy chain complementarity determining regions (HCDR1-HCDR3) and four framework regions (FR1-FR4), wherein HCDR1 comprises or consists of a polypeptide sequence sharing 70% or greater, 75% or greater, 80% or greater, 85% or greater, 90% or greater or 95% or greater sequence identity with SEQ ID NO: 57 or wherein HCDR1 comprises or consists of a polypeptide sequence having 3, 2 or 1 amino acid sequence difference with SEQ ID NO: 57, HCDR2 comprises or consists of a polypeptide sequence sharing 70% or greater, 75% or greater, 80% or greater, 85% or greater, 90% or greater or 95% or greater sequence identity with SEQ ID NO: 58 or 205, or wherein HCDR2 comprises or consists of a polypeptide sequence having 3, 2 or 1 amino acid sequence difference with SEQ ID NO: 58 or 205, and HCDR3 comprises or consists of a polypeptide sequence sharing 70% or greater, 75% or greater, 80% or greater, 85% or greater, 90% or greater or 95% or greater sequence identity with SEQ ID NO: 59 or wherein HCDR3 comprises or consists of a polypeptide sequence having 3, 2 or 1 amino acid sequence difference with SEQ ID NO: 59.
Clause 2B. The polypeptide of clause 1 B wherein HCDR1 comprises or consists of a polypeptide sequence of SEQ ID NO: 57, HCDR2 comprises or consists of a polypeptide sequence of SEQ ID NO: 58 or 205, and HCDR3 comprises or consists of a polypeptide sequence of SEQ ID NO: 59.
Clause 3B. A binding polypeptide comprising or consisting of a heavy chain variable domain which binds to LTBR wherein the heavy chain variable domain comprises or consists of three heavy chain complementarity determining regions (HCDR1-HCDR3) and four framework regions (FR1-FR4), wherein HCDR1 comprises or consists of a polypeptide sequence sharing 70% or greater, 75% or greater, 80% or greater, 85% or greater, 90% or greater or 95% or greater sequence identity with SEQ ID NO: 60 or wherein HCDR1 comprises or consists of a polypeptide sequence having 3, 2 or 1 amino acid sequence difference with SEQ ID NO: 60, HCDR2 comprises or consists of a polypeptide sequence sharing 70% or greater, 75% or greater, 80% or greater, 85% or greater, 90% or greater or 95% or greater sequence identity with SEQ ID NO: 61 or wherein HCDR2 comprises or consists of a polypeptide sequence having 3, 2 or 1 amino acid sequence difference with SEQ ID NO: 61 and HCDR3 comprises or consists of a polypeptide sequence sharing 70% or greater, 75% or greater, 80% or greater, 85% or greater, 90% or greater or 95% or greater sequence identity with SEQ ID NO: 62 or wherein HCDR3 comprises or consists of a polypeptide sequence having 3, 2 or 1 amino acid sequence difference with SEQ ID NO: 62.
Clause 4B. The polypeptide of clause 3B wherein HCDR1 comprises or consists of a polypeptide sequence of SEQ ID NO: 60, HCDR2 comprises or consists of a polypeptide sequence of SEQ ID NO: 61 and HCDR3 comprises or consists of a polypeptide sequence of SEQ ID NO: 62.
Clause 5B. A binding polypeptide comprising or consisting of a heavy chain variable domain which binds to LTBR wherein the heavy chain variable domain comprises or consists of three heavy chain complementarity determining regions (HCDR1-HCDR3) and four framework regions (FR1-FR4), wherein HCDR1 comprises or consists of a polypeptide sequence sharing 70% or greater, 75% or greater, 80% or greater, 85% or greater, 90% or greater or 95% or greater sequence identity with SEQ ID NO: 63 or wherein HCDR1 comprises or consists of a polypeptide sequence having 3, 2 or 1 amino acid sequence difference with SEQ ID NO: 63, HCDR2 comprises or consists of a polypeptide sequence sharing 70% or greater, 75% or greater, 80% or greater, 85% or greater, 90% or greater or 95% or greater sequence identity with SEQ ID NO: 64 or wherein HCDR2 comprises or consists of a polypeptide sequence having 3, 2 or 1 amino acid sequence difference with SEQ ID NO: 64 and HCDR3 comprises or consists of a polypeptide sequence sharing 70% or greater, 75% or greater, 80% or greater, 85% or greater, 90% or greater or 95% or greater sequence identity with SEQ ID NO: 65 or 207 or wherein HCDR3 comprises or consists of a polypeptide sequence having 3, 2 or 1 amino acid sequence difference with SEQ ID NO: 65 or 207.
Clause 6B. The polypeptide of clause 5B wherein HCDR1 comprises or consists of a polypeptide sequence of SEQ ID NO: 63, HCDR2 comprises or consists of a polypeptide sequence of SEQ ID NO: 64 and HCDR3 comprises or consists of a polypeptide sequence of SEQ ID NO: 65 or 207.
Clause 7B. A binding polypeptide comprising or consisting of a heavy chain variable domain which binds to LTBR wherein the heavy chain variable domain comprises or consists of three heavy chain complementarity determining regions (HCDR1-HCDR3) and four framework regions (FR1-FR4), wherein HCDR1 comprises or consists of a polypeptide sequence sharing 70% or greater, 75% or greater, 80% or greater, 85% or greater, 90% or greater or 95% or greater sequence identity with SEQ ID NO: 66 or wherein HCDR1 comprises or consists of a polypeptide sequence having 3, 2 or 1 amino acid sequence difference with SEQ ID NO: 66, HCDR2 comprises or consists of a polypeptide sequence sharing 70% or greater, 75% or greater, 80% or greater, 85% or greater, 90% or greater or 95% or greater sequence identity with SEQ ID NO: 67 or 206 or wherein HCDR2 comprises or consists of a polypeptide sequence having 3, 2 or 1 amino acid sequence difference with SEQ ID NO: 67 or 206, and HCDR3 comprises or consists of a polypeptide sequence sharing 70% or greater, 75% or greater, 80% or greater, 85% or greater, 90% or greater or 95% or greater sequence identity with SEQ ID NO: 68 or 171, or wherein HCDR3 comprises or consists of a polypeptide sequence having 3, 2 or 1 amino acid sequence difference with SEQ ID NO: 68 or 208.
Clause 8B. The polypeptide of clause 7B wherein HCDR1 comprises or consists of a polypeptide sequence of SEQ ID NO: 66, HCDR2 comprises or consists of a polypeptide sequence of SEQ ID NO: 67 or 206 and HCDR3 comprises or consists of a polypeptide sequence of SEQ ID NO: 68 or 208.
Clause 9B. A binding polypeptide comprising or consisting of a heavy chain variable domain which binds to LTBR wherein the heavy chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 23.
Clause 10B. The polypeptide of clause 9B wherein the heavy chain variable domain which binds to LTBR comprises or consists of a polypeptide sequence of SEQ ID NO: 23.
Clause 11B. A binding polypeptide comprising or consisting of a heavy chain variable domain which binds to LTBR wherein the heavy chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 24.
Clause 12B. The polypeptide of any one of clause 11 B wherein the heavy chain variable domain which binds to LTBR comprises or consists of a polypeptide sequence of SEQ ID NO: 24.
Clause 13B. A binding polypeptide comprising or consisting of a heavy chain variable domain which binds to LTBR wherein the heavy chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 25.
Clause 14B. The polypeptide of clause 13B wherein the heavy chain variable domain which binds to LTBR comprises or consists of a polypeptide sequence of SEQ ID NO: 25.
Clause 15B. A binding polypeptide comprising or consisting of a heavy chain variable domain which binds to LTBR wherein the heavy chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 26.
Clause 16B. The polypeptide of any one of clause 15B wherein the heavy chain variable domain which binds to LTBR comprises or consists of a polypeptide sequence of SEQ ID NO: 26.
Clause 17B. A binding polypeptide comprising or consisting of a heavy chain variable domain which binds to LTBR wherein the heavy chain variable domain comprises or consists of three heavy chain complementarity determining regions (HCDR1-HCDR3) and four framework regions (FR1-FR4), wherein HCDR1 comprises or consists of a polypeptide sequence sharing 70% or greater, 75% or greater, 80% or greater, 85% or greater, 90% or greater or 95% or greater sequence identity with SEQ ID NO: 163 or wherein HCDR1 comprises or consists of a polypeptide sequence having 3, 2 or 1 amino acid sequence difference with SEQ ID NO: 163, HCDR2 comprises or consists of a polypeptide sequence sharing 70% or greater, 75% or greater, 80% or greater, 85% or greater, 90% or greater or 95% or greater sequence identity with SEQ ID NO: 164 or wherein HCDR2 comprises or consists of a polypeptide sequence having 3, 2 or 1 amino acid sequence difference with SEQ ID NO: 164, and HCDR3 comprises or consists of a polypeptide sequence sharing 70% or greater, 75% or greater, 80% or greater, 85% or greater, 90% or greater or 95% or greater sequence identity with SEQ ID NO: 165 or 207, or wherein HCDR3 comprises or consists of a polypeptide sequence having 3, 2 or 1 amino acid sequence difference with SEQ ID NO: 165 or 207.
Clause 18B. The polypeptide of clause 1 B wherein HCDR1 comprises or consists of a polypeptide sequence of SEQ ID NO: 163, HCDR2 comprises or consists of a polypeptide sequence of SEQ ID NO: 164 and HCDR3 comprises or consists of a polypeptide sequence of SEQ ID NO: 165 or 207.
Clause 19B. A binding polypeptide comprising or consisting of a heavy chain variable domain which binds to LTBR wherein the heavy chain variable domain comprises or consists of three heavy chain complementarity determining regions (HCDR1-HCDR3) and four framework regions (FR1-FR4), wherein HCDR1 comprises or consists of a polypeptide sequence sharing 70% or greater, 75% or greater, 80% or greater, 85% or greater, 90% or greater or 95% or greater sequence identity with SEQ ID NO: 166 or wherein HCDR1 comprises or consists of a polypeptide sequence having 3, 2 or 1 amino acid sequence difference with SEQ ID NO: 166, HCDR2 comprises or consists of a polypeptide sequence sharing 70% or greater, 75% or greater, 80% or greater, 85% or greater, 90% or greater or 95% or greater sequence identity with SEQ ID NO: 167 or 205 or wherein HCDR2 comprises or consists of a polypeptide sequence having 3, 2 or 1 amino acid sequence difference with SEQ ID NO: 167 or 205, and HCDR3 comprises or consists of a polypeptide sequence sharing 70% or greater, 75% or greater, 80% or greater, 85% or greater, 90% or greater or 95% or greater sequence identity with SEQ ID NO: 168 or wherein HCDR3 comprises or consists of a polypeptide sequence having 3, 2 or 1 amino acid sequence difference with SEQ ID NO: 168.
Clause 20B. The polypeptide of clause 1 B wherein HCDR1 comprises or consists of a polypeptide sequence of SEQ ID NO: 166, HCDR2 comprises or consists of a polypeptide sequence of SEQ ID NO: 167 or 205, and HCDR3 comprises or consists of a polypeptide sequence of SEQ ID NO: 168.
Clause 21B. A binding polypeptide comprising or consisting of a heavy chain variable domain which binds to LTBR wherein the heavy chain variable domain comprises or consists of three heavy chain complementarity determining regions (HCDR1-HCDR3) and four framework regions (FR1-FR4), wherein HCDR1 comprises or consists of a polypeptide sequence sharing 70% or greater, 75% or greater, 80% or greater, 85% or greater, 90% or greater or 95% or greater sequence identity with SEQ ID NO: 169 or wherein HCDR1 comprises or consists of a polypeptide sequence having 3, 2 or 1 amino acid sequence difference with SEQ ID NO: 169, HCDR2 comprises or consists of a polypeptide sequence sharing 70% or greater, 75% or greater, 80% or greater, 85% or greater, 90% or greater or 95% or greater sequence identity with SEQ ID NO: 170 or 206, or wherein HCDR2 comprises or consists of a polypeptide sequence having 3, 2 or 1 amino acid sequence difference with SEQ ID NO: 170 or 206, and HCDR3 comprises or consists of a polypeptide sequence sharing 70% or greater, 75% or greater, 80% or greater, 85% or greater, 90% or greater or 95% or greater sequence identity with SEQ ID NO: 171 or 208 or wherein HCDR3 comprises or consists of a polypeptide sequence having 3, 2 or 1 amino acid sequence difference with SEQ ID NO: 171 or 208.
Clause 22B. The polypeptide of clause 1 B wherein HCDR1 comprises or consists of a polypeptide sequence of SEQ ID NO: 169, HCDR2 comprises or consists of a polypeptide sequence of SEQ ID NO: 170 or 206, and HCDR3 comprises or consists of a polypeptide sequence of SEQ ID NO: 171 or 208.
Clause 23B. A binding polypeptide comprising or consisting of a heavy chain variable domain which binds to LTBR wherein the heavy chain variable domain comprises or consists of three heavy chain complementarity determining regions (HCDR1-HCDR3) and four framework regions (FR1-FR4), wherein HCDR1 comprises or consists of a polypeptide sequence sharing 70% or greater, 75% or greater, 80% or greater, 85% or greater, 90% or greater or 95% or greater sequence identity with SEQ ID NO: 172 or wherein HCDR1 comprises or consists of a polypeptide sequence having 3, 2 or 1 amino acid sequence difference with SEQ ID NO: 172, HCDR2 comprises or consists of a polypeptide sequence sharing 70% or greater, 75% or greater, 80% or greater, 85% or greater, 90% or greater or 95% or greater sequence identity with SEQ ID NO: 173 or wherein HCDR2 comprises or consists of a polypeptide sequence having 3, 2 or 1 amino acid sequence difference with SEQ ID NO: 173, and HCDR3 comprises or consists of a polypeptide sequence sharing 70% or greater, 75% or greater, 80% or greater, 85% or greater, 90% or greater or 95% or greater sequence identity with SEQ ID NO: 174 or wherein HCDR3 comprises or consists of a polypeptide sequence having 3, 2 or 1 amino acid sequence difference with SEQ ID NO: 174.
Clause 24B. The polypeptide of clause 1 B wherein HCDR1 comprises or consists of a polypeptide sequence of SEQ ID NO: 172, HCDR2 comprises or consists of a polypeptide sequence of SEQ ID NO: 173 and HCDR3 comprises or consists of a polypeptide sequence of SEQ ID NO: 174.
Clause 25B. A binding polypeptide comprising or consisting of a heavy chain variable domain which binds to LTBR wherein the heavy chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 159.
Clause 26B. The polypeptide of clause 25B wherein the heavy chain variable domain which binds to LTBR comprises or consists of a polypeptide sequence of SEQ ID NO: 159.
Clause 27B. A binding polypeptide comprising or consisting of a heavy chain variable domain which binds to LTBR wherein the heavy chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 160.
Clause 28B. The polypeptide of any one of clause 27B wherein the heavy chain variable domain which binds to LTBR comprises or consists of a polypeptide sequence of SEQ ID NO: 160.
Clause 29B. A binding polypeptide comprising or consisting of a heavy chain variable domain which binds to LTBR wherein the heavy chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 161.
Clause 30B. The polypeptide of clause 29B wherein the heavy chain variable domain which binds to LTBR comprises or consists of a polypeptide sequence of SEQ ID NO: 161.
Clause 31B. A binding polypeptide comprising or consisting of a heavy chain variable domain which binds to LTBR wherein the heavy chain variable domain which binds to LTBR comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 162.
Clause 32B. The polypeptide of any one of clause 31 B wherein the heavy chain variable domain which binds to LTBR comprises or consists of a polypeptide sequence of SEQ ID NO: 162.
Clause 33B. A binding polypeptide comprising or consisting of a paired heavy chain variable domain and light chain variable domain which bind to LTBR wherein the heavy chain variable domain comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 69, HCDR2 comprises or consists of a polypeptide sequence sharing 70% or greater, 75% or greater, 80% or greater, 85% or greater, 90% or greater or 95% or greater sequence identity with SEQ ID NO: 70 and HCDR3 comprises or consists of a polypeptide sequence sharing 70% or greater, 75% or greater, 80% or greater, 85% or greater, 90% or greater or 95% or greater sequence identity with SEQ ID NO: 71 and the light chain variable domain comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 72, LCDR2 comprises or consists of a polypeptide sequence sharing 70% or greater, 75% or greater, 80% or greater, 85% or greater, 90% or greater or 95% or greater sequence identity with SEQ ID NO: 73 and LCDR3 comprises or consists of a polypeptide sequence sharing 70% or greater, 75% or greater, 80% or greater, 85% or greater, 90% or greater or 95% or greater sequence identity with SEQ ID NO: 74.
Clause 34B. The polypeptide of any one of clause 33B wherein HCDR1 comprises or consists of a polypeptide sequence of SEQ ID NO: 69, HCDR2 comprises or consists of a polypeptide sequence of SEQ ID NO: 70 and HCDR3 comprises or consists of a polypeptide sequence of SEQ ID NO: 71 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 72, LCDR2 comprises or consists of a polypeptide sequence of SEQ ID NO: 73 and LCDR3 comprises or consists of a polypeptide sequence of SEQ ID NO: 74.
Clause 35B. A binding polypeptide comprising or consisting of a paired heavy chain variable domain and light chain variable domain which bind to LTBR wherein the heavy chain variable domain comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 75, HCDR2 comprises or consists of a polypeptide sequence sharing 70% or greater, 75% or greater, 80% or greater, 85% or greater, 90% or greater or 95% or greater sequence identity with SEQ ID NO: 76 and HCDR3 comprises or consists of a polypeptide sequence sharing 70% or greater, 75% or greater, 80% or greater, 85% or greater, 90% or greater or 95% or greater sequence identity with SEQ ID NO: 77 and the light chain variable domain comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 78, LCDR2 comprises or consists of a polypeptide sequence sharing 70% or greater, 75% or greater, 80% or greater, 85% or greater, 90% or greater or 95% or greater sequence identity with SEQ ID NO: 79 and LCDR3 comprises or consists of a polypeptide sequence sharing 70% or greater, 75% or greater, 80% or greater, 85% or greater, 90% or greater or 95% or greater sequence identity with SEQ ID NO: 80.
Clause 36B. The polypeptide of any one of clause 35B wherein HCDR1 comprises or consists of a polypeptide sequence of SEQ ID NO: 75, HCDR2 comprises or consists of a polypeptide sequence of SEQ ID NO: 76 and HCDR3 comprises or consists of a polypeptide sequence of SEQ ID NO: 77 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 78, LCDR2 comprises or consists of a polypeptide sequence of SEQ ID NO: 79 and LCDR3 comprises or consists of a polypeptide sequence of SEQ ID NO: 80.
Clause 37B. A binding polypeptide comprising or consisting of a paired heavy chain variable domain and light chain variable domain which bind to LTBR wherein the heavy chain variable domain comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 81, HCDR2 comprises or consists of a polypeptide sequence sharing 70% or greater, 75% or greater, 80% or greater, 85% or greater, 90% or greater or 95% or greater sequence identity with SEQ ID NO: 82 and HCDR3 comprises or consists of a polypeptide sequence sharing 70% or greater, 75% or greater, 80% or greater, 85% or greater, 90% or greater or 95% or greater sequence identity with SEQ ID NO: 83 and the light chain variable domain comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 84, LCDR2 comprises or consists of a polypeptide sequence sharing 70% or greater, 75% or greater, 80% or greater, 85% or greater, 90% or greater or 95% or greater sequence identity with SEQ ID NO: 85 and LCDR3 comprises or consists of a polypeptide sequence sharing 70% or greater, 75% or greater, 80% or greater, 85% or greater, 90% or greater or 95% or greater sequence identity with SEQ ID NO: 86.
Clause 38B. The polypeptide of clause 37B wherein HCDR1 comprises or consists of a polypeptide sequence of SEQ ID NO: 81, HCDR2 comprises or consists of a polypeptide sequence of SEQ ID NO: 82 and HCDR3 comprises or consists of a polypeptide sequence of SEQ ID NO: 83 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 84, LCDR2 comprises or consists of a polypeptide sequence of SEQ ID NO: 85 and LCDR3 comprises or consists of a polypeptide sequence of SEQ ID NO: 86.
Clause 39B. A binding polypeptide comprising or consisting of a paired heavy chain variable domain and light chain variable domain which bind to LTBR wherein the heavy chain variable domain comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 87, HCDR2 comprises or consists of a polypeptide sequence sharing 70% or greater, 75% or greater, 80% or greater, 85% or greater, 90% or greater or 95% or greater sequence identity with SEQ ID NO: 88 and HCDR3 comprises or consists of a polypeptide sequence sharing 70% or greater, 75% or greater, 80% or greater, 85% or greater, 90% or greater or 95% or greater sequence identity with SEQ ID NO: 89 and the light chain variable domain comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 90, LCDR2 comprises or consists of a polypeptide sequence sharing 70% or greater, 75% or greater, 80% or greater, 85% or greater, 90% or greater or 95% or greater sequence identity with SEQ ID NO: 91 and LCDR3 comprises or consists of a polypeptide sequence sharing 70% or greater, 75% or greater, 80% or greater, 85% or greater, 90% or greater or 95% or greater sequence identity with SEQ ID NO: 92.
Clause 40B. The polypeptide of any one of clause 39B wherein HCDR1 comprises or consists of a polypeptide sequence of SEQ ID NO: 87, HCDR2 comprises or consists of a polypeptide sequence of SEQ ID NO: 88 and HCDR3 comprises or consists of a polypeptide sequence of SEQ ID NO: 89 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 90, LCDR2 comprises or consists of a polypeptide sequence of SEQ ID NO: 91 and LCDR3 comprises or consists of a polypeptide sequence of SEQ ID NO: 92.
Clause 41B. A binding polypeptide comprising or consisting of a paired heavy chain variable domain and light chain variable domain which bind to LTBR wherein the heavy chain variable domain comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 93, HCDR2 comprises or consists of a polypeptide sequence sharing 70% or greater, 75% or greater, 80% or greater, 85% or greater, 90% or greater or 95% or greater sequence identity with SEQ ID NO: 94 and HCDR3 comprises or consists of a polypeptide sequence sharing 70% or greater, 75% or greater, 80% or greater, 85% or greater, 90% or greater or 95% or greater sequence identity with SEQ ID NO: 95 and the light chain variable domain comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 96, LCDR2 comprises or consists of a polypeptide sequence sharing 70% or greater, 75% or greater, 80% or greater, 85% or greater, 90% or greater or 95% or greater sequence identity with SEQ ID NO: 97 and LCDR3 comprises or consists of a polypeptide sequence sharing 70% or greater, 75% or greater, 80% or greater, 85% or greater, 90% or greater or 95% or greater sequence identity with SEQ ID NO: 98.
Clause 42B. The polypeptide of clause 41 B wherein HCDR1 comprises or consists of a polypeptide sequence of SEQ ID NO: 93, HCDR2 comprises or consists of a polypeptide sequence of SEQ ID NO: 94 and HCDR3 comprises or consists of a polypeptide sequence of SEQ ID NO: 95 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 96, LCDR2 comprises or consists of a polypeptide sequence of SEQ ID NO: 97 and LCDR3 comprises or consists of a polypeptide sequence of SEQ ID NO: 98.
Clause 43B. A binding polypeptide comprising or consisting of a paired heavy chain variable domain and light chain variable domain which bind to LTBR wherein the heavy chain variable domain comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 99, HCDR2 comprises or consists of a polypeptide sequence sharing 70% or greater, 75% or greater, 80% or greater, 85% or greater, 90% or greater or 95% or greater sequence identity with SEQ ID NO: 100 and HCDR3 comprises or consists of a polypeptide sequence sharing 70% or greater, 75% or greater, 80% or greater, 85% or greater, 90% or greater or 95% or greater sequence identity with SEQ ID NO: 101 and the light chain variable domain comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 102, LCDR2 comprises or consists of a polypeptide sequence sharing 70% or greater, 75% or greater, 80% or greater, 85% or greater, 90% or greater or 95% or greater sequence identity with SEQ ID NO: 103 and LCDR3 comprises or consists of a polypeptide sequence sharing 70% or greater, 75% or greater, 80% or greater, 85% or greater, 90% or greater or 95% or greater sequence identity with SEQ ID NO: 104.
Clause 44B. The polypeptide of any one of clause 43B wherein HCDR1 comprises or consists of a polypeptide sequence of SEQ ID NO: 99, HCDR2 comprises or consists of a polypeptide sequence of SEQ ID NO: 100 and HCDR3 comprises or consists of a polypeptide sequence of SEQ ID NO: 101 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 102, LCDR2 comprises or consists of a polypeptide sequence of SEQ ID NO: 103 and LCDR3 comprises or consists of a polypeptide sequence of SEQ ID NO: 104.
Clause 45B. A binding polypeptide comprising or consisting of a paired heavy chain variable domain and light chain variable domain which bind to LTBR wherein the heavy chain variable domain comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 105, HCDR2 comprises or consists of a polypeptide sequence sharing 70% or greater, 75% or greater, 80% or greater, 85% or greater, 90% or greater or 95% or greater sequence identity with SEQ ID NO: 106 and HCDR3 comprises or consists of a polypeptide sequence sharing 70% or greater, 75% or greater, 80% or greater, 85% or greater, 90% or greater or 95% or greater sequence identity with SEQ ID NO: 107 and the light chain variable domain comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 108, LCDR2 comprises or consists of a polypeptide sequence sharing 70% or greater, 75% or greater, 80% or greater, 85% or greater, 90% or greater or 95% or greater sequence identity with SEQ ID NO: 109 and LCDR3 comprises or consists of a polypeptide sequence sharing 70% or greater, 75% or greater, 80% or greater, 85% or greater, 90% or greater or 95% or greater sequence identity with SEQ ID NO: 110.
Clause 46B. The polypeptide of clause 45B wherein HCDR1 comprises or consists of a polypeptide sequence of SEQ ID NO: 105, HCDR2 comprises or consists of a polypeptide sequence of SEQ ID NO: 106 and HCDR3 comprises or consists of a polypeptide sequence of SEQ ID NO: 107 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 108, LCDR2 comprises or consists of a polypeptide sequence of SEQ ID NO: 109 and LCDR3 comprises or consists of a polypeptide sequence of SEQ ID NO: 110.
Clause 47B. A binding polypeptide comprising or consisting of a paired heavy chain variable domain and light chain variable domain which bind to LTBR wherein the heavy chain variable domain comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 111, HCDR2 comprises or consists of a polypeptide sequence sharing 70% or greater, 75% or greater, 80% or greater, 85% or greater, 90% or greater or 95% or greater sequence identity with SEQ ID NO: 112 and HCDR3 comprises or consists of a polypeptide sequence sharing 70% or greater, 75% or greater, 80% or greater, 85% or greater, 90% or greater or 95% or greater sequence identity with SEQ ID NO: 113 and the light chain variable domain comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 114, LCDR2 comprises or consists of a polypeptide sequence sharing 70% or greater, 75% or greater, 80% or greater, 85% or greater, 90% or greater or 95% or greater sequence identity with SEQ ID NO: 115 and LCDR3 comprises or consists of a polypeptide sequence sharing 70% or greater, 75% or greater, 80% or greater, 85% or greater, 90% or greater or 95% or greater sequence identity with SEQ ID NO: 116.
Clause 48B. The polypeptide of any one of clause 47B wherein HCDR1 comprises or consists of a polypeptide sequence of SEQ ID NO: 111, HCDR2 comprises or consists of a polypeptide sequence of SEQ ID NO: 112 and HCDR3 comprises or consists of a polypeptide sequence of SEQ ID NO: 113 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 114, LCDR2 comprises or consists of a polypeptide sequence of SEQ ID NO: 115 and LCDR3 comprises or consists of a polypeptide sequence of SEQ ID NO: 116.
Clause 49B. A binding polypeptide comprising or consisting of a paired heavy chain variable domain and light chain variable domain which bind to LTBR, wherein the heavy chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 27 and the light chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 28.
Clause 50B. The polypeptide of any one of clause 49B wherein the heavy chain variable domain comprises or consists of a polypeptide sequence of SEQ ID NO: 27 and the light chain variable domain comprises or consists of a polypeptide sequence of SEQ ID NO: 28.
Clause 51B. A polypeptide comprising or consisting of a paired heavy chain variable domain and light chain variable domain which bind to LTBR, wherein the heavy chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 29 and the light chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 30.
Clause 52B. The polypeptide of clause 51 B wherein the heavy chain variable domain comprises or consists of a polypeptide sequence of SEQ ID NO: 29 and the light chain variable domain comprises or consists of a polypeptide sequence of SEQ ID NO: 30.
Clause 53B. A polypeptide comprising or consisting of a paired heavy chain variable domain and light chain variable domain which bind to LTBR, wherein the heavy chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 31 and the light chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 32.
Clause 54B. The polypeptide of clause 53B wherein the heavy chain variable domain comprises or consists of a polypeptide sequence of SEQ ID NO: 31 and the light chain variable domain comprises or consists of a polypeptide sequence of SEQ ID NO: 32.
Clause 55B. A polypeptide comprising or consisting of a paired heavy chain variable domain and light chain variable domain which bind to LTBR, wherein the heavy chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 33 and the light chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 34.
Clause 56B. The polypeptide of clause 55B wherein the heavy chain variable domain comprises or consists of a polypeptide sequence of SEQ ID NO: 33 and the light chain variable domain comprises or consists of a polypeptide sequence of SEQ ID NO: 34.
Clause 57B. A polypeptide comprising or consisting of a paired heavy chain variable domain and light chain variable domain which bind to LTBR, wherein the heavy chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 35 and the light chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 36.
Clause 58B. The polypeptide of clause 57B wherein the heavy chain variable domain comprises or consists of a polypeptide sequence of SEQ ID NO: 35 and the light chain variable domain comprises or consists of a polypeptide sequence of SEQ ID NO: 36.
Clause 59B. A polypeptide comprising or consisting of a paired heavy chain variable domain and light chain variable domain which bind to LTBR, wherein the heavy chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 37 and the light chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 38.
Clause 60B. The polypeptide of clause 59B wherein the heavy chain variable domain comprises or consists of a polypeptide sequence of SEQ ID NO: 37 and the light chain variable domain comprises or consists of a polypeptide sequence of SEQ ID NO: 38.
Clause 61B. A polypeptide comprising or consisting of a paired heavy chain variable domain and light chain variable domain which bind to LTBR, wherein the heavy chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 39 and the light chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 40.
Clause 62B. The polypeptide of clause 61 B wherein the heavy chain variable domain comprises or consists of a polypeptide sequence of SEQ ID NO: 39 and the light chain variable domain comprises or consists of a polypeptide sequence of SEQ ID NO: 40.
Clause 63B. A polypeptide comprising or consisting of a paired heavy chain variable domain and light chain variable domain which bind to LTBR, wherein the heavy chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 41 and the light chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 42.
Clause 64B. The polypeptide of clause 62B wherein the heavy chain variable domain comprises or consists of a polypeptide sequence of SEQ ID NO: 41 and the light chain variable domain comprises or consists of a polypeptide sequence of SEQ ID NO: 42.
Clause 65B. A polypeptide which binds to LTBR comprising or consisting of the polypeptide sequence(s) of clone 1.1, 1.2, 1.3, 1.4, 1.5, 1.6, 1.7, 1.8, 1.9, 1.10, 1.11, 1.12, 2.1, 2.2, 2.3, 2.4, 2.5, 2.6, 2.7 or 2.8.
Clause 66B. An antibody which competes for binding to LTBR with the polypeptide of any one of clauses 1 B to 65B.
Clause 67B. The polypeptide of any one of clauses 1 B to 66B wherein the binding polypeptide specifically binds to LTBR.
Clause 68B. The polypeptide of clause 67B wherein the LTBR binding polypeptide specifically binds to LTBR in that any other entity is bound to with a KD of 10⁻⁷ M or more, such as 10⁻⁶ M or more.
Clause 69B. The polypeptide of either 67B or 68B wherein the LTBR binding polypeptide specifically binds to the extracellular domain of LTBR.
Clause 70B. The polypeptide of any one of clauses 1 B to 69B, wherein the LTBR is on the surface of stromal cells.
Clause 71B. The polypeptide of any one of clauses 1 B to 70B, wherein the LTBR is on the surface of cancer cells, myeloid cells and/or macrophages.
Clause 72B. The polypeptide of any one of any one of clauses 1B to 71B wherein the LTBR binding polypeptide is an LTBR agonist.
Clause 73B. The polypeptide of clause 72B wherein the LTBR agonist is an agonist of NFKB pathways.
Clause 74B. The polypeptide of any one of clause 73B wherein the LTBR agonist (a) induces clustering of LTBR and/or (b) activates the classical NFKB pathway (such as leading to expression of adhesion molecules such as ICAMs, such as ICAM-1, VCAM-1 and/or MAdCAM-1) and/or activates the alternative NFKB pathway (such as leading to expression of chemokines, such as CCL5, CCL19, CCL21 and/or CXCL13).
Clause 75B. The polypeptide of any one of clauses 1 B to 74B wherein the LTBR is human, Macaca fascicularis or mouse LTBR.
Clause 76B. The polypeptide of clause 75B wherein the LTBR is human LTBR.
Clause 77B. The polypeptide of any one of clauses 1 to 76B wherein the LTBR binding polypeptide binds to LTBR with an affinity (K_{D}) of 20 nM or less, such as 15 nM or less, such as 10 nM or less, such as 9 nM or less, such as 8 nM or less, such as 7 nM or less, such as 6 nM or less, such as 5 nM or less, such as 4 nM or less, such as 3 nM or less, such as 2 nM or less, such as 1 nM or less.
Clause 78B. The polypeptide of any one of clauses 1 to 76B wherein the LTBR binding polypeptide binds to LTBR with an affinity (K_{D}) as recited in or less.
Clause 79B. The polypeptide of either clause 77B or 78B wherein the affinity is established as set out in Example 2.
Clause 80B. The polypeptide of any one of clauses 1 B to 79B wherein the LTBR binding polypeptide has an EC50 of 9.0E-09 M or less, such as 8.0E-09 M or less, such as 7.0E-09 M or less, such as 6.0E-09 M or less, such as 5.0E-09 M or less, such as 4.0E-09 M or less, such as 3.0E-09 M or less, such as 2.0E-09 M or less, such as 1.0E-09 M or less, such as 9.0E-10 M or less, such as 8.0E-10 M or less, such as 7.0E-10 M or less, such as 6.0E-10 M or less, such as 5.0E-10 M or less, such as 4.0E-10 M or less, such as 3.0E-10 M or less, such as 2.0E-10 M or less, such as 1.0E-10 M or less, such as 9.0E-11 M or less, such as 8.0E-11 M or less, such as 7.0E-11 M or less, such as 6.0E-11 M or less, such as 5.0E-11 M or less, such as 4.0E-11 M or less, such as 3.0E-11 M or less, such as 2.0E-11 M or less, such as 1.0E-11 M or less, such as 9.0E-12 M or less, such as 8.0E-12 M or less, such as 7.0E-12 M or less, such as 6.0E-12 M or less, such as 5.0E-12 M or less, such as 4.0E-12 M or less, such as 3.0E-12 M or less, such as 2.0E-12 M or less, such as 1.0E-12 M or less.
Clause 81B. The polypeptide of any one of clauses 1 to 79B wherein the LTBR binding polypeptide has an EC50 as recited for any of the clones specified in the tables of the Examples or less.
Clause 82B. A binding polypeptide comprising or consisting of a paired heavy chain variable domain and light chain variable domain which bind to FAP wherein the heavy chain variable domain comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 117, HCDR2 comprises or consists of a polypeptide sequence sharing 70% or greater, 75% or greater, 80% or greater, 85% or greater, 90% or greater or 95% or greater sequence identity with SEQ ID NO: 118 and HCDR3 comprises or consists of a polypeptide sequence sharing 70% or greater, 75% or greater, 80% or greater, 85% or greater, 90% or greater or 95% or greater sequence identity with SEQ ID NO: 119 and the light chain variable domain comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 120, LCDR2 comprises or consists of a polypeptide sequence sharing 70% or greater, 75% or greater, 80% or greater, 85% or greater, 90% or greater or 95% or greater sequence identity with SEQ ID NO: 121 and LCDR3 comprises or consists of a polypeptide sequence sharing 70% or greater, 75% or greater, 80% or greater, 85% or greater, 90% or greater or 95% or greater sequence identity with SEQ ID NO: 122.
Clause 83B. The polypeptide of clause 82B wherein HCDR1 comprises or consists of a polypeptide sequence of SEQ ID NO: 117, HCDR2 comprises or consists of a polypeptide sequence of SEQ ID NO: 118 and HCDR3 comprises or consists of a polypeptide sequence of SEQ ID NO: 119 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 120, LCDR2 comprises or consists of a polypeptide sequence of SEQ ID NO: 121 and LCDR3 comprises or consists of a polypeptide sequence of SEQ ID NO: 122.
Clause 84B. A binding polypeptide comprising or consisting of a paired heavy chain variable domain and light chain variable domain which bind to FAP wherein the heavy chain variable domain comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 123, HCDR2 comprises or consists of a polypeptide sequence sharing 70% or greater, 75% or greater, 80% or greater, 85% or greater, 90% or greater or 95% or greater sequence identity with SEQ ID NO: 124 and HCDR3 comprises or consists of a polypeptide sequence sharing 70% or greater, 75% or greater, 80% or greater, 85% or greater, 90% or greater or 95% or greater sequence identity with SEQ ID NO: 125 and the light chain variable domain comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 126, LCDR2 comprises or consists of a polypeptide sequence sharing 70% or greater, 75% or greater, 80% or greater, 85% or greater, 90% or greater or 95% or greater sequence identity with SEQ ID NO: 127 and LCDR3 comprises or consists of a polypeptide sequence sharing 70% or greater, 75% or greater, 80% or greater, 85% or greater, 90% or greater or 95% or greater sequence identity with SEQ ID NO: 128.
Clause 85B. The polypeptide of clause 84B wherein HCDR1 comprises or consists of a polypeptide sequence of SEQ ID NO: 123, HCDR2 comprises or consists of a polypeptide sequence of SEQ ID NO: 124 and HCDR3 comprises or consists of a polypeptide sequence of SEQ ID NO: 125 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 126, LCDR2 comprises or consists of a polypeptide sequence of SEQ ID NO: 127 and LCDR3 comprises or consists of a polypeptide sequence of SEQ ID NO: 128.
Clause 86B. A binding polypeptide comprising or consisting of a paired heavy chain variable domain and light chain variable domain which bind to FAP wherein the heavy chain variable domain comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 129, HCDR2 comprises or consists of a polypeptide sequence sharing 70% or greater, 75% or greater, 80% or greater, 85% or greater, 90% or greater or 95% or greater sequence identity with SEQ ID NO: 130 and HCDR3 comprises or consists of a polypeptide sequence sharing 70% or greater, 75% or greater, 80% or greater, 85% or greater, 90% or greater or 95% or greater sequence identity with SEQ ID NO: 131 and the light chain variable domain comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 132, LCDR2 comprises or consists of a polypeptide sequence sharing 70% or greater, 75% or greater, 80% or greater, 85% or greater, 90% or greater or 95% or greater sequence identity with SEQ ID NO: 133 and LCDR3 comprises or consists of a polypeptide sequence sharing 70% or greater, 75% or greater, 80% or greater, 85% or greater, 90% or greater or 95% or greater sequence identity with SEQ ID NO: 134.
Clause 87B. The polypeptide of clause 86B wherein HCDR1 comprises or consists of a polypeptide sequence of SEQ ID NO: 129, HCDR2 comprises or consists of a polypeptide sequence of SEQ ID NO: 130 and HCDR3 comprises or consists of a polypeptide sequence of SEQ ID NO: 131 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 132, LCDR2 comprises or consists of a polypeptide sequence of SEQ ID NO: 133 and LCDR3 comprises or consists of a polypeptide sequence of SEQ ID NO: 134.
Clause 88B. A binding polypeptide comprising or consisting of a paired heavy chain variable domain and light chain variable domain which bind to FAP wherein the heavy chain variable domain comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 135, HCDR2 comprises or consists of a polypeptide sequence sharing 70% or greater, 75% or greater, 80% or greater, 85% or greater, 90% or greater or 95% or greater sequence identity with SEQ ID NO: 136 and HCDR3 comprises or consists of a polypeptide sequence sharing 70% or greater, 75% or greater, 80% or greater, 85% or greater, 90% or greater or 95% or greater sequence identity with SEQ ID NO: 137 and the light chain variable domain comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 138, LCDR2 comprises or consists of a polypeptide sequence sharing 70% or greater, 75% or greater, 80% or greater, 85% or greater, 90% or greater or 95% or greater sequence identity with SEQ ID NO: 139 and LCDR3 comprises or consists of a polypeptide sequence sharing 70% or greater, 75% or greater, 80% or greater, 85% or greater, 90% or greater or 95% or greater sequence identity with SEQ ID NO: 140.
Clause 89B. The polypeptide of clause 88B wherein HCDR1 comprises or consists of a polypeptide sequence of SEQ ID NO: 135, HCDR2 comprises or consists of a polypeptide sequence of SEQ ID NO: 136 and HCDR3 comprises or consists of a polypeptide sequence of SEQ ID NO: 137 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 138, LCDR2 comprises or consists of a polypeptide sequence of SEQ ID NO: 139 and LCDR3 comprises or consists of a polypeptide sequence of SEQ ID NO: 140.
Clause 90B. A binding polypeptide comprising or consisting of a paired heavy chain variable domain and light chain variable domain which bind to FAP wherein the heavy chain variable domain comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 141, HCDR2 comprises or consists of a polypeptide sequence sharing 70% or greater, 75% or greater, 80% or greater, 85% or greater, 90% or greater or 95% or greater sequence identity with SEQ ID NO: 142 and HCDR3 comprises or consists of a polypeptide sequence sharing 70% or greater, 75% or greater, 80% or greater, 85% or greater, 90% or greater or 95% or greater sequence identity with SEQ ID NO: 143 and the light chain variable domain comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 144, LCDR2 comprises or consists of a polypeptide sequence sharing 70% or greater, 75% or greater, 80% or greater, 85% or greater, 90% or greater or 95% or greater sequence identity with SEQ ID NO: 145 and LCDR3 comprises or consists of a polypeptide sequence sharing 70% or greater, 75% or greater, 80% or greater, 85% or greater, 90% or greater or 95% or greater sequence identity with SEQ ID NO: 146.
Clause 91B. The polypeptide of clause 90B wherein HCDR1 comprises or consists of a polypeptide sequence of SEQ ID NO: 141, HCDR2 comprises or consists of a polypeptide sequence of SEQ ID NO: 142 and HCDR3 comprises or consists of a polypeptide sequence of SEQ ID NO: 143 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 144, LCDR2 comprises or consists of a polypeptide sequence of SEQ ID NO: 145 and LCDR3 comprises or consists of a polypeptide sequence of SEQ ID NO: 146.
Clause 92B. A binding polypeptide comprising or consisting of a paired heavy chain variable domain and light chain variable domain which bind to FAP wherein the heavy chain variable domain comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 147, HCDR2 comprises or consists of a polypeptide sequence sharing 70% or greater, 75% or greater, 80% or greater, 85% or greater, 90% or greater or 95% or greater sequence identity with SEQ ID NO: 148 and HCDR3 comprises or consists of a polypeptide sequence sharing 70% or greater, 75% or greater, 80% or greater, 85% or greater, 90% or greater or 95% or greater sequence identity with SEQ ID NO: 149 and the light chain variable domain comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 150, LCDR2 comprises or consists of a polypeptide sequence sharing 70% or greater, 75% or greater, 80% or greater, 85% or greater, 90% or greater or 95% or greater sequence identity with SEQ ID NO: 151 and LCDR3 comprises or consists of a polypeptide sequence sharing 70% or greater, 75% or greater, 80% or greater, 85% or greater, 90% or greater or 95% or greater sequence identity with SEQ ID NO: 152.
Clause 93B. The polypeptide of clause 92B wherein HCDR1 comprises or consists of a polypeptide sequence of SEQ ID NO: 147, HCDR2 comprises or consists of a polypeptide sequence of SEQ ID NO: 148 and HCDR3 comprises or consists of a polypeptide sequence of SEQ ID NO: 149 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 150, LCDR2 comprises or consists of a polypeptide sequence of SEQ ID NO: 151 and LCDR3 comprises or consists of a polypeptide sequence of SEQ ID NO: 152.
Clause 94B. A binding polypeptide comprising or consisting of a paired heavy chain variable domain and light chain variable domain which bind to FAP wherein the heavy chain variable domain comprises or consists of three complementarity determining regions (HCDR1-HCDR3) and four framework regions (HFR1-HFR4), wherein HCDR1 comprises or consists of a polypeptide sequence sharing 70% or greater, 75% or greater, 80% or greater, 85% or greater, 90% or greater or 95% or greater sequence identity with SEQ ID NO: 153, HCDR2 comprises or consists of a polypeptide sequence sharing 70% or greater, 75% or greater, 80% or greater, 85% or greater, 90% or greater or 95% or greater sequence identity with SEQ ID NO: 154 and HCDR3 comprises or consists of a polypeptide sequence sharing 70% or greater, 75% or greater, 80% or greater, 85% or greater, 90% or greater or 95% or greater sequence identity with SEQ ID NO: 155 and the light chain variable domain comprises or consists of three complementarity determining regions (LCDR1-LCDR3) and four framework regions (LFR1-LFR4), wherein LCDR1 comprises or consists of a polypeptide sequence sharing 70% or greater, 75% or greater, 80% or greater, 85% or greater, 90% or greater or 95% or greater sequence identity with SEQ ID NO: 156, LCDR2 comprises or consists of a polypeptide sequence sharing 70% or greater, 75% or greater, 80% or greater, 85% or greater, 90% or greater or 95% or greater sequence identity with SEQ ID NO: 157 and LCDR3 comprises or consists of a polypeptide sequence sharing 70% or greater, 75% or greater, 80% or greater, 85% or greater, 90% or greater or 95% or greater sequence identity with SEQ ID NO: 158.
Clause 95B. The polypeptide of clause 94B wherein HCDR1 comprises or consists of a polypeptide sequence of SEQ ID NO: 153, HCDR2 comprises or consists of a polypeptide sequence of SEQ ID NO: 154 and HCDR3 comprises or consists of a polypeptide sequence of SEQ ID NO: 155 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 156, LCDR2 comprises or consists of a polypeptide sequence of SEQ ID NO: 157 and LCDR3 comprises or consists of a polypeptide sequence of SEQ ID NO: 158.
Clause 96B. A binding polypeptide comprising or consisting of a paired heavy chain variable domain and light chain variable domain which bind to FAP, wherein the heavy chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 43 and the light chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 44.
Clause 97B. The polypeptide of clause 96B wherein the heavy chain variable domain comprises or consists of a polypeptide sequence of SEQ ID NO: 43 and the light chain variable domain comprises or consists of a polypeptide sequence of SEQ ID NO: 44.
Clause 98B. A binding polypeptide comprising or consisting of a paired heavy chain variable domain and light chain variable domain which bind to FAP, wherein the heavy chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 45 and the light chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 46.
Clause 99B. The polypeptide of clause 98B wherein the heavy chain variable domain comprises or consists of a polypeptide sequence of SEQ ID NO: 45 and the light chain variable domain comprises or consists of a polypeptide sequence of SEQ ID NO: 46.
Clause 100B. A binding polypeptide comprising or consisting of a paired heavy chain variable domain and light chain variable domain which bind to FAP, wherein the heavy chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 47 and the light chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 48.
Clause 101 B. The polypeptide of clause 100B wherein the heavy chain variable domain comprises or consists of a polypeptide sequence of SEQ ID NO: 47 and the light chain variable domain comprises or consists of a polypeptide sequence of SEQ ID NO: 48.
Clause 102B. A binding polypeptide comprising or consisting of a paired heavy chain variable domain and light chain variable domain which bind to FAP, wherein the heavy chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 49 and the light chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 50.
Clause 103B. The polypeptide of clause 102B wherein the heavy chain variable domain comprises or consists of a polypeptide sequence of SEQ ID NO: 49 and the light chain variable domain comprises or consists of a polypeptide sequence of SEQ ID NO: 50.
Clause 104B. A binding polypeptide comprising or consisting of a paired heavy chain variable domain and light chain variable domain which bind to FAP, wherein the heavy chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 51 and the light chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 52.
Clause 105B. The polypeptide of clause 104B wherein the heavy chain variable domain comprises or consists of a polypeptide sequence of SEQ ID NO: 51 and the light chain variable domain comprises or consists of a polypeptide sequence of SEQ ID NO: 52.
Clause 106B. A binding polypeptide comprising or consisting of a paired heavy chain variable domain and light chain variable domain which bind to FAP, wherein the heavy chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 53 and the light chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 54.
Clause 107B. The polypeptide of clause 106B wherein the heavy chain variable domain comprises or consists of a polypeptide sequence of SEQ ID NO: 53 and the light chain variable domain comprises or consists of a polypeptide sequence of SEQ ID NO: 54.
Clause 108B. A binding polypeptide comprising or consisting of a paired heavy chain variable domain and light chain variable domain which bind to FAP, wherein the heavy chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 55 and the light chain variable domain comprises or consists of a polypeptide sequence sharing 70% or greater, 75% or greater, 80% or greater, 85% or greater, 90% or greater, 95% or greater, 96% or greater, 97% or greater, 98% or greater or 99% or greater sequence identity with SEQ ID NO: 56.
Clause 109B. The polypeptide of any one of clause 108B wherein the heavy chain variable domain comprises or consists of a polypeptide sequence of SEQ ID NO: 55 and the light chain variable domain comprises or consists of a polypeptide sequence of SEQ ID NO: 56.
Clause 110B. A polypeptide which binds to FAP comprising or consisting of the polypeptide sequence(s) of clone 3.1, 3.2, 3.3, 3.4, 3.5, 3.6 or 3.7.
Clause 111 B. An antibody which competes for binding to FAP with the polypeptide of any one of clauses 82B to 110B.
Clause 112B. The polypeptide of any one of clauses 82B to 111B wherein the FAP binding polypeptide specifically binds to FAP.
Clause 113B. The polypeptide of clause 112B wherein the FAP binding polypeptide specifically binds to FAP in that any other entity is bound to with a KD of 10⁻⁷ M or more, such as 10⁻⁶ M or more.
Clause 114B. The polypeptide of any one of clauses 82B to 113B, wherein the FAP is on the surface of stromal cells.
Clause 115B. The polypeptide of any one of clauses 82B to 114B, wherein the FAP is on the surface of cancer cells.
Clause 116B. The polypeptide of any one of clauses 112B to 115B wherein the FAP binding polypeptide specifically binds to the extracellular domain of FAP.
Clause 117B. The polypeptide of any one of clauses 82B to 116B wherein the FAP is human, Macaca fascicularis or mouse FAP.
Clause 118B. The polypeptide of clause 117B wherein the FAP is human FAP.
Clause 119B. The polypeptide of any one of clauses 82B to 118B wherein the FAP binding polypeptide binds to FAP with an affinity (K_{D}) of 400 pM or less, such as 300 pM or less, such as 200 pM or less, such as 150 pM or less, such as 130 pM or less, such as 110 pM or less, such as 100 pM or less, such as 50 pM or less.
Clause 120B. The polypeptide of clause 119B wherein the FAP binding polypeptide binds to FAP with an affinity (K_{D}) as recited in or less.
Clause 121B. The polypeptide of either clause 119B or 120B wherein the affinity is established as set out in Example 3.
Clause 122B. The polypeptide of any one of clauses 82B to 121B wherein the FAP binding polypeptide has an EC50 of 9.0E-09 M or less, such as 8.0E-09 M or less, such as 7.0E-09 M or less, such as 6.0E-09 M or less, such as 5.0E-09 M or less, such as 4.0E-09 M or less, such as 3.0E-09 M or less, such as 2.0E-09 M or less, such as 1.0E-09 M or less, such as 9.0E-10 M or less, such as 8.0E-10 M or less, such as 7.0E-10 M or less, such as 6.0E-10 M or less, such as 5.0E-10 M or less, such as 4.0E-10 M or less, such as 3.0E-10 M or less, such as 2.0E-10 M or less, such as 1.0E-10 M or less, such as 9.0E-11 M or less, such as 8.0E-11 M or less, such as 7.0E-11 M or less, such as 6.0E-11 M or less, such as 5.0E-11 M or less, such as 4.0E-11 M or less, such as 3.0E-11 M or less, such as 2.0E-11 M or less, such as 1.0E-11 M or less, such as 9.0E-12 M or less, such as 8.0E-12 M or less, such as 7.0E-12 M or less, such as 6.0E-12 M or less, such as 5.0E-12 M or less, such as 4.0E-12 M or less, such as 3.0E-12 M or less, such as 2.0E-12 M or less, such as 1.0E-12 M or less.
Clause 123B. The polypeptide of any one of clauses 82B to 121B wherein the FAP binding polypeptide has an EC50 as recited for any of the clones specified in the tables of the Examples or less.
Clause 124B. The polypeptide of any one of clauses 1 B to 123B, wherein the polypeptide comprises an antibody.
Clause 125B. The polypeptide of any one of clauses 1B to 124B wherein the polypeptide comprises a further binding agent which is neither an LTBR binding agent nor a FAP binding agent.
Clause 126B. The polypeptide of any one of clauses 1B to 125B, wherein the polypeptide comprises one or more antibody fragments.
Clause 127B. The polypeptide of clause 126B, wherein the one or more antibody fragments are independently selected from an scFv, Fab, Fab′, F(ab′)2, variable domain (e.g. VH, VL, VNAR or VHH), diabody or minibody.
Clause 128B. The polypeptide of clause 124B, wherein the polypeptide consists of an antibody.
Clause 129B. The polypeptide of clause 126B or 127B, wherein the polypeptide consists of one or more antibody fragments.
Clause 130B. The polypeptide of any one of clauses 1 B to 127B wherein the polypeptide comprises no further binding agents.
Clause 131 B. An antibody which binds to the epitope of the polypeptide of any one of clauses 1B to 130B.
Clause 132B. The polypeptide of any one of clauses 1 B to 131 B wherein the polypeptide is for use as a medicament.
Clause 133B. The polypeptide of clause 132B for use in the treatment of cancer.
Clause 134B. A method of treating cancer comprising administering to a subject in need thereof the polypeptide of any one of clauses 1 B to 131 B.
Clause 135B. Use of a polypeptide of any one of clauses 1B to 131B in the manufacture of a medicament for the treatment of cancer.
Clause 136B. The polypeptide for use, method or use of any one of clauses 133B to 135B wherein the cancer is a tumour.
Clause 137B. The polypeptide for use, method or use of any one of clauses 133B to 135B wherein the cancer is a non-haematological cancer.
Clause 138B. The polypeptide for use, method or use of any one of clauses 133B to 137B for use in the treatment of cancer (e.g. a tumour) in combination with radiotherapy, chemotherapy or antibody-drug conjugates (ADCs).
Clause 139B. The polypeptide for use, method or use of clause 138B wherein the radiotherapy is hypo-fractionated radiotherapy.
Clause 140B. A set of one or more polynucleotides encoding the polypeptide of any one of clauses 1B to 130B.
Clause 141 B. A set of one or more expression vectors collectively comprising the polynucleotides of clause 140B.
Clause 142B. A cell comprising the expression vectors or polynucleotides of either clause 140B or 141B.
Clause 143B. A method of producing a polypeptide of any one of clauses 1 B to 130B, the method comprising culturing the cell of clause 142B under suitable conditions such that the polynucleotide is expressed, and the polypeptide is produced.
Clause 143B. A binding polypeptide comprising at least one CDR, wherein the CDR is a HCDR1, HCDR2 or HCDR3 comprised in a heavy chain variable domain of SEQ ID NOs: 23-26 or 159-162.
Clause 144B. The binding polypeptide of clause 143B comprising at least such HCDR1 and HCDR2, such HCDR1 and HCDR3, or such HCDR2 and HCDR3.
Clause 145B. The binding polypeptide of clause 144B comprising such HCDR1, HCDR2 and HCDR3.
Clause 146B. The binding polypeptide according to any one of clauses 143B to 145B wherein the HCDR1, HCDR2 and HCDR3 are delineated according to the Kabat-, Chothia-, Martin-, IMGT-, or AbM-method.
Clause 147B. The binding polypeptide according to any one of clauses 143B to 145B wherein the HCDR1 is chosen from SEQ ID NOs: 57, 60, 63, 66, 163, 166, 169 or 172 as defined by the Kabat-method; the HCDR2 is chosen from SEQ ID NOs: 58, 61, 64, 67, 164, 167, 170, 173, 205 or 206 as defined by the Kabat-method; and/or the HCDR3 is chosen from SEQ ID NOs: 59, 62, 65, 68, 165, 168, 171, 174, 207 or 208 as defined by the Kabat-method.
Clause 148B. The binding polypeptide according to any one of clauses 143B to 147B which binds to LTBR.
Clause 149B. The polypeptide for use, use or method of any one of clauses 133B to 137B wherein the polypeptide is for use in the treatment of cancer in combination with a cancer vaccine.
Clause 150B. The polypeptide for use, use or method of clause 149B wherein the polypeptide and cancer vaccine are for use in combination with an adjuvant.
Clause 151 B. The polypeptide for use, use or method of either clause 149B or 150B wherein the polypeptide, cancer vaccine and/or adjuvant are administered sequentially, separately or simultaneously.
Clause 152B. The polypeptide for use, use or method of any one of clauses 149B to 151 B wherein the cancer vaccine is an antigenic peptide or a polynucleotide encoding an antigen (such as RNA (e.g. mRNA) or DNA), optionally comprised in a pharmaceutically acceptable excipient.
Clause 153B. The polypeptide for use, use or method of any one of clauses 149B to 152B wherein the cancer vaccine is a tumour associated antigen or an encoded tumour associated antigen.
Clause 154B. The polypeptide for use, use or method of clause 153B wherein the cancer vaccine is a tumour associated antigen comprising or consisting of the polypeptide sequence KCLQDNNWDYTRYAQAFTLLKAKG (SEQ ID NO: 209).
Clause 155B. The polypeptide for use, use or method of any one of clauses 150B to 154B wherein the adjuvant is a TLR agonist.
Clause 156B. The polypeptide for use, use or method of clause 155B wherein the adjuvant is a TLR-9 agonist.
Clause 157B. The polypeptide for use, use or method of clause 156B wherein the adjuvant is a CpG oligonucleotide.

A series of clauses setting out further embodiments of the invention are as follows. These embodiments generally relate to (i) anti-LTBR binding polypeptides for use in the treatment of cancer in combination with radiotherapy or a cancer vaccine or (ii) anti-FAP binding polypeptides for use in the treatment of cancer in combination with radiotherapy or a cancer vaccine.
Clause 1C. A binding polypeptide which binds to LTBR for use in the treatment of cancer in combination with radiotherapy or a cancer vaccine.
Clause 2C. A binding polypeptide which binds to FAP for use in the treatment of cancer in combination with radiotherapy or a cancer vaccine.
Clause 3C. A method of treating cancer comprising administering to a subject in need thereof a binding polypeptide which binds to LTBR in combination with radiotherapy or a cancer vaccine.
Clause 4C. A method of treating cancer comprising administering to a subject in need thereof a binding polypeptide which binds to FAP in combination with radiotherapy or a cancer vaccine.
Clause 5C. The polypeptide for use or method of any one of clauses 1C to 4C, wherein the polypeptide comprises an antibody.
Clause 6C. The polypeptide for use or method of any one of clauses 1C to 5C wherein the polypeptide comprises a further binding agent which is neither an LTBR binding agent nor a FAP binding agent.
Clause 7C. The polypeptide for use or method of any one of clauses 1C to 6C, wherein the polypeptide comprises one or more antibody fragments.
Clause 8C. The polypeptide for use or method of clause 7C, wherein the one or more antibody fragments are independently selected from an scFv, Fab, Fab′, F(ab′)2, variable domain (e.g. VH, VL, VNAR or VHH), diabody or minibody.
Clause 9C. The polypeptide for use or method of clause 5C, wherein the polypeptide consists of an antibody.
Clause 10C. The polypeptide for use or method of clause 7C or 8C, wherein the polypeptide consists of one or more antibody fragments.
Clause 11C. The polypeptide for use or method of any one of clauses 1 B to 10C wherein the polypeptide comprises no further binding agents.
Clause 12C. The polypeptide for use or method of any one of clauses 1C to 11C wherein the cancer is a tumour.
Clause 13C. The polypeptide for use or method of any one of clauses 1C to 11C wherein the cancer is a non-haematological cancer.
Clause 14C. The polypeptide for use or method of any one of clauses 1C to 13C wherein the radiotherapy is hypo-fractionated radiotherapy.
Clause 15C. The polypeptide for use or method of any one of clauses 1C to 14C wherein the polypeptide is for use in the treatment of cancer in combination with a cancer vaccine.
Clause 16C. The polypeptide for use or method of clause 15C wherein the polypeptide and cancer vaccine are for use in combination with an adjuvant.
Clause 17C. The polypeptide for use or method of either clause 15C or 16C wherein the polypeptide, cancer vaccine and/or adjuvant are administered sequentially, separately or simultaneously.
Clause 18C. The polypeptide for use or method of any one of clauses 15C to 17C wherein the cancer vaccine is an antigenic peptide or a polynucleotide encoding an antigen (such as RNA (e.g. mRNA) or DNA), optionally comprised in a pharmaceutically acceptable excipient.
Clause 19C. The polypeptide for use or method of any one of clauses 15C to 18C wherein the cancer vaccine is a tumour associated antigen or an encoded tumour associated antigen.
Clause 20C. The polypeptide for use or method of clause 19C wherein the cancer vaccine is a tumour associated antigen comprising or consisting of the polypeptide sequence KCLQDNNWDYTRYAQAFTLLKAKG (SEQ ID NO: 209).
Clause 21C. The polypeptide for use or method of any one of clauses 16C to 20C wherein the adjuvant is a TLR agonist.
Clause 22C. The polypeptide for use or method of clause 21C wherein the adjuvant is a TLR-9 agonist.
Clause 23C. The polypeptide for use or method of clause 22C wherein the adjuvant is a CpG oligonucleotide.
   A series of clauses setting out further embodiments of the invention are as follows.
Clause 1D. A construct comprising a FAP binding agent and an LTBR binding agent.
Clause 2D. The construct of clause 1D wherein the construct comprises more than one FAP binding agent.
Clause 3D. The construct of either clause 1D or 2D wherein the construct comprises more than one LTBR binding agent.
Clause 4D. The construct of any one of clauses 1D to 3D wherein the construct comprises an antibody or antibody fragment, where one or all of the FAP binding agents is a FAP binding domain and one or all of the LTBR binding agents is an LTBR binding domain.
Clause 5D. The construct of clause 4D wherein the antibody fragment comprises an Fc region and one or all of the LTBR binding agents are linked via a linker to the Fc region of the antibody or antibody fragment.
Clause 6D. The construct of clause 5D wherein one or all of the LTBR binding agents are linked via a linker to the CH3 domain of the Fc region.
Clause 7D. The construct of any one of clauses 1D to 6D wherein the LTBR binding agent is an LTBR agonist.
Clause 8D. The construct of clause 7D wherein the LTBR agonist activates the classical NFKB pathway.
Clause 9D. The construct of clause 7D wherein the LTBR binding agent is capable of activating LTBR on cells which express both LTBR and FAP.
Clause 10D. The construct of any one of clauses 1D to 9D wherein one or all of the LTBR binding agents is a variable domain which binds to LTBR wherein the variable domain which binds to LTBR comprises or consists of three heavy chain complementarity determining regions (HCDR1-HCDR3) and four framework regions (FR1-FR4), wherein:
(a) HCDR1 comprises or consists of a polypeptide sequence of SEQ ID NO: 60 or 172, HCDR2 comprises or consists of a polypeptide sequence of SEQ ID NO: 61 or 173 and HCDR3 comprises or consists of a polypeptide sequence of SEQ ID NO: 62 or 174 or
(b) HCDR1 comprises or consists of a polypeptide sequence of SEQ ID NO: 57 or 166, HCDR2 comprises or consists of a polypeptide sequence of SEQ ID NO: 58 or 167 and HCDR3 comprises or consists of a polypeptide sequence of SEQ ID NO: 59 or 168 or
(c) HCDR1 comprises or consists of a polypeptide sequence of SEQ ID NO: 63 or 163, HCDR2 comprises or consists of a polypeptide sequence of SEQ ID NO: 64 or 164 and HCDR3 comprises or consists of a polypeptide sequence of SEQ ID NO: 65 or 165 or
(d) HCDR1 comprises or consists of a polypeptide sequence of SEQ ID NO: 66 or 169, HCDR2 comprises or consists of a polypeptide sequence of SEQ ID NO: 67 or 170 and HCDR3 comprises or consists of a polypeptide sequence of SEQ ID NO: 68 or 171.
Clause 11D. The construct of any one of clauses 1D to 9D wherein one or all of the LTBR binding agents is a paired heavy chain variable domain and light chain variable domain wherein the heavy chain variable domain which binds to LTBR comprises or consists of three complementarity determining regions (HCDR1-HCDR3) and four framework regions (HFR1-HFR4) and the light chain variable domain which binds to LTBR comprises or consists of three complementarity determining regions (LCDR1-LCDR3) and four framework regions (LFR1-LFR4), wherein:
(a) HCDR1 comprises or consists of a polypeptide sequence of SEQ ID NO: 69, HCDR2 comprises or consists of a polypeptide sequence of SEQ ID NO: 70 and HCDR3 comprises or consists of a polypeptide sequence of SEQ ID NO: 71 and wherein LCDR1 comprises or consists of a polypeptide sequence of SEQ ID NO: 72, LCDR2 comprises or consists of a polypeptide sequence of SEQ ID NO: 73 and LCDR3 comprises or consists of a polypeptide sequence of SEQ ID NO: 74 or
(b) HCDR1 comprises or consists of a polypeptide sequence of SEQ ID NO: 75, HCDR2 comprises or consists of a polypeptide sequence of SEQ ID NO: 76 and HCDR3 comprises or consists of a polypeptide sequence of SEQ ID NO: 77 and wherein LCDR1 comprises or consists of a polypeptide sequence of SEQ ID NO: 78, LCDR2 comprises or consists of a polypeptide sequence of SEQ ID NO: 79 and LCDR3 comprises or consists of a polypeptide sequence of SEQ ID NO: 80 or
(c) HCDR1 comprises or consists of a polypeptide sequence of SEQ ID NO: 81, HCDR2 comprises or consists of a polypeptide sequence of SEQ ID NO: 82 and HCDR3 comprises or consists of a polypeptide sequence of SEQ ID NO: 83 and wherein LCDR1 comprises or consists of a polypeptide sequence of SEQ ID NO: 84, LCDR2 comprises or consists of a polypeptide sequence of SEQ ID NO: 85 and LCDR3 comprises or consists of a polypeptide sequence of SEQ ID NO: 86 or
(d) HCDR1 comprises or consists of a polypeptide sequence of SEQ ID NO: 87, HCDR2 comprises or consists of a polypeptide sequence of SEQ ID NO: 88 and HCDR3 comprises or consists of a polypeptide sequence of SEQ ID NO: 89 and wherein LCDR1 comprises or consists of a polypeptide sequence of SEQ ID NO: 90, LCDR2 comprises or consists of a polypeptide sequence of SEQ ID NO: 91 and LCDR3 comprises or consists of a polypeptide sequence of SEQ ID NO: 92 or
(e) HCDR1 comprises or consists of a polypeptide sequence of SEQ ID NO: 93, HCDR2 comprises or consists of a polypeptide sequence of SEQ ID NO: 94 and HCDR3 comprises or consists of a polypeptide sequence of SEQ ID NO: 95 and wherein LCDR1 comprises or consists of a polypeptide sequence of SEQ ID NO: 96, LCDR2 comprises or consists of a polypeptide sequence of SEQ ID NO: 97 and LCDR3 comprises or consists of a polypeptide sequence of SEQ ID NO: 98 or
(f) HCDR1 comprises or consists of a polypeptide sequence of SEQ ID NO: 99, HCDR2 comprises or consists of a polypeptide sequence of SEQ ID NO: 100 and HCDR3 comprises or consists of a polypeptide sequence of SEQ ID NO: 101 and wherein LCDR1 comprises or consists of a polypeptide sequence of SEQ ID NO: 102, LCDR2 comprises or consists of a polypeptide sequence of SEQ ID NO: 103 and LCDR3 comprises or consists of a polypeptide sequence of SEQ ID NO: 104 or
(g) HCDR1 comprises or consists of a polypeptide sequence of SEQ ID NO: 105, HCDR2 comprises or consists of a polypeptide sequence of SEQ ID NO: 106 and HCDR3 comprises or consists of a polypeptide sequence of SEQ ID NO: 107 and wherein LCDR1 comprises or consists of a polypeptide sequence of SEQ ID NO: 108, LCDR2 comprises or consists of a polypeptide sequence of SEQ ID NO: 109 and LCDR3 comprises or consists of a polypeptide sequence of SEQ ID NO: 110 or
(h) HCDR1 comprises or consists of a polypeptide sequence of SEQ ID NO: 111, HCDR2 comprises or consists of a polypeptide sequence of SEQ ID NO: 112 and HCDR3 comprises or consists of a polypeptide sequence of SEQ ID NO: 113 and wherein LCDR1 comprises or consists of a polypeptide sequence of SEQ ID NO: 114, LCDR2 comprises or consists of a polypeptide sequence of SEQ ID NO: 115 and LCDR3 comprises or consists of a polypeptide sequence of SEQ ID NO: 116.
Clause 12D. The construct of any one of clauses 1D to 11D wherein one or all of the FAP binding agents is a paired heavy chain variable domain and light chain variable domain wherein the heavy chain variable domain which binds to FAP comprises or consists of three complementarity determining regions (HCDR1-HCDR3) and four framework regions (HFR1-HFR4) and the light chain variable domain which binds to FAP comprises or consists of three complementarity determining regions (LCDR1-LCDR3) and four framework regions (LFR1-LFR4), wherein:
(a) HCDR1 comprises or consists of a polypeptide sequence of SEQ ID NO: 117, HCDR2 comprises or consists of a polypeptide sequence of SEQ ID NO: 118 and HCDR3 comprises or consists of a polypeptide sequence of SEQ ID NO: 119 and wherein LCDR1 comprises or consists of a polypeptide sequence of SEQ ID NO: 120, LCDR2 comprises or consists of a polypeptide sequence of SEQ ID NO: 121 and LCDR3 comprises or consists of a polypeptide sequence of SEQ ID NO: 122 or
(b) HCDR1 comprises or consists of a polypeptide sequence of SEQ ID NO: 123, HCDR2 comprises or consists of a polypeptide sequence of SEQ ID NO: 124 and HCDR3 comprises or consists of a polypeptide sequence of SEQ ID NO: 125 and wherein LCDR1 comprises or consists of a polypeptide sequence of SEQ ID NO: 126, LCDR2 comprises or consists of a polypeptide sequence of SEQ ID NO: 127 and LCDR3 comprises or consists of a polypeptide sequence of SEQ ID NO: 128 or
(c) HCDR1 comprises or consists of a polypeptide sequence of SEQ ID NO: 129, HCDR2 comprises or consists of a polypeptide sequence of SEQ ID NO: 130 and HCDR3 comprises or consists of a polypeptide sequence of SEQ ID NO: 131 and wherein LCDR1 comprises or consists of a polypeptide sequence of SEQ ID NO: 132, LCDR2 comprises or consists of a polypeptide sequence of SEQ ID NO: 133 and LCDR3 comprises or consists of a polypeptide sequence of SEQ ID NO: 134 or
(d) HCDR1 comprises or consists of a polypeptide sequence of SEQ ID NO: 135, HCDR2 comprises or consists of a polypeptide sequence of SEQ ID NO: 136 and HCDR3 comprises or consists of a polypeptide sequence of SEQ ID NO: 137 and wherein LCDR1 comprises or consists of a polypeptide sequence of SEQ ID NO: 138, LCDR2 comprises or consists of a polypeptide sequence of SEQ ID NO: 139 and LCDR3 comprises or consists of a polypeptide sequence of SEQ ID NO: 140 or
(e) HCDR1 comprises or consists of a polypeptide sequence of SEQ ID NO: 141, HCDR2 comprises or consists of a polypeptide sequence of SEQ ID NO: 142 and HCDR3 comprises or consists of a polypeptide sequence of SEQ ID NO: 143 and wherein LCDR1 comprises or consists of a polypeptide sequence of SEQ ID NO: 144, LCDR2 comprises or consists of a polypeptide sequence of SEQ ID NO: 145 and LCDR3 comprises or consists of a polypeptide sequence of SEQ ID NO: 146 or
(f) HCDR1 comprises or consists of a polypeptide sequence of SEQ ID NO: 147, HCDR2 comprises or consists of a polypeptide sequence of SEQ ID NO: 148 and HCDR3 comprises or consists of a polypeptide sequence of SEQ ID NO: 149 and wherein LCDR1 comprises or consists of a polypeptide sequence of SEQ ID NO: 150, LCDR2 comprises or consists of a polypeptide sequence of SEQ ID NO: 151 and LCDR3 comprises or consists of a polypeptide sequence of SEQ ID NO: 152 or
(g) HCDR1 comprises or consists of a polypeptide sequence of SEQ ID NO: 153, HCDR2 comprises or consists of a polypeptide sequence of SEQ ID NO: 154 and HCDR3 comprises or consists of a polypeptide sequence of SEQ ID NO: 155 and wherein LCDR1 comprises or consists of a polypeptide sequence of SEQ ID NO: 156, LCDR2 comprises or consists of a polypeptide sequence of SEQ ID NO: 157 and LCDR3 comprises or consists of a polypeptide sequence of SEQ ID NO: 158.
Clause 13D. The construct of any one of clauses 1D to 12D wherein one or all of the LTBR binding agents are VHHs.
Clause 14D. The construct of any one of clauses 1D to 12D wherein the construct comprises:
(a) a bispecific, FAP-monovalent, LTBR-monovalent antibody comprising a heterodimer of two polypeptide chains, the first chain comprising or consisting of from N to C terminus a Fab region, a hinge region, a CH2 domain and a CH3 domain; and the second chain comprising or consisting of from N to C terminus a VHH, a hinge region and a CH2 domain and a CH3 domain, wherein the hinge regions are linked via a disulphide bond, the CH2 and CH3 domains are associated to form an Fc region, the Fab region comprises a paired VH and VL at its N-terminus which form a FAP binding site and the VHH forms an LTBR binding site;
(b) a bispecific, FAP-bivalent, LTBR-monovalent antibody comprising a heterodimer of two polypeptide chains, the first chain comprising or consisting of from N to C terminus a Fab region, a hinge region, a CH2 domain, a CH3 domain and a VHH; and the second chain comprising or consisting of from N to C terminus a Fab region, a hinge region, a CH2 domain and a CH3 domain, wherein the hinge regions are linked via a disulphide bond, the CH2 and CH3 domains are associated to form an Fc region, the Fab regions each comprise a paired VH and VL at their N-terminus which each form a FAP binding site and the VHH forms an LTBR binding site at the C-terminus;
(c) a bispecific, FAP-bivalent, LTBR-bivalent antibody comprising a homodimer of two polypeptide chains, the first chain comprising or consisting of from N to C terminus a Fab region, a hinge region, a CH2 domain, a CH3 domain and a VHH; and the second chain comprising or consisting of from N to C terminus a Fab region, a hinge region, a CH2 domain, a CH3 domain and a VHH wherein the hinge regions are linked via a disulphide bond, the CH2 and CH3 domains are associated to form an Fc region, the Fab regions each comprise a paired VH and VL at their N-terminus which each form a FAP binding site and the VHHs each form an LTBR binding site at the C-terminus; or
(d) a bispecific, FAP-bivalent, LTBR-bivalent antibody comprising a homodimer of two polypeptide chains, the first chain comprising or consisting of from N to C terminus a Fab region, a hinge region, a CH2 domain, a CH3 domain and an scFv; and the second chain comprising or consisting of from N to C terminus a Fab region, a hinge region, a CH2 domain, a CH3 domain and an scFv wherein the hinge regions are linked via a disulphide bond, the CH2 and CH3 domains are associated to form an Fc region, the Fab regions each comprise a paired VH and VL at their N-terminus which each form a FAP binding site and the scFvs each form an LTBR binding site at the C-terminus.
Clause 15D. The construct of any one of clauses 1D to 14D wherein the construct is an antibody comprising
(a) one or more Fab domains, each comprising a paired heavy chain variable domain and light chain variable domain which bind to FAP; wherein
   (i) the heavy chain variable domain comprises or consists of a polypeptide sequence of SEQ ID NO: 43 and the light chain variable domain comprises or consists of a polypeptide sequence of SEQ ID NO: 44;
   (ii) the heavy chain variable domain comprises or consists of a polypeptide sequence of SEQ ID NO: 45 and the light chain variable domain comprises or consists of a polypeptide sequence of SEQ ID NO: 46; or
   (iii) the heavy chain variable domain comprises or consists of a polypeptide sequence of SEQ ID NO: 47 and the light chain variable domain comprises or consists of a polypeptide sequence of SEQ ID NO: 48,
and
(b) one or more paired heavy chain variable domain and light chain variable domain which bind to LTBR;
   wherein the heavy chain variable domain comprises or consists of a polypeptide sequence of SEQ ID NO: 31 and the light chain variable domain comprises or consists of a polypeptide sequence of SEQ ID NO: 32
or one or more variable domains which bind to LTBR; wherein
   (i) the heavy chain variable domain comprises or consists of a polypeptide sequence of SEQ ID NO: 24; or
   (ii) the heavy chain variable domain comprises or consists of a polypeptide sequence of SEQ ID NO: 25.
Clause 16D. The construct of any one of clauses 1D to 14D wherein the construct is an antibody comprising:
(i) a heterodimer of two different heavy chains (HC1 and HC2) and two identical light chains (LC) wherein:
   (a) the HC1 polypeptide sequence comprises or consists of SEQ ID NO: 175, the HC2 polypeptide sequence comprises or consists of SEQ ID NO: 176 and the LC polypeptide sequence comprises or consists of SEQ ID NO: 177;
   (b) the HC1 polypeptide sequence comprises or consists of SEQ ID NO: 178, the HC2 polypeptide sequence comprises or consists of SEQ ID NO: 179 and the LC polypeptide sequence comprises or consists of SEQ ID NO: 180;
   (c) the HC1 polypeptide sequence comprises or consists of SEQ ID NO: 181, the HC2 polypeptide sequence comprises or consists of SEQ ID NO: 182 and the LC polypeptide sequence comprises or consists of SEQ ID NO: 183;
   (d) the HC1 polypeptide sequence comprises or consists of SEQ ID NO: 184, the HC2 polypeptide sequence comprises or consists of SEQ ID NO: 185 and the LC polypeptide sequence comprises or consists of SEQ ID NO: 186;
   (e) the HC1 polypeptide sequence comprises or consists of SEQ ID NO: 187, the HC2 polypeptide sequence comprises or consists of SEQ ID NO: 188 and the LC polypeptide sequence comprises or consists of SEQ ID NO: 189;
   (f) the HC1 polypeptide sequence comprises or consists of SEQ ID NO: 190, the HC2 polypeptide sequence comprises or consists of SEQ ID NO: 191 and the LC polypeptide sequence comprises or consists of SEQ ID NO: 192;
   (g) the HC1 polypeptide sequence comprises or consists of SEQ ID NO: 193, the HC2 polypeptide sequence comprises or consists of SEQ ID NO: 194 and the LC polypeptide sequence comprises or consists of SEQ ID NO: 195; or
   (h) the HC1 polypeptide sequence comprises or consists of SEQ ID NO: 200, the HC2 polypeptide sequence comprises or consists of SEQ ID NO: 201 and the LC polypeptide sequence comprises or consists of SEQ ID NO: 202 or
(ii) a homodimer of two identical heavy chains (HC) and two identical light chains (LC) wherein:
   (a) the HC polypeptide sequence comprises or consists of SEQ ID NO: 196 and the LC polypeptide sequence comprises or consists of SEQ ID NO: 197 or
   (b) the HC polypeptide sequence comprises or consists of SEQ ID NO: 198 and the LC polypeptide sequence comprises or consists of SEQ ID NO: 199.
Clause 17D. The construct of any one of clauses 1D to 16D for use in the treatment of a tumour.
Clause 18D. A binding polypeptide comprising or consisting of a heavy chain variable domain which binds to LTBR wherein the heavy chain variable domain comprises or consists of three heavy chain complementarity determining regions (HCDR1-HCDR3) and four framework regions (FR1-FR4), wherein
(a) HCDR1 comprises or consists of a polypeptide sequence of SEQ ID NO: 60 or 172, HCDR2 comprises or consists of a polypeptide sequence of SEQ ID NO: 61 or 173 and HCDR3 comprises or consists of a polypeptide sequence of SEQ ID NO: 62 or 174;
(b) HCDR1 comprises or consists of a polypeptide sequence of SEQ ID NO: 57 or 166, HCDR2 comprises or consists of a polypeptide sequence of SEQ ID NO: 58 or 167 and HCDR3 comprises or consists of a polypeptide sequence of SEQ ID NO: 59 or 168;
(c) HCDR1 comprises or consists of a polypeptide sequence of SEQ ID NO: 63 or 163, HCDR2 comprises or consists of a polypeptide sequence of SEQ ID NO: 64 or 164 and HCDR3 comprises or consists of a polypeptide sequence of SEQ ID NO: 65 or 165;
(d) HCDR1 comprises or consists of a polypeptide sequence of SEQ ID NO: 66 or 169, HCDR2 comprises or consists of a polypeptide sequence of SEQ ID NO: 67 or 170 and HCDR3 comprises or consists of a polypeptide sequence of SEQ ID NO: 68 or 171;
(e) HCDR1 comprises or consists of a polypeptide sequence of SEQ ID NO: 163, HCDR2 comprises or consists of a polypeptide sequence of SEQ ID NO: 164 and HCDR3 comprises or consists of a polypeptide sequence of SEQ ID NO: 165;
(f) HCDR1 comprises or consists of a polypeptide sequence of SEQ ID NO: 166, HCDR2 comprises or consists of a polypeptide sequence of SEQ ID NO: 167 and HCDR3 comprises or consists of a polypeptide sequence of SEQ ID NO: 168;
(g) HCDR1 comprises or consists of a polypeptide sequence of SEQ ID NO: 169, HCDR2 comprises or consists of a polypeptide sequence of SEQ ID NO: 170 and HCDR3 comprises or consists of a polypeptide sequence of SEQ ID NO: 171; or
(h) HCDR1 comprises or consists of a polypeptide sequence of SEQ ID NO: 172, HCDR2 comprises or consists of a polypeptide sequence of SEQ ID NO: 173 and HCDR3 comprises or consists of a polypeptide sequence of SEQ ID NO: 174.
Clause 19D. A binding polypeptide comprising or consisting of a paired heavy chain variable domain and light chain variable domain which bind to LTBR wherein the heavy chain variable domain comprises or consists of three complementarity determining regions (HCDR1-HCDR3) and four framework regions (HFR1-HFR4), wherein
(a) HCDR1 comprises or consists of a polypeptide sequence of SEQ ID NO: 69, HCDR2 comprises or consists of a polypeptide sequence of SEQ ID NO: 70 and HCDR3 comprises or consists of a polypeptide sequence of SEQ ID NO: 71 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 72, LCDR2 comprises or consists of a polypeptide sequence of SEQ ID NO: 73 and LCDR3 comprises or consists of a polypeptide sequence of SEQ ID NO: 74;
(b) HCDR1 comprises or consists of a polypeptide sequence of SEQ ID NO: 75, HCDR2 comprises or consists of a polypeptide sequence of SEQ ID NO: 76 and HCDR3 comprises or consists of a polypeptide sequence of SEQ ID NO: 77 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 78, LCDR2 comprises or consists of a polypeptide sequence of SEQ ID NO: 79 and LCDR3 comprises or consists of a polypeptide sequence of SEQ ID NO: 80;
(c) HCDR1 comprises or consists of a polypeptide sequence of SEQ ID NO: 81, HCDR2 comprises or consists of a polypeptide sequence of SEQ ID NO: 82 and HCDR3 comprises or consists of a polypeptide sequence of SEQ ID NO: 83 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 84, LCDR2 comprises or consists of a polypeptide sequence of SEQ ID NO: 85 and LCDR3 comprises or consists of a polypeptide sequence of SEQ ID NO: 86;
(d) HCDR1 comprises or consists of a polypeptide sequence of SEQ ID NO: 87, HCDR2 comprises or consists of a polypeptide sequence of SEQ ID NO: 88 and HCDR3 comprises or consists of a polypeptide sequence of SEQ ID NO: 89 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 90, LCDR2 comprises or consists of a polypeptide sequence of SEQ ID NO: 91 and LCDR3 comprises or consists of a polypeptide sequence of SEQ ID NO: 92;
(e) HCDR1 comprises or consists of a polypeptide sequence of SEQ ID NO: 93, HCDR2 comprises or consists of a polypeptide sequence of SEQ ID NO: 94 and HCDR3 comprises or consists of a polypeptide sequence of SEQ ID NO: 95 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 96, LCDR2 comprises or consists of a polypeptide sequence of SEQ ID NO: 97 and LCDR3 comprises or consists of a polypeptide sequence of SEQ ID NO: 98;
(f) HCDR1 comprises or consists of a polypeptide sequence of SEQ ID NO: 99, HCDR2 comprises or consists of a polypeptide sequence of SEQ ID NO: 100 and HCDR3 comprises or consists of a polypeptide sequence of SEQ ID NO: 101 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 102, LCDR2 comprises or consists of a polypeptide sequence of SEQ ID NO: 103 and LCDR3 comprises or consists of a polypeptide sequence of SEQ ID NO: 104;
(g) HCDR1 comprises or consists of a polypeptide sequence of SEQ ID NO: 105, HCDR2 comprises or consists of a polypeptide sequence of SEQ ID NO: 106 and HCDR3 comprises or consists of a polypeptide sequence of SEQ ID NO: 107 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 108, LCDR2 comprises or consists of a polypeptide sequence of SEQ ID NO: 109 and LCDR3 comprises or consists of a polypeptide sequence of SEQ ID NO: 110; or
(h) HCDR1 comprises or consists of a polypeptide sequence of SEQ ID NO: 111, HCDR2 comprises or consists of a polypeptide sequence of SEQ ID NO: 112 and HCDR3 comprises or consists of a polypeptide sequence of SEQ ID NO: 113 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 114, LCDR2 comprises or consists of a polypeptide sequence of SEQ ID NO: 115 and LCDR3 comprises or consists of a polypeptide sequence of SEQ ID NO: 116.
Clause 20D. A binding polypeptide comprising or consisting of a paired heavy chain variable domain and light chain variable domain which bind to FAP wherein the heavy chain variable domain comprises or consists of three complementarity determining regions (HCDR1-HCDR3) and four framework regions (HFR1-HFR4), wherein
(a) HCDR1 comprises or consists of a polypeptide sequence of SEQ ID NO: 117, HCDR2 comprises or consists of a polypeptide sequence of SEQ ID NO: 118 and HCDR3 comprises or consists of a polypeptide sequence of SEQ ID NO: 119 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 120, LCDR2 comprises or consists of a polypeptide sequence of SEQ ID NO: 121 and LCDR3 comprises or consists of a polypeptide sequence of SEQ ID NO: 122;
(b) HCDR1 comprises or consists of a polypeptide sequence of SEQ ID NO: 123, HCDR2 comprises or consists of a polypeptide sequence of SEQ ID NO: 124 and HCDR3 comprises or consists of a polypeptide sequence of SEQ ID NO: 125 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 126, LCDR2 comprises or consists of a polypeptide sequence of SEQ ID NO: 127 and LCDR3 comprises or consists of a polypeptide sequence of SEQ ID NO: 128;
(c) HCDR1 comprises or consists of a polypeptide sequence of SEQ ID NO: 129, HCDR2 comprises or consists of a polypeptide sequence of SEQ ID NO: 130 and HCDR3 comprises or consists of a polypeptide sequence of SEQ ID NO: 131 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 132, LCDR2 comprises or consists of a polypeptide sequence of SEQ ID NO: 133 and LCDR3 comprises or consists of a polypeptide sequence of SEQ ID NO: 134;
(d) HCDR1 comprises or consists of a polypeptide sequence of SEQ ID NO: 135, HCDR2 comprises or consists of a polypeptide sequence of SEQ ID NO: 136 and HCDR3 comprises or consists of a polypeptide sequence of SEQ ID NO: 137 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 138, LCDR2 comprises or consists of a polypeptide sequence of SEQ ID NO: 139 and LCDR3 comprises or consists of a polypeptide sequence of SEQ ID NO: 140;
(e) HCDR1 comprises or consists of a polypeptide sequence of SEQ ID NO: 141, HCDR2 comprises or consists of a polypeptide sequence of SEQ ID NO: 142 and HCDR3 comprises or consists of a polypeptide sequence of SEQ ID NO: 143 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 144, LCDR2 comprises or consists of a polypeptide sequence of SEQ ID NO: 145 and LCDR3 comprises or consists of a polypeptide sequence of SEQ ID NO: 146;
(f) HCDR1 comprises or consists of a polypeptide sequence of SEQ ID NO: 147, HCDR2 comprises or consists of a polypeptide sequence of SEQ ID NO: 148 and HCDR3 comprises or consists of a polypeptide sequence of SEQ ID NO: 149 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 150, LCDR2 comprises or consists of a polypeptide sequence of SEQ ID NO: 151 and LCDR3 comprises or consists of a polypeptide sequence of SEQ ID NO: 152; or
(g) HCDR1 comprises or consists of a polypeptide sequence of SEQ ID NO: 153, HCDR2 comprises or consists of a polypeptide sequence of SEQ ID NO: 154 and HCDR3 comprises or consists of a polypeptide sequence of SEQ ID NO: 155 and the light chain variable domain comprises or consists of three complementarity determining regions (LCDR1-LCDR3) and four framework regions (LFR1-LFR4), wherein LCDR1 comprises or consists of a polypeptide sequence of SEQ ID NO: 156, LCDR2 comprises or consists of a polypeptide sequence of SEQ ID NO: 157 and LCDR3 comprises or consists of a polypeptide sequence of SEQ ID NO: 158.
Clause 21D. A construct or polypeptide of any one of clauses 1D to 20D for use in the treatment of cancer (e.g. a tumour) in combination with radiotherapy, a cancer vaccine or ADCs.
Clause 22D. A construct or polypeptide of any one of clauses 1D to 20D for use in the treatment of cancer (e.g. a tumour) in combination with an anti-CTLA-4 antibody, an anti-PD-L1 antibody or an anti-PD-1 antibody.
Clause 23D. A binding polypeptide which binds to LTBR for use in the treatment of cancer (e.g. a tumour) in combination with radiotherapy, a cancer vaccine or ADCs.
Clause 24D. The binding polypeptide or construct of any one of clauses 18D to 23D wherein the binding polypeptide is an antibody or fragment thereof.

### REFERENCES

Allen, Elizabeth, et al. “Combined antiangiogenic and anti-PD-L1 therapy stimulates tumor immunity through HEV formation.” Science translational medicine 9.385 (2017): eaak9679.
Asrir, Assia, et al. “Tumor-associated high endothelial venules mediate lymphocyte entry into tumors and predict response to PD-1 plus CTLA-4 combination immunotherapy.” Cancer Cell 40.3 (2022): 318-334.
Bénézech, Cecile, et al. “Lymphotoxin-β receptor signaling through NF-κB2-RelB pathway reprograms adipocyte precursors as lymph node stromal cells.” Immunity 37.4 (2012): 721-734.
Binz, H. Kaspar, et al. “Designing repeat proteins: well-expressed, soluble and stable proteins from combinatorial libraries of consensus ankyrin repeat proteins.” Journal of molecular biology 332.2 (2003): 489-503.
Cabrita, Rita, et al. “Tertiary lymphoid structures improve immunotherapy and survival in melanoma.” Nature 577.7791 (2020): 561-565.
Castiglioni, Alessandra, et al. “Combined PD-L1/TGFβ blockade allows expansion and differentiation of stem cell-like CD8 T cells in immune excluded tumors.” Nature Communications 14.1 (2023): 4703.
Chothia, Cyrus, and Arthur M. Lesk. “Canonical structures for the hypervariable regions of immunoglobulins.” Journal of molecular biology 196.4 (1987): 901-917.
Dejardin, Emmanuel, et al. “The lymphotoxin-β receptor induces different patterns of gene expression via two NF-κB pathways.” Immunity 17.4 (2002): 525-535.
Dieu-Nosjean, Marie-Caroline, et al. “Long-term survival for patients with non-small-cell lung cancer with intratumoral lymphoid structures.” Journal of Clinical Oncology 26.27 (2008): 4410-4417.
Fernandes, Monica T., Emmanuel Dejardin, and Nuno R. dos Santos. “Context-dependent roles for lymphotoxin-β receptor signaling in cancer development.” Biochimica et Biophysica Acta (BBA)-Reviews on Cancer 1865.2 (2016): 204-219.
Golay, Josée, et al. “Design and validation of a novel generic platform for the production of tetravalent IgG1-like bispecific antibodies.” The Journal of Immunology 196.7 (2016): 3199-3211.
Hamers-Casterman, C. T. S. G., et al. “Naturally occurring antibodies devoid of light chains.” Nature 363.6428 (1993): 446-448.
Haybaeck, Johannes, et al. “A lymphotoxin-driven pathway to hepatocellular carcinoma.” Cancer cell 16.4 (2009): 295-308.
Henry, Kevin A., and C. Roger MacKenzie. “Single-domain antibodies-Biology, Engineering and Emerging applications.” Frontiers in Immunology 9 (2018): 41.
Johansson-Percival, Anna, et al. “De novo induction of intratumoral lymphoid structures and vessel normalization enhances immunotherapy in resistant tumors.” Nature immunology 18.11 (2017): 1207-1217.
Kabat, Elvin Abraham. Sequences of proteins of immunological interest. No. 91. US Department of Health and Human Services, Public Health Service, National Institutes of Health, 1991.
Martin, Andrew C R, and James Allen. “Bioinformatics tools for antibody engineering.” Handbook of therapeutic antibodies (2007): 95-117.
Merchant, A. Margaret, et al. “An efficient route to human bispecific IgG.” Nature biotechnology 16.7 (1998): 677-681.
Muyldermans, Serge. “Nanobodies: natural single-domain antibodies.” Annual review of biochemistry 82 (2013): 775-797.
Padlan, Eduardo A. “Anatomy of the antibody molecule.” Molecular immunology 31.3 (1994): 169-217.
Pardon, Els, et al. “A general protocol for the generation of Nanobodies for structural biology.” Nature protocols 9.3 (2014): 674-693.
Pasero, Christine, et al. “The HVEM network: new directions in targeting novel costimulatory/co-inhibitory molecules for cancer therapy.” Current opinion in pharmacology 12.4 (2012): 478-485.
Sautès-Fridman, Catherine, et al. “Tertiary lymphoid structures in the era of cancer immunotherapy.” Nature Reviews Cancer 19.6 (2019): 307-325.
Schlothauer, Tilman, et al. “Novel human IgG1 and IgG4 Fc-engineered antibodies with completely abolished immune effector functions.” Protein Engineering, Design and Selection 29.10 (2016): 457-466.
Schneider, Kirsten, Karen G. Potter, and Carl F. Ware. “Lymphotoxin and LIGHT signaling pathways and target genes.” Immunological reviews 202.1 (2004): 49-66.
Schrama, David, et al. “Targeting of lymphotoxin-α to the tumor elicits an efficient immune response associated with induction of peripheral lymphoid-like tissue.” Immunity 14.2 (2001): 111-121.
Schumacher, Ton N., and Daniela S. Thommen. “Tertiary lymphoid structures in cancer.” Science 375.6576 (2022): eabf9419.
Sharma, Padmanee, et al. “Primary, adaptive, and acquired resistance to cancer immunotherapy.” Cell 168.4 (2017): 707-723.
Strohl, William R., and Lila M. Strohl. Therapeutic antibody engineering: current and future advances driving the strongest growth area in the pharmaceutical industry. Elsevier, 2012.
Tang, Haidong, et al. “Lymphotoxin signalling in tertiary lymphoid structures and immunotherapy.” Cellular & molecular immunology 14.10 (2017): 809-818.
Vanhersecke, Lucile, et al. “Mature tertiary lymphoid structures predict immune checkpoint inhibitor efficacy in solid tumors independently of PD-L1 expression.” Nature cancer 2.8 (2021): 794-802.
Wang, Feng, et al. “Design and characterization of mouse IgG1 and IgG2a bispecific antibodies for use in syngeneic models.” MAbs. Vol. 12. No. 1. Taylor & Francis, 2020.
Weatherill, Eve E., et al. “Towards a universal disulphide stabilised single chain Fv format: importance of interchain disulphide bond location and vL-vH orientation.” Protein Engineering, Design & Selection 25.7 (2012): 321-329.
Weinstein, Aliyah M., and Walter J. Storkus. “Therapeutic lymphoid organogenesis in the tumor microenvironment.” Advances in cancer research 128 (2015): 197-233.
Wilkinson, Ian, et al. “Fc-engineered antibodies with immune effector functions completely abolished.” Plos one 16.12 (2021): e0260954. www.imgt.org/IMGTScientificChart/Numbering/IMGTnumbering.html
Xie, Na, et al. “Neoantigens: promising targets for cancer therapy.” Signal Transduction and Targeted Therapy 8.1 (2023): 9.
Yu, Xiaojie, et al. “Reducing affinity as a strategy to boost immunomodulatory antibody agonism.” Nature 614.7948 (2023): 539-547.
Zboralski, Dirk, et al. “Preclinical evaluation of FAP-2286 for fibroblast activation protein targeted radionuclide imaging and therapy.” European Journal of Nuclear Medicine and Molecular Imaging 49.11 (2022): 3651-3667.
Zhao, Yan, et al. “Fibroblast activation protein in the tumor microenvironment predicts outcomes of PD-1 blockade therapy in advanced non-small cell lung cancer.” Journal of Cancer Research and Clinical Oncology (2022): 1-15.
US2012/0014947
US2021/0188990
WO2004002431
WO2023117834

1.-28. (canceled)

29. An antibody comprising a fibroblast activation protein alpha (FAP) binding domain and a lymphotoxin beta receptor (LTBR) binding domain, wherein the LTBR binding domain is a VHH which comprises three heavy chain complementarity determining regions (HCDR1-HCDR3) and four framework regions (FR1-FR4), wherein
HCDR1 comprises a polypeptide sequence of SEQ ID NO: 63,
HCDR2 comprises a polypeptide sequence of SEQ ID NO: 64 and
HCDR3 comprises a polypeptide sequence of SEQ ID NO: 65;
and wherein the FAP binding domain is a paired heavy chain variable domain and light chain variable domain wherein the heavy chain variable domain comprises three complementarity determining regions (HCDR1-HCDR3) and four framework regions (HFR1-HFR4) and the light chain variable domain comprises three complementarity determining regions (LCDR1-LCDR3) and four framework regions (LFR1-LFR4), wherein
HCDR1 comprises a polypeptide sequence of SEQ ID NO: 117,
HCDR2 comprises a polypeptide sequence of SEQ ID NO: 118,
HCDR3 comprises a polypeptide sequence of SEQ ID NO: 119,
LCDR1 comprises a polypeptide sequence of SEQ ID NO: 120,
LCDR2 comprises a polypeptide sequence of SEQ ID NO: 121 and
LCDR3 comprises a polypeptide sequence of SEQ ID NO: 122.

30. The antibody of claim 29 wherein the antibody comprises more than one FAP binding domain.

31. The antibody of claim 29 wherein the antibody comprises more than one LTBR binding domain.

32. The antibody of claim 31 wherein the antibody comprises more than one FAP binding domain and more than one LTBR binding domain.

33. The antibody of claim 32 comprising two FAP binding domains and two LTBR binding domains,
wherein the LTBR binding domains are VHHs which each comprise three heavy chain complementarity determining regions (HCDR1-HCDR3) and four framework regions (FR1-FR4), wherein
HCDR1 comprises a polypeptide sequence of SEQ ID NO: 63,
HCDR2 comprises a polypeptide sequence of SEQ ID NO: 64 and
HCDR3 comprises a polypeptide sequence of SEQ ID NO: 65;
and wherein the FAP binding domains are each a paired heavy chain variable domain and a light chain variable domain wherein the heavy chain variable domains comprise three complementarity determining regions (HCDR1-HCDR3) and four framework regions (HFR1-HFR4) and the light chain variable domains comprise three complementarity determining regions (LCDR1-LCDR3) and four framework regions (LFR1-LFR4), wherein
HCDR1 comprises a polypeptide sequence of SEQ ID NO: 117,
HCDR2 comprises a polypeptide sequence of SEQ ID NO: 118,
HCDR3 comprises a polypeptide sequence of SEQ ID NO: 119,
LCDR1 comprises a polypeptide sequence of SEQ ID NO: 120,
LCDR2 comprises a polypeptide sequence of SEQ ID NO: 121 and
LCDR3 comprises a polypeptide sequence of SEQ ID NO: 122.

34. The antibody of claim 33 wherein the antibody comprises a constant region.

35. The antibody of claim 34 wherein the VHHs are each linked to the constant region.

36. The antibody of claim 35 wherein the VHHs are each linked to the CH3 domain of the constant region via a peptide linker.

37. The antibody of claim 33 wherein the FAP binding domains are Fabs.

38. The antibody of claim 37 wherein the antibody is a human IgG1 antibody.

39. The antibody of claim 34 wherein the constant region comprises 234Ala and 235Ala.

40. The antibody of claim 33 comprising two FAP binding domains and two LTBR binding domains,
wherein the LTBR binding domains are VHHs which each comprise three heavy chain complementarity determining regions (HCDR1-HCDR3) and four framework regions (FR1-FR4), wherein
HCDR1 consists of a polypeptide sequence of SEQ ID NO: 63,
HCDR2 consists of a polypeptide sequence of SEQ ID NO: 64 and
HCDR3 consists of a polypeptide sequence of SEQ ID NO: 65;
and wherein the FAP binding domains are each a paired heavy chain variable domain and a light chain variable domain wherein the heavy chain variable domains comprise three complementarity determining regions (HCDR1-HCDR3) and four framework regions (HFR1-HFR4) and the light chain variable domains comprise three complementarity determining regions (LCDR1-LCDR3) and four framework regions (LFR1-LFR4), wherein
HCDR1 consists of a polypeptide sequence of SEQ ID NO: 117,
HCDR2 consists of a polypeptide sequence of SEQ ID NO: 118,
HCDR3 consists of a polypeptide sequence of SEQ ID NO: 119,
LCDR1 consists of a polypeptide sequence of SEQ ID NO: 120,
LCDR2 consists of a polypeptide sequence of SEQ ID NO: 121 and
LCDR3 consists of a polypeptide sequence of SEQ ID NO: 122.

41. The antibody of claim 33 wherein the construct comprises a first heavy chain, a first light chain, a second heavy chain and a second light chain wherein the first heavy chain comprises from N to C terminus a VH, a CH1, a CH2, a CH3 and a VHH, the second heavy chain comprises from N to C terminus a VH, a CH1, a CH2, a CH3 and a VHH, the first light chain comprises from N to C terminus a VL and a CL and associates with the VH and CH1 of the first heavy chain to form a Fab region, the second light chain comprises from N to C terminus a VL and a CL and associates with the VH and CH1 of the second heavy chain to form a Fab region and the CH2 and CH3 domains of the first and second heavy chains associate to form an Fc region, wherein the VH and VL of each Fab region bind to FAP and the VHHs bind to LTBR.

42. The antibody of claim 33 wherein the heavy chain variable domains of the LTBR binding domains each comprise a polypeptide sequence sharing 90% or greater sequence identity with SEQ ID NO: 25, the heavy chain variable domains of the FAP binding domains each comprise a polypeptide sequence sharing 90% or greater sequence identity with SEQ ID NO: 43 and the light chain variable domains of the FAP binding domains each comprise a polypeptide sequence sharing 90% or greater sequence identity with SEQ ID NO: 44.

43. The antibody of claim 42 wherein the heavy chain variable domains of the LTBR binding domains each comprise a polypeptide sequence of SEQ ID NO: 25, the heavy chain variable domains of the FAP binding domains each comprise a polypeptide sequence of SEQ ID NO: 43 and the light chain variable domains of the FAP binding domains each comprise a polypeptide sequence of SEQ ID NO: 44.

44. The antibody of claim 29 wherein the antibody comprises a homodimer of two identical heavy chains (HC) and two identical light chains (LC) wherein the HC polypeptide sequence comprises SEQ ID NO: 196 and the LC polypeptide sequence comprises SEQ ID NO: 197.


SEQUENCE LISTING (209 sequences)

SEQ ID NO: 1 - protein, 116 aa, synthetic construct
QVQLVQSGAEVKKPGSSVKVSCKASGYTFTTYYLHWVRQAPGQGLEWMGWIYPGNVHAQYNEKFKGRVTITADKSTSTAYMELSSLRSEDTAVYYCARSWEGFPYWGQGTTVTVSS

SEQ ID NO: 2 - protein, 107 aa, synthetic construct
DIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGQGTKVEIK

SEQ ID NO: 3 - protein, 5 aa, synthetic construct
GGGGS

SEQ ID NO: 4 - protein, 20 aa, synthetic construct
GGGGSGGGGSGGGGSGGGGS

SEQ ID NO: 5 - protein, 115 aa, synthetic construct
QVQLKESGPGLVQPSQTLSLTCTVSGFSLTTYSVHWVRQHSGKSLEWMGRMWTDGDTSYNSAFTSRLSISRDTSKSQVFLKMNSLQTEDTGTYYCARGYWYFDFWGPGTMVTVSS

SEQ ID NO: 6 - protein, 107 aa, synthetic construct
DVQMTQSPSYLAASPGESVSIRCKASKSISNNLAWYQEKPGKANKLLIHSGSTLQSGTPSRFSGSGSGTDFTLTIRSLEFEDFAVYYCQQYNEYPYTFGAGTKLELK

SEQ ID NO: 7 - protein, 116 aa, synthetic construct
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 8 - protein, 108 aa, synthetic construct
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 9 - protein, 405 aa, Homo sapiens
QAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 10 - protein, 197 aa, Homo sapiens
QAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLM

SEQ ID NO: 11 - protein, 405 aa, Macaca fascicularis
QVVPPYGSENQTCRDQEKEYYEPRHRICCSRCPPGTYVSAKCSRSRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCEDQGLVEAAPGTAQSDTTCRNPSESLPPEMSGTMLMLAILLPLAFFLLLATIFACIWKSHPSLCRKLGSLLKRHPQGEGPNPVAAGRDPPKANPQYPDLVEPLLPISGDVSPVSTGLPTALVSEEGVPQQQSPLDLTTEPQLEPGEQNQVAHGTNGIHVTGGSMTITGNIYIYNGPVLGGPPGPGDLPATPDPPYPIPEEGDPGPPGLSTPHQEDGKAWHLAETEHCGATPSNRGPRSQFITYD

SEQ ID NO: 12 - protein, 197 aa, Macaca fascicularis
QVVPPYGSENQTCRDQEKEYYEPRHRICCSRCPPGTYVSAKCSRSRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCEDQGLVEAAPGTAQSDTTCRNPSESLPPEMSGTMLM

SEQ ID NO: 13 - protein, 385 aa, Mus musculus
QLVPPYRIENQTCWDQDKEYYEPMHDVCCSRCPPGEFVFAVCSRSQDTVCKTCPHNSYNEHWNHLSTCQLCRPCDIVLGFEEVAPCTSDRKAECRCQPGMSCVYLDNECVHCEEERLVLCQPGTEAEVTDEIMDTDVNCVPCKPGHFQNTSSPRARCQPHTRCEIQGLVEAAPGTSYSDTICKNPPEPGAMLLLAILLSLVLFLLFTTVLACAWMRHPSLCRKLGTLLKRHPEGEESPPCPAPRADPHFPDLAEPLLPMSGDLSPSPAGPPTAPSLEEVVLQQQSPLVQARELEAEPGEHGQVAHGANGIHVTGGSVTVTGNIYIYNGPVLGGTRGPGDPPAPPEPPYPTPEEGAPGPSELSTPYQEDGKAWHLAETETLGCQDL

SEQ ID NO: 14 - protein, 193 aa, Mus musculus
QLVPPYRIENQTCWDQDKEYYEPMHDVCCSRCPPGEFVFAVCSRSQDTVCKTCPHNSYNEHWNHLSTCQLCRPCDIVLGFEEVAPCTSDRKAECRCQPGMSCVYLDNECVHCEEERLVLCQPGTEAEVTDEIMDTDVNCVPCKPGHFQNTSSPRARCQPHTRCEIQGLVEAAPGTSYSDTICKNPPEPGAMLL

SEQ ID NO: 15 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 16 - protein, 735 aa, Homo sapiens
LRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 17 - protein, 760 aa, Macaca fascicularis
MKTWVKIVFGVATSAVLALLVMCIVLRPPRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPFVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEDYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 18 - protein, 735 aa, Macaca fascicularis
LRPPRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPFVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEDYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 19 - protein, 761 aa, Mus musculus
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 20 - protein, 736 aa, Mus musculus
LRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 21 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 22 - protein, 182 aa, Homo sapiens
LQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 23 - protein, 117 aa, synthetic construct
DVQLVESGGGLVQPGGSLRLSCAASGSSIFSFEIMAWYRQAPGKQRELVAVINKGGEANYPDSVKGRFTISRDNAKNTVYLQMNSLRPEDTAVYYCNADAVPHGSYWGQGTLVTVSS

SEQ ID NO: 24 - protein, 112 aa, synthetic construct
DVQLVESGGGLVQPGGSLRLSCKASGTTFSDRAFGWYRQAPGKQRELVATISTAGLTWYDVSVKGRFTISRDNAKNTVYLQMNSLRPEDTAVYYCNTFRGNWGQGTLVTVSS

SEQ ID NO: 25 - protein, 117 aa, synthetic construct
DVQLVESGGGLVQPGGSLRLSCAASGRFFGIYDMYWYRQAPGKQRELVAISTSGGHTNYADSVKGRFTISRDNAKNTVYLQMNSLRPEDTAVYYCNIQRVDAPGFYWGQGTLVTVSS

SEQ ID NO: 26 - protein, 120 aa, synthetic construct
DVQLVESGGGLVQPGGSLRLSCAASGRTFTNYRMAWFRQAPGKEREAVAAINWGGGGTYYADSVKGRFTISRDNAKNTVYLQMNSLRPEDTAVYYCAADFTGWGSYFDYWGQGTLVTVSS

SEQ ID NO: 27 - protein, 121 aa, synthetic construct
EVQLVESGGGLVKPGGSLRLSCAASGFTFSSYCMNWVRQAPGKGLEWVSSISTGNTYIYYSDSVKGRFTISRDNAKNSLYLQMNSLRAEDTAVYYCARARYNWNYDAFDIWGQGTMVTVSS

SEQ ID NO: 28 - protein, 107 aa, synthetic construct
DIQMTQSPSSLSASIGDRVTITCQASQDISNFLSWYQQKPGKAPNLLIFDASNLETGVPSRFSESGSGTDFSFTITSLQPEDIATYYCQQYYNVPLTFGGGTKVEIK

SEQ ID NO: 29 - protein, 121 aa, synthetic construct
EVQLVESGGGLVKPGGSLRLSCAASRFTFSSYSMNWVRQAPGKGLEWVSSISPSSSYIYYADSVKGRFTISRDNAKNSLYLQMNSLRAEDTAVYYCARDHYNWNFDALDIWGQGTMVTVSS

SEQ ID NO: 30 - protein, 107 aa, synthetic construct
DIQMTQSPSTLSASVGDRVTITCRASQSISSWLAWYQQKPGKAPKFLIYKASSLESGVPSRFSGSGSGTEFTLTISSLQPDDFATYYCQQYNSFSRTFGQGTKVEIK

SEQ ID NO: 31 - protein, 121 aa, synthetic construct
EVQLVESGGGLVQPGGSLRLSCAASGFTFSSYAMSWVRQAPGKGLEWVSGISGSGGRTNYADSVKGRFTISRDNSKNTLYLQMNSLRAEDTAVYYCAKSDNWHYLDAFDIWGQGTMVTVSS

SEQ ID NO: 32 - protein, 113 aa, synthetic construct
DIVMTQSPDSLAVSLGERATINCKSSQNVLYSSNNKNYLTWYQQKPGQPPKLIIYWASTRESGVPDRFSGSGSGTDFTLTISSLQAEDVAVYYCQQYFNTPPTFGQGTKVEIK

SEQ ID NO: 33 - protein, 121 aa, synthetic construct
QVQLVESGGGVVQPGRSLRLSCAASGFTFSSYGIHWVRQAPGKGLEWVAVIWYDGSNKYYADSVKGRFTISRDNSKNTLYLQMNSLRAEDTAVYYCARDGGTGNYYYMDVWGKGTTVTVSS

SEQ ID NO: 34 - protein, 113 aa, synthetic construct
DIVMTQSPDSLAVSLGERATINCKSSQSVFYSSNNKNYLVWYQQKPGQPPKLLIYWASTRASGVPDRFSGSGSGTDFTLTISSLQAEDVAVYYCQQYYNTPLTFGGGTKVEIK

SEQ ID NO: 35 - protein, 120 aa, synthetic construct
EVQLVESGGGLVQPGRSLRLSCAASGFTFDDYDIHWVRQAPGKGLEWVSGINWNSITIGYADSVKGRFTISRDNAKNSLYLQMNSLRAEDTALYYCAKDRGSGWRIFDYWGQGTLVTVSS

SEQ ID NO: 36 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPYTFGQGTKLEIK

SEQ ID NO: 37 - protein, 121 aa, synthetic construct
QVQLVQSGTEMKKPGASVKVSCKASGYTFSSYDISWVRQAPGQGLEWMGWFSAYNGNSNYAQKFQGRVTMTTDTSTNTAYMELRSLRSDDTAVYYCAREGYYGNSGFFDYWGQGTLVTVSS

SEQ ID NO: 38 - protein, 113 aa, synthetic construct
DIVMTQSPDSLAVSLGERATINCKSSQSVLYSSNNKNYLAWYQQKPGQPPNLLIYWASIRESGVPERFSGSGSGTDFTLTISSLQAEDVAVYYCQQYYTSPYTFGQGTKLEIK

SEQ ID NO: 39 - protein, 120 aa, synthetic construct
EVQLVESGGGLVQPGGSLRLSCAASGFIFDDYDMHWVRQAPGKGLEWVSGINWNSNRVDYADSVKGRFTISRDNARNSLYLQMNSLRPEDTAFYYCVKDKTSSWYVFDSWGQGTPVTVSS

SEQ ID NO: 40 - protein, 113 aa, synthetic construct
DIVMTQSPDSLAVSLGERATINCKSSQSVLYSSNNKNYLAWYQQKPGQPPKLLIYWASTRESGVSDRFSGSGSGTDFTLTISSLQAEDVAVYYCQQYYSIPYTFGQGTKLEIK

SEQ ID NO: 41 - protein, 120 aa, synthetic construct
QVQLQESGPGLVKPSGTLSLTCAVSGGSISSSNWWSWVRQPPGKGLEWIGDIYHDGTTYYNPSLKSRVTVSVDKSKNQFSLKLSSVTAADTAVYYCARSPSGTTDYFDYWGQGTLVTVSS

SEQ ID NO: 42 - protein, 107 aa, synthetic construct
DIQMTQSPSSLSASVGDRVNITCRASQSISSYLNWYQQKPGKAPKLLIYAPSSLQSGVPSRFSGGGSGTDFTLTISSLQPEDFATYFCQQSYSTPPTFGPGTKVDIK

SEQ ID NO: 43 - protein, 122 aa, synthetic construct
QLQLQESGPGLVKPSETLSLTCTVSGGSISSSTYYWGWIRQPPGKGLEWIGSIFYSGNTYYNPSLKSRVTISVDTSKNHFSLKLSSVTAADTAVYYCAREIIEPRPGYFDYWGQGTLVTVSS

SEQ ID NO: 44 - protein, 107 aa, synthetic construct
DIQMTQSPSSLSASVGDRVTITCRASQSISKYLNWYQQKPGKAPKLLIYAASSLQSGVPSRFSGSGSGTDFTLTISSLQPEDFATYYCQQSNSIPRTFGQGTKVEIK

SEQ ID NO: 45 - protein, 118 aa, synthetic construct
QVQLVQSGAEVKKPGASVKVSCKASGYTFTGYYMHWVRQAPGQGLEWMGWIYPNTGGTNYAQNFQGRVTMTRDTSISTAYMELSRLRSDDTAVYYCARDDYNKNLDYWGQGTLVTVSS

SEQ ID NO: 46 - protein, 107 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYNSYFRTFGQGTKVEIK

SEQ ID NO: 47 - protein, 122 aa, synthetic construct
QVQLVQSGAEVKKPGASVKVSCKASGYTFTGYYMNWVRQAPGQGLEWMGWINPQSGDVNFAQKFQGRVTMTRDTSISTAYMELSRLRSDDTAVYYCARQFRGYSYGYGMDVWGQGTTVTVSS

SEQ ID NO: 48 - protein, 110 aa, synthetic construct
DIQMTQSPSSLSASVGDRVTITCRASQNIGSYLNWYQQKPGKAPKLLIYAASSLQSGVPSRFSGSGSGTDFTLTISSLQPEDFATYYCQQANSFPFTFGPGTKVDIKRTV

SEQ ID NO: 49 - protein, 120 aa, synthetic construct
EVQLVESGGGLVKPGGSLRLSCVASGFTFSLAWMNWVRQAPGKGLEWVGHIKSRADGGSTDYAAPVKGRFTISRDDSKNTLYLHMNSLKTEDTAVYFCSRFYWNYVLDYWGQGTLVTVSS

SEQ ID NO: 50 - protein, 107 aa, synthetic construct
DIQMTQSPSSLSASVGDRVTITCRASQSINSYFNWYQQKPGKAPKLLIYAASGLQSGVPSRFSGSGSGTDFTLTISSLQPEDFATYYCQQSYSPPYTFGQGTNLEIK

SEQ ID NO: 51 - protein, 122 aa, synthetic construct
QLQLQESGPGLVKPSETLSLTCTVSGGSISSSTYYWGWIRQPPGKGLEWIGSIFYSGNTYYNPSLKSRVTISVDTSKNQFSLRLNSVTAADTAVYYCAREIIEPRPGYFDYWGQGTLVTVSS

SEQ ID NO: 52 - protein, 107 aa, synthetic construct
DIQMTQSPSSLSASVGDRVTITCRASQSISKYLNWYQQKPGKAPKFLIYAASSLQSGVPSRFSGSGSGTDFTLTISSLQPEDFATYYCQQSNSIPRTFGQGTKVEIK

SEQ ID NO: 53 - protein, 118 aa, synthetic construct
QVQLVQSGAEVKKPGASVKVSCKASGYTFTGYYMHWVRQAPGQGLEWMGWIYPNTGGTNYAQNFQGRVTMTRDTSISTAYMELSSLRSDDTAVYYCARDDYNKNLDYWGQGTLVTVSS

SEQ ID NO: 54 - protein, 107 aa, synthetic construct
DIQMTQSPSTLSASVGDRGTITCRASQSIGSWLAWYQQKPGKTPKLLIYKASSLESGVPSRFSGSGSGTEFTLTISSLQPDDFATYYCQQYNSYFRTFGQGTKVEIK

SEQ ID NO: 55 - protein, 122 aa, synthetic construct
QVQLVQSGTEVKKPGASVKVSCKASGYTFTGYYMNWVRQAPGQGLEWVGWINPNSGDTNFAQKFQGRVTMTRDSSISTAYMELSRLRSDDTAVYYCARQFRGYSYGYGMDVWGKGTTVTVSS

SEQ ID NO: 56 - protein, 107 aa, synthetic construct
DIQMTQSPSSLSASVGDRVTITCRASQNIGSYLNWYQQKPGKAPKLLIYAASSLQSGVPSRFSGSGSGTDFTLTISSLQPEDFATYYCQQANSFPFTFGPGTKVDIK

SEQ ID NO: 57 - protein, 6 aa, synthetic construct
SFEIMA

SEQ ID NO: 58 - protein, 16 aa, synthetic construct
VINKGGEANYPDSVKG

SEQ ID NO: 59 - protein, 8 aa, synthetic construct
DAVPHGSY

SEQ ID NO: 60 - protein, 5 aa, synthetic construct
DRAFG

SEQ ID NO: 61 - protein, 16 aa, synthetic construct
TISTAGLTWYDVSVKG

SEQ ID NO: 62 - protein, 4 aa, synthetic construct
FRGN

SEQ ID NO: 63 - protein, 5 aa, synthetic construct
IYDMY

SEQ ID NO: 64 - protein, 16 aa, synthetic construct
ISTSGGHTNYADSVKG

SEQ ID NO: 65 - protein, 9 aa, synthetic construct
QRVDAPGFY

SEQ ID NO: 66 - protein, 8 aa, synthetic construct
GRTFTNYR

SEQ ID NO: 67 - protein, 8 aa, synthetic construct
INWGGGGT

SEQ ID NO: 68 - protein, 13 aa, synthetic construct
AADFTGWGSYFDY

SEQ ID NO: 69 - protein, 5 aa, synthetic construct
SYCMN

SEQ ID NO: 70 - protein, 17 aa, synthetic construct
SISTGNTYIYYSDSVKG

SEQ ID NO: 71 - protein, 12 aa, synthetic construct
ARYNWNYDAFDI

SEQ ID NO: 72 - protein, 11 aa, synthetic construct
QASQDISNFLS

SEQ ID NO: 73 - protein, 7 aa, synthetic construct
DASNLET

SEQ ID NO: 74 - protein, 9 aa, synthetic construct
QQYYNVPLT

SEQ ID NO: 75 - protein, 5 aa, synthetic construct
SYSMN

SEQ ID NO: 76 - protein, 17 aa, synthetic construct
SISPSSSYIYYADSVKG

SEQ ID NO: 77 - protein, 12 aa, synthetic construct
DHYNWNFDALDI

SEQ ID NO: 78 - protein, 11 aa, synthetic construct
RASQSISSWLA

SEQ ID NO: 79 - protein, 7 aa, synthetic construct
KASSLES

SEQ ID NO: 80 - protein, 9 aa, synthetic construct
QQYNSFSRT

SEQ ID NO: 81 - protein, 5 aa, synthetic construct
SYAMS

SEQ ID NO: 82 - protein, 17 aa, synthetic construct
GISGSGGRTNYADSVKG

SEQ ID NO: 83 - protein, 12 aa, synthetic construct
SDNWHYLDAFDI

SEQ ID NO: 84 - protein, 17 aa, synthetic construct
KSSQNVLYSSNNKNYLT

SEQ ID NO: 85 - protein, 7 aa, synthetic construct
WASTRES

SEQ ID NO: 86 - protein, 9 aa, synthetic construct
QQYFNTPPT

SEQ ID NO: 87 - protein, 5 aa, synthetic construct
SYGIH

SEQ ID NO: 88 - protein, 17 aa, synthetic construct
VIWYDGSNKYYADSVKG

SEQ ID NO: 89 - protein, 12 aa, synthetic construct
DGGTGNYYYMDV

SEQ ID NO: 90 - protein, 17 aa, synthetic construct
KSSQSVFYSSNNKNYLV

SEQ ID NO: 91 - protein, 7 aa, synthetic construct
WASTRAS

SEQ ID NO: 92 - protein, 9 aa, synthetic construct
QQYYNTPLT

SEQ ID NO: 93 - protein, 5 aa, synthetic construct
DYDIH

SEQ ID NO: 94 - protein, 17 aa, synthetic construct
GINWNSITIGYADSVKG

SEQ ID NO: 95 - protein, 11 aa, synthetic construct
DRGSGWRIFDY

SEQ ID NO: 96 - protein, 17 aa, synthetic construct
KSSQSVLYSSNNKNYLA

SEQ ID NO: 97 - protein, 7 aa, synthetic construct
WASTRES

SEQ ID NO: 98 - protein, 9 aa, synthetic construct
QQYYSTPYT

SEQ ID NO: 99 - protein, 5 aa, synthetic construct
SYDIS

SEQ ID NO: 100 - protein, 17 aa, synthetic construct
WFSAYNGNSNYAQKFQG

SEQ ID NO: 101 - protein, 12 aa, synthetic construct
EGYYGNSGFFDY

SEQ ID NO: 102 - protein, 17 aa, synthetic construct
KSSQSVLYSSNNKNYLA

SEQ ID NO: 103 - protein, 7 aa, synthetic construct
WASIRES

SEQ ID NO: 104 - protein, 9 aa, synthetic construct
QQYYTSPYT

SEQ ID NO: 105 - protein, 5 aa, synthetic construct
DYDMH

SEQ ID NO: 106 - protein, 17 aa, synthetic construct
GINWNSNRVDYADSVKG

SEQ ID NO: 107 - protein, 11 aa, synthetic construct
DKTSSWYVFDS

SEQ ID NO: 108 - protein, 17 aa, synthetic construct
KSSQSVLYSSNNKNYLA

SEQ ID NO: 109 - protein, 7 aa, synthetic construct
WASTRES

SEQ ID NO: 110 - protein, 9 aa, synthetic construct
QQYYSIPYT

SEQ ID NO: 111 - protein, 6 aa, synthetic construct
SSNWWS

SEQ ID NO: 112 - protein, 16 aa, synthetic construct
DIYHDGTTYYNPSLKS

SEQ ID NO: 113 - protein, 11 aa, synthetic construct
SPSGTTDYFDY

SEQ ID NO: 114 - protein, 11 aa, synthetic construct
RASQSISSYLN

SEQ ID NO: 115 - protein, 7 aa, synthetic construct
APSSLQS

SEQ ID NO: 116 - protein, 9 aa, synthetic construct
QQSYSTPPT

SEQ ID NO: 117 - protein, 7 aa, synthetic construct
SSTYYWG

SEQ ID NO: 118 - protein, 16 aa, synthetic construct
SIFYSGNTYYNPSLKS

SEQ ID NO: 119 - protein, 12 aa, synthetic construct
EIIEPRPGYFDY

SEQ ID NO: 120 - protein, 11 aa, synthetic construct
RASQSISKYLN

SEQ ID NO: 121 - protein, 7 aa, synthetic construct
AASSLQS

SEQ ID NO: 122 - protein, 9 aa, synthetic construct
QQSNSIPRT

SEQ ID NO: 123 - protein, 5 aa, synthetic construct
GYYMH

SEQ ID NO: 124 - protein, 17 aa, synthetic construct
WIYPNTGGTNYAQNFQG

SEQ ID NO: 125 - protein, 9 aa, synthetic construct
DDYNKNLDY

SEQ ID NO: 126 - protein, 11 aa, synthetic construct
RASQSIGSWLA

SEQ ID NO: 127 - protein, 7 aa, synthetic construct
KASSLES

SEQ ID NO: 128 - protein, 9 aa, synthetic construct
QQYNSYFRT

SEQ ID NO: 129 - protein, 5 aa, synthetic construct
GYYMN

SEQ ID NO: 130 - protein, 17 aa, synthetic construct
WINPQSGDVNFAQKFQG

SEQ ID NO: 131 - protein, 13 aa, synthetic construct
QFRGYSYGYGMDV

SEQ ID NO: 132 - protein, 11 aa, synthetic construct
QASQDISNFLS

SEQ ID NO: 133 - protein, 7 aa, synthetic construct
DASNLET

SEQ ID NO: 134 - protein, 9 aa, synthetic construct
QQYYNVPLT

SEQ ID NO: 135 - protein, 5 aa, synthetic construct
LAWMN

SEQ ID NO: 136 - protein, 19 aa, synthetic construct
HIKSRADGGSTDYAAPVKG

SEQ ID NO: 137 - protein, 9 aa, synthetic construct
FYWNYVLDY

SEQ ID NO: 138 - protein, 11 aa, synthetic construct
RASQSINSYFN

SEQ ID NO: 139 - protein, 7 aa, synthetic construct
AASGLQS

SEQ ID NO: 140 - protein, 9 aa, synthetic construct
QQSYSPPYT

SEQ ID NO: 141 - protein, 7 aa, synthetic construct
SSTYYWG

SEQ ID NO: 142 - protein, 16 aa, synthetic construct
SIFYSGNTYYNPSLKS

SEQ ID NO: 143 - protein, 12 aa, synthetic construct
EIIEPRPGYFDY

SEQ ID NO: 144 - protein, 11 aa, synthetic construct
RASQSISKYLN

SEQ ID NO: 145 - protein, 7 aa, synthetic construct
AASSLQS

SEQ ID NO: 146 - protein, 9 aa, synthetic construct
QQSNSIPRT

SEQ ID NO: 147 - protein, 5 aa, synthetic construct
GYYMH

SEQ ID NO: 148 - protein, 17 aa, synthetic construct
WIYPNTGGTNYAQNFQG

SEQ ID NO: 149 - protein, 9 aa, synthetic construct
DDYNKNLDY

SEQ ID NO: 150 - protein, 11 aa, synthetic construct
RASQSIGSWLA

SEQ ID NO: 151 - protein, 7 aa, synthetic construct
KASSLES

SEQ ID NO: 152 - protein, 9 aa, synthetic construct
QQYNSYFRT

SEQ ID NO: 153 - protein, 5 aa, synthetic construct
GYYMN

SEQ ID NO: 154 - protein, 17 aa, synthetic construct
WINPNSGDTNFAQKFQG

SEQ ID NO: 155 - protein, 13 aa, synthetic construct
QFRGYSYGYGMDV

SEQ ID NO: 156 - protein, 11 aa, synthetic construct
RASQNIGSYLN

SEQ ID NO: 157 - protein, 7 aa, synthetic construct
AASSLQS

SEQ ID NO: 158 - protein, 9 aa, synthetic construct
QQANSFPFT

SEQ ID NO: 159 - protein, 115 aa, synthetic construct
EVQLVESGGGLVQPGGSLRLSCAASGRFFGIYDMYWYRQPPGKQRELVAISTSGGHTNYADSVKGRFTISRDNAKNTVYLQMNSLRPEDTAVYYCNIQRVDAPGMYWGQGTLVTV

SEQ ID NO: 160 - protein, 117 aa, synthetic construct
EVQLVESGGGLVQPGGSLRLSCAASGSSIFSFEIMAWYRQAPGKQRELVAVINKGGEANYPDSVEGRFTISRDNAKNTVYLQMNSLRPEDTAVYYCNADAVPHGSYWGQGTLVTVSS

SEQ ID NO: 161 - protein, 120 aa, synthetic construct
EVQLVESGGGLVQPGGSLRLSCAASGRTFTNYRMAWFRQAPGKEREAVAAINWNGGGTYYADSVKGRFTISRDNAKNTVYLQMNSLRPEDTAVYYCAADSTGWGSYFDYWGQGTLVTVSS

SEQ ID NO: 162 - protein, 112 aa, synthetic construct
EVQLVESGGGLVQPGGSLRLSCKASGTTFSDRAFGWYRQAPGKQRELVATISTAGLTWYDVSVKGRFIISRDNPENTVYLQMNSLRPEDTAVYYCNTFRGNWGQGTLVTVSS

SEQ ID NO: 163 - protein, 5 aa, synthetic construct
IYDMY

SEQ ID NO: 164 - protein, 16 aa, synthetic construct
ISTSGGHTNYADSVKG

SEQ ID NO: 165 - protein, 9 aa, synthetic construct
QRVDAPGMY

SEQ ID NO: 166 - protein, 6 aa, synthetic construct
SFEIMA

SEQ ID NO: 167 - protein, 16 aa, synthetic construct
VINKGGEANYPDSVEG

SEQ ID NO: 168 - protein, 8 aa, synthetic construct
DAVPHGSY

SEQ ID NO: 169 - protein, 5 aa, synthetic construct
NYRMA

SEQ ID NO: 170 - protein, 17 aa, synthetic construct
AINWNGGGTYYADSVKG

SEQ ID NO: 171 - protein, 11 aa, synthetic construct
DSTGWGSYFDY

SEQ ID NO: 172 - protein, 5 aa, synthetic construct
DRAFG

SEQ ID NO: 173 - protein, 16 aa, synthetic construct
TISTAGLTWYDVSVKG

SEQ ID NO: 174 - protein, 4 aa, synthetic construct
FRGN

SEQ ID NO: 175 - protein, 348 aa, synthetic construct
DVQLVESGGGLVQPGGSLRLSCKASGTTFSDRAFGWYRQAPGKQRELVATISTAGLTWYDVSVKGRFTISRDNAKNTVYLQMNSLRPEDTAVYYCNTFRGNWGQGTLVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 176 - protein, 451 aa, synthetic construct
QLQLQESGPGLVKPSETLSLTCTVSGGSISSSTYYWGWIRQPPGKGLEWIGSIFYSGNTYYNPSLKSRVTISVDTSKNHFSLKLSSVTAADTAVYYCAREIIEPRPGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 177 - protein, 214 aa, synthetic construct
DIQMTQSPSSLSASVGDRVTITCRASQSISKYLNWYQQKPGKAPKLLIYAASSLQSGVPSRFSGSGSGTDFTLTISSLQPEDFATYYCQQSNSIPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 353 aa, synthetic construct
DVQLVESGGGLVQPGGSLRLSCAASGRFFGIYDMYWYRQAPGKQRELVAISTSGGHTNYADSVKGRFTISRDNAKNTVYLQMNSLRPEDTAVYYCNIQRVDAPGFYWGQGTLVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 179 - protein, 451 aa, synthetic construct; identical to SEQ ID NO: 176

SEQ ID NO: 180 - protein, 214 aa, synthetic construct; identical to SEQ ID NO: 177

SEQ ID NO: 181 - protein, 348 aa, synthetic construct; identical to SEQ ID NO: 175

SEQ ID NO: 182 - protein, 447 aa, synthetic construct
QVQLVQSGAEVKKPGASVKVSCKASGYTFTGYYMHWVRQAPGQGLEWMGWIYPNTGGVNYAQNFQGRVTMTRDTSISTAYMELSRLRSDDTAVYYCARDDYNKNLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 183 - protein, 214 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYNSYFRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 184 - protein, 575 aa, synthetic construct
QVQLVQSGAEVKKPGASVKVSCKASGYTFTGYYMHWVRQAPGQGLEWMGWIYPNTGGVNYAQNFQGRVTMTRDTSISTAYMELSRLRSDDTAVYYCARDDYNKNLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDVQLVESGGGLVQPGGSLRLSCKASGTTFSDRAFGWYRQAPGKQRELVATISTAGLTWYDVSVKGRFTISRDNAKNTVYLQMNSLRPEDTAVYYCNTFRGNWGQGTLVTVSSA

SEQ ID NO: 185 - protein, 447 aa, synthetic construct; identical to SEQ ID NO: 182

SEQ ID NO: 186 - protein, 214 aa, synthetic construct; identical to SEQ ID NO: 183

SEQ ID NO: 187 - protein, 579 aa, synthetic construct
QLQLQESGPGLVKPSETLSLTCTVSGGSISSSTYYWGWIRQPPGKGLEWIGSIFYSGNTYYNPSLKSRVTISVDTSKNHFSLKLSSVTAADTAVYYCAREIIEPRPGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDVQLVESGGGLVQPGGSLRLSCKASGTTFSDRAFGWYRQAPGKQRELVATISTAGLTWYDVSVKGRFTISRDNAKNTVYLQMNSLRPEDTAVYYCNTFRGNWGQGTLVTVSSA

SEQ ID NO: 188 - protein, 451 aa, synthetic construct; identical to SEQ ID NO: 176

SEQ ID NO: 189 - protein, 214 aa, synthetic construct; identical to SEQ ID NO: 177

SEQ ID NO: 190 - protein, 580 aa, synthetic construct
QVQLVQSGAEVKKPGASVKVSCKASGYTFTGYYMHWVRQAPGQGLEWMGWIYPNTGGVNYAQNFQGRVTMTRDTSISTAYMELSRLRSDDTAVYYCARDDYNKNLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDVQLVESGGGLVQPGGSLRLSCAASGRFFGIYDMYWYRQAPGKQRELVAISTSGGHTNYADSVKGRFTISRDNAKNTVYLQMNSLRPEDTAVYYCNIQRVDAPGFYWGQGTLVTVSSA

SEQ ID NO: 191 - protein, 447 aa, synthetic construct; identical to SEQ ID NO: 182

SEQ ID NO: 192 - protein, 214 aa, synthetic construct; identical to SEQ ID NO: 183

SEQ ID NO: 193 - protein, 715 aa, synthetic construct
QVQLVQSGAEVKKPGASVKVSCKASGYTFTGYYMNWVRQAPGQGLEWMGWINPQSGDVNFAQKFQGRVTMTRDTSISTAYMELSRLRSDDTAVYYCARQFRGYSYGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLVESGGGLVQPGGSLRLSCAASGFTFSSYAMSWVRQAPGKCLEWVSGISGSGGRTNYADSVKGRFTISRDNSKNTLYLQMNSLRAEDTAVYYCAKSDNWHYLDAFDIWGQGTMVTVSSGGGGSGGGGSGGGGSGGGGSDIVMTQSPDSLAVSLGERATINCKSSQNVLYSSNNKNYLTWYQQKPGQPPKLLIYWASTRESGVPDRFSGSGSGTDFTLTISSLQAEDVAVYYCQQYFNTPPTFGCGTKVEIK

SEQ ID NO: 194 - protein, 451 aa, synthetic construct
QVQLVQSGAEVKKPGASVKVSCKASGYTFTGYYMNWVRQAPGQGLEWMGWINPQSGDVNFAQKFQGRVTMTRDTSISTAYMELSRLRSDDTAVYYCARQFRGYSYGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 195 - protein, 214 aa, synthetic construct
DIQMTQSPSSLSASVGDRVTITCRASQNIGSYLNWYQQKPGKAPKLLIYAASSLQSGVPSRFSGSGSGTDFTLTISSLQPEDFATYYCQQANSFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 196 - protein, 585 aa, synthetic construct
QLQLQESGPGLVKPSETLSLTCTVSGGSISSSTYYWGWIRQPPGKGLEWIGSIFYSGNTYYNPSLKSRVTISVDTSKNHFSLKLSSVTAADTAVYYCAREIIEPRPGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDVQLVESGGGLVQPGGSLRLSCAASGRFFGIYDMYWYRQAPGKQRELVAISTSGGHTNYADSVKGRFTISRDNAKNTVYLQMNSLRPEDTAVYYCNIQRVDAPGFYWGQGTLVTVSSA

SEQ ID NO: 197 - protein, 214 aa, synthetic construct; identical to SEQ ID NO: 177

SEQ ID NO: 198 - protein, 579 aa, synthetic construct
QLQLQESGPGLVKPSETLSLTCTVSGGSISSSTYYWGWIRQPPGKGLEWIGSIFYSGNTYYNPSLKSRVTISVDTSKNHFSLKLSSVTAADTAVYYCAREIIEPRPGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDVQLVESGGGLVQPGGSLRLSCKASGTTFSDRAFGWYRQAPGKQRELVATISTAGLTWYDVSVKGRFTISRDNAKNTVYLQMNSLRPEDTAVYYCNTFRGNWGQGTLVTVSS

SEQ ID NO: 199 - protein, 214 aa, synthetic construct; identical to SEQ ID NO: 177

SEQ ID NO: 200 - protein, 349 aa, synthetic construct
DVQLVESGGGLVQPGGSLRLSCKASGTTFSDRAFGWYRQAPGKQRELVATISTAGLTWYDVSVKGRFTISRDNAKNTVYLQMNSLRPEDTAVYYCNTFRGNWGQGTLVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - protein, 450 aa, synthetic construct
EVQLVESGGGLVKPGGSLRLSCVASGFTFSLAWMNWVRQAPGKGLEWVGHIKSRADGGSTDYAAPVKGRFTISRDDSKNTLYLHMNSLKTEDTAVYFCSRFYWNYVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 202 - protein, 214 aa, synthetic construct
DIQMTQSPSSLSASVGDRVTITCRASQSINSYFNWYQQKPGKAPKLLIYAASGLQSGVPSRFSGSGSGTDFTLTISSLQPEDFATYYCQQSYSPPYTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 10 aa, synthetic construct
DKTHTCPPCP

SEQ ID NO: 204 - protein, 12 aa, synthetic construct
EPKSCDKTHTCP

SEQ ID NO: 205 - protein, 16 aa, synthetic construct
  VARIANT 15: note = X is K or E
VINKGGEANYPDSVXG

SEQ ID NO: 206 - protein, 17 aa, synthetic construct
  VARIANT 5: note = X is G or N
AINWXGGGTYYADSVKG

SEQ ID NO: 207 - protein, 9 aa, synthetic construct
  VARIANT 8: note = X is F or M
QRVDAPGXY

SEQ ID NO: 208 - protein, 11 aa, synthetic construct
  VARIANT 2: note = X is F or S
DXTGWGSYFDY

SEQ ID NO: 209 - protein, 24 aa, synthetic construct
KCLQDNNWDYTRYAQAFTLLKAKG